SEQUENCE LISTING (172 sequences)

SEQ ID NO: 1 - protein, 387 aa, Unknown
  Environmental sample
MRILKNTLLAGLMLLFSCMKGEAQSGAKNTFSEFPKYTVFSAGDDNVNSYRIPSLLIVKDGSLLVFCEARRESWQDKSRTDVVVKRSTDNGQTWSVMQDLTKGITGAYMDPTPILDSSTGRVFLFTTFWPVDDHSGAGNRAILITSDDNGQTWSTPADVTASLMPDGRYIYGFGPGAGLQMVGEKYKGRLILPARISDMERKTAHDVAIFSDNHGETWTVGENGDTDNEFQIAESPNGVLVYNARVSGARMVAYSKDGGMTWSKAMKESALPGVSKGCQASVLGENANLYFSGIKGIPETPQYDERAGLTLYKSCNGGRTWNDGLLLCDKASGYSCMALLPDERMAIIFETADTQSFTRKSLPNIKPLKRPADWMKLDLILVPIINL

SEQ ID NO: 2 - protein, 369 aa, Unknown
  Environmental sample
MKNLNLIFVWVCLLFISTACNDVEPSISSFPVPTSKPYSINREGYAYFRIPTMVITNSGTILAFAEGRRNGPEDEGDIDIVLKRSTDKGKTWGPLITVKDDGENRCRNQVPIYLPDINRVILLSCWNPGATGTNSIFMTYSDDEGLTWAKEKDITASVTPDKYRWYATGPCHGIVKQYEPHKGRIVVPCNHNTTTSGAGRSHVIYSDDKGETWHLGGIMDVDYTNESTVTELSNGDLMLNMRKQSNTEKYRMVSTSTDGGLTWSPCKYTTLKEPICQGSILFYGLGDDGKGIILFSNPDSQTNRNNNTLKMSEDDGVTWKKQYSYTGSDYGGYSDIARYPDGTIGVLYEYGFKNIGGIAFQNVELSQFQ

SEQ ID NO: 3 - protein, 546 aa, Unknown
  Environmental sample
MKMSKKIFIIFSMILLWIPDISLGKDIRFELERPVIPVLVKKQINPTVKATLIQTNSRPYTIRQIDVDLQESTDLSDILSVAVYGTHKNGLIDESRLICKPMPAARKISFTDNIQVKDDSLSFWIAVTLKDTVPLTHRISVNCSRIKTSRGELKVSNKEAIPLRVGVALRQKGQDGIVSSRIPGLATSNNGTLLAIFDARYDLARDLQGNIDIALHRSFDKGLTWQPIQTVLDMGEWGGLPQKFNGVSDACILVDKNTNDIYIAGLWMHGALDDNGKWIEGLNENSTYWIHQWRKKGSQPGIGLKETCQFLITKSTDDGLTWSFPDNITGKTKRPEWWLFAPAPGQGITLADGTLVFPTQGRDDKGAAFSNITYSKDHGKTWTTSNPAYSNVTECNAAQLSDGTVMLNMRDNRNRGNKEVNGRRICTTTDLGETWTEHLTSRKALVEPTCMASLHRHDYTVDGEKKSILLFANPSDYKTRDKLTLKVSFDDGMTWPEKYWILFDQYRSAGYSSITSIDENSIGILYESSQANMAFIKIDLAEILNR

SEQ ID NO: 4 - protein, 543 aa, Unknown
  Environmental sample
MKKNLFLSIIFSFCVILQAFASDTVFVRETQIPVLIERQDNVLFMLRLNAKESHTLDEVVLNFGKDVNMSDIQSVKLYYSGTEARQNYGKNFFAPVSYISSHTPGKTLAANPSYSINKSQVNNPKRKVALKANQKLFPGINYFWISLQMKPDASLLDKVAAKIAAIKVDNKEALMHTVSPENIVHRVGVGVRHAGDDGSASFRIPGLVTTNKGTLLGVYDVRYNNSADLQEHVDIGLSRSVDGGKTWEKMRLPLAFGETGDLPAAQNGVGDPSILVDTKTNTVWVVAAWTHGMGNQRAWWSSYPGMDMNHTAQLVLSKSTDDGKTWSKPINITEQVKDPSWYFLLQGPGRGITMQDGTLVFPIQFIDSTRVPNAGIMYSKDRGETWKIHNYARTNTTEAQVAEVEPGVLMLNMRDNRGGSRAISTTKDLGKTWTEHSSSRKALQEPVCMASLISVKAKDNVLNKDILLFSNPNTVKGRHHITIKASLDGGVTWLPEHQVMLDEGEGWGYSCLTMIDKETIGILYESSVAHMTFQAVQLRDIIK

SEQ ID NO: 5 - protein, 423 aa, Unknown
  Environmental sample
MKHLFNVLALGIGLFFFNACSDDNVNDFRPVPYPDEVVGLNPDPDFEPIWEIASPTITVFSSKASNDISYRVPAIAVTKKGSILVFCEARYGTWQDKAGRTDILMKRSTDKGITWTEKNLTNQATSSKLSYMDPTVVVDQVTGKIFLFTSLWDAVGKESAKQGYNNRAIMYTSEDDGLNWTRKDLTDEVEIGIFSGATRMIGSFGPGSGVQMTSSEQYKNRLIVPIRTFKVNEAAGTVSNGGNTAMWSDDNGGTWETGQPNKSGEWMVTEAPDGALIGNIRYNGHRQNYVSTDGGAKPSFSDYDPIALPTPAKGCAGSVIVKDGWMYYCGAKGIIETTAHDDRGILYLAKAKFFGGHSHTFDPADHMVLYDKAAGYTCMALLPDGDMAIVAELGNEPGFQKLSTRPAEWMRLELFILSTKKPL

SEQ ID NO: 6 - protein, 539 aa, Unknown
  Environmental sample
MMKRTFWLIALSLLSLTLSASDSLRIHNPQIPILLDRMDNVLFLIRVPDAVQGNVLESLTVEFGPDTDLNSIAELRLFYSGTEAVRRQGLRFSPVEYISAHNVWNTRSANPSYSVMQEQASKIGRKVVLHSRQPMVGGINYYWVSVRMNPDASLLTELRARVSEVVVNGKKIPVACDSREVVRRMGYGVRHAGDDHSMAYRIPGLVTTNSGSLIGTYDVRWNSSVDLQERIDIGVSRSTDKGQTWEPMRIAMSFADIDGLPSGQNGVGDPAVLVDEKTGTIWVMAAWTHGMGNGRAWTNSMPGMEPMETPQLMLARSDDDGRTWSEPINITKQVKDPSWCFLLQGPGRGITMRDGTLVFAIQFIDKDRMPHAGIIFSKDHGETWHIHNPARSNTTEAQVAEVEPGVLMLNMRDNRGGARAVMTTRDLGRTWTEHVSHRTTLREPVCMASLIAVPAEKNVLGKDLLLFSNPDTTDGRRDITIKASLDGGVTWPYSLLLDEGDSWGYSCLTMIDSQTVGILYESSVAHITFQAAKLKAIVK

SEQ ID NO: 7 - protein, 373 aa, Unknown
  Environmental sample
MKRTGWLLLLVSGPALCQPPRGYSIPLIDLAAEAWRQTVVDREPGQYLGHPTTVLLEDGRTVLAVYPKGHGRGAIVYKRSRDGGRTWSARLPVPENWATSQETPTIHRVVDPRGRKRLILFSGLYPIRMSVSEDDGETWTPLAPIGNFGGVVAMASVERLRDGRYMALFHDDGRFLRGGGKPDRFVVYKTLSGDGGLTWSEPVPILSHPQAHLCEPGLLRSPDGRRIAILLRENSRKFNSFVSFSDDEGETWSEPRELPGALTGDRHTAVYARDGRLFISFRDTTHESPTRGDWVAWVGRFGDIENGREGQYRVRLMKNHKDLDCCYPGVLRLPDDTILTTTYGHWTPGEPPYIVSIRLRLSELDRRAQGAGR

SEQ ID NO: 8 - protein, 392 aa, Unknown
  Environmental sample
MVIAWMAGVQCTAAALVDLGVTILDISGEPERQVIVDREPGQYLGHVSTVLLEDDKTILAVYPKGHGRGAIVLKRSEDGGRTWSGRLPVPASWATSKETPTIHRVVDARGRRRLLLWSGLYPARRALSDDDGRTWTELEPVGEWGGIVVMGFVEATRQPGHYVAMFHDDGRYFAAQPSTNRSMTLYLTRSSDGGVSWSSPVAVWSNSAVHLCEPGGIWSPDRRRLAVLLRENRRVNNSHIMFSDDEGHTWTTPVEMPLSRAGDRHTLRYTPDGRIVCVFRAVTPVGMRGRSFGQNEDVDTLGVGSPFEGDCVAWVGTWDDLVHNRPGQYYVRLLNNRKGWDTTYPGVEVLPDGTVVVTTYGHWNAGEPPYIRSVRFRLEELDRMAAKASQKP

SEQ ID NO: 9 - protein, 404 aa, Paeniclostridium sordellii
MKKFIKILKVLSMAIVLSACNINGIFASNLNTTNEPQKTTVFNKNDNTWNAQYFRIPSLQTLADGTMLAFSDIRYNGAEDHAYIDIGAAKSTDNGQTWDYKTVMENDRIDSTFSRVMDSTTVVTDTGRIILIAGSWNKNGNWASSTTSLRSDWSVQMVYSDDNGETWSDKVDLTTNKARIKNQPSNTIGWLAGVGSGIVMSDGTIVMPIQIALRENNANNYYSSVIYSKDNGETWTMGNKVPDPKTSENMVIELDGALIMSSRNDGKNYRASYISYDMGSTWEVYDPLHNKISTGNGSGCQGSFIKVTAKDGHRLGFISAPKNTKGGYVRDNITVYMIDFDDLSKGIRELCSPYPEDGNSSGGGYSCLSFNDGKLSILYEANGNIEYKDLTDYYLSIENNKKLK

SEQ ID NO: 10 - protein, 388 aa, Brucella abortus biovar 1 (strain 9-941)
MFSLIRAKRLAIGIAALAWSTGAVMASEHWPDLPVGIKNGAAARIGNMAYVGLGSAGTDFYALDLNNPSKGWVKRANFIGPATNGAAMAAAGGKIFAFSGNGKATPDAKSPIIFDTAYVYDPGSDGWSKLDTQTPVGLSGAKAVGLADGRIAIFGGYNKELFDKYLADVGAIDKDKEPEAYRNLVDSYMGMKPEAYRWNDEVLVYDPAGNNWGSLGANPFLPNCDPAMATMGEGDFLLVSGEIKPGLRTPEAKLVKIRDGAAHWQKVSDLPPLSGSEPQEGVAGAYAGKAGDDVLVAGGANFKGAQANAAAGKWFAHDGLAKSWRDDVYAFDGKDWKVAGKLPRGLAYGAAFDAPGGLLVVGGEDRDGKARKEVFLLKWDGKALSVEN

SEQ ID NO: 11 - protein, 388 aa, Brucella melitensis biotype 1 (strain 16M)
MFSLIRAKRLAIGIAALAWSTGAVMASEHWPDLPVGIKNGAAARIGNMAYVGLGSAGTDFYALDLNNPSKGWVKRANFIGPATNGAAMAAAGGKIFAFSGNGKATPDAKSPIIFDTAYVYDPGSDGWSKLDTQTPVGLSGAKAVGLADGRIAIFGGYNKELFDKYLADVGAIDKDKEPEAYRKLVDSYMGMKPEAYRWNDEVLVYDPAGNNWGSLGANPFLPNCDPAMATMGEGDFLLVSGEIKPGLRTPEAKLVKIRDGAAHWQKVSDLPPLSGSEPQEGVAGAYAGKAGDDVLVAGGANFKGAQANAAAGKWFAHDGLAKSWRDDVYAFDGKDWKVAGKLPRGLAYGAAFDAPGGLLVVGGEDRDGKARKEVFLLKWDGKALSVEN

SEQ ID NO: 12 - protein, 388 aa, Brucella ovis (strain ATCC 25840)
MFSLIGAKRLAIGIAALAWSTGAVMASEHWPDLPVGIKNGAAARIGNMAYVGLGSAGTDFYALDLNNPSKGWVKRANFIGPATNGAAMAAAGGKIFAFSGNGKATPDAKSPIIFDTAYVYDPGSDGWSKLDTQTPVGLSGAKAVGLADGRIAIFGGYNKELFDKYLADVGAIDKDKEPEAYRKLVDSYMGMKPEAYRWNDEVLVYDPAGNNWGSLGANPFLPNCDPAMATMGEGDFLLVSGEIKPGLRTPEAKLVKIRDGAAHWQKVSDLPPLSGSEPQEGVAGAYAGKAGDDVLVAGGANFKGAQANAAAGKWFAHDGLAKSWRDDVYAFDGKDWKVAGKLPRGLAYGAAFDAPGGLLVVGGEDRDGKARKEVFLLKWDGKALSVEN

SEQ ID NO: 13 - protein, 388 aa, Brucella suis (strain ATCC 23445)
MFSLIGAKRQAIGIAALAWSTGAVMASEHWPDLPVGIKNGAAARIGNMAYVGLGSAGTDFYALDLNNPSKGWVKRANFIGPATNGAAMAAAGGKIFAFSGNGKATPDAKSPIIFDTAYVYDPGSDGWSKLDTQTPVGLSGAKAVGLADGRIAIFGGYNKELFDKYLADVGAIDKDKEPEAYRKLVDSYMGMKPEAYRWNDEVLVYDPAGNNWGSLGANPFLPNCDPAMATMGEGDFLLVSGGIKPGLRTPEAKLVKIRDGAAHWQKVSDLPPLSSSEPQEGVAGAYAGKAGDDVLVAGGANFKGAQANAAAGKWFAHDGLAKSWRDDVYAFDGKDWKVAGKLPRGLAYGAAFDAPGGLLVVGGEDRDGKARKEVFLLKWDGKALSVEN

SEQ ID NO: 14 - protein, 368 aa, Escherichia coli O139H28 (strain E24377A)
MNKTITALAIIMASFAANASVLPETPVPFKSGTGAIDNDTVYIGLGSAGTAWYKLDTQAKDKKWTALAAFPGGPRDQATSAFIDGNLYVFGGIGKNSEGLTQVFNDVHKYNPKTNSWVKLMSHAPMGMAGHVTFVHNGKAYVTGGVNQNIFNGYFEDLNEAGKDSTAVDKINAYYFDKKAEDYFFNKFLLSFDPSTQQWSYAGESPWYGTAGAAVVNKGDKTWLINGEAKPGLRTDAVFELDFTGNNLKWNKLAPVSSPDGVAGGFAGISNDSLIFAGGAGFKGSRENYQNGKNYAHEGLKKSYSTDIHLWHNGKWDKSGELSQGRAYGVSLPWNNSLLIIGGETAGGKAVTDSVFISVKDNKVTVQN

SEQ ID NO: 15 - protein, 368 aa, Escherichia coli O157H7
MNKTITALAILMASFAANASVLPETPVPFKSGTGAIDNDTVYIGLGSAGTAWYKLETQAKDKKWTALAAFPGGPRDQATSAFIDGNLYVFGGIGKNSEGLTQVFNDVHKYNPKTNSWVKLISHAPMGMAGHVTFVHNGKAYVTGGVNQNIFNGYFEDLNEAGKDSTAVDKINAHYFDKKAEDYFFNKFLLSFDPSTQQWSYAGESPWYGTAGAAVVNKGDKTWLINGEAKPGLRTDAVFELDFTGNNLKWNRLAPVSSPDGVAGGFAGISNDSLIFAGGAGFKGSRENYQNGKNYAHEGLKKSYSTDIHLWHNGKWDKSGELSQGRAYGVSLPWNNSLLIIGGETAGGKAVTDSVLISVKDNKVTVQN

SEQ ID NO: 16 - protein, 368 aa, Escherichia coli (strain K12)
MNKTITALAIMMASFAANASVLPETPVPFKSGTGAIDNDTVYIGLGSAGTAWYKLDTQAKDKKWTALAAFPGGPRDQATSAFIDGNLYVFGGIGKNSEGLTQVFNDVHKYNPKTNSWVKLMSHAPMGMAGHVTFVHNGKAYVTGGVNQNIFNGYFEDLNEAGKDSTAIDKINAHYFDKKAEDYFFNKFLLSFDPSTQQWSYAGESPWYGTAGAAVVNKGDKTWLINGEAKPGLRTDAVFELDFTGNNLKWNKLAPVSSPDGVAGGFAGISNDSLIFAGGAGFKGSRENYQNGKNYAHEGLKKSYSTDIHLWHNGKWDKSGELSQGRAYGVSLPWNNSLLIIGGETAGGKAVTDSVLITVKDNKVTVQN

SEQ ID NO: 17 - protein, 368 aa, Escherichia coli (strain UTI89)
MNKTITALTIIMASFATNASVLPETPVPFKSGTGAIDNDTVYIGLGSAGTAWYKLDTQAKDKKWTALAAFPGGPRDQATSAFIDGNLYVFGGIGKNSEGLTQVFNDVHKYNPKTNSWVKLMSHAPMGMAGHVTFVHNGKAYVTGGVNQNIFNGYFEDLNEAGKDSTTIDKINAHYFDKKAEDYFFNKFLLSFDPSTQQWSYAGESPWYGTAGAAVVNKGDKTWLINGEAKPGLRTDAVFELDFTGNNLKWNKLAPVASPDGVAGGFAGMSNDSLIFAGGAGFKGSRENYQNGKNYAHEGLKKSYSADIHLWHNGKWDKSGELSQGRAYGVSLPWNNSLLIIGGETAGGKAVTDSVLISVKDNKVTVQN

SEQ ID NO: 18 - protein, 384 aa, Vibrio cholerae serotype O1 (strain ATCC 39541)
MNMKTLLTYATLLSVTAFSHVVYADNQWPDLPTGFKDGVGAQVGSKVYVGLGSLGKSFYVLDLNALSKGWQKIADFTGAERSGATASVIGNYIYLFGGSGKAEPSDPSPILFDSVYRYDTKKDSWEKMNTTSPVGLLGASSYSPDNRQILFFGGYNKAYFDRYLRDISTTDKQVNPEVWQRIVDDYMGMTPTDYKWNRNVISYLPEKQEWRDLGVSTYLPNCGSATVIEGNKVTLISGEIKPGLRTAEVKQYEFGMDQPWKSLLPLPAPQTSNIQEGVAGAFSGKTNGVVVVAGGANFHGAKQAFENGKMFAHEGLPKAFNSEIYVEKEGIWSTVNSLPEGLAYGASFTTSEGVLIVGGEKSGKEMSHKVYMLAWNGSTVEVID

SEQ ID NO: 19 - protein, 392 aa, Yersinia enterocolitica serotype O8/biotype 1B
MTQIYHQYKKKLSTKVILLSALTLCITFSLPYANAERYPDVPVAFKYGTGARVDNHLYVGLGSAGQSWYRLDTDKASSGWQKIADFPGQPREQAVTVALSGKLYVFGGVGKNSASDTQVRALDDVYQYDPQTNQWQRLATRAPRGLVGTAATTLNGTQALLLGGVNKAIFDGYFTDLAAAGGNETQKNAVVNAYFDQAPADYFYNRDVLMYDPAKNQWKSGGQVPFLGTAGSAITAKKGDLILINGEIKPGLRTAAVWQGKTQGTELKWQQRPDLIGAEKGAVQEGLAGAFAGVSHDVVLVGGGANFPGSWQQFNAGQLYAHQGLKKQWQQPIYALVDNQWQVAGKLPQPLAYGVSIQDKDKVILLGGETSDGVATSAVTQLSWQGGKLHLE

SEQ ID NO: 20 - protein, 392 aa, Yersinia pestis bv. Antiqua (strain Nepal516)
MTQLYPQYKKQLTTKIVLFSALSLLMMASLPNTYAEQYPDVPVPFKNGTGGKVENSLYVGLGSAGVSWFRLDTDKTGAGWQKVANFPGQPREQAVTVVLAGKLYVFGGVGKTNANDTQVRALDDAYRFDPQTNQWQQLATRAPRGLVGTVATTLDGSQAVLLGGVNKAIFDGYFTDLASAGSDEVRKSAVINAYFNQAPADYFYNRDVLIYDPQKNQWKSGGLLPFLGTAGSAISRMDNRLILINGEIKPGLRTAAVWQGLMQGNVLEWQPQPDLIGAETGSAQEGLAGAFSGISHKTVLVAGGANFPGAWKQFNRGHLYAHQGLEKQWHQQVYALVDNQWRIAGKLPQPLGYGVSIQGPDKVILIGGETTGGTATSAVTQLSWQGGKLHIE

SEQ ID NO: 21 - protein, 368 aa, Shigella boydii serotype 4 (strain Sb227)
MNKTITALAIMMASFAANASVLPETPVPFKSGTGAIDNDTVYIGLGSAGTAWYKLDTQAKDKKWTALAAFPGGPRDQATSAFIDGNLYVFGGIGKNREGLTQVFNDVHKYNPKTNSWVKLMSHAPMGMAGHVTFVHNGKAYVTGGVNQNIFNGYFEDLNEAGKDSTAVDKINAYYFDKKAEDYFFNKFLLSFDPSTQQWSYAGESPWYGTAGAAVVNKGDKTWLINGEAKPGLRTDAVFELDFTGNNLKWNKLAPVSSPDGVAGGFAGISNDSLIFAGGAGFKGSRENYQHGKNYAHEGLKKSYSTDIHLWHNGKWDKSGELSQGRAYGVSLPWNNSLLIIGGETAGGKAVTDSVLISVKDNKVTVQN

SEQ ID NO: 22 - protein, 384 aa, Vibrio cholerae serotype O1 (strain ATCC 39315); identical to SEQ ID NO: 18

SEQ ID NO: 23 - protein, 1014 aa, Clostridium septicum
MNKKKIMSILVSAFLITNLSSNIIFADIKENVYINQYSEGNRSQPIAEKLVPRSEIQASATSALTGEGPEKAIDGNTSTLWHTPWAGVDIQINPQSLTLKLGKTRNISSICVTPRQEGTNGMITDYKIYSGDDVIAEGKWKSDSSDKYVVFDNPISTDNIRIEAISTVGDENNKHASIAEVEVYELADTPVKLAESNNKVINNGNGGNYEGDISEISLLEEGTAIIRFTNNGSSLFSISNNERTNEHFHVYINGGAIGYELRKQSGNLATGSVNKALNAGINTIAFKAEKGKGYSIYLNGEKILTSSSITANFLSTLEGLNTLSLGKTDRPSGSNEYNFTGEIDFFELYSKPLADRYLKERTGETTSKDLPFPEGAVKTEPVDIFTPGELGSNNFRIPALYTTKDGTVLASIDVRKGGGHDAPNNIDTGIKRSTDGGVTWDEGKIILDYPGASSAIDTSLLQDDETGRIFLIVTHFAEGYGFGNSKTGSGYVEIEGKRYLKLLGANDTIYTVREGVVYDSNGEATNYTVDNNNELYENGNRIGNVLLSNSPLKVMGTSFLSLIYSDDDGQTWSDPIDLNKEVKTDWMRFLGTGPGKGHQIKTGRYAGRLLFPVYLTNASGFQSSAVIYSDDNGATWNIGETATDGRLMDNGDRASAETITTNTSGGVGQLTECQVVEMPNGQLKMFMRNTGGNSGRVRIATSFDGGATWEDDVVRDENIKEPYCQLSVINYSQKIDGKDAIIFAIPDANYPNRVNGTVRVGLITENGSYENGEPRYDIEWRYNKVVAPGTYGYSCLSEMPNGEIGLFYEGRGSRQMSFTRMNIDYLKADLLQDVPAANIKSYTTNSENNIYDPGDKISLNVTFDQTVSLIGDRTITADIGGKEVLLTLANSKGGSEYTFEGTVPADISNGNYTITIKGKSGLKIVNVVNKVTDITEDRNTGLNVQVGEEVQSVDKTLLQDLVDSTSNLIKEDYTEESWILYEKALEVANKFLVNEIAVQEEVDAAKPTLENAYK

SEQ ID NO: 24 - protein, 1035 aa, Streptococcus pneumoniae
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 25 - protein, 382 aa, Salmonella typhimurium (strain LT2)
MTVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 26 - protein, 781 aa, Vibrio cholerae serotype O1 (strain ATCC 39541)
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 27 - protein, 1035 aa, Streptococcus pneumoniae (strain ATCC BAA-255); identical to SEQ ID NO: 24

SEQ ID NO: 28 - protein, 697 aa, Streptococcus pneumoniae serotype 4 (ATCC BAA-334)
MNKRGLYSKLGISVVGISLLMGVPTLIHANELNYGQLSISPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQLIFQSGDSTQANYFRIPTLYTLSSGRVLSSIDARYGGTHDSKSKINIATSYSDDNGKTWSEPIFAMKFNDYEEQLVYWPRDNKLKNSQISGSASFIDSSIVEDKKSGKTILLADVMPAGIGNNNANKADSGFKEINGHYYLKLKKNGDNDFRYTVRENGVVYNETTNKPTNYTINDKYEVLEGGKSLTVEQYSVDFDSGSLRERHNGKQVPMNVFYKDSLFKVTPTNYIAMTTSQNRGESWEQFKLLPPFLGEKHNGTYLCPGQGLALKSSNRLIFATYTSGELTYLISDDSGQTWKKSSASIPFKNATAEAQMVELRDGVIRTFFRTTTGKIAYMTSRDSGETWSKVSYIDGIQQTSYGTQVSAIKYSQLIDGKEAVILSTPNSRSGRKGGQLVVGLVNKEDDSIDWKYHYDIDLPSYGYAYSAITELPNHHIGVLFEKYDSWSRNELHLSNVVQYIDLEINDLTK

SEQ ID NO: 29 - protein, 191 aa, Salmonella typhimurium (strain LT2)
MPFKMSSKAQTIKIFNLRSDTNEFIGAGDAYIPPHTGLPANCTDIAPPDIPSSHIAVFDAETQTWNLKEDHRGETVYDTTTGNQMYISEPGPLPENVTSVSPGGGYKKWDSKAKAWVNDEGAEVAARLREAEGTKSRLLQMASGKIAPLQDAVDLGIATDDEKAQLDEWKKYRVLVNRVDTSNPDWPEQPV

SEQ ID NO: 30 - protein, 368 aa, Escherichia coli O6H1 (strain CFT073)
MNKTITALAILMASFAANASVLPETPVPFKSGTGVIDNDTVYIGLGSAGTAWYKLDTQAKDKRWTALAAFPGGPRDQATSAFIDGNLYVFGGIGKNSEGLTQVFNDVHKYNPKTNSWVKLMSHAPMGMAGHVTFVHNGKAYVTGGVNQNIFNGYFEDLNEAGKDSTAIDKINAYYFDKKAEDYFFNKFLLSFDPSTQQWSYAGESPWYGTAGAAVVNKGDKTWLINGEAKPGLRTDAVFELDFTGNNLKWNKLAPVASPDGVAGGFAGMSNDSLIFAGGAGFKGSRENYQNGKNYAHEGLKKSYSADIHLWHNGKWDKSGELSQGRAYGVSLLWNNSLLIIGGEAAGGKAVTDSVLISVKDNKVTVQN

SEQ ID NO: 31 - protein, 384 aa, Salmonella enteritidis PT4 (strain P125109)
MGMQMKNFKKMMTLMALCLSVAITTSGYATTLPDIPEPLKNGTGAIDNNGVIYVGLGTAGTSWYKIDLKKQHKDWERIKSFPGGAREQSVSVFLNDELYVFGGVGKKNSESPLQVYSDVYKYSPVKNTWQKVDTISPVGLTGHTGVKLNETMVLITGGVNEHIFDKYFIDIEAADESEKNKVIYNYFNKPAKDYFFNKIVFIYNAKENTWKNAGELPGAGTAGSSSVMENNFLMLINGELKPGLRTDVIYRAMWDNDKLTWLKNSQLPPSPGEQQQEGLAGAFSGYSHGVLLVGGGANFPGAKQNYTNGKFYSHEGINKKWRDEVYGLVNGHWQYMGKMKQPLGYGVSVSYGDEVFLIGGENAKGKPVSSVTSFTMRDGNLLIK

SEQ ID NO: 32 - protein, 368 aa, Escherichia coli O9H4 (strain HS)
MNKTITALAIMMASFAANASVLPETPVPFKSGTGAIDNDTVYIGLGSAGTAWYKLDTQAKDKKWTALAAFPGGPRDQATSAFIDGNLYVFGGIGKNSKGLTQVFNDVHKYNPKTNSWVKLMSHAPMGMAGHVTFVHNGKAYVTGGVNQNIFNGYFEDLNEAGKDSATIDKINAHYFDKKAEDYFFNKFLLSFDPSTQQWSYAGESPWYGTAGAAVVNKGDKTWLINGEAKPGLRTDAVFELDFTGNNLKWNKLAPVSSPDGVAGGFAGISNDSLIFAGGAGFKGSRENYQNGKNYAHEGLKKSYSTDIHLWHNGKWDKSGELSQGRAYGVSLPWNNSLLIIGGETAGGKAVTDSVLISVKDNKVTVQN

SEQ ID NO: 33 - protein, 375 aa, Haemophilus influenzae (strain 86-028NP)
MKLTKTALCTALFATFTFSANAQTYPDLPVGIKGGTGALIGDTVYVGLGSGGDKFYTLDLKDPSAQWKEIATFPGGERNQPVAAAVDGKLYVFGGLQKNEKGELQLVNDAYRYNPSDNTWMKLPTRSPRGLVGSSGASHGDKVYILGGSNLSIFNGFFQDNVAAGEDKGKKDEIVAAYFDQRPEDYFFTTELLSYEPSTNKWRNEGRIPFSGRAGAAFTIQGNDLVVVNGEIKPGLRTAETHQGKFTAKGVQWKNLPDLPAPKGKSQDGLAGALAGYSNGYYLVTGGANFPGSIKQFKEGKLHAHKGLSKAWHNEVYTLNNGKWSIVGELPMNIGYGFSVSYNNKVLLIGGETDGGKALTSVKAISYDGKKLTVE

SEQ ID NO: 34 - protein, 375 aa, Haemophilus influenzae (strain PittEE)
MKLTKTALCTALFATFTFSANAQTYPDLPVGIKGGTGALIGDTVYVGLGSGGDKFYTLDLKDPSAQWKEIATFPGGERNQPVAAAVDGKLYVFGGLQKNEKGELQLVNDAYRYNPSDNTWMKLPTRSPRGLVGSSGASHGDKVYILGGSNLSIFNGFFQDNVAAGEDQAKKDEIAAAYFDQRPEDYFFTTELLSYEPSTNKWRNEGRIPFSGRAGAAFTIQGNDLVVVNGEIKPGLRTAETHQGKFTAKGVQWKNLPDLPAPKGKSQDGLAGALSGYSNGHYLVTGGANFPGSIKQYKEGKLHAHKGLSKAWHNEVYTLNNGKWRIVGELPMNIGYGFSVSYNNKVLLIGGETDGGKALTSVKAISYDGKKLTIE

SEQ ID NO: 35 - protein, 386 aa, Salmonella choleraesuis (strain SC-B67)
MGMQMKNFKKMMTLMALCLSVAITTSGYATTLPDIPEPLKNGTGAIDNNGVIYVGLGTAGTSWYKIDLKKQHKDWERIKSFPGGAREQSVSVFLNDELYVFGGVGKKNSESPLQVYSDVYKYSPVKNTWQKVDTISPVGLTGHTGVKLNETMVLITGGVNEHIFDKYFIDIAAAAADESEKNKVIYNYFNKPAKDYFFNKIVFIYNAKENTWKNAGELPDAGTAGSSSVMENNFLMLINGELKPGLRTDVIYRAMWDNDKLTWLKNSQLPPSPGEQQQEGLAGAFSGYSHGVLLVGGGANFPGAKQNYTNGKFYSHEGINKKWRDEVYGLVNGHWQYMGKMKQPLGYGVSVSYGDEVFLIGGENAKGKPVSSVTSFTMRDGNLLIK

SEQ ID NO: 36 - protein, 386 aa, Salmonella newport (strain SL254); identical to SEQ ID NO: 35

SEQ ID NO: 37 - protein, 386 aa, Salmonella paratyphi B (strain ATCC BAA-1250); identical to SEQ ID NO: 35

SEQ ID NO: 38 - protein, 386 aa, Salmonella schwarzengrund (strain CVM19633)
MGMQMKNFKKMMTLMALCLSVAITTSGYATTLPDIPEPLKNGTGAIDNNGVIYVGLGTAGTSWYKIDLKKQHKDWERIKSFPGGAREQSVSVFLNDELYVFGGVGKKNSESPLQVYSDVYKYSPVKNTWQKVDTISPVGLTGHTGVKLNETMVLITGGVNEHIFDKYFIDIAAAAADESEKNKVIYNYFNKPAKDYFFNKIVFIYNAKENTWKNAGELPGAGTAGSSSVMENNFLMLINGELKPGLRTDVIYRAMWDNDKLTWLKNSQLPPSPGEQQQEGLAGAFSGYSHGVLLVGGGANFPGAKQNYTNGKFYSHEGINKKWRDEVYGLVNGHWQYMGKMKQPLGYGVSVSYGDEVFLIGGENAKGKPVSSVTSFTMRDGNLLIK

SEQ ID NO: 39 - protein, 368 aa, Shigella flexneri serotype 5b (strain 8401)
MNKTIMALAIMMASFAANASVLPETPVPFKSGTGAIDNDTVYIGLGSAGTAWYKLDTQAKDKKWTALAAFPGGPREQATSAFIDGNLYVFGGIGKNSEGLTQVFNDVHKYNPKTNSWVKLMSHAPMGMAGHVTFVHNGKAYVTGGVNQNIFNGYFEDLNEAGKDSTAIDKINAHYFDKKAEDYFFNKFLLSFDPSTQQWSYAGESPWYGTAGAAVVNKGDKTWLINGEAKPGLRTDAVFELDFTGNNLKWNKLDPVSSPDGVAGGFAGISNDSLIFAGGAGFKGSRENYQNGKNYAHEGLKKSYSTDIHLWHNGKWDKSGELSQGRAYGVSLPWNNSLLIIGGETAGGKAVTDSVLISVKDNKVTVQN

SEQ ID NO: 40 - protein, 392 aa, Yersinia pestis bv. Antiqua (strain Antiqua); identical to SEQ ID NO: 20

SEQ ID NO: 41 - protein, 375 aa, Haemophilus influenzae (strain PittGG)
MKLTKTALCTALFATFTFSANAQTYPDLPVGIKGGTGALIGDTVYVGLGSGGDKFYTLDLKDPSAQWKEIATFPGGERNQPVAAAVDGKLYVFGGLQKNEKGELQLVNDAYRYNPSDNTWMKLPTRSPRGLVGSSGASHGDKVYILGGSNLSIFNGFFQDTVAAGEDKAKKDEIAAAYFDQRPEDYFFTTELLSYEPSTNKWRNEGRIPFSGRAGAAFTIQGNDLVVVNGEIKPGLRTAETHQGKFTAKGVQWKNLPDLPAPKGKSQDGLAGALSGYSNGHYLVTGGANFPGSIKQFKEGKLHAHKGLSKAWHNEVYTLNNGKWRIVGELPMNIGYGFSVSYNNKVLLIGGETDGGKALTSVKAISYDGKKLTIE

SEQ ID NO: 42 - protein, 375 aa, Haemophilus influenzae (strain ATCC 51907)
MKLTKTALCTALFATFTFSANAQTYPDLPVGIKGGTGALIGDTVYVGLGSGGDKFYTLDLKDPSAQWKEIATFPGGERNQPVAAAVDGKLYVFGGLQKNEKGELQLVNDAYRYNPSDNTWMKLPTRSPRGLVGSSGASHGDKVYILGGSNLSIFNGFFQDTVAAGEDKAKKDEIAAAYFDQRPEDYFFTTELLSYEPSTNKWRNEGRIPFSGRAGAAFTIQGNELVVVNGEIKPGLRTAETHQGKFTAKGVQWKNLPDLPAPKGKSQDGLAGALSGYSNGHYLVTGGANFPGSIKQFKEGKLHAHKGLSKAWHNEVYTLNNGKWRIVGELPMNIGYGFSVSYNNKVLLIGGETDGGKALTSVKAISYDGKKLTIE

SEQ ID NO: 43 - protein, 380 aa, Pasteurella multocida (strain Pm70)
MKFTKTALFTVLAATAFAAQAGQYPDLPEGIKAGAGALIGDTVYVGLGGTGTTKFYSLNLKDPKEWKEIAEFPGGKRNQPVAAGVNGKLYVFGGFQDTDVAKNQIINDAYEYNPADNTWTKLSTRSPRSTSVGASVAADGGKIYFVGGVNHEIWNGLFQDVKAAGGDKEKEKAIFDPYFNLRAQDFFFSPEIISYEPANNVWRNEGYFPYSGRAGAAVAIKDGKLLVVNGEVKAGLRSPGTALGTIGKDGVTWKKLGDLPAPTGYDKQDGIAGGMGGYTNGHYIVTGGANFPGALANYEKGIMDAHRTGGLKKTYHKAVYALDGKTGNWKIVGELPATIGYGLAVSYNNKVLLIGGETDGGKPLSAVQTMSYDGKKLTVE

SEQ ID NO: 44 - protein, 386 aa, Salmonella arizonae (strain ATCC BAA-731)
MGMQMKNSKKMMTLMALCLSVAITTSGYATTLPDIPEPLKNGTGAIDNNGVIYVGLGSAGTSWYKIDLKKQHKDWERIKSFPGGAREQSVSVFLNGELYVFGGVGKESNEAPLQVYSDVYKYTPAKNTWQKVNTISPVGLTGHTVVKLNETMALITGGVNEHIFDKYFIDIAAAAGDDSEKNRVIYNYFNKPSKDYFFNKIVFIYNAKENTWENAGELPGAGTAGSSSAMENNSLTLINGELKPGLRTDVIYRAIWDNDKLTWLKNSQLPPSPGEQHQEGLAGAFSGYSQGVLLVGGGANFPGAKQNYTDGKFYAHEGINKKWRDEVYGLINGHWQYVGKMKQPLGYGVSVNYGDEIFLIGGENAKGKPVSSVISFAMHDGKLLIE

SEQ ID NO: 45 - protein, 384 aa, Salmonella typhi
MGMQMKNFKKMMTLMALCLSVAITTSGYATTLPDIPEPLKNGTGAIDNNGVIYVGLGTAGTSWYKIDLKKQHKDWERIKSFPGGAREQSVSVFLNDKLYVFGGVGKKNSESPLQVYSDVYKYSPVKNTWQKVDTISPVGLTGHTGVKLNETMVLITGGVNEHIFDKYFIDIAAADESEKNKVIYNYFNKPAKDYFFNKIVFIYNAKENTWKNAGELPGAGTAGSSSVMENNFLMLINGELKPGLRTDVIYRAMWDNDKLTWLKNSQLPPSPGEQQQEGLAGAFSGYSHGVLLVGGGANFPGAKQNYTNGKFYSHEGINKKWRDEVYGLINGHWQYMGKMKQPLGYGVSVSYGDEVFLIGGENAKGKPVSSVTSFTMRDGNLLIK

SEQ ID NO: 46 - protein, 386 aa, Salmonella typhimurium (strain LT2)
MGMQMKNFKKMMTLMALCFSVAITTSGYATTLPDIPEPLKNGTGAIDNNGVIYVGLGTAGTSWYKIDLKKQHKDWERIKSFPGGAREQSVSVFLNDELYVFGGVGKKNSESPLQVYSDVYKYSPVKNTWQKVDTISPVGLTGHTGVKLNETMVLITGGVNEHIFDKYFIDIAAAAADESEKNKVIYNYFNKPAKDYFFNKIVFIYNAKENTWKNAGELPGAGTAGSSSVMGNNFLMLINGELKPGLRTDVIYRAMWDNDKLTWLKNSQLPPSPGEQQQEGLAGAFSGYSHGVLLVGGGANFPGAKQNYTNGKFYSHEGINKKWRDEVYGLINGHWQYMGKMKQPLGYGVSVSYGDEVFLIGGENAKGKPVSSVTSFTMRDGNLLIK

SEQ ID NO: 47 - protein, 392 aa, Yersinia pseudotuberculosis serotype O1b; identical to SEQ ID NO: 20

SEQ ID NO: 48 - protein, 392 aa, Yersinia pestis; identical to SEQ ID NO: 20

SEQ ID NO: 49 - protein, 392 aa, Yersinia pestis (strain Pestoides F); identical to SEQ ID NO: 20

SEQ ID NO: 50 - protein, 392 aa, Yersinia pseudotuberculosis serotype I; identical to SEQ ID NO: 20

SEQ ID NO: 51 - protein, 368 aa, Shigella flexneri; identical to SEQ ID NO: 39

SEQ ID NO: 52 - protein, 384 aa, Vibrio vulnificus (strain YJ016)
MMKTKYLLLPLLASSSLLSHMAFANNHWPDLPIGVKNGVSAQIGSKVYVGLGSAEKSFYVLDTQAPQNGWTLLAEFIGPERSGATATVVGENIFVFGGSGKASDDASSPIIFDTVYRFDTQTNRWHQVKTQTPVGLLGASSYSPDGKQVLFFGGYSKPLFDKYLADITRTDKKAQPEQWQKIVDDYMGMEPLAYQWNRDVLSFNPTNDKWDVVTRSPYLPNCGSATVIDGPSITLISGEIKPGLRTAEVKTFTYGELQPWQSTYALPAAKGQAQQEGIAGAYSGVVSNTLLVAGGANFHGAKAQFESGQMFAHNGLSKAYNSEIYAKQKGVWQQVGQLPEGLAYGASFSVKGGVLMVGGERADRTASTKVYLVGLNNNQIDIVD

SEQ ID NO: 53 - protein, 544 aa, Bacteroides fragilis (strain YCH46)
MKKAVILFSLFCFLCAIPVVQAADTIFVRETRIPILIERQDNVLFYLRLDAKESQTLNDVVLNLGEGVNLSEIQSIKLYYGGTEALQDSGKKRFAPVGYISSNTPGKTLAANPSYSIKKSEVTNPGNQVVLKGDQKLFPGINYFWISLQMKPGTSLTSKVTADIASITLDGKKALLDVVSENGIEHRMGVGVRHAGDDNSAAFRIPGLVTTNKGTLLGVYDVRYNSSVDLQEHVDVGLSRSTDGGKTWEKMRLPLAFGEFGGLPAGQNGVGDPSILVDTKTNNVWVVAAWTHGMGNQRAWWSSHPGMDMNHTAQLVLAKSTDDGKTWSAPINITEQVKDPSWYFLLQGPGRGITMSDGTLVFPTQFIDSTRVPNAGIMYSKDGGKNWKMHNYARTNTTEAQVAEVEPGVLMLNMRDNRGGSRAVAITKDLGKTWTEHESSRKALPESVCMASLISVKAKDNVLGKDLLIFSNPNTTKGRYNTTIKISLDGGVTWSPEHQLLLDEGNNWGYSCLSMIDKETIGILYESSVAHMTFQAVKLKDIIK

SEQ ID NO: 54 - protein, 368 aa, Shigella sonnei (strain Ss046)
MNKTIMALAIMMASFAANASVLPETPVPFKSGTGAIDNDTVYIGLGSAGTAWYKLDTQAKDKKWTALAAFPGGPRDQATSAFIDGNLYVFGGIGKNSEGLTQVFNDVHKYNPKTNSWVKLMSLAPMGMAGHVTFVHNGKAYVTGGVNQNIFNGYFEDLNEAGKDSTAIDKINAHYFDKKAEDYFFNKFLLSFDPSTQQWSYAGESPWYGTAGAAVVNKGDKTWLINGEAKPGLRTDAVFELDFTGNNLKWNKLDPVSSPDGVAGGFAGISNDSLIFAGGAGFKGSRENYQNGKNYAHEGLKKSYSTDIHLWHNGKWDKSGELSQGRAYGVSLPWNNSLLIIGGETAGGKAVTDSVLISVKDNKVTVQN

SEQ ID NO: 55 - protein, 647 aa, Micromonospora viridifaciens
MTANPYLRRLPRRRAVSFLLAPALAAATVAGASPAQAIAGAPVPPGGEPLYTEQDLAVNGREGFPNYRIPALTVTPDGDLLASYDGRPTGIDAPGPNSILQRRSTDGGRTWGEQQVVSAGQTTAPIKGFSDPSYLVDRETGTIFNFHVYSQRQGFAGSRPGTDPADPNVLHANVATSTDGGLTWSHRTITADITPDPGWRSRFAASGEGIQLRYGPHAGRLIQQYTIINAAGAFQAVSVYSDDHGRTWRAGEAVGVGMDENKTVELSDGRVLLNSRDSARSGYRKVAVSTDGGHSYGPVTIDRDLPDPTNNASIIRAFPDAPAGSARAKVLLFSNAASQTSRSQGTIRMSCDDGQTWPVSKVFQPGSMSYSTLTALPDGTYGLLYEPGTGIRYANFNLAWLGGICAPFTIPDVALEPGQQVTVPVAVTNQSGIAVPKPSLQLDASPDWQVQGSVEPLMPGRQAKGQVTITVPAGTTPGRYRVGATLRTSAGNASTTFTVTVGLLDQARMSIADVDSEETAREDGRASNVIDGNPSTFWHTEWSRADAPGYPHRISLDLGGTHTISGLQYTRRQNSANEQVADYEIYTSLNGTTWDGPVASGRFTTSLAPQRAVFPARDARYIRLVALSEQTGHKYAAVAELEVEGQR

SEQ ID NO: 56 - protein, 372 aa, Escherichia coli O6H1 (strain CFT073)
MITMKVKNFIYLPFCLFIGTSVAGALPEIPEPFKYGVGGIENGKIYIGLGSLGNNWYMIDTNQSEKKWTKIAQWPTVPREQATATIIDGKIYVFGGIGKDTSGVITLQKDVYSYDIAKDKWEKLMTRPPVSLAGHVSFIHNGHAVSTGGVNENIFNGYFSDVELSKGNSALTEKVNRDYFSKPADDYFLNNHIISYDPSKNQWKNLGTTPFPGTAGSSVIFAEQQIYILGGERKPGLRSVRSWTGELSHDRIKWSELPPVASPEGVSGAYASVIDGNIFLAGGAYFPGAAEKYSNGEYWSHKGLDKAYSKEIYQLIKNDWKKVGSLPEGLAYGVSLPWQGGMLILGGEKKDGKAVSDVIYLKKNDKQIKIVK

SEQ ID NO: 57 - protein, 388 aa, Brucella abortus (strain 2308); identical to SEQ ID NO: 10

SEQ ID NO: 58 - protein, 368 aa, Escherichia coli O1K1 / APEC; identical to SEQ ID NO: 17

SEQ ID NO: 59 - protein, 368 aa, Escherichia coli O6K15H31 (strain 536); identical to SEQ ID NO: 17

SEQ ID NO: 60 - protein, 543 aa, Bacteroides xylanisolvens
MRRIYYLLFLILLGYSFDVKASDTVFIHETQIPVLIERQDNVLFYLRLDAKESKKLDEIILDFSKSTNLTDIQAIKLYYGGTEALQDKDKNRFAPVEYISSHRPGGTLAAIPSYSIKCAEVGSSEKVVLKGNYNLFPGVNYFWISLLMKKDASLQTKILSELCAVKVDGKELCCKSISPKNIVHRMAVGVRHAGDDGSASFRIPGLVTTNKGTLLGVYDVRYNSSVDLQEYVDVGLSRSIDGGKNWEKMRLPLSFGEYGGLPKAQNGVGDPSILVDTKTNTVWVVAAWTHGMGNQRAWWSSHPGMDLNHTAQLVLAKSTDDGKTWSKPINITEQVKDPSWYFLLQGPGRGITMSDGTLVFPTQFIDSTRVPNAGIMYSKDRGKTWKMHNMARTNTTEAQVAEIEPGVLMLNMRDNRGGSRAIAITKDLGETWTEHPSSRQALQEPVCMASLIHVDAKDNILNKDILLFSNPNTTKGRNHITIKASLDKGLTWLPEHQLMLDEAEGWGYSCLTMIDKETIGILYESSVAHITFQAIKLTDIIKE

SEQ ID NO: 61 - protein, 781 aa, Vibrio cholerae serotype O1 (ATCC 39315); identical to SEQ ID NO: 26

SEQ ID NO: 62 - protein, 382 aa, Clostridium perfringens
MCNKNNTFEKNLDISHKPEPLILFNKDNNIWNSKYFRIPNIQLLNDGTILTFSDIRYNGPDDHAYIDIASARSTDFGKTWSYNIAMKNNRIDSTYSRVMDSTTVITNTGRIILIAGSWNTNGNWAMTTSTRRSDWSVQMIYSDDNGLTWSNKIDLTKDSSKVKNQPSNTIGWLGGVGSGIVMDDGTIVMPAQISLRENNENNYYSLIIYSKDNGETWTMGNKVPNSNTSENMVIELDGALIMSTRYDYSGYRAAYISHDLGTTWEIYEPLNGKILTGKGSGCQGSFIKATTSNGHRIGLISAPKNTKGEYIRDNIAVYMIDFDDLSKGVQEICIPYPEDGNKLGGGYSCLSFKNNHLGIVYEANGNIEYQDLTPYYSLINKQ

SEQ ID NO: 63 - protein, 543 aa, Bacteroides xylanisolvens; identical to SEQ ID NO: 60

SEQ ID NO: 64 - protein, 1184 aa, Unknown
  Environmental sample
MKKRKRVLALMLAGVLTLGCFPTTVQAVNNAYDGTKPADGTTKKQPFLAGTGGSTNFRIPGLVTLNDGTLVASSDARWNGGGDGGGLDTIVSRSTDNGKTWSYTFANYLGDNGNVHNNGSTAFIDPALTTDGETVYMAADLYPAGTALNSASYAPKTGHTGYDFRHRLVLAKATEGVTAASDTAQRMNAAFTYYLEKNPDENAASYYLLKDASGNTVSGYTVDAYFNITGNGVNTNLFCGDSPYFPYPTDFIYMTKSTDGGETWSEPTFLDVKKASEQSLLVGPGRGIVISSGEHKGRIVFTCYEFTSGDKNSAAIYSDDNGVTWHRGASVKNLSSEAVVTEADGKLYMFVRRENVYYVSNDGGETWSDPQAMGISYNATCQLTATTYSEKINGKTAIFFACPSNTSSRAAGKIYVGLVQDDGSIDWAYDYSINGSAYYAYSCISEMGNGNIGLLYESNGTAITFKEIALKDIVGSASVGSVWLTDEDGNVISSDSMKSSQTKTYTVNGVAEGTSVEVTSSDESTVTAVYENGKLKVTSGQVEGLKQAAVTVHAGSFVRILKLNISDTENYKVVELNMGGTKSYTDTTGNYSAADTSSLDKTTAKVTMTGEDPQAPEVKTVAVKATATATFNGAELDLDDCEYTFEATGTDNQYKVKATTADGQTVYLGTRAAGSNNVPSSTTETSITVAKHSTDSLFTLKDNSTGSVGNILWFWSTDTSKLYFDRNSSVADNCYFELYKKSASAPEDSAVHGYQKVTALDEIQGGKKYLIVTKADANNNQYVVVPSAAAVANRYEHVGKLKTNTEGTPGSTQITFEGTGEGTTSVTIGAVTYYVVVKNEVQKVELKVGEKFLAPGKLMQQESSQKILSLTANGDQPPYVKNTEIVSGKEYLIGQGSYVLVNSQSTASGTPTGLAMKTADLESDDQSAYMWTVKAVDGGYTIQDVNGKYLSFNGSNVGLSDTAQTLTVGNGASDGFGISYGGQYLNNYGRSNTKVAGYSANDNDWYLFAPETGYFVTAEKAGTTTVVIGGVTYEIVVTETECKHENTERVGVKDPTCTEPGTTGKLVCKDCNETLEEAAEIAATGHSYGEWTVVKEATTEEEGLEKRVCEKCDAEQTRSIPKPSDTQKPSDHQKPSDNKTAGTTVTGSKAAKTGDVSNVLLPGMTMLLAGGAAAGTLLGKKRKK

SEQ ID NO: 65 - protein, 425 aa, Unknown
  Environmental sample
MIFSANIGKALCLATMGITLMMPAQVAAATPEMAVEAQAVNAQDGITVFPNKEAQYRIPAIVECKSGKLIAFTDHRYHNKDIGGGRHLDIVMKTSMDKGATWSSPEQMVAKGGNGIATSFDCAHGDAAVVVDKKSGRILLMCASGGIGYWESKRGHLLMMGRYYSDDEGKTWYGEEVTKQIYDLMPEVESAFFTSGRICQSKQIKVGKYYRVYTVLCTRSGNRILYSDDFGETWNVLGKNVAEAAPYGDEAKIEELPDGNVLLSSRAMGGRHINIYTYEDKKTATGSWGKVVASDAKNMGVAAHKNSCNGEVLMVDAKKNGKKVKLLLQSVPVGPGRNNVGIYYKALENPADYATPEAIAKNWEGCYQLSNTTSAYSTMVQGKDGSIYFLLEENAFRNPKTQTDDYYDIRFYNLSIEQITNNRYK

SEQ ID NO: 66 - protein, 431 aa, Unknown
  Environmental sample
MKKIFLTLGLALACSASWAQTLLWQTDTTRHQYYRIPAIVANGKNVIAFADNRSGVTDATSWGDVGSVGNISIEVRHSKDGGRTWTSPKCAVKGFGNGGYDQSHGDAAVVRDRETGRMLIMTGSGDVGYGRSKVTVPGDYSEALKVGRYYSDDNGNTWNGGEVTSQIYDLYRGHGNIFRLFFTSGRICQSRMIKRGEYYRIYSAVVTNEGSLVVYSDDFGDTWTPLGGADARPAPEGDEAKLEELPDGRVLLSCRRSGKDGRWFNVFSYGDNSYGQGSWAQPVASENPDGGTATINNNCNGEILLVPAKAKDGSRVYVALQSVPRGTPGNHPSNLERRSHVSVYWKAFRSAADWASPENWKDGWKRYEITDGYSAYSSMAIGDKGNICFIYEDDGVRFRLGSQSTEMYSIFYRNLTLKEITGGKYKYSKIK

SEQ ID NO: 67 - protein, 1246 aa, Unknown
  Environmental sample
MNKTIITVMAALAMLDAYAAKKPSKGTDVIRVVDLRTERMTAPLSINTPTPRLGWRIESGKKNVMQTSCRIIVASTYEKAMAGEGDLWDTTINGDQSQWVNYAGKQLRSNTPCYWRVKVATTQGESAWSDVQMWNIGLLTESDWSGQWIGFDAAKPWDKEEIHSQLSSRYIRKEFKLDKKVRRATLYISGLGMYEAFINGRKVGDQVLAPAPTDYRKTVLYNAFDVTSMLTTENAIGVALGNGRYYTMQQKKKPYKITNFGYPKLRANIIVEFEDGTRKTISTDSKWKLNADGAIRSNNEYDGEIYDARKEFGAWTSAGFDDSRWENAERVAIPEGTLRGAMAPNMKVLQQMAARSITMHGDTAIIDLGQNIAGWLKLKINDAAEGDSIKICFAETLTKDGRLYRENLRHALSTDCYIANGKENGRWWTPTFVYHGFRYAQITGLKNPKKEDFIGEVVSDEMEKTGFFASSDSILNKVYHNAEWGILANYKGMPVDCPQRDERQPWLGDRTRGCLGESFIFDNDLLYNKWDRDICEAQREDGCIPDVAPAYWNYYSDNITWPAALPFSMEMQYDQFADTSAISKFYPAVRKWISHMEYQYEKDGLMPRDKYGDWCVPPEDIKMIHSKDPKRVTDGTLIATAYYYLLNKKMQRFANILGKKNEAEAFAQKAETVKKAFNRKFLTVKPHTHEAYGGLLYPDSTFYGNNTATANLLPLAFGMTDDPYVKTEVEKNILKNIITDNKGRISCGVIGVQWLMHALSDMGRTDVAWLLATNKAYPSWGYMAENGATTTWELWNGNTASPKMNSGNHVMLLGDLVSWIYQNLGGINSDPAKPGYKHIILKPEFGVDEIDNINASFKSIYGNITSHWRKQNGTLHWDVEIPTNTTATLFMPDGTTRETGSGKYSFTQKLPTRDTHILADEFLYTKASFPECHSATIAETKNGDLVATYFGGTKERNPDVCIWVSRKPKGSSNWTLPQMVADGVMNDSVRKACWNPVVYQIPGGDLVIYFKIGSKVSDWTGWMVRSKDGGKTWSKRMPLQQDFLGPVKNKPVLSKGRLIAPTSIESDGWRLYFEYSDDMGKTWKRTPYVATDSIMQDGKMRIKDLAIQPTIIFLSDGRLAALARTRSEHVAITYSSDNGETWTKLKLLSLPNNNSGLDAVTLSNGMHVMICNEKPIPHGIKNGKGLRTPLSLMKSDDGENWHHWITLEDSPISQYSYPSIIQTADGHIHCIYTWRRQRIKHIELLP

SEQ ID NO: 68 - protein, 1365 aa, Unknown
  Environmental sample
MKRIKLITVILLFLFSLLAKGEDRIGVLQINVWQEGTCVPGGFDALVDNIIESGADVVFMQEIRNYGGRRFIPRLIEALALRGVTFYGEDTTCDSGVISRFPLAQDGRKWEGCGSVQRVRINAGGHPVALYAAHLDYTNYACYYPRGYDGNTWKKIPAPETSVEKILEMNRASGRDDTIRDFIRLSRADIDEGIDVILAGDFNEPSHLDWTAQTASLWDHNGAVIPWDCSTMLYEGGFEDSFRVMNPDPVSSPGFTFPSDNSDCNPMKLTWAHESDERDRIDFIYYYGEGSLKPVDSKVFGPAGSIVRGKRIVEENHSDIIPVTGKWPTDHKAVLTTFEFAPVARILTPSIPTIVDREHNIICEICLESFSGKDVLESVGVTVSGIDPSAIRNARLMYTGTSSAIYSRTTSYILKDQFRRIGAGQDTWRHPAFAVQTASCKAGKDGRIVLKPGKSLVRDRNYFYLSLEVDSRKVDLSGTFGVAVESVELRCPVQDGSGSLLRQARIVSEGSLEGHRFGIALRNYKDDNVFSYRIPGLVTTPKGTLIAVYDIRHTSGNDLQDNIDVGASRSTDGGRTWEKMRTIIDMGEWGGLPQGQNGCGDPSVLVDSNTGTIFVCALWTHGLGNRHAFSFASEGYEPISTGQMVLVKSTDDGKTWSKPVNITRQVKRPDWRITLQGPGRGITMQDGTLVFPMQCLDSKGIPSSGIMYSKDGGETWHCHSRACERTTEAQVAEIAPGVLMLNCRNDLRQGRVVCVTDDLGKTWKTHPSSGSLVEPVCMAGLLNIPASENIYGRDILLFSNPVSAPGKGRNHICIKASLDAGMTWNPLDCLLLDEEEGWGYSCLTSIGNDKVGILYEGSTSHLVFQQVRLSDIVKSFPAPSGLSVGEIYSDDMIFQRDVPLEISGRTAPGLEVKVTLGPSTRKCRSAQDGTWKVRMPALKAGTGIPFTVTDGRDTLSFSNVAAGEVWLCSGQSNMAFELKGADTFSKEGLDDPMLRFYDMKSHLKTWGLVWSEEDAGKVAQGKYYDRTRWSCSSEKTAGKFSAVGYYFGKMLRDSLNVPVGLICNAVGGTTTESFISRSALEKVLPGFCDGWLEDESVMEWARGRARENMSAFKGGYIQHPFMPAYLFENGIRPLERFPIKGAIWYQGESNAENIPQHEILFRTLVDCWRQTWDNPDLPFYFVQLSSIERPTWPEFRNSQRLLSLEIPHCGMAVSSDLGHPTDVHPRAKKPVGERLARLALRNDYGKQVECLAPAATKAFLNDSGNVTVLFDSPISVPGTMPVRGFRICLEDGSEIPASAEISGSDAVILSRDAGRPDAAADSKNTKADSRTAIVKVKYAWDPWTDANLTGTTGLPVSTFDIEIGE

SEQ ID NO: 69 - protein, 425 aa, Unknown
  Environmental sample
MIFSANIGKALCLATMGISLLSTVQVAVAIPAVAAETQVVNRQGGITVFPNREAQYRIPAIVECKSGKLIAFTDHRYHNQDIGGGRHLDIVMKTSMDKGATWSSPEQMVAKGGNGIATSFDCAHGDAAVVVDKESGRILLMCASGGIGYWESKRGHLLMMGRYYSDDEGKTWYGEEVTKQIYDLMPEVESAFFTSGRICQSKQIKVGKYYRVYTVLCTRSGNRILYSDDFGQTWNVLGKNVAEAAPRGDEAKVEELPDGNVLLSSRTMGGRYINIYSYDDKKTATGSWGKVVASEAQNLGVAAHKNACNGEVLIVDAKKNGKKVKLLLQSVPVGPGRTNVGIYYKALETPADYATPEAIAKNWEGCYQLSNTTSAYSTMVQGKNGSIYFLLEENAFKNPKTQTDDYYDIRFYDLSVEQITNNRYK

SEQ ID NO: 70 - protein, 489 aa, Unknown
  Environmental sample
MKKILFSLGMLLLTVAAYADGTILFSTAKGSIDVPPYRIPGITCGYGGRLVASAARLVCGTDPGFGRVDCVVKLSYDNGRTWSEHEIDVACGDTSLINAHKTPMEAAYGDPAVVMDRERNEVLIMAVAGCTVYGKPSTNRQNPNLIAAIRSTDGGLTWQKPVDQTEDIYGLFDKGNVVDAAFVGSGKLFQSRVVKMGDYYRIYAALTARPNGNRVIYSDDFGRSWHALGGASALPVPGGDEPKCEELPDGRVIITSRASGCRLLNIYTYSNTKTGEGCWDKPEKATMDGLALAPSTNPTNGEMLIVPAKRNSDGKPLYVALQSVPTGTGRNNVSIFYKELASPDDMRNAKAFASGWDGCYQVSSTVSMYSSMDLQADDRIAFFYEETLTKWGTKPNPVSTSFPQGEGEHNYDGCELVYKSFDLETITDGKYCVNRRINRGKFLKAYYEDIVDSFGLTAAQKRKVKDVIKRLPAEPTSAQMDAILEGEVR

SEQ ID NO: 71 - protein, 885 aa, Unknown
  Environmental sample
MAVTGLATDTVEAFVLVGGNYAIDITNFNVHYEPVPEKEDTTVTDTVAIPTIDINTTTGSFVSKNSAGSWASLWKSTQSSPYFNLGCGANNMDVNNSTEGKIIAYVGNQTNSCDYTLSVERGWKITGYSFDFVRVTGKNAVTLTDVNGNTFTSSDEVQHVSVDNLKKSSLKAFNLSGGNDGVVITNFKVKYVVDSAYVAADFHTFTVFDNTGSVPYRIPAIGTAQDGSLVAVADYRYSKADIGSGRVDLHIRISHDNGLTWGDVMDLEQFRGDGNLSEWRHDKAAFGDPCIVGDRESPRMMITSCSGFPGFFDATDRHQGWARWYSNDNGLTWGEPEYLDEKFIYEPLKKAGYNVQGFFVGSGKIFQSHHIKRGDYYRLYCAGSTQEGNKKNWVLYSDDFGETWEFLGGTSKAPIISTADEPKVEELPNGNVILSSRDVSGRYYNIFTFSGNDGISGSWGEKALSSSAVNGLVANNGCNGEIQIVPVVRKKDGKKTFLALQSLPTSGRTNVSIFYKDLTDEATYTTPANFAKDWDGSFVVCDFSSAYSTWSWQHDNALAFLYEEGGGTSGGYNIVYKRIDIPTITSQEYDFDTSYKYERFNIDWAQADLNMEMASLLTTVKKTSEANTSFVKGDKLITDASQLTCKFGHKEMGGTGGDAQDISNLIDGDPATYYHTYWGAGDQPNGSHFMDVTLPEGQTFQGNIEVDVTRRSGATADHITQFTISGWNNATDTTQIAVVNVPNASDGAQATCEFTIPEGKSYSSLRFNVTGTTNNRGYWHMAEFQLYPISLDPKCLNALCADAYKQLAEAIATAEAVEKVSEADVTALQTAYQAYLDALAPITSIGGVSIQPATTIGSAIYDLQGRKVQRMVKGVYIVNGKKVVR

SEQ ID NO: 72 - protein, 434 aa, Unknown
  Environmental sample
MKKILSALAMLLLALSAYADGTILFSTAQGGIDVAPYRIPGITCGYGGRLIASAGRLVCGTDPGFGRVDCVVKISTDNGATWSEREIEAAVGDASLINNVKTPMAAAYGDPAIVMDRESHEVLMMAVAGCTVYGYASTNRHNPNLIAAIRSMDGGLTWQQPIEQTEAIYGLFDQTHPIDAAFVGGGKIFQSRVVKVGRYYRIYAALTARPKGNRVIYSDDFGRTWAALGGPSALPVPDGDEAKCEELPDGRVIITSRTAGGRWFNLFTYDDVKTGSGRWDEQTKATMSGMALTPSTNPTNGEMLIVPAVRTGDGKPVHVMLQSVPTGTGRNNVSIFYKELADASDMRDVRALAEGWDGCYQVSPTVSMYSSMDLQSDHRIAFFYEETLTRWGSKPNPVSTSFPKGEGEHNYDGCENVYKSLALETITAGKYRVR

SEQ ID NO: 73 - protein, 539 aa, Unknown
  Environmental sample
MRKVMSILAALLSLPTALGAADSLEVRCPQIPILLDRIDNVFFQIRVPDAKPGDVLESVTVEFGAGTDLKSVAELRLFYSGTDAVRRGGVHFSPVECISSHNVWNTRSAISSYSVLQDHTLKIGRRVTLRSGQPMVEGINYFWVSVRMSPEATVLTRLGARVSEIVVNGERKPFARTAEDVVRRMGYGVRHAGDDHAMAYRIPGLVTTRSGSLIGVYDVRWNSSVDLQERIDIGVSRSTDKGQTWEPMRIAMSFADKGGLPAAQNGVGDPAVLVDENTGTIWVVAVWAHGMGNGRAWTNSRPGMEPIRTPQLMMVRSDDDGRTWSEPVNVTGQVKDPSWRFLLQGPGRGITMRDGTLVFAIQFIGGDGMPSAGIMFSKDHGKTWNIHNPARSNTTEAQVAEVEPGVLMLNMRDNRGGARAVMTTKDLGRTWTEHVSNRTALREPVCMASLIAVPADRNALGKDLLLFSNPDTTEGRCDITVKASLDGGVTWPYSLLLDEGDSWGYSCLSMIDPQTVGILYESSAAHITFQAIELNEIVR

SEQ ID NO: 74 - protein, 887 aa, Unknown
  Environmental sample
MKNHLLLTTILCGCSLTAWSAMPIEQPWQTITMAEEVTPETPTGEVVIDVNTTTGTFVSKNPNGTWASAWKSTQTSPFFNLTCGANNMDVAKSVNGDLVAYVGNQTNSCDYMLSVERGWKITGYSFDFVRVSGKNPVTLTDAKGTALKSSDTKQSIRVDGLNASSVKAFNLSGGNDGVVISNFKVSYCIDKAYVQTDFRTFTVFDNTGSVPYRIPAIGTAQDGSLVAVADYRYSKADIGSGRVDLHLRISHDNGKTWGNVIDLEQFRGDGNLKEWRHDKAAFGDPCIVGDRESPRMMITSCSGFPGFFDATDRHQGWARWYSEDNGKTWGEPEYLDEEFIYAPLKKAGYNVQGFFVGSGKIFQSHHIKRGEYYRLYCAGSTQEGGGNKKNWVLYSDDFGKTWDFLGGTSKAPIEITADEPKVEELPNGNIILSSRDVSGRYYNIFTFSGKDGVSGSWGEKALSSSAVGGLVANNGCNGEIQIVPVVRKKDGVKTFLALQSLPTSGRTNVSIFYKDLSDATSYATPANFAKSWDGSFVVCDFSSAYSTWSWQHDNALAFLYEEGGGTSGGYNIVYKRIDIPTITEQKYDFDASYEYKRFDIDWTQADLNKEMSTLLNTAKKNSDANTVYVVSGSKLVTDAKQLECKFGHKEMGGTGGDAQDISNLIDGDPATYYHTYWGAGNQPNGSHFMDVTHPEGALFQGMIKVDLIRRSNATADHITQFTISGINGTDEPTEIAVVAVPNAAAGKEATCSFTIPAGVSYEKLRFNVTGTTNNRGYWHMAEFQLYPMSLDPKCLNALCADAYKQLVDAIATAEAVDATTVSQADVTALKTAYQSYLDALQPIVSGINGVSIAPATSGSTSIYDLQGRKLQRMDKGVYIVNGKKVVR

SEQ ID NO: 75 - protein, 434 aa, Unknown
  Environmental sample
MNKKMMLSANASKALFLAAMGCSLLMPAQMMAAQLVGDAPVMAVEAQAVNAQGGLTVFSNKEAQYRIPAIVECKSGKLIAFTDHRYHNKDIGGGRHLDIVMKTSMDKGATWSSPEQMVAKGGNGIATSFDCAHGDAAVVVDKKSGRILLMCASGGIGYWESKRGNLLMMGRYYSDDEGKTWYGEEVTKQIYDLIPEVESAFFTSGRICQSKQIKVGKYYRVYTVLCTRSGNRILYSDDFGGTWNVLGKNVAEAAPYGDEAKVEELPNGNVLLSSRAMGGRHINIYTYDDKKTATGSWDKVVASEAKNMGVAAHKNSCNGEVLMVDAKKNGKKVKLLLQSVPVGPGRNNVGIYYKALETPADYATPEAIAKNWEGCYQLSNTTSAYSTMVQGKNGSIYFLLEENAFKNPKTQTDDYYDIRFYNLSIEQITNNRYK

SEQ ID NO: 76 - protein, 1211 aa, Unknown
  Environmental sample
MLKRARLAVTLALAGGLVASGAVGVTSAFAQGGVRPDDGVTQGQPFAAGTGGSAKFRIPAMVTLDDGTIVAATDARWNTTGDGGGLDTIVSRSTDGGATWNYTFANYLGDNGNVWSGYSTAFIDPALATDGQTTWMVVDLFPAGIALNSANWAPQVGAGFDAKGRLRLRDAGQVEFGCDYYSWYAARASYDYYLDLDTLTIHKADDSKVEGYTVDGFFNITDAEGKVTNLFCSDAPFQVFPTDYLYLTKSTDGGVTWSEPTLIDVKDDDEQTLLVGPGTGTVLEDGTLMFSVYEHTSGKSELASVIYSKDKGKTWTRSSDATPSSHWSSEAVTVPLDNDTVRQFYRDGMTTLYYTDYDLKNNVWTPGQPVDSGEVKTSGNQLSAIRYSKKIDGKDAIIVSMASSGSSARTDGKLHVGLINDDQTITWQYVYDMVEPGGSYSYSTLAELENGDIAVLYENSGGTELFEVVPFADLVRDETNVDMTVTAVDLVAGEQAVLEDPSGDYTTADISELDTSVATLKLEQGEPGAPTVKPGNSSGYTGAATQISDCLYTFTKSGSGFIISAKASDGSTVYCAPGISTSAGFPNSATSSVVTLSAGYEANTVYLQDTTKGSYLYFDKSSQQLNRVNTLDNNTTWMKNCSWYLYRAAAAGEASSKELPGYVKVENPEAIEGGSYLIVSHANSGSDYVLYPTLSVGSARDQVAQAVVATNSTTVLTFTGVATGTTSVHVGATRYDVTVADYHDQAVTVEMGKTVEVTDAKGDFSGKVDDGSFNKGIASYEVKGADGATTISFTGIMPGTTTLIVGTTRYTVTVPGEVVEADQELGSTWSTTAAGAISADDLGDYDSSIIDVKLTATDGAVTGNESILLDDTAYTFTKAEDGTFTVSGATSDGKTVYLAPGTNGPGFPNATGAAAITVTPASGKNGAEGLFNLRDSGRYLYYKSGAFDRVSSPSGFEDGCTFKLYRAVDVQTLANGEGEAIEGFEQVMSVDDVTDGTYLIVAANAADGDLPVLHPSTSTTSKPAHVAYVKKGGATQADVTLTAVGTTGIRLGNQYVRFTVRAPRFTVTLDPAGGAVDQEAIEVVQGEPYGELPTPVRDGYEFQGWFDAAGNKVDAETVFEGNADATLTAQWKKAETVQPGPGTGNTGGTGNTGNTGNAGNAQKPTTQKPAKKGGTLPKSGDATNGAAVAVVALAGAAALTAGIVVQRRKHQ

SEQ ID NO: 77 - protein, 448 aa, Unknown
  Environmental sample
MSGEKVIFCGNNPDFLDKKLTTLSDGSNGGNAFRIPSLIRSGNTLIAAIDRQSCGADWGYIELAIRRSEDGGETWSDIQIIAIPPARKTMLSDECYGSAFYIDPCMAIAPNGDVLLLVDFYPECKGLHKRSILDKKKAPYALYNKEMRPTLYDRDGKFYVIDKSGHVLSHRYTDTGMTVDYESGELYSGEAYLGNIHLNGKSPSNINEAGAKTTFGAPLKAPKRSYLYLLRSSDNGKTWSKPKDITGQVLIKQDGTFLGVAPGVGLTTHKGRVIMPLYVDRKQTVSIYSIDDGENWHRMAGHPYAENVDEWQMVQAPSGAIIGLGRAKRYGKTPMSISYDDGKTWINAGKTDLYAPKCQKSVISIGDYVFCSHPNDKTRSNGVITVGKFYKAKGRVAGINWYSETEVNKGFFAYSCLTRIDDEHIGILYESEPSSFILFKKYKIKDLI

SEQ ID NO: 78 - protein, 379 aa, Unknown
  Environmental sample
MAAVIIATTASCGEKERTTPSPAPGKDDNTSVVKAPHIFVARTNGYHTFRIPALVKSRKGTLLAFCEGRKISGSDTGDINLILRRSTDGGSTWGNIITVWDDGLNTCGNPSPVVDPVTGRIHLLMTWNYQKDGQSAGDFNKDGVTKDTRRVWYTCSDDDGLTWAEPREITSSVKKDDWGWYATGPCHGIVMTKAPYEGRIVIPCDYNQKGGSGYSHVIYSDDNGATWKIGGSVKGGNESCVAETGDGRLIISCRVGGGKRMIAYSADGGETFTTGTANNSLPDPRCQGTILSAVKDGKEVLLHCNCSNSSSRTQLTLKGSTDSGATWCGGQVIWEKAAAYSDIVLVKDEIVGVLYENGDNGSYERISFERVPLGYAFIK

SEQ ID NO: 79 - protein, 465 aa, Unknown
  Environmental sample
MSQTTKRVFTRKSKLFLILASMILALSCKNPLGDESGGSGGAGGGGSSGGQTITNEQGKVFPAWYLTEQQQQQDFSMGPTILFQSTKAPTQHYRIPAIIVADNGNIIAIADNRYNHGGDIGAVKGLIDVVYKISRDGGKNWSDEKIMGNRSTDHNYSKALNKGDALVFRANDGTLVCMAVSGGGFANATVSTPSRMLRSESKDNGETWSDWKEVGQSLIDKIKNSFGKGIPKAFAPAGRGLTLKDGTFAAAMIGQVNNGGFYGLYIYSTDKGLNWNCADNFIASTGGHGSWNEPKVIAELNDGKLLMSVRNGMSTANKNDPRMYAVSTDAPVNGKCAWPNSLSAWTLIRCGGVDAEGVVWTREREQDKNRMLHIQAGPYGRHGLKLFLSTEEGKTDNSWTEAKVILGGQTGASYSSLDVCGDGTIVTLAEEKSKTGQHYDIVFRRYNMKAITQEVYKTKWYKEVK

SEQ ID NO: 80 - protein, 1288 aa, Unknown
  Environmental sample
MKNRKRLLSLLMAGVLTFGCFPVTAGAANNSKDGAKPADGTTAEQPFPKTLFLEEHNSSSGFTRFRIPALTTAFDGTLIAATDIRWDICGDGGGLDTLVSRSSDNGATWNYTVANYLGDNGNKFNRYSTAFIDPSLVVDGNTIYLACDLFPGGVAVASAASTPSQAGSTGYDSEGHLLLAATTTSVNGISSSTLRAAADYAYHLEKKADATAADYYEIKANSTGEVVDGEYTIDDHFNITNADGTVDTNLFCGDTPYFQYPTDYIYIVKSTDNGATWSAPTLANVKNAEEQVFLIGPGRGITTSSGRIIFPCYQYTNGVQRSATIYSDDHGVTWKRGSTVTGNSSEDVIVEADGKLYMFVRMSNVYYVSEDDGTTWSEPKSMGIKFHNNCQLSAITYSKKVNGKTAIVFAGPSDAGGRNSGRIWLGLVQEDGSIEWQDNPYVVTEGTHYAYSCITETADGDIGLVYEYEDEKIKFEKISFADIAGEATTSRVWVENEKGAIVNSAVMKSDATASYIVKKLDADTEVTVSSSNRAVLEASYTDGKLILTSKAGVTGLKQVKVTVKAGEETFVVNVNVTDAENYKVINLEKDETKTVTVKGANYADADTSSVDTEVAGVTVTGKDVQDTYEKTVAQLATAAATFDGSKVDLSKCMYTLGAGSSDSTFKVKAATANGSSIYLGPKASSTAGIPNITTSQPQITFTKNASDATFSLMDNTSGTAGRYLYFYHTDATKLYFDRYGTADEHCWFELYTPSKDAKDYDLINGFKKVESLKELTAGSSYLIVTKADASGNRYILLPSTGTQKYNHVAKLVNETVSATEEAAYVATDVATFEEKSAKGLSECLYTFKKMSDGTYAISGHAADGATTFVTIGTVGIPNKSEKVDITVEDIADSNGKVSLYDGTASRYLYFWRDTAKLYFDRYGTQDTTACAFELFKKSATASSDSPIKGYEKLNGVSEIEDGRQYLIAAQVDGTYYVMNPVLGDSKFSHVAKVTNTTYRNPAATASTTIDFTGVAAGTTSVKIGDVVYYVIVTEDGAECTHKTTVVLDAKDATCTEPGSTGEVVCVDCGAVVQEAQEIAAKGHTFGEWVTVKEPTTKEEGSKERICSVCQTKETETIAKLPDTEDPDQPTTPSAPENVKDSEVTKDSIKITWDAPASTEGIAGYKIYVNGAVYATDISRDAVGYTLSGLKAGETYEIQVVAVGTDENATEYAAAAFSVTTKSEDKNDNHGGNDNKNATTTDKNQNSTVNNANKAAKTGDTANVVFPAAVMMLAGAAVAGILFRRKWMK

SEQ ID NO: 81 - protein, 453 aa, Unknown
  Environmental sample
MKSKKISKLFYLSFILIFWILIFTLTCKNPSGPGIDFNLRNWYIMSGMAKGPGFTSNITYGNNTIIYPSNYMKKEKDMVFSNGTYNYRIPELLALPNGDLLAFADRRYNGAGDLPNRIEVYVKKSTNNGTNWGAEKQVTPTSLSKSDGYGDTAAVLDRKTGNVIAVVAHGQGFFQSTSKDPIRIITIRSRNNGESWEAPIDITSQIYGAGCGDLTRRNWQAAFVTSGNGLQMRNGRIIFVLNVRQTTSAFVENYILYSDDGGYKWRVSRGSPKNPKGGNEAKVVELNDGSLLMHIRWTPNRLQSRSYDYGETWTEATVINGIPASHSNGDLIVYTSTKNGYDKDRLITLVDRRPWGNNQAKCSSCKSGGNCTPGYPGCPTFYVSYDEGKTWTNSRRLYTGHAGYSSLAILKDGSIGILAELGNSWNGPIYFLKTSIEWCNPNDNPCSPTNAKK

SEQ ID NO: 82 - protein, 293 aa, Unknown
  Environmental sample
MSASALPWSQQQLTVAPHPYQLVKSDGVYLHLDAKVTGLGGASCGQGGPLKPDRTLSDSYSFGFAIRPVTNGNAPAVVNASLSGMRPITIVRNAKGDVTLRSADASRTIMYSVSGKKKAQVYTQPFNFLQEGTVTAWYKDSPYFKMNMSFDKITSIPLDVMFCSSFEPKEGEATFLVDGNPNTYWHTMYSVTVANYPHWIDFDASQTKTLKGFVYQPRQDSANGRIKDYEIYVSNDGKNWGSPVNKGSFKNSSETQRIMFDKPVKARYIRLRALNEQRGQDFASGAEFSLIAE

SEQ ID NO: 83 - protein, 457 aa, Unknown
  Environmental sample
MKHKERGNTHMAKEQLIFCGSSETFLDKALTADSNGDNGGNAFRIPSLVNADGTLIAAIDRASCGADWGYIELAVRRSEDGGKTWSPIETIAAPPARETKISADCYASAFYIDPCMAVAPNGDVILLADFWPECKGLHNQRLLDKHKVPYAQLSGRMRPLIYDRDGNFYYIEESGAVLDSQKQPTPYRLGSGFGDLYKGDEYVGNIYLNGAVGKNEAGAVTTFGAPLKAPKRCYVFMLRSSDRGKTWSDPVDITNMVLHETDGTFLGVAPGAGITTTDGRIIMPLYVDRKSTVSIYSVDNGDTWHRMTSQPYAENTDEWQMVEAPDGSIVGFGRQKRFGKTPMSISANKGKSWSKCGKTGLYAPKCQKSVIAVGDTVFCSHPSGHTRENGMLSVGKLRRKKGGYTHVDWVRHVPINRGFFAYSCLAQIDAHTLGVLYEDRPSSHIQFQTFELAELMG

SEQ ID NO: 84 - protein, 548 aa, Unknown
  Environmental sample
MRNTFLLIGMLALSSSVYAADTLFVRAPEIPVLTNQQDNALFEIRVNADKGDVLNQLDISLDAEDLSEVEAVRLFYSGTEGVSRRGVHFSPTDNQSYSSPSVLLGKSGQATLKEVFADYSVKQDEKSSPGKRLALSSEQPMLKGINYFWVSLQMKPKTQLSHTITAAIESAEINKSKAVVAGNNLSPKRRMGYAVRSAWDDGVAAYRIPGIVTTNNGTLVGCYDVRYHSSVDLQEKIDIGVSRSEDKGQTWEPMQIAMTFKNEGGLPHAQNGVGDPSILVDEQTNTLWIVAAWCHGMGNQRAWFNSRQGMTPDETAQLVLVKSTDDGKTWSKPINITKQVKDPSWHFLLQGPGRGITMHDGTLVFPIQFIDSERMPHAGIMYSKDRGETWAIHQPARSNTTESQVAEIAPGVLMLNMRDNRGGSRAVSITKDLGKTWEEHISTRTALQEPVCMASLIHVDAKKNILGKDLLLFSNPNTTANRNHITIKASLDGGVTWLKSHQILLDEEDGWGYSCLTMVDEETVGILYESSVAHMTFQVVKLKELVKE

SEQ ID NO: 85 - protein, 444 aa, Unknown
  Environmental sample
MSDSKIIFCGNSKDFLAEELTKASNAENGGTAFRIPSLVNADGTLVAIADKAATGSDWGYIELAVRTSADGGKTWTPIKTIAAPPAREINSSVENTKTAFFIDPCAVVAPNGDVVVLATFFPESKGIHDKKILEPKKVAYADFNGKNCQLIYDREGKFYYVLDDGSVLDSAKSKTDYTVKGIGELYKGSEYVGNIYLNGEMGKSEDENGKTTFGAPLKAPKRSYVYMLKSSDKGATWSEPVNITGAVYNFDDGAFFAVAPGAGLAVDNRIIMPIYTLKGTVCIYSDDNGATWQRNPQFQYTDNVDEWCAVKAADNKIYAFGRAKKYAATPVTVSEDNGLGWYRTKNAKFKAPKCQKSAVAIGDKIYVAHPSGKKRENGIISVGTLTYNKKGKFKGIVWTKDKDSVKINDGFFAYSSMAKLGDNKIAVLYEDQPSSHIVFDVINL

SEQ ID NO: 86 - protein, 425 aa, Unknown
  Environmental sample
MNKSILRFAFILLVSFLFTVGTKAQTAGAAISKANLPTPVTVFDDPYQGIPVRIPAMTITRDGGVIAMVDYRHNFSDIGWNSGGTYRLDILMRRSDDGGRTWSPINTVVEGRKDFGYGDPSVVADRDSAGLILMQAVSGNVPYVHATAKNPQHAMFFRSTDGGKTWDKGHDVAPSIYNLYRDEQPLTQEKGFFLTSGKITQSRIIKKDKYYRLYIAHPVLHVGTFVIYSDDFGKTWQVLGGAATIPGPDGDESKVEELPDGSVLLSCRKFSSLGRRFNVFVYKNKKRAEGAWSEAKDAKALANCTRCNGDVVVVPVRRTIDGRKTWLAIQSLLQSKTRDHLGFYYREIPRRGADLAPALADNWQRGVMIHEGTAAYSTMSVFDDGTVGILYEADLDANNTGYKILFSRLSVSQITGGTYEAMPAK

SEQ ID NO: 87 - DNA, 1164 bp, Unknown
  Environmental sample
atgcgtatattgaaaaacacattgttagcaggacttatgcttctcttttcttgtatgaaaggagaagcacagtccggtgctaaaaacactttttctgagtttcctaaatacaccgtgttttctgccggggatgataatgtgaatagttatcgtatcccttcattattgatagttaaagatggttccttacttgtgttttgtgaagcacgtagagaaagttggcaagataaaagtcgtacagatgtagtagtgaagcgtagtacagataatggtcagacatggtcggttatgcaggatttgacaaaaggaatcactggtgcttatatggatccaactcccattttggattcaagtactggcagagtctttctttttacgactttctggccggtggatgatcattcaggagctggcaaccgtgccattcttattacttcggatgataatggtcagacatggtctactcctgccgatgttactgcatctttaatgccagatggacgttatatatatggttttggtccgggagcaggattgcagatggtaggtgagaagtacaaaggacgtttaattctgccagcccgtatttctgatatggaaagaaaaacagcacacgacgttgctattttttctgacaatcatggtgaaacgtggacggtaggagagaatggggatacagataatgagttccagattgcagaatctccaaatggagttttagtgtataatgctcgagtatcaggtgcacgtatggtagcatatagcaaagatggaggcatgacttggagtaaagcaatgaaagagtcggctctgcctggtgtgtctaaaggctgccaagctagtgtgttaggggaaaatgcaaatctttatttctccggaataaagggaattccggaaacaccgcaatatgacgaaagagctggattgactttatataaaagctgtaatggcggaagaacttggaatgatggtctgcttttatgtgacaaagcttccggatatagctgtatggctttactaccagatgaacgaatggcgataatctttgaaacagcagatacgcaatcgtttactcgcaaatcattacctaatataaaacctcttaaacgtccggctgattggatgaagctagatttaattttagtgcctattattaatctttaa

SEQ ID NO: 88 - DNA, 1110 bp, Unknown
  Environmental sample
atgaagaatttaaacttaatatttgtatgggtgtgcctgttgtttatatcgacagcatgcaatgatgtggagccgagtatatcttcttttccggttcccacaagcaagccttattctatcaatagggaaggatatgcttatttcagaattcctactatggtgattactaatagtggaacgatattggcatttgccgagggacgtcgtaatggtcctgaagatgaaggggatatcgacatcgtactaaagcgttctactgataaagggaagacttggggaccattgataacggtcaaggatgatggcgaaaatcgttgccgtaatcaagtgcctatttatcttccggatatcaatcgcgtcattttattgtcttgctggaatccgggagcaactgggactaattccatatttatgacttattcggatgatgagggattgacatgggcaaaagaaaaagacataaccgcttccgttacccctgacaaatacagatggtatgctacaggaccttgccatggaatagtaaaacagtatgaacctcataagggaagaatcgtggttccgtgtaatcacaataccactacttccggtgcggggcgttctcatgtgatatattcggatgataaaggggaaacctggcatttgggtggtatcatggacgtggactatacgaatgaaagtaccgtaaccgaactaagtaacggtgatttaatgctgaatatgcgtaagcagtccaatacagagaaatatagaatggtttccactagtacggatggtggactgacatggagtccgtgtaagtacactacattgaaagagccgatatgtcagggttccatactcttttacggtttgggagatgatggtaaaggaattattttgttctcgaatccggatagccagaccaatcgcaataataatacgttgaagatgagcgaggacgacggcgtaacctggaagaagcaatattcttatacaggcagtgattatggaggatattctgatattgcccgttatcctgacggtacgattggagtattatatgaatatggattcaagaatataggcggcattgcttttcagaatgttgagttgtcacagtttcaataa

SEQ ID NO: 89 - DNA, 1641 bp, Unknown
  Environmental sample
atgaaaatgagtaagaaaatatttatcatattttcgatgattttgctatggataccagatatatctttgggtaaagatataaggttcgaactggaaaggcctgtcatccctgttttggtcaaaaagcagattaatccgactgttaaggcaactttgattcaaacaaacagccgtccttacactatccggcagattgatgtcgacttgcaagaaagtaccgacttgtctgatatcctttctgttgcggtatatggaactcataaaaacgggctgatagatgaatcccgtctgatatgtaaaccgatgcccgctgcacggaagatatcttttacagataatatacaagtgaaagatgattccttgtctttttggatagcagtgacattgaaggatacggttccactgacacaccgcattagtgtgaattgcagccggataaaaaccagccggggagaactgaaagtctcgaataaggaggctattcccctaagggtgggagtggcattaagacagaaaggacaggatggtatcgtttcttcacgtattccgggattggctaccagtaacaacggaactctgctggctatatttgatgcccgttatgatttggctcgcgatttgcagggaaacatagatatcgctcttcatcgtagctttgacaaaggactgacatggcaacccatccagacggtgttggatatgggagaatggggaggacttccgcaaaagtttaatggagtgagtgatgcgtgcatccttgtggataaaaacacaaatgatatttatatagccggattgtggatgcacggtgcattggatgataatggcaagtggatagagggcttgaatgaaaacagtacttactggatacaccagtggcgaaagaagggctcacaacccggaatcggtctgaaagagacctgtcagttcctgataactaaaagcacggatgacggactgacatggagtttccccgataatataactggtaaaaccaaacgtcctgaatggtggttgtttgctccggctcccggacaaggaataacactggcggatggtactttggtctttcctacacaaggacgtgatgataagggagcagcattctccaatattacatatagtaaagaccacggaaagacatggacaaccagcaatccggcttattcgaatgtgacggaatgtaatgcagcccaattaagtgacggtactgtaatgctgaatatgcgcgataacagaaaccgggggaataaagaagtgaatggccggagaatctgtacgacaacagacttgggcgagacatggacagagcatctcacgtcacgaaaagcattggtggaaccgacgtgtatggcaagtttacatcgccatgattatactgtggacggagaaaagaaaagcatactattatttgccaatcccagtgattataagacacgggataaattgaccttgaaagttagttttgacgacggaatgacatggcctgagaaatactggattctgtttgaccaatatcgtagtgcaggttattccagcatcacgtcaatagatgaaaattcaatagggatattatatgaaagcagtcaggcaaatatggcttttattaaaattgatttggcggaaattttaaacaggtag

SEQ ID NO: 90 - DNA, 1632 bp, Unknown
  Environmental sample
atgaaaaagaatttatttttatcaatcatcttttctttctgtgtgattttgcaggcttttgcgtcagatactgtatttgtgcgtgaaactcagattcctgttttgattgaacgacaagacaatgtgttgtttatgctgcgtttgaatgcaaaagagagtcatactttggacgaagttgttttgaattttggcaaagatgtaaatatgtctgatatacagtccgttaagttgtattatagtggtacggaagccagacagaactacggtaagaatttctttgctcctgtatcttatatttccagccatacaccgggaaagacattggcggcgaatccttcttattcgattaacaagtcgcaagtgaataatccgaagcggaaagttgcgttgaaggctaatcagaaacttttccccggaatcaactatttctggattagtttgcagatgaagccggatgcatctttgttggataaggttgcggctaagattgctgcgattaaggttgataacaaagaagcccttatgcacactgtttcaccggagaatatagttcatagggtaggggtaggcgtgcgtcatgccggtgacgacggttcggcttccttccgtattccgggattggtgactaccaataagggaacattactgggagtatatgatgtacgttataataacagtgccgatttgcaggaacatgtagacataggtttgagccgcagtgtagacggcggtaaaacctgggagaagatgcgtttgcctttagcttttggtgagaccggtgatttgcctgctgcacagaacggtgtcggcgacccctcaattctagtggataccaagaccaacacagtatgggtggtggcagcgtggacgcacggaatgggtaaccaacgtgcctggtggagctcatatccgggaatggatatgaatcatacagcccagttggtgttgtcaaagagtacggacgacggtaagacatggtctaaacccatcaatatcacagagcaggtgaaagacccttcctggtatttccttctgcaaggcccgggacgtggcatcaccatgcaggatggtacattggtatttccgattcagtttatcgactctacccgtgtcccgaatgcgggaatcatgtatagcaaagaccgtggcgagacatggaaaatccataactacgcacgtaccaatacaacagaagcccaagtggcggaagtagaaccgggcgtgttgatgctgaacatgagagacaaccgtggcggtagccgtgcaatctccactacaaaggatttaggcaagacatggacagaacattcctcttcccgcaaggctttgcaggagccggtttgtatggcaagcctgattagcgtgaaagcgaaagacaatgtattaaacaaagatatccttttgttctccaacccgaatacagttaaaggccgccatcatatcactatcaaagcaagcctggacggaggtgtcacatggttgcccgaacatcaggtgatgctggacgagggtgaaggatggggatattcttgcctgacaatgattgataaggagacaattggtatattatatgaaagtagtgtagcccacatgactttccaggctgtccaactaagggatataataaaatag

SEQ ID NO: 91 - DNA, 1275 bp, Unknown
  Environmental sample
atgaaacacttatttaatgttttagctttaggcattggattgttcttttttaatgcctgctcggatgataatgttaacgacttcaggccggtaccttatccggacgaagtagtgggtctaaatcctgatcctgattttgaaccgatatgggagatagcttctccaacaattactgtattcagttcgaaagcaagtaatgacatttcatatcgtgttcctgctatcgcggttacaaagaaaggtagcatactcgttttttgtgaagctcgctatggaacttggcaagacaaagctggtcggacggatattttgatgaagcgtagtactgataaggggataacatggactgaaaagaatttaactaatcaagctacttcgtcgaagttgtcatatatggacccgactgttgtcgttgaccaagttacagggaaaatctttttgtttacttctttgtgggatgctgtaggcaaggagtctgccaaacaaggctataataatagagctatcatgtacacttcggaagatgatggattaaattggacaagaaaagatctcacagacgaggtggaaattggtatttttagcggtgcgacaagaatgattggtagcttcggtcctggctccggagtgcaaatgacatcaagtgagcaatataagaatcgtctgattgtccctatacgcactttcaaagtgaatgaagccgcagggaccgtttcaaatggcgggaatacagctatgtggtcggatgataacggggggacatgggaaaccggacaacctaataagtctggtgaatggatggtaacggaagctcctgatggtgcattaattggaaatatccgttataatggacaccgtcaaaattatgtaagtacagatggtggggccaaatggccttctttttcagattatgatccgatagcactaccaactccagccaaaggctgtgcgggaagtgttatcgtaaaggatggctggatgtattactgtggggcgaaagggattatcgagacaacagctcatgatgatcgtggtatactatatttggcgaaagccaaattctttggtggccattcgcatacattcgatccggcagatcatatggtgctttatgacaaagcggcaggatacacatgtatggcacttctgcctgatggtgatatggctattgttgccgaactcggtaatgaaccgggattccaaaaactatcaactcgtccggccgaatggatgagattggaattatttatattatctacaaaaaaacctctttaa

SEQ ID NO: 92 - DNA, 1620 bp, Unknown
  Environmental sample
atgatgaaaagaaccttttggctcattgccttgtcgttgctgtcgttgacattgtcggcatcagattcgttgaggatccataatcctcagattcccatcctgttggatcgcatggacaatgtcctgttcctgatccgtgtccctgacgctgtgcaagggaatgtgcttgaatccttgaccgtggagttcggaccggacactgatctgaattccatagccgagctgcgtcttttctattccggaacagaggcagtgcggcgtcaagggctgcgtttcagcccggtggaatatatctccgcccataatgtgtggaacacacgttccgccaatccgtcatattcagtgatgcaggagcaggcctcgaagatcgggcgcaaggtggttcttcattcccgccagcctatggtcggtggcatcaattattattgggtcagcgtccggatgaatcccgatgcgtctttgctgacagagttgcgcgcgagggtgtctgaggttgtggttaacgggaagaaaatccctgtggcctgtgattcgagggaggttgtccgcaggatgggctacggcgtgcgtcacgcgggtgacgaccattcgatggcttaccgtattccgggcttggtgacaaccaactcgggaagcctgatcgggacctatgacgttcgttggaacagcagcgttgaccttcaggaacgcatcgacatcggtgtgagccgaagcacggacaaggggcagacctgggaaccgatgcgtatcgctatgtctttcgcggacatcgacggactcccttccggtcagaacggggtaggggatccggcggttctggttgatgagaaaacgggaaccatctgggtgatggccgcttggactcacggaatgggtaacggcagagcctggactaactcgatgcctgggatggaaccgatggagaccccgcagctgatgctggcgcgcagcgatgacgacggacgcacttggagtgagcctatcaatatcacgaagcaagtcaaggacccgtcctggtgcttcctcctgcaaggtcccggacgaggcataacgatgcgtgacggcacattggtgttcgcgatccagttcatcgacaaggacaggatgccgcacgccggaatcatattcagcaaggaccatggcgagacctggcatattcataatcccgcccgttccaacacgacggaggcccaggtggctgaggttgagcctggagtgctgatgctgaatatgcgggacaatcgtggcggtgcgcgcgccgtgatgacaacccgtgacctgggacgcacctggaccgagcacgtatcgcacaggacaaccctccgcgagccagtctgtatggccagcctgatcgcggttccggctgagaaaaatgtgttgggcaaggatttgttgctgttctccaatccggacacgaccgacggccgccgcgacatcacgatcaaggccagtctcgacggtggagtgacgtggccatacagcctgctgctcgatgaaggggacagttggggatattcgtgcctcactatgattgattcacagacagtcggcatcctttacgaatcaagcgtggcgcacattacatttcaggcggccaaattgaaagcgattgtaaaatga

SEQ ID NO: 93 - DNA, 1122 bp, Unknown
  Environmental sample
atgaaacgtaccggttggctgctgctgctggtttctggtccggcgctgtgccagccgccgcgtggttactctatcccgctgatcgacctggcggcggaagcgtggcgtcagaccgttgttgaccgtgaaccgggtcagtacctgggtcacccgaccaccgttctgctggaagacggtcgtaccgttctggcggtttacccgaaaggtcacggtcgtggtgcgatcgtttacaaacgttctcgtgacggtggtcgtacctggtctgcgcgtctgccggttccggaaaactgggcgacctctcaggaaaccccgaccatccaccgtgttgttgacccgcgtggtcgtaaacgtctgatcctgttctctggtctgtacccgatccgtatgtctgtttctgaagacgacggtgaaacctggaccccgctggcgccgatcggtaacttcggtggtgttgttgcgatggcgtctgttgaacgtctgcgtgacggtcgttacatggcgctgttccacgacgacggtcgtttcctgcgtggtggtggtaaaccggaccgtttcgttgtttacaaaaccctgtctggtgacggtggtctgacctggtctgaaccggttccgatcctgtctcacccgcaggcgcacctgtgcgaaccgggtctgctgcgttctccggacggtcgtcgtatcgcgatcctgctgcgtgaaaactctcgtaaattcaactctttcgtttctttctctgacgacgaaggtgaaacctggtctgaaccgcgtgaactgccgggtgcgctgaccggtgaccgtcacaccgcggtttacgcgcgtgacggtcgtctgttcatctctttccgtgacaccacccacgaatctccgacccgtggtgactgggttgcgtgggttggtcgtttcggtgacatcgaaaacggtcgtgaaggtcagtaccgtgttcgtctgatgaaaaaccacaaagacctggactgctgctacccgggtgttctgcgtctgccggacgacaccatcctgaccaccacctacggtcactggaccccgggtgaaccgccgtacatcgtttctatccgtctgcgtctgtctgaactggaccgtcgtgcgcagggtgcgggtcgttaa

SEQ ID NO: 94 - DNA, 1176 bp, Unknown
  Environmental sample
atggttatcgcgtggatggcgggtgttcagtgcaccgcggcggcgctggttgacctgggtgttaccatcctggacatctctggtgaaccggaacgtcaggttatcgttgaccgtgaaccgggtcagtacctgggtcacgtttctaccgttctgctggaagacgacaaaaccatcctggcggtttacccgaaaggtcacggtcgtggtgcgatcgttctgaaacgttctgaagacggtggtcgtacctggtctggtcgtctgccggttccggcgtcttgggcgacctctaaagaaaccccgaccatccaccgtgttgttgacgcgcgtggtcgtcgtcgtctgctgctgtggtctggtctgtacccggcgcgtcgtgcgctgtctgacgacgacggtcgtacctggaccgaactggaaccggttggtgaatggggtggtatcgttgttatgggtttcgttgaagcgacccgtcagccgggtcactacgttgcgatgttccacgacgacggtcgttacttcgcggcgcagccgtctaccaaccgttctatgaccctgtacctgacccgttcttctgacggtggtgtttcttggtcttctccggttgcggtttggtctaactctgcggttcacctgtgcgaaccgggtggtatctggtctccggaccgtcgtcgtctggcggttctgctgcgtgaaaaccgtcgtgttaacaactctcacatcatgttctctgacgacgaaggtcacacctggaccaccccggttgaaatgccgctgtctcgtgcgggtgaccgtcacaccctgcgttacaccccggacggtcgtatcgtttgcgttttccgtgcggttaccccggttggtatgcgtggtcgttctttcggtcagaacgaagacgttgacaccctgggtgttggttctccgttcgaaggtgactgcgttgcgtgggttggtacctgggacgacctggttcacaaccgtccgggtcagtactacgttcgtctgctgaacaaccgtaaaggttgggacaccacctacccgggtgttgaagttctgccggacggtaccgttgttgttaccacctacggtcactggaacgcgggtgaaccgccgtacatccgttctgttcgtttccgtctggaagaactggaccgtatggcggcgaaagcgtctcagaaaccg

SEQ ID NO: 95 - DNA, 1215 bp, Paeniclostridium sordellii
atgaaaaaatttattaaaattttgaaagtattaagtatggcaattgttttgtcagcatgtaatatcaatggtatctttgcaagtaatttaaacacaactaatgaacctcaaaaaacaactgtatttaataaaaatgacaatacgtggaatgcacagtattttagaatacctagtcttcagactttggcagatggtactatgctagcattttcagatattcgttataatggagcagaagatcatgcatacatagatattggagctgctaaaagcacggataatggacaaacatgggactataaaacagtaatggaaaatgatcgtattgattcaactttttctcgtgtaatggattcaacaactgttgttacagataccggaagaataatattaattgcaggatcatggaataaaaatggaaattgggcaagtagtacaacttctttaagaagtgattggtctgttcaaatggtttattctgatgataatggagaaacatggtctgataaagttgatttaactacaaataaagcaagaataaaaaatcaaccaagtaacactattggatggttagccggtgttggaagtggtattgtaatgtctgatggtacaatagtaatgccaatacaaattgcattaagagaaaacaatgctaacaactattattcatcagttatatattcaaaggataatggagaaacatggacaatgggaaataaagttcctgatccaaaaacttctgaaaatatggtaattgaattggatggagctttaattatgagttctagaaatgatggaaagaattatagagcatcatatatatcttatgatatgggatctacttgggaagtatacgatcctttacataataaaatatctactggtaatgggtctggatgccaaggttcatttatcaaagttactgcaaaagatggacatagattaggatttatttcagcaccaaaaaatacaaaaggtggatatgttagagataacataactgtttatatgattgactttgatgatttatctaaaggtattagagaactatgtagcccataccctgaagatggaaattcaagtggtggtggatactcttgtttatccttcaatgatggaaaactaagtatactttatgaagctaatggaaatatagaatataaagatttaacagattattatttatctattgagaataataaaaaattaaaataa

SEQ ID NO: 96 - DNA, 1167 bp, Brucella abortus biovar 1 (strain 9-941)
atgttcagtcttattcgcgcaaaacgactggcgatcggtatcgcggctcttgcatggtctacaggtgccgtgatggcatctgaacattggccggatctgcccgtggggatcaagaatggtgcagccgcccggatcggcaatatggcctatgtcggcctcggctccgccggaactgatttttatgcgcttgacctgaacaatccctccaagggctgggtaaagcgggcaaacttcatcggccccgcaacaaacggagcagcaatggctgcggcaggcggaaaaatcttcgctttcagcggtaatggcaaggcaacgccggatgcgaaatcccccatcattttcgatacggcctatgtttatgatccgggttcggatggctggtccaagctcgacacgcaaacgccggttgggctttccggcgcaaaagccgttgggcttgctgacggccggattgccatctttggcggatataataaggaactcttcgacaaatatctggctgatgtgggcgcgatcgataaagacaaggagccggaagcttacaggaatctggtcgattcctatatgggcatgaagccggaggcctatcgatggaacgacgaggttctggtttacgatcctgctggcaataattggggttcgcttggcgccaatccgtttctgcccaattgcgatccggcaatggcaacaatgggtgagggtgattttctgcttgtgagcggtgagatcaaaccgggcctgcgaacacctgaagccaaattggtgaaaatccgggatggtgcggcgcactggcagaaagtgagtgatcttccgccgctctccggttccgaaccacaggagggagtggctggcgcctatgccggaaaggctggcgatgatgtgcttgtcgctggtggagcgaatttcaagggtgcacaggccaatgctgcggctggaaaatggtttgcgcatgacggccttgcgaaaagctggcgtgatgatgtctatgcttttgatggcaaggactggaaggtggccggcaagctgccgcgcggtctggcctatggcgcagccttcgatgcgccgggcggtcttctggtcgtgggcggtgaagatagagacggcaaggcccgcaaggaggttttcctcctcaaatgggatggcaaggccctctcggtcgaaaactga

SEQ ID NO: 97 - DNA, 1185 bp, Brucella melitensis biotype 1 (strain 16M)
atggagtggaggaaatgcatgttcagtcttattcgcgcaaaacgactggcgatcggtatcgcggctcttgcatggtctacaggtgccgtgatggcatctgaacattggccggatctgcccgtggggatcaagaatggtgcagccgcccggatcggcaatatggcctatgtcggcctcggctccgccggaactgatttttatgcgcttgacctgaacaatccctccaagggctgggtaaagcgggcaaacttcatcggccccgcaacaaacggagcagcaatggctgcggcaggcggaaaaatcttcgctttcagcggtaatggcaaggcaacgccggatgcgaaatcccccatcattttcgatacggcctatgtttatgatccgggttcggatggctggtccaagctcgacacgcaaacgccggttgggctttccggcgcaaaagccgttgggcttgctgacggccggattgccatctttggcggatataataaggaactcttcgacaaatatctggctgatgtgggcgcgatcgataaagacaaggagccggaagcttacaggaagctggtcgattcctatatgggcatgaagccggaggcctatcgatggaacgacgaggttctggtttatgatcctgctggcaataattggggttcgcttggcgccaatccgtttctgcccaattgcgatccggcaatggcaacaatgggtgagggtgattttctgcttgtgagcggtgagatcaaaccgggcctgcgaacacctgaagccaaattggtgaaaatccgggatggtgcggcgcactggcagaaagtgagtgatcttccgccgctctccggttccgaaccacaggagggagtggctggcgcctatgccggaaaggctggcgatgatgtgcttgtcgctggtggagcgaatttcaagggtgcacaggccaatgctgcggctggaaaatggtttgcgcatgacggccttgcgaaaagctggcgtgatgatgtctatgcttttgatggcaaggactggaaggtggccggcaagctgccgcgcggtctggcctatggcgcagccttcgatgcgccgggcggtcttctggtcgtgggcggtgaagatagagacggcaaggcccgcaaggaggttttcctcctcaaatgggatggcaaggccctctcggtcgaaaactga

SEQ ID NO: 98 - DNA, 1167 bp, Brucella ovis (strain ATCC 25840)
atgttcagtcttattggcgcaaaacgactggcgatcggtatcgcggctcttgcatggtctacaggtgccgtgatggcatctgaacattggccggatctgcccgtggggatcaagaatggtgcagccgcccggatcggcaatatggcctatgtcggcctcggctccgccggaactgatttttatgcgcttgacctgaacaatccctccaagggctgggtaaagcgggcaaacttcatcggccccgcaacaaacggagcagcaatggctgcggcaggcggaaaaatcttcgctttcagcggtaatggcaaggcaacgccggatgcgaaatcccccatcattttcgatacggcctatgtttatgatccgggttcggatggctggtccaagctcgacacgcaaacgccggttgggctttccggcgcaaaagccgttgggcttgctgacggccggattgccatctttggcggatataataaggaactcttcgacaaatatctggctgatgtgggcgcgatcgataaagacaaggagccggaagcttacaggaagctggtcgattcctatatgggcatgaagccggaggcctatcgatggaacgacgaggttctggtttacgatcctgctggcaataattggggttcgcttggcgccaatccgtttctgcccaattgcgatccggcaatggcaacaatgggtgagggtgattttctgcttgtgagtggtgagatcaaaccgggcctgcgaacacctgaagccaaattggtgaaaatccgggatggtgcggcgcactggcagaaagtgagtgatcttccgccgctctccggttccgaaccacaggagggagtggctggcgcctatgccggaaaggctggcgatgatgtgcttgtcgctggtggagcgaatttcaagggtgcacaggccaatgctgcggctggaaaatggtttgcgcatgacggccttgcgaaaagctggcgtgatgatgtctatgcttttgatggcaaggactggaaggtggccggcaagctgccgcgcggtctggcctatggcgcagccttcgatgcgccgggcggtcttctggtcgtgggcggtgaagatagagacggcaaggcccgcaaggaggttttcctcctcaaatgggatggcaaggccctctcggtcgaaaactga

SEQ ID NO: 99 - DNA, 1167 bp, Brucella suis (strain ATCC 23445)
atgttcagtcttattggcgcaaaacgacaggcgatcggtatcgcggctcttgcatggtctacaggtgccgtgatggcatctgaacattggccggatctgcccgtggggatcaagaatggtgcagccgcccggatcggcaatatggcctatgtcggcctcggctccgccggaactgatttttatgcgcttgacctgaacaatccctccaagggctgggtaaagcgggcaaacttcatcggccccgcaacaaacggagcagcaatggctgcggcaggcggaaaaatcttcgctttcagcggtaatggcaaggcaacgccggatgcgaaatcccccatcattttcgatacggcctatgtttatgatccgggttcggatggctggtccaagctcgacacgcaaacgccggttgggctttccggcgcaaaagccgttgggcttgctgacggccggattgccatctttggcggatataataaggaactcttcgacaaatatctggctgatgtgggcgcgatcgataaagacaaggagccggaagcttacaggaagctggtcgattcctatatgggcatgaagccggaggcctatcgatggaacgacgaggttctggtttacgatcctgctggcaataattggggttcgcttggcgccaatccgtttctgcccaattgcgatccggcaatggcaacaatgggtgagggtgattttctgcttgtgagcggtgggatcaaaccgggcctgcgaacacctgaagccaaattggtgaaaatccgggatggtgcggcgcactggcagaaagtgagtgatcttccgccgctctccagttccgaaccacaggagggagtggctggcgcctatgccggaaaggctggcgatgatgtgcttgtcgctggtggagcgaatttcaagggtgcacaggccaatgctgcggctggaaaatggtttgcgcatgacggccttgcgaaaagctggcgtgatgatgtctatgcttttgatggcaaggactggaaggtggccggcaagctgccgcgcggtctggcctatggcgcagccttcgatgcgccgggcggtcttctggtcgtgggcggtgaagatagagacggcaaggcccgcaaggaggttttcctcctcaaatgggatggcaaggccctctcggtcgaaaactga

SEQ ID NO: 100 - DNA, 1107 bp, Escherichia coli O139H28 (strain E24377A)
atgaataaaacaataacggcgcttgctatcataatggcttcatttgccgcaaacgcgtctgtattaccggaaactcctgtgccatttaaaagtggtaccggagcaattgataacgacactgtctacattggtttaggtagcgcaggtacggcatggtacaagctggatacacaggccaaagataaaaaatggacagcgttagctgcattccctggcggaccaagagatcaagcaacctctgcatttattgatggcaatctgtatgtgtttggcggcattggcaaaaacagcgagggcttgactcaggtatttaatgatgtacacaaatacaaccccaaaaccaatagctgggttaaattgatgtcgcacgcaccgatgggcatggcgggccatgtgacttttgtacacaacggcaaggcttatgttactggcggtgttaaccagaatatcttcaatggctattttgaagatctcaacgaagctggaaaagattcaaccgctgtagataaaatcaatgcctactattttgacaaaaaagcagaagattattttttcaataagtttctgttgtcttttgatccctcaacacagcaatggagttacgctggcgaatcgccctggtacggaacggctggtgcggcggttgtgaataaaggtgataaaacctggcttattaatggcgaagccaaacccggattgcgaacggatgccgtatttgaacttgatttcaccggtaataatttaaaatggaataagcttgctcccgtctcatcaccagatggcgtcgctggcggttttgcggggataagcaatgattctcttatatttgccggaggggccggattcaaaggttcacgagaaaattaccagaacggtaagaactatgcgcatgaaggcctgaaaaaatcatatagcactgatattcatctttggcataacgggaaatgggataaatcgggtgaattatcgcaaggtcgggcctacggagtatcattgccctggaataatagtctattgattattggcggtgaaactgcaggcggcaaagcggtgacggattcagtttttatctctgtgaaggataataaagttacagtacaaaactaa

SEQ ID NO: 101 - DNA, 1215 bp, Escherichia coli O157H7
atgactacctggaccgtcagggtgtttacaacggcagagataatttatcggaaaacagttatcgcattggtgtgtcatttaaactgtagtagacaggagacagtcacaatgaataaaacaataacggcgcttgctatcctaatggcttcatttgccgcaaacgcgtctgtattaccggaaactcctgtaccatttaaaagtggtaccggagcaattgataacgacactgtctacattggtttaggtagcgcaggtacggcatggtacaagctggagacacaggccaaagataaaaaatggacagcgttagctgcattccctggtggaccaagagatcaagcaacctcggcatttattgatggcaatctgtatgtgtttggcggcattggcaaaaacagcgagggcttgactcaggtatttaatgacgtacacaaatacaaccccaaaaccaatagctgggttaaattgatatcgcacgcgccgatgggcatggcgggccatgtgacttttgtacacaacggcaaggcttatgttactggcggtgttaaccagaatatcttcaatggctattttgaagatctcaacgaagctggaaaagattcaaccgctgtagataaaatcaatgcacactattttgacaaaaaagcagaagattatttctttaataagtttctgttgtcttttgatccctctacacagcaatggagttacgctggcgaatctccctggtacgggacggctggtgcggcggttgtgaataaaggtgataaaacctggcttattaatggcgaagccaaacccggattgcgaacggatgccgtatttgaacttgatttcaccggtaataatttaaaatggaataggcttgctcccgtctcatcaccagatggcgtcgctggcggttttgcggggataagcaatgattctcttatatttgccggaggggccggattcaaaggttcacgagaaaattaccaaaacggtaagaactatgcgcatgaaggcctgaaaaaatcatatagcactgatattcatctttggcataacgggaaatgggataaatcgggtgaattatcgcaaggtcgggcctacggagtatcattgccctggaataatagtctattgattattggcggtgaaactgcaggcggcaaagcggtgacggattcagttttgatctctgtgaaggataataaagtcacagtacaaaactaa

SEQ ID NO: 102 - DNA, 1215 bp, Escherichia coli (strain K12)
atgactaccttgaccgtcagggtgtttacaacggcagagataatttatcggaaaacagttatcgcattggtgtgtcatttaaactgtagtagacaggagacagtcacaatgaataaaacaataacggcgcttgctatcatgatggcttcatttgccgcaaacgcgtctgtattaccggaaactcctgtgccatttaaaagtggtaccggagcaattgataacgacactgtctacattggtttaggtagcgcaggtacggcatggtacaagctggatacacaggccaaagataaaaaatggacagcgttagctgcattccctggcggaccaagagatcaagcaacctctgcatttattgatggcaatctgtatgtgtttggcggcattggcaaaaacagcgagggcttgactcaggtatttaatgacgtacacaaatacaaccccaaaaccaatagttgggttaaattgatgtcgcacgcgccgatgggcatggcgggccatgtgacttttgtacacaacggcaaggcttatgttactggtggtgttaaccagaatatcttcaatggctattttgaagatctcaacgaggctggaaaagattcaaccgctatagataaaatcaatgctcactattttgacaaaaaagcagaagattatttcttcaataagtttctgttgtcttttgatccctcaacacagcaatggagttacgctggcgaatcgccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgctcccgtctcatcaccagatggcgtagctggcggttttgcggggataagcaatgattctcttatatttgccggaggggccggattcaaaggttcacgagaaaattaccagaacggtaagaactatgcgcatgaaggcctgaaaaaatcatatagcactgatattcatctttggcataacgggaaatgggataaatcgggtgaattatcgcaaggtcgggcctacggagtatcattgccctggaataatagtctattgattattggcggtgaaactgcaggcggcaaagcggtgacggattcagttttgatcactgtgaaggataataaagtcacagtacaaaactaa

SEQ ID NO: 103 - DNA, 1215 bp, Escherichia coli (strain UTI89)
atgactaccttgaccgtcagggtgtttacaacggcagagataatttatcggaaaacagttatcgcattggtgtgtcatttaaactgtagtagacaggagacagtcacaatgaataaaacaataacggcgcttactatcataatggcttcatttgccacaaacgcgtctgtattaccggaaactcctgtgccatttaaaagtggtaccggagcaattgataacgacactgtctacattgggttaggtagcgcaggtacggcatggtacaagctggatacacaggccaaagataaaaaatggacggcgttagctgcattccctggcggaccgagagatcaagcaacctctgcatttattgatggcaatctgtatgtgtttggcggcattggcaaaaacagcgagggcttgactcaggtatttaatgacgtacacaaatacaaccccaaaaccaatagctgggttaaattgatgtcgcacgcgccgatgggcatggcgggccatgtgacttttgtacacaacggcaaggcttatgttactggcggtgttaaccagaatatcttcaatggctattttgaagatctcaacgaagctggaaaagattcaaccactatagataaaatcaacgcccactattttgacaaaaaagcagaagattatttcttcaataagtttctgttatcttttgatccctctacacagcaatggagttacgctggcgaatctccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgctcccgtcgcatcaccagatggcgtcgctggcggttttgcaggcatgagcaatgactcacttatatttgccggaggggccggattcaaaggttcacgagaaaattaccagaacggtaagaactatgcgcatgaaggcctgaaaaaatcatatagcgctgatattcatctttggcataacgggaaatgggataaatcgggcgaattatcgcaaggtcgggcctacggagtatcattgccctggaataatagtttattaattattggcggtgaaactgcaggcggcaaagcggtgacggattcagttttgatttctgtgaaggataataaagtcacagtacaaaactaa

SEQ ID NO: 104 - DNA, 1155 bp, Vibrio cholerae serotype O1 (ATCC 39541)
atgaatatgaaaactctattgacttacgctacgttactttctgtgacagcatttagtcatgtggtctatgccgataatcaatggcctgatttacctactggatttaaagatggggttggtgcacaagttggttcaaaagtgtatgtaggacttggatctctggggaaaagcttttatgtacttgatttgaatgcattatcgaaaggatggcaaaagattgctgattttaccggtgctgaacgaagtggagcgacggcatctgtaataggaaattatatttatctctttggtggctccggcaaagcagaaccgtctgatccatcgcctattttgtttgattcggtatatcgctacgacaccaagaaagattcatgggagaaaatgaatacgacctcgcctgtaggacttcttggtgcttcttcttattctccggacaatagacagatcctcttttttggtggttataacaaagcctattttgatcgctatctgcgagatatatcgaccactgacaagcaagtaaatcctgaagtatggcaacgtattgttgatgattatatgggaatgacgcctacggattataaatggaaccgaaacgtgatcagttatttgcctgaaaaacaagagtggcgtgatttaggcgtgtctacgtatctccctaattgtggtagcgcaaccgttattgaagggaacaaagtaactctcatcagtggtgagataaaaccgggtttacgcaccgctgaagttaagcaatatgaatttggaatggatcaaccgtggaaaagtttgttgcccttacctgcaccccaaacgagcaatatccaagagggtgttgcaggtgcattttctgggaagacaaatggagttgtagttgttgctggtggagcgaatttccatggtgcgaaacaagcatttgaaaacggaaaaatgttcgcacatgaagggctgcctaaggcgtttaattccgaaatttatgtagaaaaagagggcatttggtcaacggtaaattcgcttcctgagggcttggcatatggggcttcatttactacatcagagggtgttttaattgttggtggagaaaaaagtggtaaagaaatgagtcataaggtttatatgttggcttggaatggctctactgttgaagtgatagactaa

SEQ ID NO: 105 - DNA, 1179 bp, Yersinia enterocolitica serotype O8/biotype 1B
atgactcagatataccaccaatataagaaaaaattgagtacaaaagtcatcctattgagcgcgctgactctttgcatcacgttctcgctaccttacgctaatgccgaacgctatccggatgtacctgttgcctttaagtatgggacgggcgctcgggttgataaccatttatatgttggcttaggtagcgccggtcaatcgtggtatcgccttgatacggataaagccagcagtggttggcagaaaatagctgacttcccagggcagcctcgtgaacaggctgtcactgtagcattaagcggtaagctgtatgtttttggtggcgtgggtaaaaatagcgccagtgacacgcaggtacgtgcgctggatgatgtgtatcaatatgatccacaaactaatcagtggcaacggttggcaacgcgagcgccgcgcggcttggtgggtaccgccgcaaccacattgaatggcactcaggcgttgctgctggggggcgtcaataaagctattttcgacggctacttcacggatctggccgctgcgggcggcaatgaaacccagaaaaatgcagttgttaatgcgtattttgaccaggctccggcagactatttctataaccgcgatgtgctgatgtatgacccagctaaaaatcagtggaaaagtggcgggcaagtaccatttttaggtacggctgggtcagctatcaccgctaaaaagggtgatttgatattaataaatggtgaaattaaacccggattacgcacggcagcggtatggcagggcaaaacgcagggcacagagctgaaatggcaacagcgaccagatttgatcggcgctgaaaaaggcgcggtgcaagaagggctggcaggggcttttgcgggtgtgagccatgatgtggtactggtaggcggtggggccaatttccccggttcatggcaacaattcaatgcggggcagttatatgcccatcaagggctaaaaaagcagtggcaacagcccatttatgcgctggtggataatcaatggcaggtggcaggcaaattacctcagccgctggcttacggcgtatccattcaagataaagataaagtgattttgctgggaggagaaacaagcgacggtgttgcaacctcagcagtgactcagctctcctggcaaggtggcaagctgcatcttgaataa

SEQ ID NO: 106 - DNA, 1179 bp, Yersinia pestis bv. Antiqua (strain Nepal516)
atgacccagttatacccacaatataagaaacaattgacgacaaaaatagtgctgtttagtgcgttatctctattgatgatggcatcgctacctaatacctatgcggaacaatatcctgacgttccggtaccatttaaaaacggtacgggtggcaaagtggagaacagcttatatgtggggctggggagcgccggtgtgtcgtggttccgtctggatacagataaaaccggtgctgggtggcagaaagtggctaatttcccaggccagcctcgtgaacaggctgtgacggtggtattggcagggaaactgtacgtgttcggtggggtggggaaaacgaacgccaacgatacccaagtacgggcactggatgatgcgtatcgttttgatccacaaactaatcaatggcagcaactggctacccgcgctccccgaggtctggtggggaccgtggccacgacactggacggctcccaggcggtgctgttaggcggtgtaaataaagcgatttttgacggttattttactgatcttgcgtcagccggaagtgatgaagtacgcaagagtgcggtaataaacgcctattttaatcaggctccggctgattatttctacaaccgtgatgtccttatctatgatccgcaaaaaaatcagtggaaaagcggcggtttgctgccatttttagggacagccgggtcagccatcagccgcatggataatcgcttgatattaatcaatggcgaaattaaacctggtttacgcacagcggctgtatggcaggggctaatgcaagggaatgtactggagtggcagccacaaccggacttaatcggagctgaaacgggatccgcacaggaaggattggctggcgcattttccggtatcagtcataaaacagtcttagtcgccggaggcgctaatttccctggtgcctggaagcaatttaatcgcggtcacctatatgctcaccaagggctggaaaaacagtggcaccagcaggtgtatgcgttggtcgataatcagtggagaatcgccgggaagttgcctcaaccgctgggttatggcgtctctattcaggggccagataaggtcattttgattggcggcgaaaccaccggaggtacggccacttcagcggtcacacagctatcttggcagggaggaaaactgcatattgagtaa

SEQ ID NO: 107 - DNA, 1215 bp, Shigella boydii serotype 4 (strain Sb227)
atgactaccttgaccgtcagggtgtttacaacggcagagataatttatcggaaaacagttatcgcattggtgtgtcatttaaactgtagtagacaggagacagtcacaatgaataaaacaataacggcgcttgctatcatgatggcttcatttgccgcaaacgcgtctgtattaccggaaactcctgtgccatttaaaagtggtaccggagcaattgataacgacactgtctacattggtttaggtagcgcaggtacggcatggtacaagctggatacacaggccaaagataaaaaatggacagcgttagctgcattccctggtggaccaagagatcaagcaacctctgcatttattgatggcaatctgtatgtgtttggcggcattggcaaaaacagagagggcttgactcaggtatttaatgacgtacacaaatacaaccccaaaaccaatagctgggttaaattgatgtcgcacgcaccgatgggcatggcgggtcatgtgacttttgtacacaacggcaaggcttatgttactggcggtgttaaccagaatatcttcaatggctattttgaagatctcaacgaggctggaaaagattcaaccgctgtagataaaatcaatgcctactattttgacaaaaaagcagaagattatttcttcaataagtttctgttgtcttttgatccctcaacacagcaatggagttacgctggcgaatcgccctggtacggaacggctggtgcggcggttgtgaataaaggtgataaaacctggcttattaatggcgaagccaaacccggattgcgaacggatgccgtatttgaacttgatttcaccggtaataatttaaaatggaataagcttgctcccgtctcatcaccagatggcgtcgctggcggttttgcggggataagcaatgattctcttatatttgccggaggggccggattcaaaggttcacgagaaaattaccagcacggaaagaactatgcgcatgaaggcctgaaaaaatcatatagcactgatattcatctttggcataacgggaaatgggataaatcgggtgaattatcgcaaggtcgggcctacggagtatcattgccctggaataatagtctattgattattggcggtgaaactgcaggcggcaaagcggtgacggattcagttttgatctctgtgaaggataataaagtcacagtacaaaactaa

SEQ ID NO: 108 - DNA, 1155 bp, Vibrio cholerae serotype O1 (ATCC 39315); identical to SEQ ID NO: 104

SEQ ID NO: 109 - DNA, 3045 bp, Clostridium septicum
atgaataagaaaaagattatgtcaatattagtatctgcttttttaataactaacttatcaagtaatataatttttgctgacattaaagagaatgtttatattaatcaatattcagaggggaataggtcacaaccaattgcagaaaaattagttcctagatcagaaattcaagcaagtgcaacatcagctcttacaggagaaggacctgaaaaagctatagatggtaatacgtccacactttggcatactccatgggctggcgtagatattcaaattaatcctcaaagcttaacattaaaattggggaaaactagaaatatttcatctatttgcgtaactccaagacaagagggaactaatggaatgataacggattataaaatttactcaggagatgatgtaattgcagaaggaaaatggaaatcagattcatcagataaatatgtagtttttgataatcctatatcaacagacaatataagaattgaggctatatcaactgttggggatgagaataataagcatgctagtatagcagaagtggaagtttatgaacttgctgatactccggttaaattagcagaaagtaataacaaagttatcaataatggaaatggtggaaattatgaaggtgatatatcagaaatttcattattagaagagggaactgcaataattagatttactaataatggttcaagcttatttagtattagtaataatgaaagaacaaatgaacattttcatgtatatataaatggaggagctattggatatgaattaagaaaacaaagtggaaatttagcaacaggtagtgtaaataaggcattaaatgcaggaataaatacaattgcctttaaagcagagaaagggaaaggatattctatttacttaaatggagaaaagatattaactagtagtagtataacggctaattttttatcaacattagaaggtttaaatactttaagtttaggaaaaactgatagaccgtctggatctaatgaatataattttacaggagaaatagatttttttgagttatattcaaaaccattagcagataggtatttaaaagagagaactggagaaactacatcaaaggatttaccatttccagaaggtgcagtaaaaacagaacctgttgatatatttactccaggagaattaggaagcaataattttagaatacctgctttatatacaactaaagatggtacagttttagcatctatagatgttagaaaaggtggaggacatgatgcccctaataatattgatacaggaataaaaaggagtacagatggaggtgtaacttgggatgaaggaaaaataatattagattatcctggtgcatcttcagctatagatacatcattactccaagatgatgaaacaggaagaatatttttaatagtaactcattttgcagaaggatatggatttggaaattctaaaacaggaagtggatatgtagaaatagaaggtaaacgttatcttaaattattaggagctaatgatactatttatacagtaagagaaggtgttgtgtacgatagtaatggagaggctacaaattatacagtagataacaacaatgagctttatgaaaatggtaatcgtataggtaatgttttattaagtaattcaccgttaaaagtaatgggaacatcatttttatcattaatttatagtgatgatgatggacaaacttggagtgatccaatagatttaaataaagaagtaaagacagactggatgagatttctaggaacaggtccaggaaaaggacatcaaataaaaactggtagatacgcaggaagattattattccctgtatatttaacaaatgcatctggtttccaatcaagtgctgttatatatagtgatgataatggtgcaacttggaatataggagaaacagctactgatggtagattaatggataatggagatagagctagtgcagaaacaataacaacaaatacaagtggtggagtaggacaattaacagaatgtcaagttgttgaaatgccaaatggacagttaaaaatgtttatgagaaatactggaggaaatagtggacgtgtaagaatagcaacaagctttgatggaggagcaacttgggaagatgatgtagtaagggatgaaaatataaaagaaccttattgtcaactaagtgttattaattacagtcaaaagatagatggtaaagatgctataatttttgcaattccagatgctaattatccaaatagagtaaatggaactgttagagtaggtttaataactgaaaatggatcttatgaaaatggtgaaccacgttatgatattgagtggagatataataaagtagtggcaccgggaacttatggatattcctgtttatcagaaatgccaaatggtgaaattggattattctatgaaggtagaggaagcagacaaatgtcctttactagaatgaatatagattatttaaaagcagatttattacaagatgtaccagctgccaatataaaatcatatacaacgaatagtgaaaataatatttatgatccgggagataaaatatctttaaatgtaacttttgatcaaacagtatcacttataggtgatagaactataacagctgatattggaggaaaagaagttttattaactttagctaatagtaaaggtggatccgaatatacatttgaaggaacagtaccagctgatatatcaaatggtaattatactataactataaaaggaaaatcaggtttaaagatagttaatgtagttaataaggtaacagatataactgaagatagaaacacaggcttaaatgtacaagttggagaagaagttcaatcagtagataaaactttattgcaagatttggtagatagtacatctaatctaataaaggaagattatacagaagaaagctggattttatatgaaaaagctttggaagtagcaaataaatttttagtaaatgaaattgcagtgcaagaagaggttgatgctgcaaaacctacattagaaaatgcttataaataa

SEQ ID NO: 110 - DNA, 3108 bp, Streptococcus pneumoniae
atgtcttatttcagaaatcgggatatagatatagagaggaacagtatgaatcggagtgttcaagaacgtaagtgtcgttatagcattaggaaactatcggtaggagcggtttctatgattgtaggagcagtggtatttggaacgtctcctgttttagctcaagaaggggcaagtgagcaacctctggcaaatgaaactcaactttcgggggagagctcaaccctaactgatacagaaaagagccagccttcttcagagactgaactttctggcaataagcaagaacaagaaaggaaagataagcaagaagaaaaaattccaagagattactatgcacgagatttggaaaatgtcgaaacagtgatagaaaaagaagatgttgaaaccaatgcttcaaatggtcagagagttgatttatcaagtgaactagataaactaaagaaacttgaaaacgcaacagttcacatggagtttaagccagatgccaaggccccagcattctataatctcttttctgtgtcaagtgctactaaaaaagatgagtacttcactatggcagtttacaataatactgctactctagaggggcgtggttcggatgggaaacagttttacaataattacaacgatgcacccttaaaagttaaaccaggtcagtggaattctgtgactttcacagttgaaaaaccgacagcagaactacctaaaggccgagtgcgcctctacgtaaacggggtattatctcgaacaagtctgagatctggcaatttcattaaagatatgccagatgtaacgcatgtgcaaatcggagcaaccaagcgtgccaacaatacggtttgggggtcaaatctacagattcggaatctcactgtgtataatcgtgctttaacaccagaagaggtacaaaaacgtagtcaactttttaaacgctcagatttagaaaaaaaactacctgaaggagcggctttaacagagaaaacggacatattcgaaagcgggcgtaacggtaaaccaaataaagatggaatcaagagttatcgtattccagcacttctcaagacagataaaggaactttgatcgcaggtgcagatgaacgccgtctccattcgagtgactggggtgatatcggtatggtcatcagacgtagtgaagataatggtaaaacttggggtgaccgagtaaccattaccaacttacgtgacaatccaaaagcttctgacccatcgatcggttcaccagtgaatatcgatatggtgttggttcaagatcctgaaaccaaacgaatcttttctatctatgacatgttcccagaagggaagggaatctttggaatgtcttcacaaaaagaagaagcctacaaaaaaatcgatggaaaaacctatcaaatcctctatcgtgaaggagaaaagggagcttataccattcgagaaaatggtactgtctatacaccagatggtaaggcgacagactatcgcgttgttgtagatcctgttaaaccagcctatagcgacaagggggatctatacaagggtaaccaattactaggcaatatctacttcacaacaaacaaaacttctccatttagaattgccaaggatagctatctatggatgtcctacagtgatgacgacgggaagacatggtcagcgcctcaagatattactccgatggtcaaagccgattggatgaaattcttgggtgtaggtcctggaacaggaattgtacttcggaatgggcctcacaagggacggattttgataccggtttatacgactaataatgtatctcacttaaatggctcgcaatcttctcgtatcatctattcagatgatcatggaaaaacttggcatgctggagaagcggtcaacgataaccgtcaggtagacggtcaaaagatccactcttctacgatgaacaatagacgtgcgcaaaatacagaatcaacggtggtacaactaaacaatggagatgttaaactctttatgcgtggtttgactggagatcttcaggttgctacaagtaaagacggaggagtgacttgggagaaggatatcaaacgttatccacaggttaaagatgtctatgttcaaatgtctgctatccatacgatgcacgaaggaaaagaatacatcatcctcagtaatgcaggtggaccgaaacgtgaaaatgggatggtccacttggcacgtgtcgaagaaaatggtgagttgacttggctcaaacacaatccaattcaaaaaggagagtttgcctataattcgctccaagaattaggaaatggggagtatggcatcttgtatgaacatactgaaaaaggacaaaatgcctataccctatcatttagaaaatttaattgggactttttgagcaaagatctgatttctcctaccgaagcgaaagtgaagcgaactagagagatgggcaaaggagttattggcttggagttcgactcagaagtattggtcaacaaggctccaacccttcaattggcaaatggtaaaacagcacgcttcatgacccagtatgatacaaaaaccctcctatttacagtggattcagaggatatgggtcaaaaagttacaggtttggcagaaggtgcaattgaaagtatgcataatttaccagtctctgtggcgggcactaagctttcgaatggaatgaacggaagtgaagctgctgttcatgaagtgccagaatacacaggcccattagggacatccggcgaagagccagctccaacagtcgagaagccagaatacacaggcccactagggacatccggcgaagagccagccccgacagtcgagaagccagaatacacaggcccactagggacagctggtgaagaagcagctccaacagtcgagaagccagaatttacagggggagttaatggtacagagccagctgttcatgaaatcgcagagtataagggatctgattcgcttgtaactcttactacaaaagaagattatacttacaaagctcctcttgctcagcaggcacttcctgaaacaggaaacaaggagagtgacctcctagcttcactaggactaacagctttcttccttggtctgtttacgctagggaaaaagagagaacaataa

SEQ ID NO: 111 - DNA, 1149 bp, Salmonella typhimurium (strain LT2)
atgactgtagaaaaatccgtggtttttaaagctgaaggtgagcattttactgatcaaaaaggcaatactattgtcggtagcggctctgggggaacaaccaaatattttcgaattccagcaatgtgtacaacctcaaaaggtaccattgttgtatttgctgatgcgcgccataacactgccagcgatcagtcctttattgacacagcggcggcccgtagcacagatggagggaaaacctggaataaaaaaattgcgatttataatgaccgtgtcaacagtaaactttcccgcgtcatggatcccacatgcatcgtggcgaatattcagggaagagaaacaatcttagtcatggttggcaaatggaataataatgataaaacatggggcgcatacagagataaggctccagacactgactgggatttggttttatataaatcaaccgatgatggcgttaccttttcaaaggtggaaacgaacatccatgatatagtaactaaaaatggaacgatatctgcaatgcttggcggtgttggatctggtcttcagctaaatgacggtaaactggttttcccggtacaaatggttcgcactaaaaatatcaccacagttctgaatacatcattcatatactccactgatggaataacatggtcattgccatcaggatactgtgagggttttggttcagaaaataacatcatagaatttaatgcctctttggttaataacatcagaaactctggcctccgccgttcttttgaaacaaaagattttggaaaaacatggactgagtttcctccgatggacaagaaggttgacaacaggaatcatggagtacaaggttcaacaattactattccatcaggcaataaacttgtcgcggctcactcatccgcgcagaacaagaataatgactataccagaagtgatatatctctctacgctcacaatttatactccggggaagtaaagttaattgatgctttttatccgaaagtcggtaatgccagtggcgcgggttactcttgtctttcatatcgtaaaaatgtagataaagaaacactgtatgttgtctatgaagccaatggaagtattgagttccaggaccttagccgtcatttaccagtaataaaatcatacaattaa

SEQ ID NO: 112 - DNA, 2346 bp, Vibrio cholerae serotype O1 (ATCC 39541)
atgcgtttcaaaaacgtaaagaaaaccgctttaatgcttgcaatgttcggtatggcgacaagctcaaacgccgcactttttgactataacgcaacgggtgacactgagtttgacagtccagccaaacagggatggatgcaagacaacacgaataatggcagcggcgttttaaccaatgcagatggaatgcccgcttggttggtgcaaggtattggagggagagctcaatggacatattctctctctactaatcaacatgcccaagcatcaagtttcggttggcgaatgacgacagaaatgaaagtgctcagtggtggaatgatcacaaactactacgccaacggcactcagcgtgtcttacccatcatttcattagatagcagtggtaacttagttgttgagtttgaagggcaaactggacgcaccgttttggcaaccggcacagcagcaacggaatatcataaatttgaattggtattccttcctggaagtaacccatccgctagcttttacttcgatggcaaactcattcgtgacaacatccagccgactgcatcaaaacaaaatatgatcgtatgggggaatggctcatcaaatacggatggtgtcgccgcttatcgtgatattaagtttgaaattcaaggcgacgtcatcttcagaggcccagaccgtataccgtccattgtagcaagtagcgtaacaccaggggtggtaaccgcatttgcagagaaacgtgtggggggaggagatcccggtgctctgagtaataccaatgacataatcactcgtacctcacgagatggcggtataacttgggataccgagctcaacctcactgagcaaatcaatgtcagtgatgagtttgatttctccgatcctcggcctatctatgatccttcctccaatacggttcttgtctcttatgctcgatggccgaccgatgccgctcaaaacggagatcgaataaaaccatggatgccaaacggtattttttacagcgtctatgatgttgcatcagggaactggcaagcgcctatcgatgttaccgatcaggtgaaagaacgcagtttccaaatcgctggttggggtggttcagagctgtatcgccgaaataccagcctaaatagccagcaagactggcaatcaaacgctaagatccgaattgttgatggtgcagcgaaccagatacaagttgccgatggtagccgaaaatatgttgtcacactgagtattgatgaatcaggtggtctagtcgctaatctaaacggtgttagtgctccgattatcctgcaatctgaacacgcaaaggtacactctttccatgactacgaacttcaatattcggcgttaaaccacaccacaacgttattcgtggatggtcagcaaatcacaacttgggctggcgaagtatcgcaggagaacaacattcagtttggtaatgcggatgcccaaattgacggcagactgcatgtgcaaaaaattgttctcacacagcaaggccataacctcgtggagtttgatgctttctatttagcacagcaaacccctgaagtagagaaagaccttgaaaagcttggttggacaaaaattaaaacgggcaacaccatgagtttgtatggaaatgccagtgtcaacccaggaccgggtcatggcatcacccttactcgacaacaaaatatcagtggcagccaaaacggccgcttgatctacccagcgattgtgcttgatcgtttcttcttgaacgtcatgtctatttacagtgatgatggcggttcaaactggcaaaccggttcaacactccctatcccctttcgctggaagagttcgagtatcctagaaactctcgaacctagtgaagctgatatggttgaactccaaaacggtgatctactccttactgcacgccttgattttaaccaaatcgttaatggtgtgaactatagcccacgccagcaatttttgagtaaagatggtggaatcacgtggagcctacttgaggctaacaacgctaacgtctttagcaatatcagtactggtaccgttgatgcttctattactcggttcgagcaaagtgacggtagccatttcttactctttactaacccacaaggaaaccctgcggggacaaatggcaggcaaaatctaggcttatggtttagcttcgatgaaggggtgacatggaaaggaccaattcaacttgttaatggtgcatcggcatattctgatatttatcaattggattcggaaaatgcgattgtcattgttgaaacggataattcaaatatgcgaattcttcgtatgcctatcacattgctaaaacagaagctgaccttatcgcaaaactaa

SEQ ID NO: 113 - DNA, 3108 bp, Streptococcus pneumoniae (strain ATCC BAA-255); identical to SEQ ID NO: 110

SEQ ID NO: 114 - DNA, 2094 bp, Streptococcus pneumoniae serotype 4 (ATCC BAA-334)
atgaataaaagaggtctttattcaaaactaggaatttctgttgtaggcattagtcttttaatgggagtccccactttgattcatgcgaatgaattaaactatggtcaactgtccatatctcctatttttcaaggaggttcatatcaactgaacaataagagtatagatatcagctctttgttattagataaattgtctggagagagtcagacagtagtaatgaaatttaaagcagataaaccaaactctcttcaagctttgtttggcctatctaatagtaaagcaggctttaaaaataattacttttcaattttcatgagagattctggtgagataggtgtagaaataagagacgcccaagagggaataaattatttattttctagaccagcttcattatggggaaagcataaaggacaggcagttgaaaatacactagtatttgtatctgattctaaagataaaacatacacaatgtatgttaatggaatagaagtgttctctgaaacagttgatacatttttgccaatttcaaatataaatggtatagataaggcaacactaggagctgttaatcgtgaaggtaaggaacattacctcgcaaaaggaagtattggtgaaatcagtctatttaacaaagcaattagtgatcaggaagtttcaaatattcccttgtcaaatccatttcagttaattttccaatcaggagattctactcaagctaactattttagaataccgacactatatacattaagtagtggaagagttctatcaagtattgatgcacgttatggtgggactcatgattctaaaagtaagattaatattgccacttcttatagtgatgataatgggaaaacgtggagtgagccaatttttgctatgaagtttaatgactatgaggagcagttagtttactggccacgagataataaattaaagaatagtcaaattagtggaagtgcttcattcatagattcatccattgttgaagataaaaaatctgggaaaacgatattactagctgatgttatgcctgcgggtattggaaataataatgcaaataaagccgactcaggttttaaagaaataaatggtcattattatttaaaactaaagaagaatggagataacgatttccgttatacagttagagaaaatggtgtcgtttatgatgaaacaactaataaacctacaaattatactataaatgataagtatgaagttttggagggaggaaagtctttaacagtcgaacaatattcggttgattttgatagtggctctttaagagaaaggcataatggaaaacaggttcctatgaatgttttctacaaagattcgttatttaaagtgactcctactaattatatagcaatgacaactagtcagaatagaggagagagttgggaacaatttaagttgttgcctccgttcttaggagaaaaacataatggaacttacttgtgtcctggacaaggtttagcattaaaatcaagtaacagattgatttttgcaacatatactagtggagaactaacctatctcatttcggatgatagtggtcaaacatggaagaaatcctcagcttcaattccgtttaaaaatgcaacagcagaagcacaaatggttgaactgagagatggtgtgattagaacattctttagaaccactacaggtaagatagcttatatgactagtagagattctggagaaacatggtcgaaagtttcgtatattgatggaattcaacaaacttcatatggcacacaagtatctgcaattaaatactctcaattaattgatggaaaagaagcagtcattttgagtacaccaaattctagaagtggccgtaagggaggccaattagttgtcggtttggtcaataaagaagatgatagtattgattggagataccactatgatattgatttgccttcgtatggttatgcctattctgcgattacagaattgccaaatcatcacataggtgtactgtttgaaaaatatgattcgtggtcgagaaatgaattgcatttaagcaatgtagttcagtatatagatttggaaattaatgatttaacaaaataa

SEQ ID NO: 115 - DNA, 576 bp, Salmonella typhimurium (strain LT2)
atgccttttaaaatgagcagcaaagcgcagactattaaaattttcaatctgcgttcagatacaaacgaatttattggggcaggtgatgcgtatatcccgccgcacactggattaccggcaaactgtactgatatagcccctcctgatattccctccagtcatattgctgtatttgacgctgaaacccagacgtggaacctgaaagaagaccatcgcggagaaacggtatacgacacaacaaccggtaatcagatgtatatctccgaacccggcccgttgcccgaaaatgtcacatcagtttcaccaggtggtggatacaaaaaatgggatagtaaggctaaggcctgggtgaatgatgaaggtgcggaggttgcagccagacttcgtgaagctgaaggaaccaaaagccgtcttttgcaaatggcatcggggaaaattgcgccgcttcaggatgcggttgatcttggaattgcaacagatgatgaaaaagcgcagctcgacgaatggaaaaaatacagggtactggtaaaccgggttgatacctcaaatcctgattggccggaacaaccagtctga

SEQ ID NO: 116 - DNA, 1215 bp, Escherichia coli O6H1 (strain CFT073)
atgactaccttgaccgtcagggtgtttacaacggcagagataatttatcggaaaacagttatcgcattggtgtgtcatttaaactgtagtagacaggagacagtcacaatgaataaaacaataacagcgcttgctatcctaatggcttcatttgccgcaaacgcgtctgtattaccggaaactcctgtgccatttaaaagtggtaccggagtaattgataacgacactgtctacattggtttaggtagcgcaggtacggcatggtacaagctggatacacaggccaaagataaaagatggacagcgttagctgcattccctggcggaccgagagatcaagcaacctctgcatttattgatggcaatctgtatgtgtttggcggcattggcaaaaacagcgagggcttgactcaggtatttaatgacgtacacaaatacaaccccaaaaccaatagctgggttaaattgatgtcgcacgcgccgatgggcatggcaggccatgtgacttttgtacacaacggcaaggcttatgttactggcggtgttaaccagaatatcttcaatggctattttgaagatctcaacgaagctggaaaagattcaaccgctatagataaaattaacgcctactattttgacaaaaaagcagaagattatttcttcaataagtttctgttgtcttttgatccctctacacagcaatggagttacgctggcgaatctccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgctcccgtcgcatcaccagatggcgtcgctggcggttttgcaggcatgagcaatgactcacttatatttgccggaggggccggattcaaaggttcacgagaaaattaccagaacggtaagaactatgcgcatgaaggcctgaaaaaatcatatagcgctgatattcatctttggcataacgggaaatgggataaatcaggcgaattatcgcaaggtcgggcctacggagtatcattgctctggaataatagtttattaattattggcggtgaagctgcaggcggcaaagcggtgacggattcagttttgatttctgtgaaggataataaagtcacagtacaaaactaa

SEQ ID NO: 117 - DNA, 1155 bp, Salmonella enteritidis PT4 (strain P125109)
atggggatgcagatgaaaaattttaagaaaatgatgacgctaatggcgctatgtttatcagtcgctatcaccacatcaggatatgcaacgacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatcagtgtcggtatttttaaatgatgagctttatgtttttggtggcgtagggaaaaaaaacagtgaatcaccgttgcaggtttatagcgatgtgtacaaatactcaccggtgaaaaatacatggcaaaaagttgacactatatctccagttggattaacagggcatacgggagtaaaattaaacgaaacgatggtacttattaccggaggggttaatgagcatatctttgataagtattttattgatatagaagctgcggatgaaagtgaaaaaaataaagtcatctataattattttaataaacctgccaaagattatttttttaataaaatcgtatttatctacaatgctaaagagaacacatggaagaatgccggtgagctgccaggcgcggggacggcaggatcgtcatcggtaatggaaaataatttcctgatgctgattaatggtgagctcaaaccgggtttacgtaccgatgtgatttaccgcgccatgtgggataacgataagctaacatggttgaagaacagccagttaccgccatcgcctggagaacaacagcaagaagggttggccggagcattttcgggctatagtcacggtgtcctgcttgtcggtggtggcgcgaattttccgggagcaaaacaaaattataccaatggaaagttttattcccacgaaggaataaataaaaaatggcgagatgaagtctatggtttggttaatggccattggcaatatatgggtaaaatgaaacaacctctcggctatggtgtatcagtaagttatggtgatgaagttttccttattggtggtgaaaatgctaaagggaaacctgtttcgtctgtaacctcctttaccatgcgtgatggtaatttattaataaaataa

SEQ ID NO: 118 - DNA, 1107 bp, Escherichia coli O9H4 (strain HS)
atgaataaaacaataacggcgcttgctatcatgatggcttcatttgccgcaaacgcgtctgtattacctgaaactcctgtgccatttaaaagtggtaccggagcaattgataacgacactgtctacattggtttaggtagcgcaggtacggcatggtacaagctggatacacaggccaaagataaaaaatggacagcgttagctgcattccctggtggaccaagagatcaagcaacctcggcatttattgatggcaatctgtatgtgtttggcggcattggcaaaaatagcaagggcttgactcaggtatttaatgacgtacacaaatacaaccccaaaaccaatagttgggttaaattgatgtcgcatgcaccgatgggcatggcgggtcatgtgacttttgtacacaacggcaaggcttatgttactggcggtgttaaccagaatatcttcaatggctattttgaagatctcaacgaggctggaaaagattcagcaactatagacaagatcaatgcccattattttgacaaaaaagcagaagattatttcttcaataagtttctgttgtcttttgatccctcaacacagcaatggagttacgctggcgaatctccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgctcccgtctcatcaccagatggcgtcgctggcggttttgcggggataagcaatgattctcttatatttgccggaggggccggattcaaaggttcacgagaaaattaccagaacggtaagaactatgcgcatgaaggcctaaaaaaatcatatagcactgatattcatctttggcataacgggaaatgggataaatcgggtgaattatcgcaaggtcgggcctacggagtatcattgccctggaataatagtctattgattattggcggtgaaactgcaggcggcaaagcggtgacggattcagttttgatctctgtgaaggataataaagtcacagtacaaaattaa

SEQ ID NO: 119 - DNA, 1140 bp, Haemophilus influenzae (strain 86-028NP)
atggggtttattatgaaactaaccaaaacagcattatgcaccgcactttttgccacctttactttttcagcgaacgcacaaacttatcctgatttgccagtgggaatcaaaggaggaacaggtgcattaattggcgacaccgtttatgtgggattaggctctggtggcgataaattctataccttagacctaaaagatccttcagcacaatggaaagaaattgctacatttccgggtggcgaacgtaatcaacctgttgcagctgcggtagatggcaaactttatgtattcggtggtttacaaaaaaatgaaaaaggcgaacttcaactcgttaatgatgcttatcgctataacccaagcgataatacttggatgaaacttcctactcgttctcctcgcggtttagtaggatcaagtggtgcatctcacggagataaagtttatattttaggcggctctaacctctctatatttaatggcttcttccaagacaacgttgcagcaggagaagataaagggaaaaaagatgaaatcgttgcagcttacttcgatcaacgtccagaagattatttctttacaacagaattattgagctatgaaccctcaaccaacaaatggcgcaatgaaggtcgcattccattctctggtcgtgctggtgctgcctttacaattcaaggtaatgatctagtggttgtcaatggcgaaattaaacctggacttcgcaccgctgaaacccatcaaggtaaattcactgctaaaggtgtgcaatggaaaaacttacctgacttacctgcgccaaaaggcaaatcacaagatggtttagctggtgcacttgcaggctatagtaacggttattatttagtcactggtggcgcaaattttccaggttcaatcaaacaattcaaagaaggaaaacttcacgcacataaaggtttaagcaaagcttggcataacgaagtttatactttaaataatggtaaatggagcatcgttggggaattaccaatgaatattggctatggtttttctgtatcttacaataataaagttttactgattggcggcgaaactgatggaggtaaggctttaactagcgtcaaagcaatcagctatgatggtaaaaaattaaccgtcgaataa

SEQ ID NO: 120 - DNA, 1128 bp, Haemophilus influenzae (strain PittEE)
atgaaactaactaaaacagcattatgtaccgcactttttgctacctttactttttcagcgaacgcacaaacttatcctgatttaccagtggggatcaaaggaggaacaggcgcattaattggcgacacagtttatgtgggattaggttctggtggcgataaattctataccttagacctaaaagatccttcagcacaatggaaagaaattgctacatttccgggtggcgaacgcaatcaacctgttgcagccgcagtggatggaaaactttatgtattcggtggtttacaaaaaaatgaaaaaggcgaacttcaactcgttaatgacgcttatcgctataacccaagcgataatacttggatgaaacttcctactcgttctccccgtggtttagtaggatcaagtggcgcatctcacggagataaagtttatattttaggcggctctaatctctctatatttaatggcttcttccaagacaacgttgcagcaggtgaagatcaagcgaaaaaagatgaaatcgcagcagcttatttcgatcaacgtccagaagattatttctttacaacagaattattgagctatgaaccatcaaccaacaaatggcgcaatgaaggtcgcattccattctctggtcgtgctggtgcagcatttacaattcaaggtaatgatctagtggttgtcaatggcgaaattaaaccaggacttcgcaccgctgaaacccatcaaggtaaattcactgctaaaggtgtgcaatggaaaaacttacctgacttgcccgcaccaaaaggcaaatcacaagatggtttagctggtgcactttcaggttatagtaacggtcattatttagtcactggtggtgcaaattttccaggttcaatcaaacaatacaaagaaggaaaacttcacgcacataaaggtttaagcaaagcttggcataacgaagtttatacgttgaataatggtaaatggcgcatcgttggggaattaccaatgaatattggttatggtttttctgtatcttacaacaataaagttttactgattggcggtgaaactgatggaggtaaggctttaactagcgtcaaagcaataagctatgacggtaaaaaattaaccatcgaataa

SEQ ID NO: 121 - DNA, 1161 bp, Salmonella choleraesuis (strain SC-B67)
atggggatgcagatgaaaaattttaagaaaatgatgacgctaatggcgctatgtttatcagttgctatcaccacatcaggatatgcaacgacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatccgtgtcggtatttttaaatgatgagctttatgtttttggtggcgtagggaaaaaaaacagtgaatcaccgttgcaggtttatagcgatgtgtacaaatactcaccggtgaaaaatacatggcaaaaagttgacactatatctccagttggattaacagggcatacgggagtaaaattaaacgaaacgatggtacttattaccggaggggttaatgagcatatctttgataagtattttattgatatagcggctgcggctgcggatgaaagtgaaaaaaataaagtcatctataattattttaataaacctgccaaagattatttttttaataaaatcgtatttatctacaatgctaaagagaacacatggaagaatgccggtgagctgccagacgcggggacggcaggatcgtcatcggtaatggaaaataatttcctgatgctgattaatggtgagctcaaaccgggtttacgtaccgatgtgatttaccgcgccatgtgggataacgacaagctaacatggttgaagaacagccagttaccgccatcgcctggagaacaacagcaagaagggttggccggagcattttcgggctatagtcacggtgtcctgcttgtcggtggtggcgcgaattttccgggagcaaaacaaaattataccaatggaaagttttactcccacgaaggaataaataaaaaatggcgagatgaagtctatggtttggttaatggccattggcaatatatgggtaaaatgaaacaacctctcggctatggtgtatcagtaagttatggtgatgaagttttccttattggtggtgaaaatgctaaagggaaacctgtttcgtctgtaacctcctttaccatgcgtgatggtaatttattaataaaataa

SEQ ID NO: 122 - DNA, 1161 bp, Salmonella newport (strain SL254)
atggggatgcagatgaaaaattttaagaaaatgatgacgctaatggcgctatgtttatcagtcgctatcaccacatcaggatatgcaacgacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatccgtgtcggtatttttaaatgatgagctttatgtttttggtggcgtagggaaaaaaaacagtgaatcaccgttgcaggtttatagcgatgtgtacaaatactcaccggtgaaaaatacatggcaaaaagttgacactatatctccagttggattaacagggcatacgggagtaaaattaaacgaaacgatggtacttattaccggaggggttaatgagcatatctttgataagtattttattgatatagcggctgcggctgcggatgaaagtgaaaaaaataaagtcatctataattattttaataaacctgccaaagattatttttttaataaaatcgtatttatctacaatgctaaagagaacacatggaagaatgccggtgagctgccagacgcggggacggcaggatcgtcatcggtaatggaaaataatttcctgatgctgattaatggtgagctcaaaccgggtttacgtaccgatgtgatttaccgcgccatgtgggataacgacaagctaacatggttgaagaacagccagttaccgccatcgcctggagaacaacagcaagaagggttggccggagcattttcgggctatagtcacggtgtcctgcttgtcggtggtggcgcgaattttccgggagcaaaacaaaattataccaatggaaagttttattcccacgaaggaataaataaaaaatggcgagatgaagtttatggtttggttaatggccattggcaatatatgggtaaaatgaaacaacctctcggctatggtgtatcagtaagttatggtgatgaagttttccttattggtggtgaaaatgctaaagggaaacctgtttcgtctgtaacctcctttaccatgcgtgatggtaatttattaataaaataa

SEQ ID NO: 123 - DNA, 1149 bp, Salmonella paratyphi B (strain ATCC BAA-1250)
atgaaaaattttaagaaaatgatgacgctaatggcgctatgtttatcagtcgctatcaccacatcaggatatgcaacgacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatccgtgtcggtatttttaaatgatgagctttatgtttttggtggcgtagggaaaaaaaacagtgaatcaccgttgcaggtttatagcgatgtgtacaaatactcaccggtgaaaaatacatggcaaaaagttgacactatatctccagttggattaacagggcatacgggagtaaaattaaacgaaacgatggtacttattaccggaggggttaatgagcatatctttgataagtattttattgatatagcggctgcggctgcggatgaaagtgaaaaaaataaagtcatctataattattttaataaacctgccaaagattatttttttaataaaatcgtatttatctacaatgctaaagagaacacatggaagaatgccggtgagctgccagacgcggggacggcaggatcgtcatcggtaatggaaaataatttcctgatgctgattaatggtgagctcaaaccgggtttacgtaccgatgtgatttaccgcgccatgtgggataacgacaagctaacatggttgaagaacagccagttaccgccatcgcctggagaacaacagcaagaagggttggccggagcattttcgggctatagtcacggtgtcctgcttgtcggtggtggcgcgaattttccgggagcaaaacaaaattataccaatggaaagttttattcccacgaaggaataaataaaaaatggcgagatgaagtctatggtttggttaatggccattggcaatatatgggtaaaatgaaacaacctctcggctatggtgtatcagtaagttatggtgatgaagttttccttattggtggtgaaaatgctaaagggaaacctgtttcgtctgtaacctcctttaccatgcgtgatggtaatttattaataaaataa

SEQ ID NO: 124 - DNA, 1161 bp, Salmonella schwarzengrund (strain CVM19633)
atggggatgcagatgaaaaactttaagaaaatgatgacgctaatggcgctatgtttatcagtcgctatcaccacatcaggatatgcaacgacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatccgtgtcggtatttttaaatgatgagctttatgtttttggtggcgtagggaaaaaaaacagtgaatcaccgttgcaggtttatagcgatgtgtacaaatactcaccggtgaaaaatacatggcaaaaagttgacactatatctccagttggattaacagggcatacgggagtaaaattaaacgaaacgatggtacttattaccggaggggttaatgagcatatctttgataagtattttattgatatagcggctgcggctgcggatgaaagtgaaaaaaataaagtcatctataattattttaataaacctgccaaagattatttttttaataaaatcgtatttatctacaatgctaaagagaacacatggaagaatgccggtgagctgccaggcgcggggacggcaggatcgtcatcggtaatggaaaataatttcctgatgctgattaatggtgagctcaaaccgggtttacgtaccgatgtgatttaccgcgccatgtgggataacgacaagctaacatggttgaagaacagccagttaccgccatcgcctggagaacaacagcaagaagggttggccggagcattttcgggctatagtcacggtgtcctgcttgtcggtggtggcgcgaattttccgggagcaaaacaaaattataccaatggaaagttttattcccacgaaggaataaataaaaaatggcgagatgaagtctatggtttggttaatggccattggcaatatatgggtaaaatgaaacaacctctcggctatggtgtatcagtaagttatggtgatgaagttttccttatcggtggtgaaaatgctaaagggaaacctgtttcgtctgtaacctcctttaccatgcgtgatggtaatttattaataaaataa

SEQ ID NO: 125 - DNA, 1215 bp, Shigella flexneri serotype 5b (strain 8401)
atgactaccttgaccgccagggtgtttacaacggcagagataatttatcggaaaacagttatcgcattggtgtgtcatttaaactgtagtagacaggagacagtcacaatgaataaaactataatggcgcttgctatcatgatggcttcatttgccgcaaacgcgtctgtattaccggaaactcctgtgccatttaaaagtggtaccggagcaattgataacgacactgtctacattggtttaggtagcgcaggtacagcatggtacaagctagatacacaggccaaagataaaaaatggactgcgttagctgcattccctggtggaccgagagagcaagcaacctctgcatttattgatggcaatctgtatgtgtttggcggcattggcaaaaacagcgagggattgactcaggtatttaatgacgtacacaaatacaaccccaaaaccaatagctgggttaaattgatgtcgcatgcgccgatgggcatggcgggtcatgtgacttttgtacacaacggcaaggcttatgttactggcggtgttaaccagaatatcttcaatggctattttgaagatctcaacgaggctggaaaagattcaaccgctatagataaaatcaacgcccactattttgacaaaaaagcagaagattatttcttcaataagtttctgttgtcttttgatccctcaacacagcaatggagttacgctggcgaatcgccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgatcccgtctcatcaccagatggcgtcgctggcggttttgcggggataagcaatgattctcttatatttgccggaggggccggattcaaaggttcacgagaaaattaccagaacggtaagaactatgcgcatgaaggcctgaaaaaatcatatagcactgatattcatctttggcataacgggaaatgggataaatcgggtgaattatcgcaaggtcgggcctacggagtatcattgccctggaataatagtttattaattattggcggtgaaactgcaggcggcaaagcggtgacggattcagttttaatttctgtgaaggataataaagtcacagtacaaaactaa

SEQ ID NO: 126 - DNA, 1179 bp, Yersinia pestis bv. Antiqua (strain Antiqua); identical to SEQ ID NO: 106

SEQ ID NO: 127 - DNA, 1128 bp, Haemophilus influenzae (strain PittGG)
atgaaactaactaaaacagcattatgcactgcactttttgccacctttactttttcagcgaacgcacaaacttatcctgatttgccagtggggatcaaaggaggaacaggcgcattaattggcgacacagtttatgtgggattaggttctggtggcgataaattctataccttagacctaaaagatccttcagcacaatggaaagaaattgctacatttccgggtggcgaacgcaatcaacctgttgcagccgcagtggatggaaaactttatgtattcggtggtttacaaaaaaatgaaaaaggcgaacttcaactcgttaatgacgcttatcgctataatccaagcgataatacttggatgaaacttcctactcgttctccccgtggtttagtaggatcaagtggtgcatctcacggagataaagtttatattttaggcggttctaatctctctatatttaatggtttcttccaagacactgtggcagcaggtgaagataaagcgaaaaaagatgaaatcgcagcagcttacttcgatcaacgtccagaagattatttctttacaacagaattattgagctatgaaccctcaaccaacaaatggcgcaatgaaggtcgtattccattctctggtcgtgctggtgcagcctttacaattcaaggtaatgatctcgtggttgtcaatggcgaaattaaacctggacttcgcaccgctgaaacccatcaaggtaaattcactgctaaaggtgtgcaatggaaaaacttacctgacttacctgctccaaaaggcaaatcacaagatggtttagctggtgcgctttcaggctatagtaacggtcattatttagtcactggtggagcaaattttccaggttcaatcaaacaattcaaagaaggaaaacttcacgcacataaaggtttaagcaaagcttggcataacgaagtttatacgttgaataatggtaaatggcgcattgttggggaattaccaatgaatattggttatggtttttctgtatcttacaacaataaagttttactgattggcggtgaaactgatggaggtaaggctttaactagcgtcaaagcaataagctatgacggtaaaaaattgaccatcgaataa

SEQ ID NO: 128 - DNA, 1140 bp, Haemophilus influenzae (strain ATCC 51907)
atggggtttattatgaaactaactaaaacagcattatgtactgcactttttgccacctttactttttcagcgaacgcacaaacttatcctgatttgccagtggggatcaaaggaggaacaggcgcattaattggcgacacagtttatgtgggattaggttctggtggcgataaattctataccttagacctaaaagatccttcagcacaatggaaagaaattgctacatttccgggtggcgaacgcaatcaacctgttgcagccgcagtggatggaaaactttatgtattcggtggtttacaaaaaaatgaaaaaggcgaacttcaactcgttaatgacgcttatcgctataacccaagcgataatacttggatgaaacttcctactcgttctccccgtggtttagtaggatcaagtggtgcatctcacggagataaagtttatattttaggcggctctaatctctctatatttaatggcttcttccaagacactgtggcagcaggtgaagataaagcgaaaaaagatgaaatcgcagcagcttatttcgatcaacgtccagaagattatttctttacaacagaattattgagctatgaaccatcaaccaataaatggcgcaatgaaggtcgtattccattctctggtcgtgctggtgcagcctttacaattcaaggtaatgagctagtggttgtcaatggcgaaattaaacctggacttcgcaccgctgaaacccatcaaggtaaattcactgctaaaggtgtgcaatggaaaaacttacctgacttacctgctccaaaaggcaaatcacaagatggtttagctggtgcgctttcaggctatagtaacggtcattatttagtcactggcggcgcaaattttccaggttcaatcaaacaattcaaagaaggaaaacttcacgcacataaaggtttaagcaaagcttggcataacgaagtttatacgttgaataatggtaaatggcgcattgttggggaattaccaatgaatattggttatggtttttctgtatcttacaacaataaagttttactgattggcggtgaaactgatggaggtaaggctttaactagcgtcaaagcaataagctatgacggtaaaaaattaaccatcgaataa

SEQ ID NO: 129 - DNA, 1140 bp, Pasteurella multocida (strain Pm70); identical to SEQ ID NO: 128

SEQ ID NO: 130 - DNA, 1161 bp, Salmonella arizonae (strain ATCC BAA-731)
atggggatgcagatgaaaaattctaagaaaatgatgacgctaatggcgctatgtttatcagttgctatcaccacctcagggtatgcaacgacgcttcctgatattccagaaccactgaaaaatggtacaggcgctattgataataatggcgtgatttatgtcggcttaggttccgcagggacatcctggtataaaattgatcttaaaaagcaacataaagactgggaacgtataaagtcgtttcctggcggagctcgcgaacaatcagtgtcggtatttttaaatggtgagttatatgtttttggtggtgtaggaaaagaaagcaatgaagcaccattgcaggtttatagcgatgtgtacaaatacacaccagcaaaaaacacatggcagaaagttaatactatatctccagttgggttgacaggacatacggtagtaaaattaaacgaaacgatggcgctcattaccggaggagttaatgagcatatctttgataagtattttattgatatagcggctgcggctggcgacgatagtgaaaaaaatagagttatttataattatttcaataaaccgtctaaagattatttttttaataaaatcgtattcatctataacgctaaagaaaatacatgggagaatgctggtgagctacctggagcggggacggcgggatcgtcatcggcgatggaaaataattctctgacgttgatcaatggcgagcttaaaccaggtttacgtaccgatgtaatttaccgtgccatttgggataacgataaactaacgtggttgaagaacagtcagttaccgccatcgcctggggaacaacatcaggaaggtttggccggagcattttctggatacagccagggtgttctgcttgttggtggcggcgcgaattttccgggagcaaaacaaaactataccgatggaaaattttatgctcacgaaggaataaataaaaaatggcgagatgaagtctatggtttaattaatggccattggcaatatgtgggtaaaatgaaacagcctctcggctatggtgtatcagtaaattatggtgatgagattttccttataggtggtgaaaatgctaaagggaaacctgtttcatctgtaatatcctttgccatgcatgatggtaaattattaatagaataa

SEQ ID NO: 131 - DNA, 1155 bp, Salmonella typhi
atggggatgcagatgaaaaattttaagaaaatgatgacgctaatggcgctatgtttatcagttgctatcaccacatcaggatatgcaaccacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatccgtgtcggtatttttaaatgataagctgtatgtttttggtggcgtagggaaaaaaaacagtgaatcaccgttgcaggtttatagcgatgtgtacaaatactcaccggtgaaaaatacatggcaaaaagttgatactatatctccagttggattaacagggcatacgggagtaaaattaaacgaaacgatggtacttattaccggaggggttaatgagcatatctttgataagtattttattgatatagcggctgcggatgaaagtgaaaaaaataaagtcatctataattattttaataaacctgccaaagattatttttttaataaaatcgtatttatctacaatgctaaagagaacacatggaagaatgccggtgagctgccaggcgcggggacggcaggatcgtcatcggtaatggaaaataatttcttgatgctgattaatggtgagctcaaaccgggtttacgtaccgatgtgatttaccgcgccatgtgggataacgataagctaacatggttgaagaacagccagttaccgccatcgcctggagaacaacagcaggaagggttggccggagcattttcgggctatagccacggtgtcctgcttgtcggtggtggcgcgaattttccgggagcaaaacaaaattatactaatggaaagttttattcccacgaagggataaataaaaaatggcgagatgaagtctatggtttgattaatggccattggcaatatatgggtaaaatgaaacaacctctcggctatggtgtatcagtaagttatggtgatgaagttttccttattggtggtgaaaatgctaaagggaaacctgtttcgtctgtaacctcctttaccatgcgtgatggtaatttattaataaaataa

SEQ ID NO: 132 - DNA, 1161 bp, Salmonella typhimurium (strain LT2)
atggggatgcagatgaaaaattttaagaaaatgatgacgctaatggcgctatgtttctcagttgctatcaccacatcaggatatgcaacgacgcttcctgatataccagaaccactgaaaaatggtactggcgctattgataataatggcgtgatttatgtcggcttaggtaccgcagggacatcctggtataaaattgatcttaaaaagcaacataaagactgggagcgtataaagtcgtttcctggtggagctcgtgagcaatccgtgtcggtatttttaaatgatgagctttatgtttttggtggcgtagggaaaaaaaacagtgaatcaccgttgcaggtttatagcgatgtgtacaaatactcaccggtgaaaaatacatggcaaaaagttgacactatatctccagttggattaacagggcatacgggagtaaaattaaatgaaacgatggtacttattaccggaggggttaatgagcatatctttgataagtattttattgatatagcggctgcggctgcggatgaaagtgaaaaaaataaagtcatctataattattttaataaacctgccaaagattatttttttaataaaatcgtatttatctacaatgctaaagagaacacatggaagaatgccggtgagctgccaggcgcggggacggcaggatcgtcatcggtaatgggaaataatttcctgatgctgattaatggtgagctcaaaccgggtttacgtaccgatgtgatttaccgcgccatgtgggataacgataagctaacatggttgaagaacagccagttaccgccatcgcctggagaacaacagcaggaagggttggccggagcattttcgggctatagtcacggtgtcctgcttgtcggtggtggcgcgaattttccgggagcaaaacaaaattatactaatggaaagttttattcccacgaagggataaataaaaaatggcgagatgaagtctatggtttgattaatggtcattggcaatatatgggtaaaatgaaacaacctctcggctatggtgtatcagtaagttatggtgatgaagttttccttattggtggtgaaaatgctaaagggaaacctgtttcgtctgtaacctcctttaccatgcgtgatggtaatttattaataaaataa

SEQ ID NO: 133 - DNA, 1179 bp, Yersinia pseudotuberculosis serotype O1b (IP 31758)
atgacccagttatacccacaatataagaaacaattgacgacaaaaatagtgctgtttagtgcgttatctctattgatgatggcatcgctacctaatacctatgcggaacaatatcctgacgttccggtaccatttaaaaacggtacgggtggcaaagtggagaacagcttatatgtggggctggggagcgccggtgtatcgtggttccgtctggatacagataaaaccggtgctgggtggcagaaagtggctaatttcccaggccagcctcgtgaacaggctgtgacggtggtattggcagggaaactgtacgtgttcggtggggtggggaaaacgaacgccaacgatacccaagtacgggcactggatgatgcgtatcgttttgatccacaaactaatcaatggcagcaactggctacccgcgctccccgaggtctggtggggaccgtggccacgacactggacggctcccaggcggtgctgttaggcggtgtaaataaagcgatttttgacggttattttactgatcttgcgtcagccggaagtgatgaagtacgtaagagtgcggtaataaacgcctattttaatcaggctccggctgattatttctacaaccgcgatgtccttatctatgatccgcaaaaaaatcagtggaaaagcggcggtttgctgccatttttagggacagccgggtcagccatcagccgcatggataatcgcttgatattaatcaatggcgaaattaaacctggtttacgcacagcggctgtatggcaggggctaatgcaagggaatgtactggagtggcagccacaaccggacttaatcggagctgaaacgggatccgcacaggaaggattggctggcgcattttccggtatcagtcataaaacagtcttagtcgccggaggcgctaatttccctggtgcctggaagcaatttaatcgcggtcacctatatgctcaccaagggctggaaaaacagtggcaccagcaggtgtatgcgttggtcgataatcagtggagaatcgccgggaagttgcctcaaccgctgggttatggcgtctctattcaggggccagataaggtcattttgattggcggcgaaaccaccggaggtacggccacttcagcggtcacacagctatcttggcagggaggaaaactgcatattgagtaa

SEQ ID NO: 134 - DNA, 1179 bp, Yersinia pestis; identical to SEQ ID NO: 106

SEQ ID NO: 135 - DNA, 1179 bp, Yersinia pestis (strain Pestoides F); identical to SEQ ID NO: 106

SEQ ID NO: 136 - DNA, 1179 bp, Yersinia pseudotuberculosis serotype I (IP32953)
atgacccagttatacccacaatataagaaacaattgacgacaaaaatagtgctgtttagtgcgttatctctattgatgatggcatcgctacctaatacctatgcggaacaatatcctgacgttccggtaccatttaaaaacggtacgggtggcaaagtggagaacagcttatatgtggggctggggagcgccggtgtatcgtggttccgtctggatacagataaaaccggtgctgggtggcagaaagtggctaatttcccaggccagcctcgtgaacaggctgtgacggtggtattggcagggaaactgtacgtgttcggtggggtggggaaaacgaacgccaacgatacccaagtacgggcactggatgatgcgtatcgttttgatccacaaactaatcaatggcagcaactggctacccgcgctccccgaggtctggtggggaccgtggccacgacactggacggctcccaggcggtgctgttaggcggtgtaaataaagcgatttttgacggttattttactgatcttgcgtcagccggaagtgatgaagtacgcaagagtgcggtaataaacgcctattttaatcaggctccggctgattatttctacaaccgcgatgtccttatctatgatccgcaaaaaaatcagtggaaaagcggcggtttgctgccatttttagggacagccgggtcagccatcagccgcatggataatcgcttgatattaatcaatggcgaaattaaacctggtttacgcacagcggctgtatggcaggggctaatgcaagggaatgtactggagtggcagccacaaccggacttaatcggagctgaaacgggatccgcacaggaaggattggctggcgcattttccggtatcagtcataaaacagtcttagtcgccggaggcgctaatttccctggtgcctggaagcaatttaatcgcggtcacctatatgctcaccaagggctggaaaaacagtggcaccagcaggtgtatgcgttggtcgataatcagtggagaatcgccgggaagttgcctcaaccgctgggttatggcgtctctattcaggggccagataaggtcattttgattggcggcgaaaccaccggaggtacggccacttcagcggtcacacagctatcttggcagggaggaaaactgcatattgagtaa

SEQ ID NO: 137 - DNA, 1215 bp, Shigella flexneri
atgactaccttgaccgccagggtgtttacaacggcagagataatttatcggaaaacagttatcgcattggtgtgtcatttaaactgtagtagacaggagacagtcacaatgaataaaactataatggcgcttgctatcatgatggcttcatttgccgcaaacgcgtctgtattaccggaaactcctgtgccatttaaaagtggtaccggagcaattgataacgacactgtctacattggtttaggtagcgcaggtacagcatggtacaagctagatacacaggccaaagataaaaaatggactgcgttagctgcattccctggtggaccgagagagcaagcaacctctgcatttattgatggcaatctgtatgtgtttggcggcattggcaaaaacagcgagggattgactcaggtatttaatgacgtacacaaatacaaccccaaaaccaatagctgggttaaattgatgtcgcacgcgccgatgggcatggcgggtcatgtgacttttgtacacaacggcaaggcttatgttactggcggtgttaaccagaatatcttcaatggctattttgaagatctcaacgaggctggaaaagattcaaccgctatagataaaatcaacgcccactattttgacaaaaaagcagaagattatttcttcaataagtttctgttgtcttttgatccctcaacacagcaatggagttacgctggcgaatcgccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgatcccgtctcatcaccagatggcgtcgctggcggttttgcggggataagcaatgattctcttatatttgctggaggggccggattcaaaggttcacgagaaaattaccagaacggtaagaactatgcgcatgaaggcctgaaaaaatcatatagcactgatattcatctttggcataacgggaaatgggataaatcgggtgaattatcgcaaggtcgggcctacggagtatcattgccctggaataatagtttattaattattggcggtgaaactgcaggcggcaaagcggtgacggattcagttttaatttctgtgaaggataataaagtcacagtacaaaactaa

SEQ ID NO: 138 - DNA, 1155 bp, Vibrio vulnificus (strain YJ016)
atgatgaaaacaaagtacttactgttaccgctgctggcttcttcctctttactctctcatatggcgttcgcgaacaatcactggccagatctgcccataggggtaaaaaatggggttagcgcccaaataggcagcaaggtttatgttggtttaggttcggcagaaaaatccttttatgtattggacacccaagcgccgcaaaacggttggacattgcttgctgagtttattggtccggaacgcagcggcgcgacagcaactgtggttggtgaaaacatttttgtttttggtggttcgggcaaagcgtctgatgacgcaagttcccccattatttttgatacggtttatcgctttgatacccagacgaaccgctggcatcaagtgaaaactcaaacacctgttgggctacttggtgcgtcttcttattcgcctgatggtaaacaggtgctgtttttcggtggttacagtaaaccgttgtttgataagtaccttgctgacattactcgaacggataagaaagctcagcctgaacaatggcagaaaattgtcgatgattacatggggatggagccgttagcgtatcaatggaatcgcgatgttctgagttttaatcctactaacgataaatgggacgttgtgactcgctctccctatcttcctaattgtggcagtgcaacagtcattgacggcccgagtatcacgctcatttctggcgaaatcaaaccaggtttgagaaccgcggaagtaaaaacctttacttacggtgaattgcagccatggcaaagtacctatgctctgccagcggccaagggacaagcgcaacaagaaggaattgcaggagcatattctggagtggtgagtaacacccttctcgtcgcgggtggtgcaaattttcatggcgcgaaagcacagtttgaatcgggccagatgttcgcgcacaacggtttgagtaaagcttacaacagcgaaatatacgccaagcaaaaaggcgtttggcaacaagtgggtcaactgcctgaaggattggcttatggcgcatcattcagtgttaaaggcggtgtattgatggtgggtggagaacgggccgatcgcactgcgagcaccaaagtgtaccttgttggcctcaacaataaccaaatagacatcgtcgattag

SEQ ID NO: 139 - DNA, 1635 bp, Bacteroides fragilis (strain YCH46)
atgaaaaaagccgtaattctattttcgcttttctgttttctgtgtgctataccggtagttcaagcagcagataccatttttgttcgtgaaacacgtattcctattttgatagaaaggcaggataatgtcttattctatctccgtttggatgctaaagagagccagaccctgaacgatgtagtgttaaacttgggtgaaggagtgaatctttctgaaattcagtccataaagctttattatggaggtactgaagcattgcaggatagtggtaaaaaacgttttgctcctgttggatatatttccagtaacactccggggaaaacacttgccgccaatccgtcttattcaatcaaaaaatcagaggtgactaatcccggtaatcaggttgttctgaaaggagatcagaaactgtttcccggaatcaattatttctggattagtttgcaaatgaagccgggcacttcgcttaccagcaaggtgaccgcggatatcgcttcaattactttggatggcaaaaaagccctgttggatgtagtttcagaaaatgggatagaacaccgtatgggagtaggagtgcgccatgccggagcagataattctgccgcattccgtattccgggattggtgactactaataaaggtacgttactgggagtttatgatgtacgttataacagtagtgttgacttgcaggagcatgttgatgttggtttaagccgcagtacagatggtggaaaaacttgggaaaaaatgcgtttgcctttggcttttggagaattcggtggtttgcctgccggtcagaatggggtaggagacccttctattcttgttgatacaaaaacaaataatgtttgggtggttgctgcctggacacatggtatgggtaatcagcgggcatggtggagttcacatccgggtatggatatgaaccatacagcacaacttgttttagctaaaagtacagatgatggtaaaacatggtctgcacctattaatattacagagcaggtgaaagatccttcttggtatttcttgttgcagggaccgggtaggggtatcactatgagtgacggtactttggtattcccaactcagtttatcgattcgacacgtgtgcccaatgccggtattatgtacagtaaagatggtggcaagaactggaagatgcacaattatgcacgtacgaacaccacagaagcccaggtagctgaggtcgaacccggagtgttgatgttgaatatgcgtgataatcgtggaggaagccgcgctgtggctattacaaaagatttgggtaaaacatggacggaacatgaatcttctcgtaaggcattgccggaatctgtttgtatggctagtttgatcagtgtgaaagcaaaagataatgtgttgggcaaggatttattgattttctctaatcctaatacgacaaaaggacgctataatactactattaaaataagtttggatggcggtgtgacttggtcacccgaacatcagctgttgttggatgaaggcaataactggggttattcttgcctctctatgattgataaagagacgattggtattctttatgaaagtagtgtggcgcacatgacattccaggctgtgaagttgaaagacattattaaatga

SEQ ID NO: 140 - DNA, 1215 bp, Shigella sonnei (strain Ss046)
atgactaccttgaccgccagggtgtttacaacggcagagataatttatcggaaaacagttatcgcattggtgtgtcatttaaactgtagtagacaggagacagtcacaatgaataaaactataatggcgcttgctatcatgatggcttcatttgccgcaaacgcgtctgtattaccggaaactcctgtgccatttaaaagtggtaccggagcaattgataacgacactgtctacattggtttaggtagcgcaggtacagcatggtacaagctagatacacaggccaaagataaaaaatggactgcgttagctgcattccctggtggaccgagagatcaagcaacctctgcatttattgatggcaatctatatgtgtttggcggcattggcaaaaacagcgagggattgactcaggtatttaatgacgtacacaaatacaaccccaaaaccaatagctgggttaaattgatgtcgctcgcgccgatgggcatggcgggtcatgtgacttttgtacacaacggcaaggcttatgttactggcggtgttaaccagaatatcttcaatggctattttgaagatctcaacgaggctggaaaagattcaaccgctatagataaaatcaacgcccactattttgacaaaaaagcagaagattatttcttcaataagtttctgttgtcttttgatccctcaacacagcaatggagttacgctggcgaatcgccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgatcccgtctcatcaccagatggcgtcgctggcggttttgcggggataagcaatgattctcttatatttgccggaggggccggattcaaaggttcacgagaaaattaccagaacggtaagaactatgcgcatgaaggcctgaaaaaatcatatagcactgatattcatctttggcataacgggaaatgggataaatcgggtgaattatcgcaaggtcgggcctacggagtatcattgccctggaataatagtttattaattattggcggtgaaactgcaggcggcaaagcggtgacggattcagttttaatttctgtgaaggataataaagtcacagtacaaaactaa

SEQ ID NO: 141 - DNA, 1944 bp, Micromonospora viridifaciens
atgactgcgaatccgtacctccgccgcctgccccggcgccgagccgtcagcttcctgctcgcaccagcgctggcggccgccacggtcgccggcgcgtcccccgcacaggccatcgccggggcacccgtcccgcccggcggcgagccgctctacacggagcaggacctcgccgtgaacggcagggagggctttccgaactaccgcatcccagcgctgaccgtcacgcccgacggggacctgctggcctcgtacgacggccgcccgaccggtatcgacgcgcccggccccaactccatcctccaacgccgcagcaccgacggcggccggacgtggggcgagcaacaggtcgtcagcgccggccagaccaccgcgccgatcaaggggttctccgaccccagctaccttgtcgaccgggaaaccgggaccatcttcaacttccacgtctactcccagcggcagggcttcgccggcagccggcccggcaccgacccggcagaccccaacgtgctccacgccaacgtcgcgacctcgaccgacggcggtctgacctggtcgcaccggaccatcacggccgacatcaccccggatccgggctggcgcagccgcttcgccgcctccggcgaaggcatccagctccgctatggaccccacgccggtcgactcatccagcagtacacgatcatcaacgctgccggcgccttccaggcggtgagcgtgtacagcgacgaccacggaaggacctggcgcgccggcgaagccgtcggggtcggcatggacgagaacaagaccgtggaactctccgatggccgggtcctgctcaacagccgcgactcggcccgcagcggataccgtaaggtggccgtctccactgacggcggccacagctacggcccggtgaccatcgaccgcgacctccccgacccgacgaacaacgcatcgatcatccgggccttccctgacgccccggccggctccgcgcgggccaaggtcctgctcttctccaacgccgccagccagacctcgcgcagtcagggcaccatccggatgtcctgcgacgatggccagacctggccggtttcgaaggtcttccagcccggctcgatgtcgtactccaccctgaccgcactgcccgacggcacctacgggctgctgtacgagccgggcaccggcatcagatacgccaacttcaacctcgcctggctgggcggcatctgcgcgcccttcacgattccggatgtggcgctcgagccgggccagcaggtcactgttccggtggccgtcacgaaccagtccggtatcgcggtaccgaagccgagccttcagctcgacgcatcgccggactggcaggttcagggttccgtcgagcccctcatgcccggacggcaggccaagggccaggtgaccatcacggttcccgccggcaccacccccggtcgctaccgggtcggtgcgacgctgcgcacctccgcgggtaacgcgtcgacgaccttcacggtcacggttggactgctcgaccaggcccggatgagcatcgcggacgtcgacagcgaggagaccgcccgcgaagacgggcgggcgagcaacgtgatcgacggcaacccctcgacgttctggcacaccgaatggtcgcgtgccgatgctcctggctacccgcaccgcatcagcctcgacctcggtggcacgcacacgatcagcggcctccagtacacccgacggcagaacagcgccaacgagcaggtcgcggactacgagatctacaccagcctgaacggcacgacctgggatggcccggttgccagcgggcgcttcaccacgtccctcgcgccgcagcgcgcggtcttcccggcgcgggacgccaggtacatccggttggtggccctcagcgagcagaccgggcacaagtacgccgcggtcgctgagctggaggtggaaggccagcgctga

SEQ ID NO: 142 - DNA, 1119 bp, Escherichia coli O6H1 (strain CFT073)
gtgattacaatgaaagtgaaaaactttatatatcttcccttctgcttgtttattggaacgtcggtagccggagctttaccggaaattcctgaaccatttaaatatggtgtaggagggatcgagaatggtaaaatatatataggactgggaagtctgggaaataactggtatatgatagatacaaaccagagtgaaaaaaaatggactaagatagcgcaatggccgacagttcccagagagcaggcaacagctactattattgatggtaagatctatgtatttggaggaattggaaaagatacaagtggagtcataaccttacaaaaagatgtctattcctatgatattgctaaagataaatgggaaaaattgatgacccgtccccctgtatctcttgccggtcatgtttcttttatccataatggtcatgcggtatctactggtggtgtaaatgagaatatttttaatggatacttttcagatgtcgagttatcaaaagggaattccgcgctgacagagaaagtcaacagggattattttagtaaacctgctgatgattattttttaaataatcatataatttcttatgacccgtcaaaaaaccagtggaagaacctgggaacgactccatttccgggaactgctggttcatcggtgatttttgcagaacagcaaatttatatactgggaggggagagaaaaccggggcttcgttctgttagatcctggacgggggagctttcgcatgatcgaattaaatggtctgaacttccaccagtagcttcacccgagggggtgtcaggagcatatgcctctgttattgatggaaatatctttctggccggtggtgcatatttcccgggagcggcagaaaagtatagcaatggtgaatactggtctcataaaggactggataaagcctattcaaaagagatatatcagctaataaaaaatgactggaaaaaagttggttctttgccggaaggacttgcttatggtgtaagcctgccctggcagggggggatgctcattctggggggggagaaaaaagatggtaaagctgtttcagatgttatttatctgaaaaaaaatgacaaacaaataaagattgttaaataa

SEQ ID NO: 143 - DNA, 1167 bp, Brucella abortus (strain 2308); identical to SEQ ID NO: 96

SEQ ID NO: 144 - DNA, 1215 bp, Escherichia coli O1K1 / APEC; identical to SEQ ID NO: 103

SEQ ID NO: 145 - DNA, 1107 bp, Escherichia coli O6K15H31 (strain 536)
atgaataaaacaataacggcgcttactatcataatggcttcatttgccacaaacgcgtctgtattaccggaaactcctgtgccatttaaaagtggtaccggagcaattgataacgacactgtctacattgggttaggtagcgcaggtacggcatggtacaagctggatacacaggccaaagataaaaaatggacggcgttagctgcattccctggcggaccgagagatcaagcaacctctgcatttattgatggcaatctgtatgtgtttggcggcattggcaaaaacagcgagggcttgactcaggtatttaatgacgtacacaaatacaaccccaaaaccaatagctgggttaaattgatgtcgcacgcgccgatgggcatggcgggccatgtgacttttgtacacaacggcaaggcttatgttactggcggtgttaaccagaatatcttcaatggctattttgaagatctcaacgaagctggaaaagattcaaccactatagataaaatcaacgcccactattttgacaaaaaagcagaagattatttcttcaataagtttctgttatcttttgatccctctacacagcaatggagttacgctggcgaatctccctggtacggaacggctggtgcggcggttgtgaataaaggtgataaaacctggcttattaatggcgaagccaaaccaggattgcgaacggatgccgtatttgaacttgatttcaccggtaataatttaaaatggaataagcttgctcccgtcgcatcaccagatggcgtcgctggcggttttgcaggcatgagcaatgactcacttatatttgccggaggggccggattcaaaggttcacgagaaaattaccagaacggtaagaactatgcgcatgaaggcctgaaaaaatcatatagcgctgatattcatctttggcataacgggaaatgggataaatcgggcgaattatcgcaaggtcgggcctacggagtatcattgccctggaataatagtttattaattattggcggtgaaactgcaggcggcaaagcggtgacggattcagttttgatttctgtgaaggataataaagtcacagtacaaaactaa

SEQ ID NO: 146 - DNA, 1632 bp, Bacteroides xylanisolvens
atgagaagaatttattatttacttttccttatccttttaggatattcttttgacgtaaaagcttcagacacagtatttatccatgaaacacaaatcccggttttgatagagcggcaagacaatgttctgttctatcttcgtttggatgcaaaagaaagtaagaagctcgatgaaatcattttagattttagtaaaagtacgaatctgactgatattcaggcaatcaaactctattatggaggaacagaggcgttgcaggacaaagacaaaaatcgctttgctccagtagagtacatatccagccaccgtccgggtgggacattggcggctattccttcctattcgatcaaatgtgcagaggtgggatcatcagaaaaagttgtattaaaaggtaattacaatttatttccgggtgtaaactatttttggattagtttgctgatgaaaaaagatgcctctttgcagactaagatactctctgaattgtgtgccgttaaagtagatgggaaagaactatgttgtaaatctatttctccaaagaatattgttcaccggatggcagttggagttcgtcatgccggtgatgacggttctgcttctttccgtattcccgggttggtgactaccaataaaggaaccttattaggcgtgtatgatgttcgatacaacagtagtgtcgatttacaggaatatgttgacgtgggtttgagccgtagcatcgatggaggaaagaattgggagaagatgcgtcttccgctttcatttggcgagtatggtggtctgcctaaagcacagaatggggtaggagacccttctattttagtagatactaagacgaataccgtctgggtggttgccgcatggacacatggtatgggtaatcaacgcgcctggtggagttcccatccgggaatggatctgaatcatacggcacagttagtattggctaaaagcacagacgatggtaaaacctggtcgaagcctatcaatataacagagcaggtgaaagatccttcctggtacttcctgcttcaaggtcccggacgtggaataacgatgagtgatggcacattggtatttccaacgcaatttatagactctacccgtgttccgaatgcaggaatcatgtatagtaaagaccgggggaaaacatggaaaatgcacaatatggcacgaaccaatacaacggaagcgcaagtggcggaaatagaaccgggagtattgatgctgaatatgcgtgataatagaggaggaagccgggctattgccataacgaaagatttgggagaaacatggacagaacacccatcttcccgccaagcacttcaggaaccggtatgcatggctagtctgattcatgtagatgccaaagacaacatattaaataaagatatcctgctgttttcgaacccgaatacgaccaaaggacgtaatcatataacaataaaagcaagtctggacaaaggacttacctggttgccggaacatcaattaatgctcgacgaggcagaaggatgggggtatagttgtctgacaatgattgataaagagacaattggtatactatatgagagtagcgtcgcacatataacctttcaagcgataaaattgacagatataataaaggaataa

SEQ ID NO: 147 - DNA, 2424 bp, Vibrio cholerae serotype O1 (strain ATCC 39315)
ttgtcaatcaagatgacttcacaacgaagaagagcatcgattcacaaggaaacagattctaatataaagggagtagatatgcgtttcaaaaacgtaaagaaaaccgctttaatgcttgcaatgttcggtatggcgacaagctcaaacgccgcactttttgactataacgcaacgggtgacactgagtttgacagtccagccaaacagggatggatgcaagacaacacgaataatggcagcggcgttttaaccaatgcagatggaatgcccgcttggttggtgcaaggtattggagggagagctcaatggacatattctctctctactaatcaacatgcccaagcatcaagtttcggttggcgaatgacgacagaaatgaaagtgctcagtggtggaatgatcacaaactactacgccaacggcactcagcgtgtcttacccatcatttcattagatagcagtggtaacttagttgttgagtttgaagggcaaactggacgcaccgttttggcaaccggcacagcagcaacggaatatcataaatttgaattggtattccttcctggaagtaacccatccgctagcttttacttcgatggcaaactcattcgtgacaacatccagccgactgcatcaaaacaaaatatgatcgtatgggggaatggctcatcaaatacggatggtgtcgccgcttatcgtgatattaagtttgaaattcaaggcgacgtcatcttcagaggcccagaccgtataccgtccattgtagcaagtagcgtaacaccaggggtggtaaccgcatttgcagagaaacgtgtggggggaggagatcccggtgctctgagtaataccaatgacataatcactcgtacctcacgagatggcggtataacttgggataccgagctcaacctcactgagcaaatcaatgtcagtgatgagtttgatttctccgatcctcggcctatctatgatccttcctccaatacggttcttgtctcttatgctcgatggccgaccgatgccgctcaaaacggagatcgaataaaaccatggatgccaaacggtattttttacagcgtctatgatgttgcatcagggaactggcaagcgcctatcgatgttaccgatcaggtgaaagaacgcagtttccaaatcgctggttggggtggttcagagctgtatcgccgaaataccagcctaaatagccagcaagactggcaatcaaacgctaagatccgaattgttgatggtgcagcgaaccagatacaagttgccgatggtagccgaaaatatgttgtcacactgagtattgatgaatcaggtggtctagtcgctaatctaaacggtgttagtgctccgattatcctgcaatctgaacacgcaaaggtacactctttccatgactacgaacttcaatattcggcgttaaaccacaccacaacgttattcgtggatggtcagcaaatcacaacttgggctggcgaagtatcgcaggagaacaacattcagtttggtaatgcggatgcccaaattgacggcagactgcatgtgcaaaaaattgttctcacacagcaaggccataacctcgtggagtttgatgctttctatttagcacagcaaacccctgaagtagagaaagaccttgaaaagcttggttggacaaaaattaaaacgggcaacaccatgagtttgtatggaaatgccagtgtcaacccaggaccgggtcatggcatcacccttactcgacaacaaaatatcagtggcagccaaaacggccgcttgatctacccagcgattgtgcttgatcgtttcttcttgaacgtcatgtctatttacagtgatgatggcggttcaaactggcaaaccggttcaacactccctatcccctttcgctggaagagttcgagtatcctagaaactctcgaacctagtgaagctgatatggttgaactccaaaacggtgatctactccttactgcacgccttgattttaaccaaatcgttaatggtgtgaactatagcccacgccagcaatttttgagtaaagatggtggaatcacgtggagcctacttgaggctaacaacgctaacgtctttagcaatatcagtactggtaccgttgatgcttctattactcggttcgagcaaagtgacggtagccatttcttactctttactaacccacaaggaaaccctgcggggacaaatggcaggcaaaatctaggcttatggtttagcttcgatgaaggggtgacatggaaaggaccaattcaacttgttaatggtgcatcggcatattctgatatttatcaattggattcggaaaatgcgattgtcattgttgaaacggataattcaaatatgcgaattcttcgtatgcctatcacattgctaaaacagaagctgaccttatcgcaaaactaa

SEQ ID NO: 148 - DNA, 1149 bp, Clostridium perfringens
atgtgtaacaaaaacaatacctttgaaaagaatctagatataagccataaaccagaaccactaatactatttaacaaggataataacatatggaattcaaagtattttagaattcccaatatacaattattaaatgatggtacaattttaaccttttcagatattcgttataatggtcctgatgaccatgcttatatagacatagcttctgcacgtagtactgattttggaaagacatggagctataacatagcaatgaaaaataatcgtattgactctacttattctcgtgtaatggactccacaacagttattacaaatacaggtagaataatattaattgcaggctcatggaatacaaatggaaactgggcaatgactacttctacaagaagaagtgattggtctgtccaaatgatttattctgatgacaatggattaacttggtctaataaaatagatttaactaaggactcttcaaaagtaaaaaatcaaccaagtaatacaattggatggctaggaggagttggctcaggtattgtaatggatgatggaacaatagttatgccagcacaaatttccttaagagaaaataatgaaaataactattattcattaattatctattcaaaggataatggtgaaacatggacaatgggaaacaaggttcctaattcaaacacctccgaaaatatggtaatagaattagatggcgctttaattatgagtacaagatatgattactctggctatagggcagcatacatctctcatgatttaggaaccacttgggaaatatatgaacctttaaacggtaaaattttaactggtaagggctctggatgccaaggttcctttattaaggctactacttcaaatggacatagaataggattaatttcagcacctaaaaacactaaaggtgaatatataagagacaatattgccgtttatatgattgactttgatgatttatctaaaggagttcaagaaatatgcattccttatcctgaagacggtaacaaattaggtggtggctattcttgtctatcatttaaaaataaccatctaggcattgtttatgaagccaatggaaatatagaatatcaagacttaacaccttattactcactaattaataaacaataa

SEQ ID NO: 149 - DNA, 1632 bp, Bacteroides xylanisolvens; identical to SEQ ID NO: 146

SEQ ID NO: 150 - DNA, 3549 bp, Unknown
  Environmental sample
atgaaaaaacgtaaacgtgttctggcgctgatgctggcgggtgttctgaccctgggttgcttcccgaccaccgttcaggcggttaacaacgcgtacgacggtaccaaaccggcggacggtaccaccaaaaaacagccgttcctggcgggtaccggtggttctaccaacttccgtatcccgggtctggttaccctgaacgacggtaccctggttgcgtcttctgacgcgcgttggaacggtggtggtgacggtggtggtctggacaccatcgtttctcgttctaccgacaacggtaaaacctggtcttacaccttcgcgaactacctgggtgacaacggtaacgttcacaacaacggttctaccgcgttcatcgacccggcgctgaccaccgacggtgaaaccgtttacatggcggcggacctgtacccggcgggtaccgcgctgaactctgcgtcttacgcgccgaaaaccggtcacaccggttacgacttccgtcaccgtctggttctggcgaaagcgaccgaaggtgttaccgcggcgtctgacaccgcgcagcgtatgaacgcggcgttcacctactacctggaaaaaaacccggacgaaaacgcggcgtcttactacctgctgaaagacgcgtctggtaacaccgtttctggttacaccgttgaccgtacttcaacatcaccggtaacggtgttaacaccaacctgttctgcggtgactctccgtacttcccgtacccgaccgacttcatctacatgaccaaatctaccgacggtggtgaaacctggtctgaaccgaccttcctggacgttaaaaaagcgtctgaacagtctctgctggttggtccgggtcgtggtatcgttatctcttctggtgaacacaaaggtcgtatcgttttcacctgctacgaattcacctctggtgacaaaaactctgcggcgatctactctgacgacaacggtgttacctggcaccgtggtgcgtctgttaaaaacctgtcttctgaagcggttgttaccgaagcggacggtaaactgtacatgttcgttcgtcgtgaaaacgtttactacgtttctaacgacggtggtgaaacctggtctgacccgcaggcgatgggtatctcttacaacgcgacctgccagctgaccgcgaccacctactctgaaaaaatcaacggtaaaaccgcgatcttcttcgctgcccgtctaacacctcttctcgtgcggcgggtaaaatctacgttggtctggttcaggcgacggttctatcgactgggcgtacgactactctatcaacggttctgcgtactacgcgtactcttgcatctctgaaatgggtaacggtaacatcggtctgctgtacgaatctaacggtaccgcgatcaccttcaaagaaatcgcgctgaaagacatcgttggttctgcgtctgttggttctgtttggctgaccgacgaagacggtaacgttatctcttctgactctatgaaatcttctcagaccaaaacctacaccgttaacggtgttgcggaaggtacctctgttgaagttacctcttctgacgaatctaccgttaccgcggtttacgaaaacggtaaactgaaagttacctctggtcaggttgaaggtctgaaacaggcggcggttaccgttcacgcgggttctttcgttcgtatcctgaaactgaacatctctgacaccgaaaactacaaagttgttgaactgaacatgggtggtaccaaatcttacaccgacaccaccggtaactactctgcggcggacacctcttctctggacaaaaccaccgcgaaagttaccatgaccggtgaagacccgcaggcgccggaagttaaaaccgttgcggttaaagcgaccgcgaccgcgaccttcaacggtgcggaactggacctggacgactgcgaatacaccttcgaagcgaccggtaccgacaaccagtacaaagttaaagcgaccaccgcggacggtcagaccgtttacctgggtacccgtgcggcgggttctaacaacgttccgtcttctaccaccgaaacctctatcaccgttgcgaaacactctaccgactctctgttcaccctgaaagacaactctaccggttctgttggtaacatcctgtggttctggtctaccgacacctctaaactgtacttcgaccgtaactcttctgttgcggacaactgctacttcgaactgtacaaaaaatctgcgtctgcgccggaagactctgcggttcacggttaccagaaagttaccgcgctggacgaaatccagggtggtaaaaaatacctgatcgttaccaaagcggacgcgaacaacaaccagtacgttgttgttccgtctgcggcggcggttaacgacaacgactggtacctgttcgcgccggaaaccggttacttcgttaccgcggaaaaagcgggtaccaccaccgttgttatcggtggtgttacctacgaaatcgttgttaccgaaaccgaatgcaaacacgaaaacaccgaacgtgttggtgttaaagacccgacctgcaccgaaccgggtaccaccggtaaactggtttgcaaagactgcaacgaaaccctggaagaagcggcggaaatcgcggcgaccggtcactcttacggtgaatggaccgttgttaaagaagcgaccaccgaagaagaaggtctggaaaaacgtgtttgcgaaaaatgcgacgcggaacagacccgttctatcccgaaaccgtctgacacccagaaaccgtctgaccaccagaaaccgtctgacaacaaaaccgcgggtaccaccgttaccggttctaaagcggcgaaaaccggtgacgtttctaacgttctgctgccgggtatgaccatgctgctggcgggtggtgcggcggcgggtaccctgctgggtaaaaaacgtaaaaaagcgaaccgttacgaacacgttggtaaactgaaaaccaacaccgaaggtaccccgggttctacccagatcaccttcgaaggtaccggtgaaggtaccacctctgttaccatcggtgcggttacctactacgttgttgttaaaaacgaagttcagaaagttgaactgaaagttggtgaaaaattcctggcgccgggtaaactgatgcagcaggaatcttctcagaaaatcctgtctctgaccgcgaacggtgaccagccgccgtacgttaaaaacaccgaaatcgtttctggtaaagaatacctgatcggtcagggttcttacgttctggttaactctcagtctaccgcgtctggtaccccgaccggtctggcgatgaaaaccgcggacctggaatctgacgaccagtctgcgtacatgtggaccgttaaagcggttgacggtggttacaccatccaggacgttaacggtaaatacctgtctttcaacggttctaacgttggtctgtctgacaccgcgcagaccctgaccgttggtaacggtgcgtctgacggtttcggtatctcttacggtggtcagtacctgaacaactacggtcgttctaacaccaaagttgcgggttactctgcg

SEQ ID NO: 151 - DNA, 1275 bp, Unknown
  Environmental sample
atgatcttctctgcgaacatcggtaaagcgctgtgcctggcgaccatgggtatcaccctgatgatgccggcgcaggttgcggcggcgaccccggaaatggcggttgaagcgcaggcggttaacgcgcaggacggtatcaccgttttcccgaacaaagaagcgcagtaccgtatcccggcgatcgttgaatgcaaatctggtaaactgatcgcgttcaccgaccaccgttaccacaacaaagacatcggtggtggtcgtcacctggacatcgttatgaaaacctctatggacaaaggtgcgacctggtcttctccggaacagatggttgcgaaaggtggtaacggtatcgcgacctctttcgactgcgcgcacggtgacgcggcggttgttgttgacaaaaaatctggtcgtatcctgctgatgtgcgcgtctggtggtatcggttactgggaatctaaacgtggtcacctgctgatgatgggtcgttactactctgacgacgaaggtaaaacctggtacggtgaagaagttaccaaacagatctacgacctgatgccggaagttgaatctgcgttcttcacctctggtcgtatctgccagtctaaacagatcaaagttggtaaatactaccgtgtttacaccgttctgtgcacccgttctggtaaccgtatcctgtactctgacgacttcggtgaaacctggaacgttctgggtaaaaacgttgcggaagcggcgccgtacggtgacgaagcgaaaatcgaagaactgccggacggtaacgttctgctgtcttctcgtgcgatgggtggtcgtcacatcaacatctacacctacgaagacaaaaaaaccgcgaccggttcttggggtaaagttgttgcgtctgacgcgaaaaacatgggtgttgcggcgcacaaaaactcttgcaacggtgaagttctgatggttgacgcgaaaaaaaacggtaaaaaagttaaactgctgctgcagtctgttccggttggtccgggtcgtaacaacgttggtatctactacaaagcgctggaaaacccggcggactacgcgaccccggaagcgatcgcgaaaaactgggaaggttgctaccagctgtctaacaccacctctgcgtactctaccatggttcagggtaaagacggttctatctacttcctgctggaagaaaacgcgttccgtaacccgaaaacccagaccgacgactactacgacatccgtttctacaacctgtctatcgaacagatcaccaacaaccgttacaaa

SEQ ID NO: 152 - DNA, 1293 bp, Unknown
  Environmental sample
atgaaaaaaatcttcctgaccctgggtctggcgctggcgtgctctgcgtcttgggcgcagaccctgctgtggcagaccgacaccacccgtcaccagtactaccgtatcccggcgatcgttgcgaacggtaaaaacgttatcgcgttcgcggacaaccgttctggtgttaccgacgcgacctcttggggtgacgttggttctgttggtaacatctctatcgaagttcgtcactctaaagacggtggtcgtacctggacctctccgaaatgcgcggttaaaggtttcggtaacggtggttacgaccagtctcacggtgacgcggcggttgttcgtgaccgtgaaaccggtcgtatgctgatcatgaccggttctggtgacgttggttacggtcgttctaaagttaccgttccgggtgactactctgaagcgctgaaagttggtcgttactactctgacgacaacggtaacacctggaacggtggtgaagttacctctcagatctacgacctgtaccgtggtcacggtaacatcttccgtctgttcttcacctctggtcgtatctgccagtctcgtatgatcaaacgtggtgaatactaccgtatctactctgcggttgttaccaacgaaggttctctggttgtttactctgacgacttcggtgacacctggaccccgctgggtggtgcggacgcgcgtccggcgccggaaggtgacgaagcgaaactggaagaactgccggacggtcgtgttctgctgtcttgccgtcgttctggtaaagacggtcgttggttcaacgttttctcttacggtgacaactcttacggtcagggttcttgggcgcagccggttgcgtctgaaaacccggacggtggtaccgcgaccatcaacaacaactgcaacggtgaaatcctgctggttccggcgaaagcgaaagacggttctcgtgtttacgttgcgctgcagtctgttccgcgtggtaccccgggtaaccacccgtctaacctggaacgtcgttctcacgtttctgtttactggaaagcgttccgttctgcggcggactgggcgtctccggaaaactggaaagacggttggaaacgttacgaaatcaccgacggttactctgcgtactcttctatggcgatcggtgacaaaggtaacatctgcttcatctacgaagacgacggtgttcgtttccgtctgggttctcagtctaccgaaatgtactctatcttctaccgtaacctgaccctgaaagaaatcaccggtggtaaatacaaatactctaaaatcaaa

SEQ ID NO: 153 - DNA, 4095 bp, Unknown
  Environmental sample
atgaaacgtatcaaactgatcaccgttatcctgctgttcctgttctctctgctggcgaaaggtgaagaccgtatcggtgttctgcagatcaacgtttggcaggaaggtacctgcgttccgggtggtttcgacgcgctggttgacaacatcatcgaatctggtgcggacgttgttttcatgcaggaaatccgtaactacggtggtcgtcgtttcatcccgcgtctgatcgaagcgctggcgctgcgtggtgttaccttctacggtgaagacaccacctgcgactctggtgttatctctcgtttcccgctggcgcaggacggtcgtaaatgggaaggttgcggttctgttcagcgtgttcgtatcaacgcgggtggtcacccggttgcgctgtacgcggcgcacctggactacaccaactacgcgtgctactacccgcgtggttacgacggtaacacctggaaaaaaatcccggcgccggaaacctctgttgaaaaaatcctggaaatgaaccgtgcgtctggtcgtgacgacaccatccgtgacttcatccgtctgtctcgtgcggacatcgacgaaggtatcgacgttatcctggcgggtgacttcaacgaaccgtctcacctggactggaccgcgcagaccgcgtctctgtgggaccacaacggtgcggttatcccgtgggactgctctaccatgctgtacgaaggtggtttcgaagactctttccgtgttatgaacccggacccggtttcttctccgggtttcaccttcccgtctgacaactctgactgcaacccgatgaaactgacctgggcgcacgaatctgacgaacgtgaccgtatcgacttcatctactactacggtgaaggttctctgaaaccggttgactctaaagttttcggtccggcgggttctatcgttcgtggtaaacgtatcgttgaagaaaaccactctgacatcatcccggttaccggtaaatggccgaccgaccacaaagcggttctgaccaccttcgaattcgcgccggttgcgcgtatcctgaccccgtctatcccgaccatcgttgaccgtgaacacaacatcatctgcgaaatctgcctggaatctttctctggtaaagacgttctggaatctgttggtgttaccgtttctggtatcgacccgtctgcgatccgtaacgcgcgtctgatgtacaccggtacctcttctgcgatctactctcgtaccacctcttacatcctgaaagaccagttccgtcgtatcggtgcgggtcaggacacctggcgtcacccggcgttcgcggttcagaccgcgtcttgcaaagcgggtaaagacggtcgtatcgttctgaaaccgggtaaatctctggttcgtgaccgtaactacttctacctgtctctggaagttgactctcgtaaagttgacctgtctggtaccttcggtgttgcggttgaatctgttgaactgcgttgcccggttcaggacggttctggttctctgctgcgtcaggcgcgtatcgtttctgaaggttctctggaaggtcaccgtttcggtatcgcgctgcgtaactacaaagacgacaacgttttctcttaccgtatcccgggtctggttaccaccccgaaaggtaccctgatcgcggtttacgacatccgtcacacctctggtaacgacctgcaggacaacatcgacgttggtgcgtctcgttctaccgacggtggtcgtacctgggaaaaaatgcgtaccatcatcgacatgggtgaatggggtggtctgccgcagggtcagaacggttgcggtgacccgtctgttctggttgactctaacaccggtaccatcttcgtttgcgcgctgtggacccacggtctgggtaaccgtcacgcgttctctttcgcgtctgaaggttacgaaccgatctctaccggtcagatggttctggttaaatctaccgacgacggtaaaacctggtctaaaccggttaacatcacccgtcaggttaaacgtccggactggcgtatcaccctgcagggtccgggtcgtggtatcaccatgcaggacggtaccctggttttcccgatgcagtgcctggactctaaaggtatcccgtcttctggtatcatgtactctaaagacggtggtgaaacctggcactgccactctcgtgcgtgcgaacgtaccaccgaagcgcaggttgcggaaatcgcgccgggtgttctgatgctgaactgccgtaacgacctgcgtcagggtcgtgttgtttgcgttaccgacgacctgggtaaaacctggaaaacccacccgtcttctggttctctggttgaaccggtttgcatggcgggtctgctgaacatcccggcgtctgaaaacatctacggtcgtgacatcctgctgttctctaacccggtttctgcgccgggtaaaggtcgtaaccacatctgcatcaaagcgtctctggacgcgggtatgacctggaacccgctggactgcctgctgctggacgaagaagaaggttggggttactcttgcctgacctctatcggtaacgacaaagttggtatcctgtacgaaggttctacctctcacctggttttccagcaggttcgtctgtctgacatcgttaaatctttcccggcgccgtctggtctgtctgttggtgaaatctactctgacgacatgatcttccagcgtgacgttccgctggaaatctctggtcgtaccgcgccgggtctggaagttaaagttaccctgggtccgtctacccgtaaatgccgttctgcgcaggacggtacctggaaagttcgtatgccggcgctgaaagcgggtaccggtatcccgttcaccgttaccgacggtcgtgacaccctgtctttctctaacgttgcggcgggtgaagtttggctgtgctctggtcagtctaacatggcgttcgaactgaaaggtgcggacaccttctctaaagaaggtctggacgacccgatgctgcgtttctacgacatgaaatctcacctgaaaacctggggtctggtttggtctgaagaagacgcgggtaaagttgcgcagggtaaatactacgaccgtacccgttggtcttgctcttctgaaaaaaccgcgggtaaattctctgcggttggttactacttcggtaaaatgctgcgtgactctctgaacgttccggttggtctgatctgcaacgcggttggtggtaccaccaccgaatctttcatctctcgttctgcgctggaaaaagttctgccgggtttctgcgacggttggctggaagacgaatctgttatggaatgggcgcgtggtcgtgcgcgtgaaaacatgtctgcgttcaaaggtggttacatccagcacccgttcatgccggcgtacctgttcgaaaacggtatccgtccgctggaacgtttcccgatcaaaggtgcgatctggtaccagggtgaatctaacgcggaaaacatcccgcagcacgaaatcctgttccgtaccctggttgactgctggcgtcagacctgggacaacccggacctgccgttctacttcgttcagctgtcttctatcgaacgtccgacctggccggaattccgtaactctcagcgtctgctgtctctggaaatcccgcactgcggtatggcggtttcttctgacctgggtcacccgaccgacgttcacccgcgtgcgaaaaaaccggttggtgaacgtctggcgcgtctggcgctgcgtaacgactacggtaaacaggttgaatgcctggcgccggcggcgaccaaagcgttcctgaacgactctggtaacgttaccgttctgttcgactctccgatctctgttccgggtaccatgccggttcgtggtttccgtatctgcctggaagacggttctgaaatcccggcgtctgcggaaatctctggttctgacgcggttatcctgtctcgtgacgcgggtcgtccggacgcggcggcggactctaaaaacaccaaagcggactctcgtaccgcgatcgttaaagttaaatacgcgtgggacccgtggaccgacgcgaacctgaccggtaccaccggtctgccggtttctaccttcgacatcgaaatcggtgaa

SEQ ID NO: 154 - DNA, 4095 bp, Unknown; identical to SEQ ID NO: 153
  Environmental sample

SEQ ID NO: 155 - DNA, 1275 bp, Unknown
  Environmental sample
atgatcttctctgcgaacatcggtaaagcgctgtgcctggcgaccatgggtatctctctgctgtctaccgttcaggttgcggttgcgatcccggcggttgcggcggaaacccaggttgttaaccgtcagggtggtatcaccgttttcccgaaccgtgaagcgcagtaccgtatcccggcgatcgttgaatgcaaatctggtaaactgatcgcgttcaccgaccaccgttaccacaaccaggacatcggtggtggtcgtcacctggacatcgttatgaaaacctctatggacaaaggtgcgacctggtcttctccggaacagatggttgcgaaaggtggtaacggtatcgcgacctctttcgactgcgcgcacggtgacgcggcggttgttgttgacaaagaatctggtcgtatcctgctgatgtgcgcgtctggtggtatcggttactgggaatctaaacgtggtcacctgctgatgatgggtcgttactactctgacgacgaaggtaaaacctggtacggtgaagaagttaccaaacagatctacgacctgatgccggaagttgaatctgcgttcttcacctctggtcgtatctgccagtctaaacagatcaaagttggtaaatactaccgtgtttacaccgttctgtgcacccgttctggtaaccgtatcctgtactctgacgacttcggtcagacctggaacgttctgggtaaaaacgttgcggaagcggcgccgcgtggtgacgaagcgaaagttgaagaactgccggacggtaacgttctgctgtcttctcgtaccatgggtggtcgttacatcaacatctactcttacgacgacaaaaaaaccgcgaccggttcttggggtaaagttgttgcgtctgaagcgcagaacctgggtgttgcggcgcacaaaaacgcgtgcaacggtgaagttctgatcgttgacgcgaaaaaaaacggtaaaaaagttaaactgctgctgcagtctgttccggttggtccgggtcgtaccaacgttggtatctactacaaagcgctggaaaccccggcggactacgcgaccccggaagcgatcgcgaaaaactgggaaggttgctaccagctgtctaacaccacctctgcgtactctaccatggttcagggtaaaaacggttctatctacttcctgctggaagaaaacgcgttcaaaaacccgaaaacccagaccgacgactactacgacatccgtttctacgacctgtctgttgaacagatcaccaacaaccgttacaaa

SEQ ID NO: 156 - DNA, 1467 bp, Unknown
  Environmental sample
atgaaaaaaatcctgttctctctgggtatgctgctgctgaccgttgcggcgtacgcggacggtaccatcctgttctctaccgcgaaaggttctatcgacgttccgccgtaccgtatcccgggtatcacctgcggttacggtggtcgtctggttgcgtctgcggcgcgtctggtttgcggtaccgacccgggtttcggtcgtgttgactgcgttgttaaactgtcttacgacaacggtcgtacctggtctgaacacgaaatcgacgttgcgtgcggtgacacctctctgatcaacgcgcacaaaaccccgatggaagcggcgtacggtgacccggcggttgttatggaccgtgaacgtaacgaagttctgatcatggcggttgcgggttgcaccgtttacggtaaaccgtctaccaaccgtcagaacccgaacctgatcgcggcgatccgttctaccgacggtggtctgacctggcagaaaccggttgaccagaccgaagacatctacggtctgttcgacaaaggtaacgttgttgacgcggcgttcgttggttctggtaaactgttccagtctcgtgttgttaaaatgggtgactactaccgtatctacgcggcgctgaccgcgcgtccgaacggtaaccgtgttatctactctgacgacttcggtcgttcttggcacgcgctgggtggtgcgtctgcgctgccggttccgggtggtgacgaaccgaaatgcgaagaactgccggacggtcgtgttatcatcacctctcgtgcgtctggttgccgtctgctgaacatctacacctactctaacaccaaaaccggtgaaggttgctgggacaaaccggaaaaagcgaccatggacggtctggcgctggcgccgtctaccaacccgaccaacggtgaaatgctgatcgttccggcgaaacgtaactctgacggtaaaccgctgtacgttgcgctgcagtctgttccgaccggtaccggtcgtaacaacgtttctatcttctacaaagaactggcgtctccggacgacatgcgtaacgcgaaagcgttcgcgtctggttgggacggttgctaccaggtttcttctaccgtttctatgtactcttctatggacctgcaggcggacgaccgtatcgcgttcttctacgaagaaaccctgaccaaatggggtaccaaaccgaacccggtttctacctctttcccgcagggtgaaggtgaacacaactacgacggttgcgaactggtttacaaatctttcgacctggaaaccatcaccgacggtaaatactgcgttaaccgtcgtatcaaccgtggtaaattcctgaaagcgtactacgaagacatcgttgactctttcggtctgaccgcggcgcagaaacgtaaagttaaagacgttatcaaacgtctgccggcggaaccgacctctgcgcagatggacgcgatcctggaaggtgaagttcgt

SEQ ID NO: 157 - DNA, 2655 bp, Unknown
  Environmental sample
atggcggttaccggtctggcgaccgacaccgttgaagcgttcgttctggttggtggtaactacgcgatcgacatcaccaacttcaacgttcactacgaaccggttccggaaaaagaagacaccaccgttaccgacaccgttgcgatcccgaccatcgacatcaacaccaccaccggttctttcgtttctaaaaactctgcgggttcttgggcgtctctgtggaaatctacccagtcttctccgtacttcaacctgggttgcggtgcgaacaacatggacgttaacaactctaccgaaggtaaaatcatcgcgtacgttggtaaccagaccaactcttgcgactacaccctgtctgttgaacgtggttggaaaatcaccggttactctttcgacttcgttcgtgttaccggtaaaaacgcggttaccctgaccgacgttaacggtaacaccttcacctcttctgacgaagttcagcacgtttctgttgacaacctgaaaaaatcttctctgaaagcgttcaacctgtctggtggtaacgacggtgttgttatcaccaacttcaaagttaaatacgttgttgactctgcgtacgttgcggcggacttccacaccttcaccgttttcgacaacaccggttctgttccgtaccgtatcccggcgatcggtaccgcgcaggacggttctctggttgcggttgcggactaccgttactctaaagcggacatcggttctggtcgtgttgacctgcacatccgtatctctcacgacaacggtctgacctggggtgacgttatggacctggaacagttccgtggtgacggtaacctgtctgaatggcgtcacgacaaagcggcgttcggtgacccgtgcatcgttggtgaccgtgaatctccgcgtatgatgatcacctcttgctctggtttcccgggtttcttcgacgcgaccgaccgtcaccagggttgggcgcgttggtactctaacgacaacggtctgacctggggtgaaccggaatacctggacgaaaaattcatctacgaaccgctgaaaaaagcgggttacaacgttcagggtttcttcgttggttctggtaaaatcttccagtctcaccacatcaaacgtggtgactactaccgtctgtactgcgcgggttctacccaggaaggtaacaaaaaaaactgggttctgtactctgacgacttcggtgaaacctgggaattcctgggtggtacctctaaagcgccgatcatctctaccgcggacgaaccgaaagttgaagaactgccgaacggtaacgttatcctgtcttctcgtgacgtttctggtcgttactacaacatcttcaccttctctggtaacgacggtatctctggttcttggggtgaaaaagcgctgtcttcttctgcggttaacggtctggttgcgaacaacggttgcaacggtgaaatccagatcgttccggttgttcgtaaaaaagacggtaaaaaaaccttcctggcgctgcagtctctgccgacctctggtcgtaccaacgtttctatcttctacaaagacctgaccgacgaagcgacctacaccaccccggcgaacttcgcgaaagactgggacggttctttcgttgtttgcgacttctcttctgcgtactctacctggtcttggcagcacgacaacgcgctggcgttcctgtacgaagaaggtggtggtacctctggtggttacaacatcgtttacaaacgtatcgacatcccgaccatcacctctcaggaatacgacttcgacacctcttacaaatacgaacgtttcaacatcgactgggcgcaggcggacctgaacatggaaatggcgtctctgctgaccaccgttaaaaaaacctctgaagcgaacacctctttcgttaaaggtgacaaactgatcaccgacgcgtctcagctgacctgcaaattcggtcacaaagaaatgggtggtaccggtggtgacgcgcaggacatctctaacctgatcgacggtgacccggcgacctactaccacacctactggggtgcgggtgaccagccgaacggttctcacttcatggacgttaccctgccggaaggtcagaccttccagggtaacatcgaagttgacgttacccgtcgttctggtgcgaccgcggaccacatcacccagttcaccatctctggttggaacaacgcgaccgacaccacccagatcgcggttgttaacgttccgaacgcgtctgacggtgcgcaggcgacctgcgaattcaccatcccggaaggtaaatcttactcttctctgcgtttcaacgttaccggtaccaccaacaaccgtggttactggcacatggcggaattccagctgtacccgatctctctggacccgaaatgcctgaacgcgctgtgcgcggacgcgtacaaacagctggcggaagcgatcgcgaccgcggaagcggttgaaaaagtttctgaagcggacgttaccgcgctgcagaccgcgtaccaggcgtacctggacgcgctggcgccgatcacctctatcggtggtgtttctatccagccggcgaccaccatcggttctgcgatctacgacctgcagggtcgtaaagttcagcgtatggttaaaggtgtttacatcgttaacggtaaaaaagttgttcgt

SEQ ID NO: 158 - DNA, 1302 bp, Unknown
  Environmental sample
atgaaaaaaatcctgtctgcgctggcgatgctgctgctggcgctgtctgcgtacgcggacggtaccatcctgttctctaccgcgcagggtggtatcgacgttgcgccgtaccgtatcccgggtatcacctgcggttacggtggtcgtctgatcgcgtctgcgggtcgtctggtttgcggtaccgacccgggtttcggtcgtgttgactgcgttgttaaaatctctaccgacaacggtgcgacctggtctgaacgtgaaatcgaagcggcggttggtgacgcgtctctgatcaacaacgttaaaaccccgatggcggcggcgtacggtgacccggcgatcgttatggaccgtgaatctcacgaagttctgatgatggcggttgcgggttgcaccgtttacggttacgcgtctaccaaccgtcacaacccgaacctgatcgcggcgatccgttctatggacggtggtctgacctggcagcagccgatcgaacagaccgaagcgatctacggtctgttcgaccagacccacccgatcgacgcggcgttcgttggtggtggtaaaatcttccagtctcgtgttgttaaagttggtcgttactaccgtatctacgcggcgctgaccgcgcgtccgaaaggtaaccgtgttatctactctgacgacttcggtcgtacctgggcggcgctgggtggtccgtctgcgctgccggttccggacggtgacgaagcgaaatgcgaagaactgccggacggtcgtgttatcatcacctctcgtaccgcgggtggtcgttggttcaacctgttcacctacgacgacgttaaaaccggttctggtcgttgggacgaacagaccaaagcgaccatgtctggtatggcgctgaccccgtctaccaacccgaccaacggtgaaatgctgatcgttccggcggttcgtaccggtgacggtaaaccggttcacgttatgctgcagtctgttccgaccggtaccggtcgtaacaacgtttctatcttctacaaagaactggcggacgcgtctgacatgcgtgacgttcgtgcgctggcggaaggttgggacggttgctaccaggtttctccgaccgtttctatgtactcttctatggacctgcagtctgaccaccgtatcgcgttcttctacgaagaaaccctgacccgttggggttctaaaccgaacccggtttctacctctttcccgaaaggtgaaggtgaacacaactacgacggttgcgaaaacgtttacaaatctctggcgctggaaaccatcaccgcgggtaaataccgtgttcgt

SEQ ID NO: 159 - DNA, 1617 bp, Unknown
  Environmental sample
atgcgtaaagttatgtctatcctggcggcgctgctgtctctgccgaccgcgctgggtgcggcggactctctggaagttcgttgcccgcagatcccgatcctgctggaccgtatcgacaacgttttcttccagatccgtgttccggacgcgaaaccgggtgacgttctggaatctgttaccgttgaattcggtgcgggtaccgacctgaaatctgttgcggaactgcgtctgttctactctggtaccgacgcggttcgtcgtggtggtgttcacttctctccggttgaatgcatctcttctcacaacgtttggaacacccgttctgcgatctcttcttactctgttctgcaggaccacaccctgaaaatcggtcgtcgtgttaccctgcgttctggtcagccgatggttgaaggtatcaactacttctgggtttctgttcgtatgtctccggaagcgaccgttctgacccgtctgggtgcgcgtgtttctgaaatcgttgttaacggtgaacgtaaaccgttcgcgcgtaccgcggaagacgttgttcgtcgtatgggttacggtgttcgtcacgcgggtgacgaccacgcgatggcgtaccgtatcccgggtctggttaccacccgttctggttctctgatcggtgtttacgacgttcgttggaactcttctgttgacctgcaggaacgtatcgacatcggtgtttctcgttctaccgacaaaggtcagacctgggaaccgatgcgtatcgcgatgtctttcgcggacaaaggtggtctgccggcggcgcagaacggtgttggtgacccggcggttctggttgacgaaaacaccggtaccatctgggttgttgcggtttgggcgcacggtatgggtaacggtcgtgcgtggaccaactctcgtccgggtatggaaccgatccgtaccccgcagctgatgatggttcgttctgacgacgacggtcgtacctggtctgaaccggttaacgttaccggtcaggttaaagacccgtcttggcgtttcctgctgcagggtccgggtcgtggtatcaccatgcgtgacggtaccctggttttcgcgatccagttcatcggtggtgacggtatgccgtctgcgggtatcatgttctctaaagaccacggtaaaacctggaacatccacaacccggcgcgttctaacaccaccgaagcgcaggttgcggaagttgaaccgggtgttctgatgctgaacatgcgtgacaaccgtggtggtgcgcgtgcggttatgaccaccaaagacctgggtcgtacctggaccgaacacgtttctaaccgtaccgcgctgcgtgaaccggtttgcatggcgtctctgatcgcggttccggcggaccgtaacgcgctgggtaaagacctgctgctgttctctaacccggacaccaccgaaggtcgttgcgacatcaccgttaaagcgtctctggacggtggtgttacctggccgtactctctgctgctggacgaaggtgactcttggggttactcttgcctgtctatgatcgacccgcagaccgttggtatcctgtacgaatcttctgcggcgcacatcaccttccaggcgatcgaactgaacgaaatcgttcgt

SEQ ID NO: 160 - DNA, 2661 bp, Unknown
  Environmental sample
atgaaaaaccacctgctgctgaccaccatcctgtgcggttgctctctgaccgcgtggtctgcgatgccgatcgaacagccgtggcagaccatcaccatggcggaagaagttaccccggaaaccccgaccggtgaagttgttatcgacgttaacaccaccaccggtaccttcgtttctaaaaacccgaacggtacctgggcgtctgcgtggaaatctacccagacctctccgttcttcaacctgacctgcggtgcgaacaacatggacgttgcgaaatctgttaacggtgacctggttgcgtacgttggtaaccagaccaactcttgcgactacatgctgtctgttgaacgtggttggaaaatcaccggttactctttcgacttcgttcgtgtttctggtaaaaacccggttaccctgaccgacgcgaaaggtaccgcgctgaaatcttctgacaccaaacagtctatccgtgttgacggtctgaacgcgtcttctgttaaagcgttcaacctgtctggtggtaacgacggtgttgttatctctaacttcaaagtttcttactgcatcgacaaagcgtacgttcagaccgacttccgtaccttcaccgttttcgacaacaccggttctgttccgtaccgtatcccggcgatcggtaccgcgcaggacggttctctggttgcggttgcggactaccgttactctaaagcggacatcggttctggtcgtgttgacctgcacctgcgtatctctcacgacaacggtaaaacctggggtaacgttatcgacctggaacagttccgtggtgacggtaacctgaaagaatggcgtcacgacaaagcggcgttcggtgacccgtgcatcgttggtgaccgtgaatctccgcgtatgatgatcacctcttgctctggtttcccgggtttcttcgacgcgaccgaccgtcaccagggttgggcgcgttggtactctgaagacaacggtaaaacctggggtgaaccggaatacctggacgaagaattcatctacgcgccgctgaaaaaagcgggttacaacgttcagggtttcttcgttggttctggtaaaatcttccagtctcaccacatcaaacgtggtgaatactaccgtctgtactgcgcgggttctacccaggaaggtggtggtaacaaaaaaaactgggttctgtactctgacgacttcggtaaaacctgggacttcctgggtggtacctctaaagcgccgatcgaaatcaccgcggacgaaccgaaagttgaagaactgccgaacggtaacatcatcctgtcttctcgtgacgtttctggtcgttactacaacatcttcaccttctctggtaaagacggtgtttctggttcttggggtgaaaaagcgctgtcttcttctgcggttggtggtctggttgcgaacaacggttgcaacggtgaaatccagatcgttccggttgttcgtaaaaaagacggtgttaaaaccttcctggcgctgcagtctctgccgacctctggtcgtaccaacgtttctatcttctacaaagacctgtctgacgcgacctcttacgcgaccccggcgaacttcgcgaaatcttgggacggttctttcgttgtttgcgacttctcttctgcgtactctacctggtcttggcagcacgacaacgcgctggcgttcctgtacgaagaaggtggtggtacctctggtggttacaacatcgtttacaaacgtatcgacatcccgaccatcaccgaacagaaatacgacttcgacgcgtcttacgaatacaaacgtttcgacatcgactggacccaggcggacctgaacaaagaaatgtctaccctgctgaacaccgcgaaaaaaaactctgacgcgaacaccgtttacgttgtttctggttctaaactggttaccgacgcgaaacagctggaatgcaaattcggtcacaaagaaatgggtggtaccggtggtgacgcgcaggacatctctaacctgatcgacggtgacccggcgacctactaccacacctactggggtgcgggtaaccagccgaacggttctcacttcatggacgttacccacccggaaggtgcgctgttccagggtatgatcaaagttgacctgatccgtcgttctaacgcgaccgcggaccacatcacccagttcaccatctctggtatcaacggtaccgacgaaccgaccgaaatcgcggttgttgcggttccgaacgcggcggcgggtaaagaagcgacctgctctttcaccatcccggcgggtgtttcttacgaaaaactgcgtttcaacgttaccggtaccaccaacaaccgtggttactggcacatggcggaattccagctgtacccgatgtctctggacccgaaatgcctgaacgcgctgtgcgcggacgcgtacaaacagctggttgacgcgatcgcgaccgcggaagcggttgacgcgaccaccgtttctcaggcggacgttaccgcgctgaaaaccgcgtaccagtcttacctggacgcgctgcagccgatcgtttctggtatcaacggtgtttctatcgcgccggcgacctctggttctacctctatctacgacctgcagggtcgtaaactgcagcgtatggacaaaggtgtttacatcgttaacggtaaaaaagttgttcgt

SEQ ID NO: 161 - DNA, 1302 bp, Unknown
  Environmental sample
atgaacaaaaaaatgatgctgtctgcgaacgcgtctaaagcgctgttcctggcggcgatgggttgctctctgctgatgccggcgcagatgatggcggcgcagctggttggtgacgcgccggttatggcggttgaagcgcaggcggttaacgcgcagggtggtctgaccgttttctctaacaaagaagcgcagtaccgtatcccggcgatcgttgaatgcaaatctggtaaactgatcgcgttcaccgaccaccgttaccacaacaaagacatcggtggtggtcgtcacctggacatcgttatgaaaacctctatggacaaaggtgcgacctggtcttctccggaacagatggttgcgaaaggtggtaacggtatcgcgacctctttcgactgcgcgcacggtgacgcggcggttgttgttgacaaaaaatctggtcgtatcctgctgatgtgcgcgtctggtggtatcggttactgggaatctaaacgtggtaacctgctgatgatgggtcgttactactctgacgacgaaggtaaaacctggtacggtgaagaagttaccaaacagatctacgacctgatcccggaagttgaatctgcgttcttcacctctggtcgtatctgccagtctaaacagatcaaagttggtaaatactaccgtgtttacaccgttctgtgcacccgttctggtaaccgtatcctgtactctgacgacttcggtggtacctggaacgttctgggtaaaaacgttgcggaagcggcgccgtacggtgacgaagcgaaagttgaagaactgccgaacggtaacgttctgctgtcttctcgtgcgatgggtggtcgtcacatcaacatctacacctacgacgacaaaaaaaccgcgaccggttcttgggacaaagttgttgcgtctgaagcgaaaaacatgggtgttgcggcgcacaaaaactcttgcaacggtgaagttctgatggttgacgcgaaaaaaaacggtaaaaaagttaaactgctgctgcagtctgttccggttggtccgggtcgtaacaacgttggtatctactacaaagcgctggaaaccccggcggactacgcgaccccggaagcgatcgcgaaaaactgggaaggttgctaccagctgtctaacaccacctctgcgtactctaccatggttcagggtaaaaacggttctatctacttcctgctggaagaaaacgcgttcaaaaacccgaaaacccagaccgacgactactacgacatccgtttctacaacctgtctatcgaacagatcaccaacaaccgttacaaa

SEQ ID NO: 162 - DNA, 3633 bp, Unknown
  Environmental sample
atgctgaaacgtgcgcgtctggcggttaccctggcgctggcgggtggtctggttgcgtctggtgcggttggtgttacctctgcgttcgcgcagggtggtgttcgtccggacgacggtgttacccagggtcagccgttcgcggcgggtaccggtggttctgcgaaattccgtatcccggcgatggttaccctggacgacggtaccatcgttgcggcgaccgacgcgcgttggaacaccaccggtgacggtggtggtctggacaccatcgtttctcgttctaccgacggtggtgcgacctggaactacaccttcgcgaactacctgggtgacaacggtaacgtttggtctggttactctaccgcgttcatcgacccggcgctggcgaccgacggtcagaccacctggatggttgttgacctgttcccggcgggtatcgcgctgaactctgcgaactgggcgccgcaggttggtgcgggtttcgacgcgaaaggtcgtctgcgtctgcgtgacgcgggtcaggttgaattcggttgcgactactactcttggtacgcggcgcgtgcgtcttacgactactacctggacctggacaccctgaccatccacaaagcggacgactctaaagttgaaggttacaccgttgacggtttcttcaacatcaccgacgcggaaggtaaagttaccaacctgttctgctctgacgcgccgttccaggttttcccgaccgactacctgtacctgaccaaatctaccgacggtggtgttacctggtctgaaccgaccctgatcgacgttaaagacgacgacgaacagaccctgctggttggtccgggtaccggtaccgttctggaagacggtaccctgatgttctctgtttacgaacacacctctggtaaatctgaactggcgtctgttatctactctaaagacaaaggtaaaacctggacccgttcttctgacgcgaccccgtcttctcactggtcttctgaagcggttaccgttccgctggacaacgacaccgttcgtcagttctaccgtgacggtatgaccaccctgtactacaccgactacgacctgaaaaacaacgtttggaccccgggtcagccggttgactctggtgaagttaaaacctctggtaaccagctgtctgcgatccgttactctaaaaaaatcgacggtaaagacgcgatcatcgtttctatggcgtcttctggttcttctgcgcgtaccgacggtaaactgcacgttggtctgatcaacgacgaccagaccatcacctggcagtacgtttacgacatggttgaaccgggtggttcttactcttactctaccctggcggaactggaaaacggtgacatcgcggttctgtacgaaaactctggtggtaccgaactgttcgaagttgttccgttcgcggacctggttcgtgacgaaaccaacgttgacatgaccgttaccgcggttgacctggttgcgggtgaacaggcggttctggaagacccgtctggtgactacaccaccgcggacatctctgaactggacacctctgttgcgaccctgaaactggaacagggtgaaccgggtgcgccgaccgttaaaccgggtaactcttctggttacaccggtgcggcgacccagatctctgactgcctgtacaccttcaccaaatctggttctggtttcatcatctctgcgaaagcgtctgacggttctaccgtttactgcgcgccgggtatctctacctctgcgggtttcccgaactctgcgacctcttctgttgttaccctgtctgcgggttacgaagcgaacaccgtttacctgcaggacaccaccaaaggttcttacctgtacttcgacaaatcttctcagcagctgaaccgtgttaacaccctggacaacaacaccacctggatgaaaaactgctcttggtacctgtaccgtgcggcggcggcgggtgaagcgtcttctaaagaactgccgggttacgttaaagttgaaaacccggaagcgatcgaaggtggttcttacctgatcgtttctcacgcgaactctggttctgactacgttctgtacccgaccctgtctgttggttctgcgcgtgaccaggttgcgcaggcggttgttgcgaccaactctaccaccgttctgaccttcaccggtgttgcgaccggtaccacctctgttcacgttggtgcgacccgttacgacgttaccgttgcggactaccacgaccaggcggttaccgttgaaatgggtaaaaccgttgaagttaccgacgcgaaaggtgacttctctggtaaagttgacgacggttctttcaacaaaggtatcgcgtcttacgaagttaaaggtgcggacggtgcgaccaccatctctttcaccggtatcatgccgggtaccaccaccctgatcgttggtaccacccgttacaccgttaccgttccgggtgaagttgttgaagcggaccaggaactgggttctacctggtctaccaccgcggcgggtgcgatctctgcggacgacctgggtgactacgactcttctatcatcgacgttaaactgaccgcgaccgacggtgcggttaccggtaacgaatctatcctgctggacgacaccgcgtacaccttcaccaaagcggaagacggtaccttcaccgtttctggtgcgacctctgacggtaaaaccgtttacctggcgccgggtaccaacggtccgggtttcccgaacgcgaccggtgcggcggcgatcaccgttaccccggcgtctggtaaaaacggtgcggaaggtctgttcaacctgcgtgactctggtcgttacctgtactacaaatctggtgcgttcgaccgtgtttcttctccgtctggtttcgaagacggttgcaccttcaaactgtaccgtgcggttgacgttcagaccctggcgaacggtgaaggtgaagcgatcgaaggtttcgaacaggttatgtctgttgacgacgttaccgacggtacctacctgatcgttgcggcgaacgcggcggacggtgacctgccggttctgcacccgtctacctctaccacctctaaaccggcgcacgttgcgtacgttaaaaaaggtggtgcgacccaggcggacgttaccctgaccgcggttggtaccaccggtatccgtctgggtaaccagtacgttcgtttcaccgttcgtgcgccgcgtttcaccgttaccctggacccggcgggtggtgcggttgaccaggaagcgatcgaagttgttcagggtgaaccgtacggtgaactgccgaccccggttcgtgacggttacgaattccagggttggttcgacgcggcgggtaacaaagttgacgcggaaaccgttttcgaaggtaacgcggacgcgaccctgaccgcgcagtggaaaaaagcggaaaccgttcagccgggtccgggtaccggtaacaccggtggtaccggtaacaccggtaacaccggtaacgcgggtaacgcgcagaaaccgaccacccagaaaccggcgaaaaaaggtggtaccctgccgaaatctggtgacgcgaccaacggtgcggcggttgcggttgttgcgctggcgggtgcggcggcgctgaccgcgggtatcgttgttcagcgtcgtaaacaccag

SEQ ID NO: 163 - DNA, 1344 bp, Unknown
  Environmental sample
atgtctggtgaaaaagttatcttctgcggtaacaacccggacttcctggacaaaaaactgaccaccctgtctgacggttctaacggtggtaacgcgttccgtatcccgtctctgatccgttctggtaacaccctgatcgcggcgatcgaccgtcagtcttgcggtgcggactggggttacatcgaactggcgatccgtcgttctgaagacggtggtgaaacctggtctgacatccagatcatcgcgatcccgccggcgcgtaaaaccatgctgtctgacgaatgctacggttctgcgttctacatcgacccgtgcatggcgatcgcgccgaacggtgacgttctgctgctggttgacttctacccggaatgcaaaggtctgcacaaacgttctatcctggacaaaaaaaaagcgccgtacgcgctgtacaacaaagaaatgcgtccgaccctgtacgaccgtgacggtaaattctacgttatcgacaaatctggtcacgttctgtctcaccgttacaccgacaccggtatgaccgttgactacgaatctggtgaactgtactctggtgaagcgtacctgggtaacatccacctgaacggtaaatctccgtctaacatcaacgaagcgggtgcgaaaaccaccttcggtgcgccgctgaaagcgccgaaacgttcttacctgtacctgctgcgttcttctgacaacggtaaaacctggtctaaaccgaaagacatcaccggtcaggttctgatcaaacaggacggtaccttcctgggtgttgcgccgggtgttggtctgaccacccacaaaggtcgtgttatcatgccgctgtacgttgaccgtaaacagaccgtttctatctactctatcgacgacggtgaaaactggcaccgtatggcgggtcacccgtacgcggaaaacgttgacgaatggcagatggttcaggcgccgtctggtgcgatcatcggtctgggtcgtgcgaaacgttacggtaaaaccccgatgtctatctcttacgacgacggtaaaacctggatcaacgcgggtaaaaccgacctgtacgcgccgaaatgccagaaatctgttatctctatcggtgactacgttttctgctctcacccgaacgacaaaacccgttctaacggtgttatcaccgttggtaaattctacaaagcgaaaggtcgtgttgcgggtatcaactggtactctgaaaccgaagttaacaaaggtttcttcgcgtactcttgcctgacccgtatcgacgacgaacacatcggtatcctgtacgaatctgaaccgtcttctttcatcctgttcaaaaaatacaaaatcaaagacctgatc

SEQ ID NO: 164 - DNA, 1137 bp, Unknown
  Environmental sample
atggcggcggttatcatcgcgaccaccgcgtcttgcggtgaaaaagaacgtaccaccccgtctccggcgccgggtaaagacgacaacacctctgttgttaaagcgccgcacatcttcgttgcgcgtaccaacggttaccacaccttccgtatcccggcgctggttaaatctcgtaaaggtaccctgctggcgttctgcgaaggtcgtaaaatctctggttctgacaccggtgacatcaacctgatcctgcgtcgttctaccgacggtggttctacctggggtaacatcatcaccgtttgggacgacggtctgaacacctgcggtaacccgtctccggttgttgacccggttaccggtcgtatccacctgctgatgacctggaactaccagaaagacggtcagtctgcgggtgacttcaacaaagacggtgttaccaaagacacccgtcgtgtttggtacacctgctctgacgacgacggtctgacctgggcggaaccgcgtgaaatcacctcttctgttaaaaaagacgactggggttggtacgcgaccggtccgtgccacggtatcgttatgaccaaagcgccgtacgaaggtcgtatcgttatcccgtgcgactacaaccagaaaggtggttctggttactctcacgttatctactctgacgacaacggtgcgacctggaaaatcggtggttctgttaaaggtggtaacgaatcttgcgttgcggaaaccggtgacggtcgtctgatcatctcttgccgtgttggtggtggtaaacgtatgatcgcgtactctgcggacggtggtgaaaccttcaccaccggtaccgcgaacaactctctgccggacccgcgttgccagggtaccatcctgtctgcggttaaagacggtaaagaagttctgctgcactgcaactgctctaactcttcttctcgtacccagctgaccctgaaaggttctaccgactctggtgcgacctggtgcggtggtcaggttatctgggaaaaagcggcggcgtactctgacatcgttctggttaaagacgaaatcgttggtgttctgtacgaaaacggtgacaacggttcttacgaacgtatctctttcgaacgtgttccgctgggttacgcgttcatcaaa

SEQ ID NO: 165 - DNA, 1395 bp, Unknown
  Environmental sample
atgtctcagaccaccaaacgtgttttcacccgtaaatctaaactgttcctgatcctggcgtctatgatcctggcgctgtcttgcaaaaacccgctgggtgacgaatctggtggttctggtggtgcgggtggtggtggttcttctggtggtcagaccatcaccaacgaacagggtaaagttttcccggcgtggtacctgaccgaacagcagcagcagcaggacttctctatgggtccgaccatcctgttccagtctaccaaagcgccgacccagcactaccgtatcccggcgatcatcgttgcggacaacggtaacatcatcgcgatcgcggacaaccgttacaaccacggtggtgacatcggtgcggttaaaggtctgatcgacgttgtttacaaaatctctcgtgacggtggtaaaaactggtctgacgaaaaaatcatgggtaaccgttctaccgaccacaactactctaaagcgctgaacaaaggtgacgcgctggttttccgtgcgaacgacggtaccctggtttgcatggcggtttctggtggtggtttcgcgaacgcgaccgtttctaccccgtctcgtatgctgcgttctgaatctaaagacaacggtgaaacctggtctgactggaaagaagttggtcagtctctgatcgacaaaatcaaaaactctttcggtaaaggtatcccgaaagcgttcgcgccggcgggtcgtggtctgaccctgaaagacggtaccttcgcggcggcgatgatcggtcaggttaacaacggtggtttctacggtctgtacatctactctaccgacaaaggtctgaactggaactgcgcggacaacttcatcgcgtctaccggtggtcacggttcttggaacgaaccgaaagttatcgcggaactgaacgacggtaaactgctgatgtctgttcgtaacggtatgtctaccgcgaacaaaaacgacccgcgtatgtacgcggtttctaccgacgcgccggttaacggtaaatgcgcgtggccgaactctctgtctgcgtggaccctgatccgttgcggtggtgttgacgcggaaggtgttgtttggacccgtgaacgtgaacaggacaaaaaccgtatgctgcacatccaggcgggtccgtacggtcgtcacggtctgaaactgttcctgtctaccgaagaaggtaaaaccgacaactcttggaccgaagcgaaagttatcctgggtggtcagaccggtgcgtcttactcttctctggacgtttgcggtgacggtaccatcgttaccctggcggaagaaaaatctaaaaccggtcagcactacgacatcgttttccgtcgttacaacatgaaagcgatcacccaggaagtttacaaaaccaaatggtacaaagaagttaaa

SEQ ID NO: 166 - DNA, 3864 bp, Unknown
  Environmental sample
atgaaaaaccgtaaacgtctgctgtctctgctgatggcgggtgttctgaccttcggttgcttcccggttaccgcgggtgcggcgaacaactctaaagacggtgcgaaaccggcggacggtaccaccgcggaacagccgttcccgaaaaccctgttcctggaagaacacaactcttcttctggtttcacccgtttccgtatcccggcgctgaccaccgcgttcgacggtaccctgatcgcggcgaccgacatccgttgggacatctgcggtgacggtggtggtctggacaccctggtttctcgttcttctgacaacggtgcgacctggaactacaccgttgcgaactacctgggtgacaacggtaacaaattcaaccgttactctaccgcgttcatcgacccgtctctggttgttgacggtaacaccatctacctggcgtgcgacctgttcccgggtggtgttgcggttgcgtctgcggcgtctaccccgtctcaggcgggttctaccggttacgactctgaaggtcacctgctgctggcggcgaccaccacctctgttaacggtatctcttcttctaccctgcgtgcggcggcggactacgcgtaccacctggaaaaaaaagcggacgcgaccgcggcggactactacgaaatcaaagcgaactctaccggtgaagttgttgacggtgaatacaccatcgacgaccacttcaacatcaccaacgcggacggtaccgttgacaccaacctgttctgcggtgacaccccgtacttccagtacccgaccgactacatctacatcgttaaatctaccgacaacggtgcgacctggtctgcgccgaccctggcgaacgttaaaaacgcggaagaacaggttttcctgatcggtccgggtcgtggtatcaccacctcttctggtcgtatcatcttcccgtgctaccagtacaccaacggtgttcagcgttctgcgaccatctactctgacgaccacggtgttacctggaaacgtggttctaccgttaccggtaactcttctgaagacgttatcgttgaagcggacggtaaactgtacatgttcgttcgtatgtctaacgtttactacgtttctgaagacgacggtaccacctggtctgaaccgaaatctatgggtatcaaattccacaacaactgccagctgtctgcgatcacctactctaaaaaagttaacggtaaaaccgcgatcgttttcgcgggtccgtctgacgcgggtggtcgtaactctggtcgtatctggctgggtctggttcaggaagacggttctatcgaatggcaggacaacccgtacgttgttaccgaaggtacccactacgcgtactcttgcatcaccgaaaccgcggacggtgacatcggtctggtttacgaatacgaagacgaaaaaatcaaattcgaaaaaatctctttcgcggacatcgcgggtgaagcgaccacctctcgtgtttgggttgaaaacgaaaaaggtgcgatcgttaactctgcggttatgaaatctgacgcgaccgcgtcttacatcgttaaaaaactggacgcggacaccgaagttaccgtttcttcttctaaccgtgcggttctggaagcgtcttacaccgacggtaaactgatcctgacctctaaagcgggtgttaccggtctgaaacaggttaaagttaccgttaaagcgggtgaagaaaccttcgttgttaacgttaacgttaccgacgcggaaaactacaaagttatcaacctggaaaaagacgaaaccaaaaccgttaccgttaaaggtgcgaactacgcggacgcggacacctcttctgttgacaccgaagttgcgggtgttaccgttaccggtaaagacgttcaggacacctacgaaaaaaccgttgcgcagctggcgaccgcggcggcgaccttcgacggttctaaagttgacctgtctaaatgcatgtacaccctgggtgcgggttcttctgactctaccttcaaagttaaagcggcgaccgcgaacggttcttctatctacctgggtccgaaagcgtcttctaccgcgggtatcccgaacatcaccacctctcagccgcagatcaccttcaccaaaaacgcgtctgacgcgaccttctctctgatggacaacacctctggtaccgcgggtcgttacctgtacttctaccacaccgacgcgaccaaactgtacttcgaccgttacggtaccgcggacgaacactgctggttcgaactgtacaccccgtctaaagacgcgaaagactacgacctgatcaacggtttcaaaaaagttgaatctctgaaagaactgaccgcgggttcttcttacctgatcgttaccaaagcggacgcgtctggtaaccgttacatcctgctgccgtctaccggtacccagaaatacaaccacgttgcgaaactggttaacgaaaccgtttctgcgaccgaagaagcggcgtacgttgcgaccgacgttgcgaccttcgaagaaaaatctgcgaaaggtctgtctgaatgcctgtacaccttcaaaaaaatgtctgacggtacctacgcgatctctggtcacgcggcggacggtgcgaccaccttcgttaccatcggtaccgttggtatcccgaacaaatctgaaaaagttgacatcaccgttgaagacatcgcggactctaacggtaaagtttctctgtacgacggtaccgcgtctcgttacctgtacttctggcgtgacaccgcgaaactgtacttcgaccgttacggtacccaggacaccaccgcgtgcgcgttcgaactgttcaaaaaatctgcgaccgcgtcttctgactctccgatcaaaggttacgaaaaactgaacggtgtttctgaaatcgaagacggtcgtcagtacctgatcgcggcgcaggttgacggtacctactacgttatgaacccggttctgggtgactctaaattctctcacgttgcgaaagttaccaacaccacctaccgtaacccggcggcgaccgcgtctaccaccatcgacttcaccggtgttgcggcgggtaccacctctgttaaaatcggtgacgttgtttactacgttatcgttaccgaagacggtgcggaatgcacccacaaaaccaccgttgttctggacgcgaaagacgcgacctgcaccgaaccgggttctaccggtgaagttgtttgcgttgactgcggtgcggttgttcaggaagcgcaggaaatcgcggcgaaaggtcacaccttcggtgaatgggttaccgttaaagaaccgaccaccaaagaagaaggttctaaagaacgtatctgctctgtttgccagaccaaagaaaccgaaaccatcgcgaaactgccggacaccgaagacccggaccagccgaccaccccgtctgcgccggaaaacgttaaagactctgaagttaccaaagactctatcaaaatcacctgggacgcgccggcgtctaccgaaggtatcgcgggttacaaaatctacgttaacggtgcggtttacgcgaccgacatctctcgtgacgcggttggttacaccctgtctggtctgaaagcgggtgaaacctacgaaatccaggttgttgcggttggtaccgacgaaaacgcgaccgaatacgcggcggcggcgttctctgttaccaccaaatctgaagacaaaaacgacaaccacggtggtaacgacaacaaaaacgcgaccaccaccgacaaaaaccagaactctaccgttaacaacgcgaacaaagcggcgaaaaccggtgacaccgcgaacgttgttttcccggcggcggttatgatgctggcgggtgcggcggttgcgggtatcctgttccgtcgtaaatggatgaaa

SEQ ID NO: 167 - DNA, 1359 bp, Unknown
  Environmental sample
atgaaatctaaaaaaatctctaaactgttctacctgtctttcatcctgatcttctggatcctgatcttcaccctgacctgcaaaaacccgtctggtccgggtatcgacttcaacctgcgtaactggtacatcatgtctggtatggcgaaaggtccgggtttcacctctaacatcacctacggtaacaacaccatcatctacccgtctaactacatgaaaaaagaaaaagacatggttttctctaacggtacctacaactaccgtatcccggaactgctggcgctgccgaacggtgacctgctggcgttcgcggaccgtcgttacaacggtgcgggtgacctgccgaaccgtatcgaagtttacgttaaaaaatctaccaacaacggtaccaactggggtgcggaaaaacaggttaccccgacctctctgtctaaatctgacggttacggtgacaccgcggcggttctggaccgtaaaaccggtaacgttatcgcggttgttgcgcacggtcagggtttcttccagtctacctctaaagacccgatccgtatcatcaccatccgttctcgtaacaacggtgaatcttgggaagcgccgatcgacatcacctctcagatctacggtgcgggttgcggtgacctgacccgtcgtaactggcaggcggcgttcgttacctctggtaacggtctgcagatgcgtaacggtcgtatcatcttcgttctgaacgttcgtcagaccacctctgcgttcgttgaaaactacatcctgtactctgacgacggtggttacaaatggcgtgtttctcgtggttctccgaaaaacccgaaaggtggtaacgaagcgaaagttgttgaactgaacgacggttctctgctgatgcacatccgttggaccccgaaccgtctgcagtctcgttcttacgactacggtgaaacctggaccgaagcgaccgttatcaacggtatcccggcgtctcactctaacggtgacctgatcgtttacacctctaccaaaaacggttacgacaaagaccgtctgatcaccctggttgaccgtcgtccgtggggtaacaaccaggcgaaatgctcttcttgcaaatctggtggtaactgcaccccgggttacccgggttgcccgaccttctacgtttcttacgacgaaggtaaaacctggaccaactctcgtcgtctgtacaccggtcacgcgggttactcttctctggcgatcctgaaagacggttctatcggtatcctggcggaactgggtaactcttggaacggtccgatctacttcctgaaaacctctatcgaatggtgcaacccgaacgacaacccgtgctctccgaccaacgcgaaaaaa

SEQ ID NO: 168 - DNA, 1359 bp, Unknown; identical to SEQ ID NO: 167
  Environmental sample

SEQ ID NO: 169 - DNA, 1371 bp, Unknown
  Environmental sample
atgaaacacaaagaacgtggtaacacccacatggcgaaagaacagctgatcttctgcggttcttctgaaaccttcctggacaaagcgctgaccgcggactctaacggtgacaacggtggtaacgcgttccgtatcccgtctctggttaacgcggacggtaccctgatcgcggcgatcgaccgtgcgtcttgcggtgcggactggggttacatcgaactggcggttcgtcgttctgaagacggtggtaaaacctggtctccgatcgaaaccatcgcggcgccgccggcgcgtgaaaccaaaatctctgcggactgctacgcgtctgcgttctacatcgacccgtgcatggcggttgcgccgaacggtgacgttatcctgctggcggacttctggccggaatgcaaaggtctgcacaaccagcgtctgctggacaaacacaaagttccgtacgcgcagctgtctggtcgtatgcgtccgctgatctacgaccgtgacggtaacttctactacatcgaagaatctggtgcggttctggactctcagaaacagccgaccccgtaccgtctgggttctggtttcggtgacctgtacaaaggtgacgaatacgttggtaacatctacctgaacggtgcggttggtaaaaacgaagcgggtgcggttaccaccttcggtgcgccgctgaaagcgccgaaacgttgctacgttttcatgctgcgttcttctgaccgtggtaaaacctggtctgacccggttgacatcaccaacatggttctgcacgaaaccgacggtaccttcctgggtgttgcgccgggtgcgggtatcaccaccaccgacggtcgtatcatcatgccgctgtacgttgaccgtaaatctaccgtttctatctactctgttgacaacggtgacacctggcaccgtatgacctctcagccgtacgcggaaaacaccgacgaatggcagatggttgaagcgccggacggttctatcgttggtttcggtcgtcagaaacgtttcggtaaaaccccgatgtctatctctgcgaacaaaggtaaatcttggtctaaatgcggtaaaaccggtctgtacgcgccgaaatgccagaaatctgttatcgcggttggtgacaccgttttctgctctcacccgtctggtcacacccgtgaaaacggtatgctgtctgttggtaaactgcgtcgtaaaaaaggtggttacacccacgttgactgggttcgtcacgttccgatcaaccgtggtttcttcgcgtactcttgcctggcgcagatcgacgcgcacaccctgggtgttctgtacgaagaccgtccgtcttctcacatccagttccagaccttcgaactggcggaactgatgggt

SEQ ID NO: 170 - DNA, 1644 bp, Unknown
  Environmental sample
atgcgtaacaccttcctgctgatcggtatgctggcgctgtcttcttctgtttacgcggcggacaccctgttcgttcgtgcgccggaaatcccggttctgaccaaccagcaggacaacgcgctgttcgaaatccgtgttaacgcggacaaaggtgacgttctgaaccagctggacatctctctggacgcggaagacctgtctgaagttgaagcggttcgtctgttctactctggtaccgaaggtgtttctcgtcgtggtgttcacttctctccgaccgacaaccagtcttactcttctccgtctgttctgctgggtaaatctggtcaggcgaccctgaaagaagttttcgcggactactctgttaaacaggacgaaaaatcttctccgggtaaacgtctggcgctgtcttctgaacagccgatgctgaaaggtatcaactacttctgggtttctctgcagatgaaaccgaaaacccagctgtctcacaccatcaccgcggcgatcgaatctgcggaaatcaacaaatctaaagcggttgttgcgggtaacaacctgtctccgaaacgtcgtatgggttacgcggttcgttctgcgtgggacgacggtgttgcggcgtaccgtatcccgggtatcgttaccaccaacaacggtaccctggttggttgctacgacgttcgttaccactcttctgttgacctgcaggaaaaaatcgacatcggtgtttctcgttctgaagacaaaggtcagacctgggaaccgatgcagatcgcgatgaccttcaaaaacgaaggtggtctgccgcacgcgcagaacggtgttggtgacccgtctatcctggttgacgaacagaccaacaccctgtggatcgttgcggcgtggtgccacggtatgggtaaccagcgtgcgtggttcaactctcgtcagggtatgaccccggacgaaaccgcgcagctggttctggttaaatctaccgacgacggtaaaacctggtctaaaccgatcaacatcaccaaacaggttaaagacccgtcttggcacttcctgctgcagggtccgggtcgtggtatcaccatgcacgacggtaccctggttttcccgatccagttcatcgactctgaacgtatgccgcacgcgggtatcatgtactctaaagaccgtggtgaaacctgggcgatccaccagccggcgcgttctaacaccaccgaatctcaggttgcggaaatcgcgccgggtgttctgatgctgaacatgcgtgacaaccgtggtggttctcgtgcggtttctatcaccaaagacctgggtaaaacctgggaagaacacatctctacccgtaccgcgctgcaggaaccggtttgcatggcgtctctgatccacgttgacgcgaaaaaaaacatcctgggtaaagacctgctgctgttctctaacccgaacaccaccgcgaaccgtaaccacatcaccatcaaagcgtctctggacggtggtgttacctggctgaaatctcaccagatcctgctggacgaagaagacggttggggttactcttgcctgaccatggttgacgaagaaaccgttggtatcctgtacgaatcttctgttgcgcacatgaccttccaggttgttaaactgaaagaactggttaaagaa

SEQ ID NO: 171 - DNA, 1332 bp, Unknown
  Environmental sample
atgtctgactctaaaatcatcttctgcggtaactctaaagacttcctggcggaagaactgaccaaagcgtctaacgcggaaaacggtggtaccgcgttccgtatcccgtctctggttaacgcggacggtaccctggttgcgatcgcggacaaagcggcgaccggttctgactggggttacatcgaactggcggttcgtacctctgcggacggtggtaaaacctggaccccgatcaaaaccatcgcggcgccgccggcgcgtgaaatcaactcttctgttgaaaacaccaaaaccgcgttcttcatcgacccgtgcgcggttgttgcgccgaacggtgacgttgttgttctggcgaccttcttcccggaatctaaaggtatccacgacaaaaaaatcctggaaccgaaaaaagttgcgtacgcggacttcaacggtaaaaactgccagctgatctacgaccgtgaaggtaaattctactacgttctggacgacggttctgttctggactctgcgaaatctaaaaccgactacaccgttaaaggtatcggtgaactgtacaaaggttctgaatacgttggtaacatctacctgaacggtgaaatgggtaaatctgaagacgaaaacggtaaaaccaccttcggtgcgccgctgaaagcgccgaaacgttcttacgtttacatgctgaaatcttctgacaaaggtgcgacctggtctgaaccggttaacatcaccggtgcggtttacaacttcgacgacggtgcgttcttcgcggttgcgccgggtgcgggtctggcggttgacaaccgtatcatcatgccgatctacaccctgaaaggtaccgtttgcatctactctgacgacaacggtgcgacctggcagcgtaacccgcagttccagtacaccgacaacgttgacgaatggtgcgcggttaaagcggcggacaacaaaatctacgcgttcggtcgtgcgaaaaaatacgcggcgaccccggttaccgtttctgaagacaacggtctgggttggtaccgtaccaaaaacgcgaaattcaaagcgccgaaatgccagaaatctgcggttgcgatcggtgacaaaatctacgttgcgcacccgtctggtaaaaaacgtgaaaacggtatcatctctgttggtaccctgacctacaacaaaaaaggtaaattcaaaggtatcgtttggaccaaagacaaagactctgttaaaatcaacgacggtttcttcgcgtactcttctatggcgaaactgggtgacaacaaaatcgcggttctgtacgaagaccagccgtcttctcacatcgttttcgacgttatcaacctg

SEQ ID NO: 172 - DNA, 1275 bp, Unknown
  Environmental sample
atgaacaaatctatcctgcgtttcgcgttcatcctgctggtttctttcctgttcaccgttggtaccaaagcgcagaccgcgggtgcggcgatctctaaagcgaacctgccgaccccggttaccgttttcgacgacccgtaccagggtatcccggttcgtatcccggcgatgaccatcacccgtgacggtggtgttatcgcgatggttgactaccgtcacaacttctctgacatcggttggaactctggtggtacctaccgtctggacatcctgatgcgtcgttctgacgacggtggtcgtacctggtctccgatcaacaccgttgttgaaggtcgtaaagacttcggttacggtgacccgtctgttgttgcggaccgtgactctgcgggtctgatcctgatgcaggcggtttctggtaacgttccgtacgttcacgcgaccgcgaaaaacccgcagcacgcgatgttcttccgttctaccgacggtggtaaaacctgggacaaaggtcacgacgttgcgccgtctatctacaacctgtaccgtgacgaacagccgctgacccaggaaaaaggtttcttcctgacctctggtaaaatcacccagtctcgtatcatcaaaaaagacaaatactaccgtctgtacatcgcgcacccggttctgcacgttggtaccttcgttatctactctgacgacttcggtaaaacctggcaggttctgggtggtgcggcgaccatcccgggtccggacggtgacgaatctaaagttgaagaactgccggacggttctgttctgctgtcttgccgtaaattctcttctctgggtcgtcgtttcaacgttttcgtttacaaaaacaaaaaacgtgcggaaggtgcgtggtctgaagcgaaagacgcgaaagcgctggcgaactgcacccgttgcaacggtgacgttgttgttgttccggttcgtcgtaccatcgacggtcgtaaaacctggctggcgatccagtctctgctgcagtctaaaacccgtgaccacctgggtttctactaccgtgaaatcccgcgtcgtggtgcggacctggcgccggcgctggcggacaactggcagcgtggtgttatgatccacgaaggtaccgcggcgtactctaccatgtctgttttcgacgacggtaccgttggtatcctgtacgaagcggacctggacgcgaacaacaccggttacaaaatcctgttctctcgtctgtctgtttctcagatcaccggtggtacctacgaagcgatgccggcgaaa

(19) United States
(12) Patent Application Publication
(10) Pub. No.: US 2021/0062171 A1
(43) Pub. Date: Mar. 4, 2021

(54) NOVEL SIALIDASES AND USES THEREOF

(71) Applicant: The Regents of the University of California, Oakland, CA (US)

(72) Inventors: Karsten Zengler, La Jolla, CA (US); Ajit Varki, La Jolla, CA (US); Frederico Da Silva, La Jolla, CA (US); Sandra Diaz, La Jolla, CA (US); Cameron Martino, La Jolla, CA (US); Geoffrey Chang, La Jolla, CA (US); Steven Rees, La Jolla, CA (US); Livia Soares Zaramela, La Jolla, CA (US)

(73) Assignee: The Regents of the University of California, Oakland, CA (US)

(21) Appl. No.: 17/006,293

(22) Filed: Aug. 28, 2020

Related U.S. Application Data
(60) Provisional application No. 62/893,103, filed on Aug. 28, 2019.

(51) Int. Cl. C12N 9/24; A61K 38/00; A61K 35/741; A61K 35/745; A61K 38/47; A23L 33/135; A23L 29/00; A23L 5/20; A23L 13/40; A23C 7/04

(52) U.S. Cl. CPC C12N 9/2402; A23Y 2300/29; A61K 38/00; A61K 35/741; A61K 35/745; A61K 38/47; A23L 33/135; A23L 29/06; A23L 5/25; A23L 13/48; A23C 7/043; A23V 2002/00; A23Y 2300/25; A23Y 2300/55; A23Y 2300/45; C12Y 302/01018

(57) ABSTRACT

The present disclosure relates to novel sialidase enzymes having a preference for cleaving N-glycolylneuraminic acid (Neu5Gc) from glycoconjugates over cleaving N-acetylneuraminic acid (Neu5Ac). The present disclosure further provides the polypeptide of such novel sialidase and its catalytic pockets; the recombinant host cells expressing the same, as well as the enzymatic, pharmaceutical, and consumable compositions comprising such novel sialidases. Probiotic compositions comprising a bacterium that expresses the sialidase having a preference for Neu5Gc over Neu5Ac, and methods for removing Neu5Gc from consumable products are also provided. Also disclosed are methods for treating or preventing various diseases, disorders, and inflammatory conditions in a subject using the novel sialidases.

### CROSS REFERENCE TO RELATED APPLICATIONS

This application claims the benefit of U.S. Provisional Application No. 62/893,103, filed on Aug. 28, 2019, which is incorporated herein by reference in its entirety.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The Sequence Listing, filed in electronic form as an ASCII.txt file entitled 942103-1040_CORRECTED_sequence_listing_ST25.txt in written computer readable format (CRF) having file size 549 kilobytes, created on Aug. 27, 2020 and corrected on Sep. 17, 2020, is incorporated by reference in its entirety.

### FIELD OF THE INVENTION

This disclosure relates to sialic acid chemistry, metabolism, and antigenicity. More particularly, the present disclosure relates to novel sialidases, bacteria producing such novel sialidases, and methods of use thereof, for treating inflammation and related diseases.

### BACKGROUND OF THE INVENTION

All cells are covered with a dense and complex array of sugar chains. Sialic acids (Sias) are a family of nine-carbon sugars that are typically present at the outermost units of these sugar chains. By virtue of their terminal position, sialic acids act as binding sites for many exogenous and endogenous receptors such as the Influenza viruses and the Siglec family of endogenous proteins. Such sugars are thus useful drug targets for the prevention and treatment of infection. They are also involved in various biological and pathological processes such as neuronal plasticity and cancer metastasis. In many of these instances, the precise structures of the sialic acid and the residues it is attached to play critical roles. Thus, studying sialic acid functions is of great biological importance. In addition, many sialic acids are obtained through certain dietary sources (red meat and dairy products), and may also be associated with certain disease states, such as cancer and heart disease.

The most common Sias on the mammalian cellular glycocalyx and secreted glycoproteins are N-acetylneuraminic acid (Neu5Ac) and N-glycolylneuraminic acid (Neu5Gc). Cellular Neu5Gc is generated by hydroxylation of the sugar nucleotide donor CMP-Neu-5Ac to CMP-Neu-5Gc, catalyzed by CMP-Neu5Ac hydroxylase (CMAH). Although Neu5Gc is a major Sia in most mammals (including our closest evolutionary relatives, the great apes), it is thought to be absent in healthy humans. Indeed, humans generate immune responses against intravenously administered molecules carrying Neu5Gc, e.g. the “serum sickness” reaction to equine anti-thymocyte globulin therapy. These findings are explained by a human-specific inactivating mutation in the CMAH gene that occurred 2.5-3 million years ago. Treatment for serum sickness has traditionally been aimed at reducing symptoms. However, experiencing serum sickness can lead to eventual nervous system disorders and/or anaphylaxis later in life, and no preventive therapies currently exist.

Dietary habits have been associated with alterations of the human gut resident microorganisms contributing to obesity, diabetes, and cancer. In Western diets, red meat is one of the most frequently eaten foods, but long-term consumption has been associated with increased risk of disease. Red meat is enriched in N-glycolylneuraminic acid (Neu5Gc), which cannot be synthesized by humans due to an evolutionary loss of a functional CMP-Neu5Ac hydroxylase (CMAH). However, consumption can cause Neu5Gc incorporation into cell surface glycans, especially in carcinomas. As a consequence, an inflammatory response is triggered when Neu5Gc-containing glycans encounter circulating anti-Neu5Gc antibodies.

Metabolic incorporation of Neu5Gc into tissues requires glycosidically bound-Neu5Gc for reasons currently unknown. In contrast, free-Neu5Gc is utilized by gut microbes or cleared rapidly by the kidneys through the urine (FIG. 4). Intestinal bacteria can release host-derived sialic-acids from mucosal mucins and glycolipids by expressing sialidases. It is also known that gut commensal and pathogenic bacteria can metabolically process sialic acids. However, currently known bacterial sialidases prefers Neu5Ac to Neu5Gc as a carbon source. How bound Neu5Gc is metabolized in the gut is therefore currently unknown. Once free, sialic-acids can be taken up through membrane-associated transporters and utilized as carbon, nitrogen, or energy sources, or used to sialilate bacterial cell surface glycans. In addition, changes in the intestinal concentration of sialic acids, for example induced by inflammation, can alter the expression of bacterial genes involved in sialic acid catabolism, promoting intestinal dysbiosis.

Despite the absence of any known alternative pathway for the synthesis of Neu5Gc in humans, antibodies have been used to claim the expression of Neu5Gc in human tumors, particularly in various carcinomas. Recent studies show Neu5Gc expression in human cancers and extend the finding to normal human tissues, detecting small amounts of Neu5Gc in epithelial and endothelial cells of healthy humans. Definitive confirmation resulted from releasing and purifying sialic acids from such tissues and identifying a fluorescent derivatized form of Neu5Gc by HPLC and mass spectrometry analysis. It has also been shown that exogenously added free Neu5Gc can be incorporated into cultured human carcinoma cells in vitro. Oral ingestion studies of Neu5Gc in human volunteers have also carried out, providing evidence that the Neu5Gc found in human tissues and carcinomas could originate from dietary sources, particularly red meat and milk products.

Neu5Gc-containing glycans can act as xeno-autoantigens that can be targeted by naturally circulating anti-Neu5Gc antibodies, leading to an inflammatory response called xenosialitis that is known to influence liver tumor progression. Corroborating these in vitro findings, feeding a Neu5Gc-rich diet to human-like cmah−/− mice that develop CRC (APC-CPC cmah−/−) increased the number and size of colon polyps. Thus, the dietary intake of Neu5Gc has been implicated as a mechanism involved in the increased risk of CRC associated with red meat. Currently, there are no known or proposed methods to prevent or eliminate these harmful effects mediated by Neu5Gc in red meat-eaters, a group that includes the majority of US population.

Thus, it would be desirable to develop a new solution for treating or preventing development and progression of cancers including, but not limited to, colorectal cancer and liver cancer; cardiovascular disease; serum sickness; intestinal dysbiosis; and other inflammatory conditions related to red meat consumption and/or exposure to non-human-origin sialic acids. These needs and other needs are satisfied by the present disclosure.

### SUMMARY OF THE INVENTION

In accordance with the purpose(s) of the present disclosure, as embodied and broadly described herein, the disclosure, in one aspect, relates to novel sialidase enzymes having a preference for cleaving N-glycolylneuraminic acid (Neu5Gc) from glycoconjugates over cleaving N-acetylneuraminic acid (Neu5Ac). In one aspect, the novel sialidase exhibits a preference for cleaving N-glycolylneuraminic acid (Neu5Gc) from glycoconjugates over cleaving N-acetylneuraminic acid (Neu5Ac). The novel sialidase further comprises: an Asp-box comprising RIP/RLP motif and four Asp containing consensus sequences (Ser/Thr-Xaa-Asp-[Xaa]-Gly-XaaThr-Trp/Phe), which electrostatically interact with the carboxylate groups of sialic acids. These consensus sequence also comprises a conserved nucleophilic tyrosine and acid/based glutamic acid residues at C-terminal portion of said sialidase, a N-terminal signal peptide which flags them for secretion across the membrane, and at least one catalytic pocket, with Neu5Gc preferential activity.

In one aspect, the novel sialidase enzymes comprise at least one polypeptide sequence selected from SEQ ID NOs. 1-86. In another aspect, the novel sialidase enzyme is selected from SEQ ID NO. 1-8. Also disclosed are polynucleotides selected from SEQ ID NOs. 87-172 and derivatives and variants thereof encoding the novel sialidases and vectors comprising the polynucleotides. In another aspect, the disclosure relates to recombinant host cells comprising the polynucleotide sequences and vectors. In another aspect, disclosed herein is a non-virulent polypeptide having sialidase activity with a preference for Neu5Gc. In some aspects, the polypeptide having sialidase activity can be a bacterial sialidase. In a further aspect, the polypeptide includes a binding pocket that preferably engages Neu5Gc over Neu5Ac. In a still further aspect, the binding pocket includes at least the following residues or conservative substitutions thereof:
1. Aspartic acid (D) or alanine (A) separated by 25-40 amino acids from—
2. Aspartic acid (D), asparagine (N) or threonine (T), separated by 15-25 amino acids from—
3. Tyrosine (Y) or tryptophan (W), separated by 30-40 amino acids from—
4. Arginine (R), tryptophan (W), leucine (L), phenylalanine (F), or isoleucine (1), separated by 5-30 amino acids from—
5. Leucine, (L), glutamine (Q), asparagine (N), or arginine (R), separated by 30-50 amino acids from—
6. Aspartic acid (D) or glutamic acid (E), separated by 10-20 amino acid from—
7. Arginine (R), separated by 25-80 amino acids from—
8. Arginine (R), separated by 5-40 amino acids from—
9. Tyrosine (Y)

In one aspect, additional sialidase proteins having the desired features (i.e., binding pocket residues) have been identified in the UniProt database and are included herein as SEQ ID NOs. 9-63 or have been isolated from the environment and are included herein as SEQ ID NOs. 64-86.

In another aspect, the disclosure relates to enzyme compositions comprising the novel sialidases and consumable products such as foods, beverages, dietary supplements, and pharmaceutical compositions comprising the enzyme compositions.

In still another aspect, the disclosure relates to a probiotic composition comprising a bacterium expressing one or more sialidases having at least 60% sequence identity with the novel sialidases disclosed herein.

In a further aspect, the disclosure relates to a method for treating or preventing a disease or disorder associated with gastrointestinal disorders and/or inflammation. Examples of such diseases or disorders include, but are not limited to, inflammatory bowel disease (e.g., Crohn's disease, ulcerative colitis, indeterminate colitis), HIV enteropathy, colitis, necrotizing enterocolitis (NEC), colon cancer, colitis-associated colon cancer, chronic fatigue syndrome, leaky gut syndrome, nutrient-induced inflammation, cystic fibrosis, bacterial gastroenteritis, celiac disease, systemic lupus erythematosus, atherosclerosis, inflammatory heart disease, endocarditis, valvular heart disease, peripheral arterial disease, cardiomyopathy, and the like. Moreover, a chronic gastrointestinal inflammation comprises a gastrointestinal disorder. A gastrointestinal disorder includes, without limitation, irritable bowel disease, an inflammatory bowel disease including Crohn's disease and an ulcerative colitis like ulcerative proctitis, left-sided colitis, pancolitis and fulminant colitis. Chronic gastrointestinal Inflammation orchestrates the microenvironment around tumors, contributing to proliferation, survival and migration. For example, fibrinous inflammation results from a large increase in vascular permeability which allows fibrin to pass through the blood vessels. If an appropriate procoagulative stimulus is present, such as cancer cells, a fibrinous exudate is deposited. This is commonly seen in serous cavities, where the conversion of fibrinous exudate into a scar can occur between serous membranes, limiting their function. In another example, a cancer is an inflammatory cancer such as a NF-κB-driven inflammatory cancer.

In a further aspect, the present disclosure further provides a method for treating or preventing other disorders, including but not limited to, neurological disorders, ocular disorders, metabolic disorders, auto-immune disorders, and cardiovascular disorders.

The present disclosure further provides a method of treating an individual with a chronic inflammation. In one embodiment, the method comprises the step of administering to an individual in need thereof a pharmaceutical composition disclosed herein, wherein administration reduces a symptom associated with the chronic inflammation, thereby treating the individual.

In another aspect, the disclosure relates to a method for removing Neu5Gc from a food product such as, for example, red meat or dairy, the method comprising contacting the consumable product with the disclosed enzyme composition. Optionally, the enzyme composition can be removed before the food product is consumed.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1E show the composition of gut microbial community of mice fed on a soy, PSM (porcine submaxillary mucin), or EBN (edible bird's nest) diet. FIG. 1A: Beta-diversity analysis of wild-type (WT) vs Cmah^{-/-} mouse. FIG. 1B: Beta-diversity analysis of Cmah^{-/-} mouse fed with soy, PSM, or EBN diet. FIG. 1C: Differentially abundant bacterial genera on PSM and EBN diet. FIG. 1D: Sialidases diet-dependent using CAZYme database. FIG. 1E: Relative abundance ratio (PSM/SOY and EBN/SOY) of the bins with annotated sialidases.

FIGS. 2A-2F show screening for Neu5G-preferring sialidases in human and environmental samples. FIG. 2A: Relative abundance of bin13 in samples from Hadza hunter-gatherer population (n=20 biologically independent samples per season) FIG. 2B: Relative abundance of binHz19 in samples from Hadza hunter-gatherers population (n=20 biologically independent samples per season). FIG. 2C: Activity of sialidaseHz136 retrieved from Hadza shotgun metagenome at three different pH levels (6.5, 7.0, and 8.0) using 0.5 μg of each enzyme. Data are represented as triplicates (n=3 biologically independent experiments) and the statistical analysis were performed using Students 2-tailed t test. FIG. 2D: Crystal structure of sialidaseHz136 aligned to the catalytic site and carbohydrate-binding motif of sialidase26. FIG. 2E: Sialidase26 and sialidaseHz136 activity on beef (New York steak), pork (pork sausage), and PSM chow. Data are represented as triplicates (n=3 biologically independent experiments). FIG. 2F: Comparison of composting sialidases activity using fluorogenic 4MU-α-Neu5Gc and 4MU-α-Neu5Ac substrates. The experiments were performed in duplicates (n=2 biologically independent experiments). Au—Arthrobacter ureafaciens, C19—composting sialidase C19, C22—composting sialidase C22, Cp—Clostridium perfringens, Mv—Micromonospora viridifaciens, St—Salmonella typhimurium, and Vc—Vibo cholerae. Statistical significances were determined using the non-parametric two-sided Wilcoxon rank sum test with Holm correction for multiple hypotheses. Boxes and whiskers indicate quartiles and middle maker indicate the median (n=20) (FIGS. 2A-2B). Statistical significance was determined by Student's 2-tailed t test. Bars represent geometric mean±s.e.m. The significance levels are indicated as follow: p-values 0.05 (*), p-values 0.01 (**), and p-values 0.001 (***) (FIGS. 2C, 2D, 2F)

FIGS. 3A-3D show characterization of sialidase preference for Neu5Ac or Neu5Gc-containing substrates. FIG. 3A: Sialidase activity assay using 0.5 μg of each sialidase at three different pH values (6.5, 7.0, and 8.0). A mix of WT and Cmah^{-/-} mouse serum containing similar amount of bound-Neu5Ac and -Neu5Gc was used as substrate. Data are represented as triplicates (n=3 biologically independent experiments). FIG. 3B: Crystal structures of sialidase26 co-crystallized with DANA-Ac. FIG. 3C: Crystal structures of sialidase26 co-crystallized with DANA-Gc. Interacting side chains are represented as sticks, as well as the ligand. Amino acid residues and position interacting with the sialic-acid are indicated with labels. FIG. 3D: Overall ratio sialidase activity in clarified fecal samples from mice fed with PSM (n=4 biologically independent experiments) or Sia-free soy (n=3 biologically independent experiments). Sialidase activity assay was performed using 2 μg of total protein and a mix of WT and Cmah^{-/-} mouse serum containing similar amount of bound-Neu5Ac and -Neu5Gc. Sialidase activity is presented as a ratio of Neu5Gc (pmoles) and Neu5Ac (pmoles). Statistical significance was determined by Student's 2-tailed t test. The significance levels are indicated as follow: p-values 0.05 (*), p-value≤0.01 (**), and p-values 0.001 (***). Bars represent geometric mean±s.e.m.

FIG. 4 shows a proposed scheme for the incorporation of bound-Neu5Gc. In the absence of sialidases, Neu5Gc is incorporated into human tissue triggering xenosialitis. In the presence of bacterial sialidases, Neu5Gc is released from red meat glycoproteins and metabolized by bacteria or excreted in the urine.

FIG. 5 shows beta-diversity analysis of the microbiome of WT vs Cmah^{-/-} mice fed with Sias-free (soy), Neu5G-rich (PSM), or Neu5Ac-rich (EBN) diet. Pairwise Bray-Curtis dissimilarities were plotted against the first and second principal coordinates (ANOSIM R=0.979, pvalue=0.001). Significant vectors in grey (R>=0.7, p value=<0.01) were obtained using the envfit function from the R package vegan version 2.5-2 and the most representative taxa are indicated on the plot. WT samples enclose n=5 biologically independent animals and n=15 independent experiments per diet. Cmah^{-/-} samples enclose n=3 biologically independent animals and n=9 independent experiments.

FIGS. 6A-6B show the microbiome of Cmah^{-/-} mice and WT mice fed soy, PSM, and EBN diets was determined through 16S rRNA gene amplicon sequencing. FIG. 6A: Alpha-diversity analysis of WT vs Cmah^{-/-} mouse fed with a Sias-free (soy), Neu5Gc-rich (PSM), or Neu5Ac-rich (EBN) diet. Statistical differences in diversity were calculated using the nonparametric Wilcoxon rank sum test with Holm correction for multiple hypotheses. The significance levels are indicated as follow: p-value≤0.05 (*), p-value≤0.01 (**), and p-value≤0.001 (***). Boxes and whiskers indicate quartiles and middle maker indicate the median (cmah^{-/-} n=9; wt n=15). FIG. 6B: Relative abundance at family level of Cmah^{-/-} mouse fed with an Sias-free (soy), Neu5Gc-rich (PSM), or Neu5Ac-rich (EBN) diet.

FIG. 7 shows OTUs taxonomic relative abundance at genus level. 16S rRNA gene amplicon sequencing was performed for Cmah^{-/-} mice fed soy, PSM, and EBN diets. Bacteria genus with relative abundance below than 1% were not considered for differential abundance analysis. Relative abundance is log 2 transformed. Columns are colored label according with diet (red—PSM, blue—EBN, and green—SOY). Hierarchical clustering using Euclidian distance were applied to both row and column as indicated for the phylogenetic trees. From top to bottom, the organism classifications are as follows: Allobaculum, Romboutsia, Saccharibacteria, Lactobacillus, Oscillibacter, Helicobacter, Chlamydia, Parabacteroides, Turicibacter, Clostridium, Clostridium, Flavonifractor, Anaerotruncus, Anaerovorax, Allopevotella, Akkermansia, Olsenella, Acetatifactor, Deinococcus, Alkaliphilus, Lachnospiracea, Mucispirillum, Odoribacter, Parapedobacter, Paraprevotella, Rikenella, Vallitalea, Alistipes, Clostridium, Parasutterella, Brevibacillus, Natronoflexus, Bifidobacterium, Eubacterium, Intestinimonas, Mycoplasma, Clostridium, Roseburia, Anaeroplasma, Vampirovibrio, Desulfovibrio, Clostridium, Bacteroides, Barnesiella.

FIGS. 8A-8C show evaluation of growth phenotypes associated with the exo-α-sialidase activity in the gut microbiome. Genome-scale network reconstructions combine detailed biochemical and physiological information, providing new insights into the metabolism for subsequent manipulation strategies to enhance productivity or to control the metabolism. The scope of these models encompasses the characterization of the metabolic behaviour of target microorganisms. FIG. 8A: Gut-microbiome microorganisms with genome-scale metabolic models available based on 773 bacterial genomes. FIG. 8B: 17 microorganisms containing putative sialidase activity were identified. Most of the identified organisms fall into the taxonomic group of Bacteroidetes. FIG. 8C: Phenotypes regarding growth rate showed that microorganism carrying sialidase activity can growth around 0.1-0.51 1/h, with Bacteroidetes being the most efficient growing microorganisms with an average growth rate of 4.8±0.35 1/h. Modeling predictions show that six out of seven Bacteroidetes can reshape their growth rate depending on the sialidase activity. The maximum change in the growth rate is 11±2% of the wild type growth rate. The growth change due to sialidase activity is shown in shaded red (top of barplot) in FIG. 8C.

FIG. 9 shows principal coordinate analysis (PCoA) applied to CAZymes of Cmah^{-/-} mice fed with Sias-free (soy), Neu5Gc-rich (PSM), or Neu5Ac-rich (EBN) diet. Pairwise Bray-Curtis dissimilarities were plotted against the first and second principal coordinates (ANOSIM R=0.246, pvalue=0.06). N=3 biologically independent animals were fed each diet.

FIGS. 10A-10B show pan-genome analysis indicating the presence of 1,371 sialidases genes in bacterial genomes available in the PATRIC database. FIG. 10A: Number of sialidases per bacterial genome available at PATRIC database. While most bacterial genomes contain only one sialidase, hundreds of examples of bacterial genomes with multiple sialidases were identified in PATRIC. FIG. 10B: Evaluation of contamination, strain heterogeneity, and completeness's of all 51 bins retrieve from the shotgun metagenome. The presence of multiple sialidases per genome is likely to be a result of bacterial strains or close related species co-assembling. Although the ability to correctly assemble genomes from complex microbial communities has dramatically improved, the distinction between genomes from different strains is still a challenge. Evaluating the 51 bins assembled from our shotgun metagenome, bin13 containing the 5 sialidase genes characterized here, has shown one of the highest strain heterogeneity (36.36%) among all the bins.

FIG. 11 shows an amino acid sequence alignment between exemplary sialidase enzymes (sialidase26 (SEQ ID NO. 4) and sialidase CUA18247.1 (SEQ ID NO. 53) disclosed herein.

FIG. 12 shows sialidase26 sequence alignment between the amino acid sequence obtained from the metagenomics data (de novo genome assembling, SEQ ID NO. 26) and the amino acid sequence obtained from the PCR amplified gene (Sanger sequencing, SEQ ID NO. 26 with truncation and added His tag). For PCR, DNA extracted from mouse fecal samples was used as template.

FIG. 13 shows sialidase substrate preference. To the best of our knowledge, no previously characterized exo-sialidases have been shown to prefer Neu5Gc over Neu5Ac. A single report has shown that a sialidase from the oyster species Crassostrea virginica cleaves the very rare glycan structure Neu5Gcα2,5-O(glycolyl)Neu5Gc more efficiently than Neu5Acα2-8Neu5Ac. Because enzyme substrate preference using a glycan structure with same terminal linkage but differing in Neu5Gc vs Neu5Ac was not evaluated, it cannot be concluded that the oyster sialidase actually prefers Neu5Gc-containing substrates. Similarly, the sialyltransferase from the pathogenic bacteria Pasteurella multocida has been shown to cleave Neu5Gc more efficiently than Neu5Ac, however, no assay was performed in order to evaluate preferential cleavage. In fact, previous studies revealed that many bacterial species such as Arthrobacter ureafaciens, Clostridium perfringens, Streptococcus sp., Salmonella typhimurium, Streptococcus pneumonia, Ruminococcus gnavus, and Vibrio cholera express sialidases that hydrolyze Neu5Ac-terminated glycans more efficiently than Neu5Gc-terminated glycans, a substrate preference that is also shared by the murine sialidases Neu1, Neu2 and Neu4. Sialidase activity was evaluated using different enzyme concentrations (0.5 μg, 2.5 μg, 5 μg, and 10 μg). For protein expression and purification, sialidase genes were PCR amplified using DNA extracted from mouse fecal samples as the template. Independent experiments were performed in duplicate with similar results.

FIG. 14 shows that salidase26 (SEQ ID NO. 26) shares common sequence motifs with other sialidases. BTSA (sialidase from Bacteroides thetaiotomicron, SEQ ID No. 87) is provided as an example, aligned to Sialidase26. Common sialidase traits include a RIP/RLP motif (AAs 203-205) and four Asp-containing consensus sequences (AAs 240-247, 319-326, 379-387, and 486-493; S/T-x-D-x-G-x-T-W/F), which electrostatically interact with the carboxylate groups of sialic acids. It also contains a conserved nucleophilic tyrosine and acid/based glutamic acid residues at AAs 509 and 398, respectively, and a N-terminal signal peptide, which flags them for secretion across the membrane. Gray shades indicate the signal sequence, RIP/RLP motif, and Asp boxes as described on the left.

FIG. 15 shows the overall structure of GH33 (glycoside hydrolase family 33) sialidases with labelled catalytic site and carbohydrate binding motif.

FIG. 16 shows a comparison between sialidase26 and existing sialidases structures. The sialidase26 identified in this study was compared with published sialidase structures. Despite this novel substrate preference, sequence residues that are predicted to interact with terminal Sias in the catalytic site are highly also fully conserved with structurally studied sialidases exhibiting no known Neu5Gc preference over Neu5Ac, including those from B. caccae (PDB 4Q6K), B. thetaiotamicron, and P. distasonis (PDB 4FJ6). Other known sialidases with very low sequence identity (<30%) nevertheless shared 50-75% identity with residues predicted to interact with sialic acid, including NanA and NanB from S. pneumoniae NanI from C. perfringens, an IT-sialidase from R. gnavus, and a trans-sialidase from M. decora, but suggest no clear sequence motif or mutation causing Neu5Gc preference. Sialidase26 exhibits high similarity to other sialidases, including a sialidase of B. thetaiotomicron (BTSA). Both sialidases contain a wide binding site for broad substrate accommodation; BTSA exhibits hydrolysis activity for α2,6-, 2,3-, and 2,8-linked sialic acid moieties. For both proteins, the CBM is found N-terminally. Missing from both sialidases is an insertion motif (circle), found in the Macrobedella decora sialidase.

FIG. 17 shows Hadza shotgun metagenome analysis. Normalized read counts (counts per million) of the bins with annotated sialidases. p-values were computed using the two-sided Wilcoxon rank sum test with Holm correction for multiple hypotheses. n=20 biological independent sample per condition (dry, left bar for each condition; or wet, right bar for each condition). Boxes and whiskers indicate quartiles and middle maker indicate the median (n=20).

FIG. 18 shows amino acid sequence alignment between the sialidase26 (SEQ ID NO. 4) and the sialidaseHz136 (SEQ ID NO. 6). The crystal structure suggests that the D271 residue (rectangle on fourth row ending with amino acids 298 and 296) plays a key role in substrate preference. However, all amino acids residues in contact with the Sias (all other rectangles) might play a contributing role due to contact with the substrate.

FIG. 19 shows a schematic of fosmid library construction, screening, enzyme selection, and characterization.

FIG. 20 shows an amino acid sequence alignment between exemplary sialidase enzymes (sialidaseC19 (SEQ ID NO. 7) and sialidaseC22 (SEQ ID NO. 8)) disclosed herein.

FIGS. 21A-21B show sialidase alignments with the compost samples. Rectangles show the amino acids in contact with the sialic acid. Two R (arginine) residues and one Y (tyrosine) at the —COOH portion of the proteins are conserved. SEQ ID NOs. are as follows: sialidaseC19 (SEQ ID NO. 7), sialidaseC22 (SEQ ID NO. 8), sialidase60 (SEQ ID NO. 3), sialidase26 (SEQ ID NO. 4), sialidaseHz136 (SEQ ID NO. 6), sialidase65 (SEQ ID NO. 2), sialidase23 (SEQ ID NO. 1), sialidase24 (SEQ ID NO. 5).

FIG. 22 shows a schematic of the xenosialitis hypothesis described herein. Neu5Gc is metabolically incorporated from the diet (primarily red meats) into cellular glycans to form xeno-autoantigens.

FIG. 23 shows a graph showing sialidase activity, including a comparison of enzyme preference for Neu5Gc (black rectangles) or Neu5Ac (white rectangles).

Additional advantages will be set forth in part in the description which follows, and in part will be obvious from the description, or can be learned by practice of the invention. The advantages will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

Disclosed herein are novel sialidase enzymes having a preference for cleaving N-glycolylneuraminic acid (Neu5Gc) from glycoconjugates over cleaving N-acetylneuraminic acid (Neu5Ac), polynucleotides encoding the same, vectors comprising the polynucleotides, and recombinant host cells expressing the same. In one aspect, the present disclosure provides novel sialidase enzymes having a preference for cleaving N-glycolylneuraminic acid (Neu5Gc) from glycoconjugates over cleaving N-acetylneuraminic acid (Neu5Ac). In one aspect, the novel sialidase exhibits a preference for cleaving N-glycolylneuraminic acid (Neu5Gc) from glycoconjugates over cleaving N-acetylneuraminic acid (Neu5Ac). The novel sialidase further comprises: an Asp-box comprising RIP/RLP motif and four Asp containing consensus sequences (Ser/Thr-Xaa-Asp-[Xaa]-Gly-XaaThr-Trp/Phe), which electrostatically interact with the carboxylate groups of sialic acids. These consensus sequence also comprises a conserved nucleophilic tyrosine and acid/based glutamic acid residues at C-terminal portion of said sialidase, a N-terminal signal peptide which flags them for secretion across the membrane, and at least one catalytic pocket, with Neu5Gc preferential activity.

In one aspect, the novel sialidase enzymes comprise at least one polypeptide sequence selected from SEQ ID NOs. 1-86. In another aspect, the novel sialidase enzyme is selected from SEQ ID NOs. 1-8. In another aspect, the novel sialidase enzymes are encoded by at least one polynucleotide sequence selected from SEQ ID NOs. 87-172 or a conservative variant thereof. In a still further aspect, the catalytic pockets of sialidase enzymes disclosed herein, with Neu5Gc preferential activity, contain the following conserved amino acids:
sialidase23 (SEQ ID NO. 1): R51, I52, D76, M109, D110, T127, W129, R195, N228, E229,
R245, R306, Y334 sialidase65 (SEQ ID NO. 2): R49, I50, R68, D74, R107, N108, C125, A167, N225, E226, R242, Q244, I275, C276, Y333
sialidase60 (SEQ ID NO. 3): R181, I182, R200, D206, L207, D249, L266, G276, K277, L339, A341, Q360, R362, T393, E394, T449, C450, R480, Y511
sialidase26 (SEQ ID NO. 4): R203, I204, R222, D228, L229, D271, A288, A298, W299, F343, L345, Q364, T397, E398, R414, N416, R478, W507, Y509
sialidase24 (SEQ ID NO. 5): R71, V72, R90, D96, K97, M132, D133, W152, S203, R227, E265, R281, R344, Y377
sialidaseHz136 (SEQ ID NO. 6): R201, I202, R220, D226, L227, D269, A286, A296, W297, F341, L343, Q362, T395, E396, R412, N414, R476, W503, Y505
sialidaseC19 (SEQ ID NO. 7): H50, E102, T103, V152, M154, G173, F185, E215, R232, R266, H345
sialidaseC22 (SEQ ID NO: 8): H46, K97, T99, Y121, I147, M150, M183, C212, E213, R230, N232, D263, T343

In another aspect, disclosed herein is a non-virulent polypeptide having sialidase activity with a preference for Neu5Gc. In some aspects, the polypeptide having sialidase activity can be a bacterial sialidase. In a further aspect, the polypeptide includes a binding pocket that preferably engages Neu5Gc over Neu5Ac. In a still further aspect, the binding pocket includes at least the following residues or conservative substitutions thereof:
1. Aspartic acid (D) or alanine (A) separated by 25-40 amino acids from—
2. Aspartic acid (D), asparagine (N) or threonine (T), separated by 15-25 amino acids from—
3. Tyrosine (Y) or tryptophan (W, separated by 30-40 amino acids from—
4. Arginine (R), tryptophan (W, leucine (L), phenylalanine (F), or isoleucine (1), separated by 5-30 amino acids from—
5. Leucine, (L), glutamine (Q), asparagine (N), or arginine (R), separated by 30-50 amino acids from—
6. Aspartic acid (D) or glutamic acid (E), separated by 10-20 amino acid from—
7. Arginine (R), separated by 25-80 amino acids from—
8. Arginine (R), separated by 5-40 amino acids from—
9. Tyrosine (Y)

In one aspect, additional sialidase proteins having the desired features (i.e., binding sites residues) have been identified in the UniProt database and are included herein as SEQ ID NOs. 9-63 or have been isolated from the environment and are included herein as SEQ D NOs. 64-86.

Further disclosed are enzyme compositions that include the sialidases of the present disclosure, and pharmaceutical compositions and consumable products that include the same. The disclosure further relates to a probiotic composition comprising a bacterium that expresses a sialidase having a preference for Neu5Gc over Neu5Ac as well as to methods for removing Neu5Gc from consumable products and methods for treating or preventing various diseases and/or disorders associated with or without acute and/or chronic inflammatory conditions in a subject using the disclosed novel sialidases, which comprise at least one polypeptide sequence selected from SEQ ID NOs. 1-86, preferably SEQ ID Nos: 1-8, or are encoded by at least one polynucleotide sequence selected from SEQ ID NOs. 87-172 or a conservative variant thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a sialidase,” “an amino acid residue,” or “an inflammatory condition,” include, but are not limited to, mixtures or combinations of two or more such sialidases, amino acid residues, or inflammatory conditions, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a sialidase enzyme refers to an amount that is sufficient to achieve the desired improvement in the property modulated by the formulation component, e.g. achieving the desired level of reduction of inflammation. The specific level in terms of wt % in a composition required as an effective amount will depend upon a variety of factors including the enzyme identity and amount, patient diet, frequency of dosage with the formulations, and the like.

As used herein, the term “preference for Neu5Gc” or “Neu5Gc preferring” refers to sialidase activity of a sialidase to preferably bind and cleave bound Neu5Gc over bound Neu5Ac.

As used herein, the term “inflammatory diseases” includes arthritis, specifically rheumatoid arthritis, reactive arthritis and bacterial arthritis; inflammatory bowel disease, and autoimmune conditions. In one aspect, provided herein are methods and compositions useful for treating and/or preventing a disease or disorder associated with gastrointestinal inflammation, including, but not limited to, inflammatory bowel diseases (e.g., Crohn's disease, ulcerative colitis, indeterminate colitis), HIV enteropathy, colitis, necrotizing enterocolitis (NEC), colon cancer, colitis-associated colon cancer, chronic fatigue syndrome, leaky gut syndrome, nutrient-induced inflammation, cystic fibrosis, bacterial gastroenteritis, celiac disease, systemic lupus erythematosus, and the like. In some aspects, chronic gastrointestinal inflammation comprises a gastrointestinal disorder. A “gastrointestinal disorder” includes, without limitation, irritable bowel disease, an inflammatory bowel disease such as Crohn's disease and/or an ulcerative colitis like ulcerative proctitis, left-sided colitis, pancolitis, and/or fulminant colitis. Without wishing to be bound by theory, chronic gastrointestinal inflammation orchestrates the microenvironment around tumors, contributing to proliferation, survival, and migration. In one aspect, fibrinous inflammation can result from a large increase in vascular permeability, which allows fibrin to pass through the blood vessels. Further in this aspect, if an appropriate procoagulative stimulus, such as cancer cells, is present, a fibrinous exudate can be deposited. Still further in this aspect, this phenomenon is commonly seen in serous cavities, where the conversion of fibrinous exudate into a scar can occur between serous membranes, limiting their function. In one aspect, the cancer cells can be from an inflammatory cancer like a NF-κB-driven inflammatory cancer.

In some aspects, the methods and compositions disclosed herein are useful for treating diseases and disorders caused in whole or in part by chronic, low-level inflammation. Further non-limiting examples of such inflammatory disorders and diseases include, but are not limited to, neurological disorders such as, for example, mild or severe brain trauma, depression, attention deficit hyperactivity disorder (ADHD), attention deficit disorder (ADD), Parkinson's disease, Alzheimer's disease, neuropathy, and combinations thereof. In another aspect, inflammatory disorders and diseases associated with chronic, low-level inflammation include ocular disorders such as, for example, age-related macular degeneration, dry eye syndrome, optic nerve syndrome, diabetic retinopathy, and combinations thereof. In still another aspect, inflammatory diseases and disorders associated with chronic, low-level inflammation include gastrointestinal disorders such as, for example, colitis, leaky gut syndrome, colorectal cancer, Crohn's disease, irritable bowel disease, inflammatory bowel disease, and combinations thereof. In yet another aspect, inflammatory diseases and disorders associated with chronic, low-level inflammation can include metabolic disorders including, but not limited to, type 2 diabetes, metabolic syndrome, obesity, asthma, allergies, and combinations thereof. In still another aspect, inflammatory diseases and disorders associated with chronic, low-level inflammation include auto-immune disorders such as, for example, type 1 diabetes, lupus, cancers, heart disease, and the like.

As used herein, “inflammatory bowel disease” (IBD) refers to inflammatory conditions of the colon and small intestine. IBD can include, but is not limited to, Crohn's disease, ulcerative colitis, collagenous colitis, lymphocytic colitis, ischemic colitis, diversion colitis, Behçet's disease, and/or indeterminate colitis. In one aspect, inflammatory bowel diseases (BD) are becoming increasingly common. In some aspects, the inflammatory reaction is localized only on the mucosa (e.g. ulcerative colitis), but in other aspects, e.g. in Crohn's disease, inflammation can occur in all layers of the bowel wall from the mucosa to the serosa (transmuralic). In one aspect, without wishing to be bound by theory, during the inflammatory process, neutrophils and endothelial cells begin to produce remarkable quantities of inflammatory-causing cytokines (IL-1, IL-6, TNF-α). The main aims of medicinal treatments used for the inflammatory bowel diseases are to ease symptoms of the disease, to suppress the inflammatory process, and to prevent the complications (e.g. abscess, fistula).

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer, cardiovascular disease, and/or serum sickness, or another inflammatory disease or disorder as described herein. The effect can be therapeutic in terms of a partial or complete cure of a disease, condition, symptom or adverse effect attributed to the disease, disorder, or condition. The term “treatment” as used herein can include any treatment of cancer, cardiovascular disease, and/or serum sickness or other inflammatory disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

In one aspect, disclosed herein is a method of treating an individual with a chronic inflammation. In one embodiment, the method comprises the step of administering to an individual in need thereof a pharmaceutical composition disclosed herein, wherein administration reduces a symptom associated with the chronic inflammation compared to an initial state of the symptom, thereby treating the individual.

As used herein, the term “treating,” further refers to reducing or eliminating in an individual a clinical symptom of a chronic inflammation; or delaying or preventing in an individual the onset of a clinical symptom of a chronic inflammation. For example, the term “treating” can mean reducing a symptom of a condition characterized by a chronic inflammation by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%, compared to an initial state of the symptom. In one aspect, the actual symptoms associated with chronic inflammation are well known and can be determined by a person of ordinary skill in the art by taking into account factors, including, without limitation, the location of the chronic inflammation, the cause of the chronic inflammation, the severity of the chronic inflammation, and/or the tissue or organ affected by the chronic inflammation. Those of skill in the art will know the appropriate symptoms or indicators associated with a specific type of chronic inflammation and will know how to determine if an individual is a candidate for treatment as disclosed herein.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosed compositions (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

A “host cell” as used herein refers to a microbial cell that expresses a protein of interest. In one aspect, the host cell can be cultured under conditions designed to maximize production of the protein of interest. In some aspects, the host cell can be a “recombinant” host cell wherein the protein of interest is an exogenous protein from another organism and/or an environmental source. In one aspect, the exogenous protein can be introduced into the host cell using any technique known in the art such as, for example, inserting a polynucleotide sequence that codes for the protein of interest into a plasmid and introducing the plasmid to the host cell, making it a recombinant host cell. In one aspect, the host cell or recombinant host cell can be Escherichia coli or another bacterial cell.

Unless otherwise specified, temperatures referred to herein are based on atmospheric pressure (i.e. one atmosphere).

### Role of N-Glycolylneuraminic Acid (Neu5Gc) in Disease Processes

Sialic acids are nine-carbon sugars found on the surface of most mammalian cells. The two most common forms of sialic acids are N-acetylneuraminic acid (Neu5Ac) and N-glycolylneuraminic acid (Neu5Gc), which differ by only one oxygen atom:

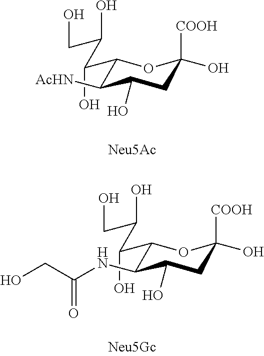

Unlike all other mammals, humans are unable to synthesize Neu5Gc due to a mutation in the CMP-Neu5Ac hydroxylase (CMAH) gene, which converts Neu5Ac to Neu5Gc. Despite the lack of expression of functional CMAH, small amounts of Neu5Gc have been found in healthy human tissues as well as malignant tissues. This Neu5Gc may originate from dietary sources. Red meat like beef, pork and lamb are particularly rich in Neu5Gc and should be considered the primary sources of Neu5Gc in human diet. Dairy products also contain Neu5Gc, although at somewhat lower levels than in red meat.

Neu5Gc has been associated with cell proliferative disorders, cancer, tissue rejection, and inflammation. Most humans have antibodies to Neu5Gc and an immune response to Neu5Gc is hypothesized to be one of the factors contributing to the health risks associated with high consumption of red meat (e.g., inflammation, heart disease, certain types of cancers). The presence of Neu5Gc in animal products is also a potential source of allergenicity.

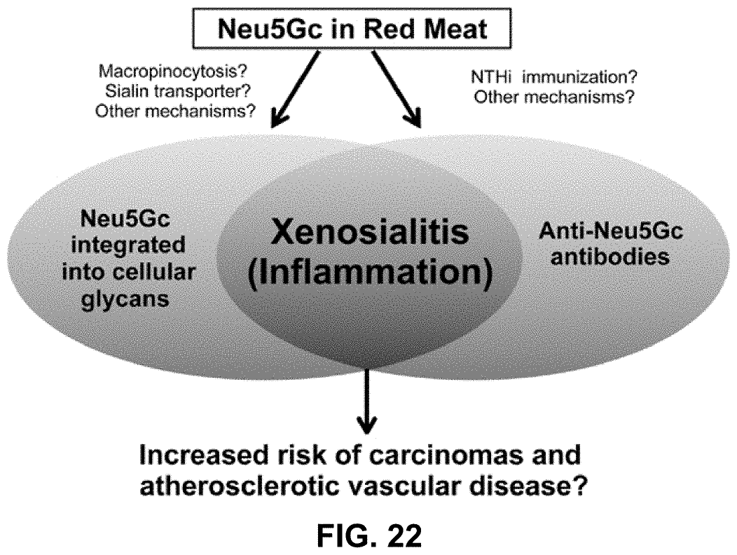

Accordingly, in one aspect, the present disclosure relates to a sialidase formulation comprising one or more sialidase enzymes with a preference for Neu5Gc, wherein the sialidase formulation is capable of immunomodulation, immunoregulation, reducing inflammation or xenosialitis, and reducing tumor progression. The formulation can be used in the manufacture of pharmaceutical compositions, dietary supplements, dietary foods for medical purposes, and nutraceuticals. The process for producing the material is also described, as are its use in the modulation, and regulation of immune reactions, reducing inflammation and/or tumor progression. The formulation can also be used in treatment of several inflammatory disorders. In one embodiment, the present disclosure relates to pre- or probiotic formulations to prevent incorporation or to eliminate Neu5Gc from tissues of red meat eaters therefore reducing the risk of xenosialitis and other diseases associated with red meat consumption, wherein the pre- or probiotic formulation comprises bacteria expressing one or more sialidase with a preference for Neu5Gc (see FIG. 22).

### Isolated Polypeptides

In one aspect, disclosed herein is an isolated polypeptide having sialidase activity with a preference for Neu5Gc. In another aspect, the polypeptide can have an amino acid sequence with at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% sequence identity, or has 100% sequence identity with any of amino acids: 1 to 373 of SEQ ID NO. 1; amino acids 1 to 392 of SEQ ID NO. 2, amino acids 1 to 546 of SEQ ID NO. 3; amino acids 1 to 543 of SEQ ID NO. 4; amino acids 1 to 539 of SEQ ID NO. 5; amino acids 1 to 369 of SEQ ID NO. 6; amino acids 1 to 387 of SEQ ID NO. 7; or amino acids 1 to 424 of SEQ ID NO. 8.

In one aspect, common sialidase sequences include a RIP/RLP motif and four Asp containing consensus sequences (S/T-x-D-x-G-x-T-W/F) called Asp-box, present as a sub-sequence of any of SEQ ID NOs. 1-86, which electrostatically interact with the carboxylate groups of sialic acids. In a further aspect, these sequences also contain a conserved nucleophilic tyrosine and acid/based glutamic acid residues at C-terminal portion of the enzyme. In a still further aspect, the binding site of sialidase enzymes disclosed herein, with Neu5Gc preferential activity, contain the following conserved amino acids:

sialidase23 (SEQ ID NO. 1): includes the following conserved binding site residues R51, I52, D76, M109, D110, T127, W129, R195, N228, E229, R245, R306, Y334
sialidase65 (SEQ ID NO. 2): includes the following conserved binding site residues R49, I50, R68, D74, R107, N108, C125, A167, N225, E226, R242, Q244, I275, C276, Y333
sialidase60 (SEQ ID NO. 3): includes the following conserved binding site residues R181, I182, R200, D206, L207, D249, L266, G276, K277, L339, A341, Q360, R362, T393, E394, T449, C450, R480, Y511
sialidase26 (SEQ ID NO. 4): includes the following conserved binding site residues R203, I204, R222, D228, L229, D271, A288, A298, W299, F343, L345, Q364, T397, E398, R414, N416, R478, W507, Y509
sialidase24 (SEQ ID NO. 5): includes the following conserved binding site residues R71, V72, R90, D96, K97, M132, D133, W152, S203, R227, E265, R281, R344, Y377
sialidaseHz136 (SEQ ID NO. 6): includes the following conserved binding site residues R201, I202, R220, D226, L227, D269, A286, A296, W297, F341, L343, Q362, T395, E396, R412, N414, R476, W503, Y505
sialidaseC19 (SEQ ID NO. 7): includes the following conserved binding site residues H50, E102, T103, V152, M154, G173, F185, E215, R232, R266, H345
sialidaseC22 (SEQ ID NO. 8): includes the following conserved binding site residues H46, K97, T99, Y121, I147, M150, M183, C212, E213, R230, N232, D263, T343

In another aspect, disclosed herein is a non-virulent polypeptide having sialidase activity with a preference for Neu5Gc. In some aspects, the polypeptide having sialidase activity can be a bacterial sialidase. In a further aspect, the polypeptide includes a binding pocket that preferably engages Neu5Gc over Neu5Ac. In a still further aspect, the binding pocket includes at least the following residues or conservative substitutions thereof:
1. Aspartic acid (D) or alanine (A) separated by 25-40 amino acids from—
2. Aspartic acid (D), asparagine (N) or threonine (T), separated by 15-25 amino acids from—
3. Tyrosine (Y) or tryptophan (W), separated by 30-40 amino acids from—
4. Arginine (R), tryptophan (W), leucine (L), phenylalanine (F), or isoleucine (1), separated by 5-30 amino acids from—
5. Leucine, (L), glutamine (Q), asparagine (N), or arginine (R), separated by 30-50 amino acids from—
6. Aspartic acid (D) or glutamic acid (E), separated by 10-20 amino acid from—
7. Arginine (R), separated by 25-80 amino acids from—
8. Arginine (R), separated by 5-40 amino acids from—
9. Tyrosine (Y)

In one aspect, additional sialidase proteins having the desired features (i.e., binding sites residues) have been identified in the UnProt database and are included herein as SEQ ID NOs, 9-63 or have been isolated from the environment and are included herein as SEQ ID NOs 64-86,

### Expression of Polypeptides

In one aspect, disclosed herein is a recombinant host cell expressing at least one sialidase having a preference for cleaving Neu5Gc from glycoconjugates over cleaving Neu5Ac. In some aspects, the at least one sialidase has the binding pocket features disclosed herein. In another aspect, the at least one sialidase includes at least one protein having at least 60% sequence identity with a protein selected from SEQ ID NOs. 1-86 and combinations thereof. In an alternative aspect, the at least one sialidase is selected from SEQ ID NOs. 1-86. In one aspect, the recombinant host cell can be E. coli. In another aspect, the at least one sialidase can be encoded by a polynucleotide selected from SEQ ID NOs. 87-172 or a conservative variant thereof.

### Bacterial Strains and Bacterial Culture

In one aspect, the disclosed polypeptide is from cultivated Bacteroidales, Planctomycetales, Micrococcales, Vibrionales, or Enterobacterales. In another aspect, the disclosed sialidase is extracted from bacteria selected from Bacteroides caccae, Bacteroides fragilis, Bacteroides thetaiotamicron, Bacteroides xylanosolvens, Bacteroides plebeius, Bifidobacterium longum, Prevotella bivia, Galbibacter marinus, or Planctomycetacea bacterium.

According to another aspect, disclosed herein is a method for producing the disclosed polypeptide, the method including at least the steps of cultivating a strain or recombinant host cell capable of expressing the disclosed polypeptide to produce a supernatant and/or cells containing the polypeptide and recovering the polypeptide. According to a further aspect, disclosed herein is a method for producing the polypeptide, the method including at least the steps of cultivating a host cell that includes a nucleic acid construct that further includes a polynucleotide encoding the disclosed polypeptide under conditions suitable for production of the polypeptide and recovering the polypeptide. In one aspect, the polypeptide having sialidase activity is non-virulent. In another aspect, the polypeptide can be used in the preparation of food or as a medicament or part of a medicament.

### Uses of the Disclosed Polypeptides

In some aspects, the disclosed compositions can be used to prevent incorporation of Neu5Gc into human tissues and/or to cleave or eliminate Neu5Gc from red meats. In a further aspect, the disclosed compositions can thus improve human health. In another aspect, the disclosed compositions can be used to condition animal serum used to culture human cells in laboratories. In still another aspect, the disclosed compositions can be used in the biotechnology industry or for animal products currently used in cosmetics, thereby reducing the risk of immune responses against such products.

Thus, in one aspect, the removal or reduction in Neu5Gc from humans and products consumed by humans can be used to reduce immunogenic responses and disease development. The disclosure provides methods for removal or reduction of existing Neu5Gc from a human subject, removal or reduction of Neu5Gc from a non-human biological material, removal or reduction of Neu5Gc from dietary consumables, and removal or reduction of Neu5Gc from therapeutics. In one aspect, this method can be coupled with methods that reduce the uptake of Neu5Gc by a human or decrease the symptoms of inflammation caused by Neu5Gc, in particular in the digestive tract. Thus, in one aspect, the disclosed compositions can be used as a pharmaceutical, dietary supplement, a dietary food for medical purposes, a supplementary food additive, or any combination thereof.

Reducing Risks Associated with Red Meat Consumption

Red and processed meats have been shown to be associated with increased rates of premature death. One of the most consistent epidemiological associations between diet and human disease risk is the impact of red meat consumption (beef, pork, and lamb, particularly in processed forms). While risk estimates vary, associations are reported with all-cause mortality, colorectal (CRC) and other carcinomas, atherosclerotic cardiovascular disease, type II diabetes, and possibly other inflammatory processes. In one aspect, a mechanistic explanation for the human propensity for risk of red meat-associated diseases that is the metabolic incorporation of Neu5Gc into the tissues of red meat consumers.

In another aspect, consumption of red meat has long linked to an increased incidence of cardiovascular disease (CVD), the leading cause of death worldwide. Atherosclerosis is the most common underlying process leading to CVD in humans, but red meat feeding in non-human mammals rarely leads to CVD due to atherosclerosis, including in carnivores with a red meat-heavy diet. In one aspect, the uniquely human association between red meat and atherosclerosis represents a current gap in our understanding of CVD and how to effectively treat it. In a further aspect, one proposed human-specific explanation is that atherosclerosis is at least partly driven by an antibody-mediated reaction Neu5Gc. Neu5Gc is structurally similar to the human sialic acid Neu5Ac, but humans cannot produce Neu5Gc due to an evolutionary loss of the enzyme expressed by the Cmah gene. However, ingested Neu5Gc can be incorporated into the glycoconjugates of human cells, including endothelial cells. In one aspect, human anti-Neu5Gc antibodies isolated from serum can bind cultured endothelial cells in a Neu5Gc-dependent manner, thereby triggering complement deposition and endothelial activation. Furthermore, Neu5Gc feeding in a humanized (Cmah^{-/-}, i.e. Neu5Gc-deficient) atherosclerosis mouse model can lead to up to a 3-fold increase in atherosclerosis lesion volume and 5-fold increase in necrotic core size, indicating advanced lesion development. In one aspect, these advanced lesions cannot be explained by lipoprotein or glucose changes but only by Neu5Gc feeding. In one aspect, the enhanced atherosclerosis development with Neu5Gc feeding is only seen in Neu5Gc-deficient mice that have been immunized against Neu5Gc, mimicking the situation found in humans.

Accordingly, in one aspect, the disclosed compositions can be used to treat a variety of conditions including inflammatory diseases and to inhibit or prevent tumor progression. Further in this aspect, the compositions can be used to treat and/or prevent inflammatory conditions, cardiovascular diseases, and cancer.

### Pharmaceutical Compositions

In one aspect, disclosed herein are pharmaceutical compositions including the disclosed isolated polypeptides and/or bacterial organisms or extracts from bacterial cultures, wherein the bacterial organisms or cultured bacteria express sialidase enzymes displaying a preference for Neu5Gc over Neu5Ac. In another aspect, the pharmaceutical compositions further include at least one excipient, diluent, or carrier.

In another aspect, the disclosed compositions may be adapted for administration by any appropriate route, including, but not limited to, oral (e.g., buccal or sublingual), rectal, nasal, topical (e.g., buccal, sublingual, or transdermal), vaginal, or parenteral (e.g., subcutaneous, intramuscular, intravenous or intradermal) route. In another aspect, such formulations can be prepared by any method known in the art of pharmacy, for example by bringing into association the active ingredient with one or more carrier(s) or excipient(s).

In some aspects, pharmaceutical formulations can be administered orally in unit dosage form as an aqueous composition (e.g. oral drops) or in any of the usual solid forms including, but not limited to, pills, tablets, capsules, liquid or solid cachets, or powders, including sustained release preparations. In another aspect, such pills, tablets, capsules, cachets and powders incorporating the disclosed composition can be prepared by standard methods known in the art.

In one aspect, pharmaceutical formulations adapted for oral administration can be presented as discrete units such as solutions or suspensions in aqueous or non-aqueous liquids; edible foams or whips; or oil-in-water liquid emulsions or water-in-oil liquid emulsions.

In a further aspect, it should be understood that, in addition to the ingredients particularly mentioned above, the formulations can also include other agents conventional in the art having regard to the type of formulation in question: for example, those suitable for oral administration may include flavoring agents.

The term “unit dosage form” as used herein refers to physically discrete units to be administered in single or multiple doses to animals including humans, each unit containing a predetermined quantity of active material, i.e. sialidase, optionally in association with one or more carriers including pre-/probiotic formulations. In another aspect, the quantity of active material is that amount calculated to produce the desired nutritional or therapeutic effect upon administration of one or more of such units.

In one aspect, unit doses can be administered in any convenient amount or dosing schedule to achieve the stated beneficial effects. For example, the doses can be taken 1, 2, 3, 4, 5 or more times daily. In one aspect, the doses are taken at meal times when ingestion of NeuG5c-containing foods is anticipated and can be taken prior to or during ingestion of NeuG5c-containing foods.

In one aspect, preferred unit dosage formulations are those containing a daily dose or sub-dose, or an appropriate fraction thereof, of an active ingredient. Further in this aspect, suitable daily dosages can be between about 0,001 g and 100 g, or between about 0.01 g and 50 g, or between about 0.1 g and 40 g per kg body weight.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present disclosure may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present disclosure include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present disclosure therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present disclosure include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the disclosure formulated in a pharmaceutically acceptable carrier may also be prepared, placed in an appropriate container, and labeled for treatment of an indicated condition.

Another patch system configuration which can be used by the present disclosure is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present disclosure is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require sialidase activity, wherein the sialidase preferentially cleaves Neu5Gc over Neu5Ac, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating inflammatory activity and/or xenosialitis (e.g., treatment of one or more inflammatory disorders associated with consumption of red meat, dairy, and/or other food products expressing Neu5Gc) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Enzyme Compositions

In one aspect, disclosed herein is an enzyme composition that includes at least one sialidase having a preference for cleaving Neu5Gc from glycoconjugates over cleaving Neu5Ac. In another aspect, the at least one sialidase can be extracted from the host cells or recombinant host cells disclosed herein. In some aspects, disclosed herein are pharmaceutical compositions including the disclosed enzyme compositions and at least one excipient as disclosed herein. In another aspect, the pharmaceutical composition can be formulated for oral administration.

### Methods of Using the Pharmaceutical Compositions

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present disclosure also features such kits further containing instructions for use.

In a further aspect, the present disclosure also provides a pharmaceutical pack or kit comprising one or more containers filled with one or more of the ingredients of the disclosed pharmaceutical compositions. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### Dietary Supplements and Functional Foods and Beverages

In one aspect, disclosed herein is a consumable product that includes the disclosed enzyme composition. In another aspect, the consumable product can be a food, a beverage, a dietary supplement, or a combination thereof.

In yet another aspect, the disclosed compositions can be incorporated into dietary supplements, food products, or beverages by methods known in the art. In one aspect, examples of suitable food products include, but are not limited to, baked goods and non-baked goods such as nutritional bars, muffins, breads, cakes, energy gels, drink mixes, and the like. In another aspect, examples of beverages include flavored and unflavored waters, energy drinks, sports drinks, carbonated and non-carbonated soft drinks, teas, and the like. In some aspects, the compositions can also be used as an additional ingredient in dietary formulations of processed and non-processed foods.

In one aspect, a dietary supplement containing the disclosed composition and “foods” or “beverages” containing the disclosed composition can be the same form, differing only by the amount of sialidase composition included in each. In one aspect, when the amount of the sialidase composition incorporated in a food or beverage rises to a level of providing recommended daily values, the food or beverage can be referred to as a dietary supplement. Thus, in one aspect, dietary supplements can also be pills, tablets, capsules, powders, baked and non-baked goods, or carbonated and non-carbonated beverages, and the like, depending on the amount of sialidase composition incorporated.

In another aspect, the disclosed compositions can be formed into a form suitable for oral consumption (e.g., a food or beverage) by optionally blending thereto various kinds of additives including, but not limited to, sweeteners, spices, seasonings, food additives, preservatives, anti-oxidants, and combinations thereof, and also can be formed into a food or beverage including the composition for food or beverage by further processing the composition. In one aspect, the composition for food or beverage or the food or beverage can be formed into various shapes such as a solution, a suspension, a syrup, a granule, a cream, a paste, a jelly, or the like, and can be formed into a desired shape if necessary. In another aspect, the food or beverage can be formed into various shapes such as bread, noodle, confectionary, beverage, soup, and/or fabricated food. In one aspect, preparation of the composition for food or beverage and the food or beverage can be carried out by any known method.

In one aspect, the disclosed compositions can be used for prevention, amelioration, and/or maintenance of remission diseases and conditions associated with the presence of Neu5Gc as described herein. In one aspect, the amount of the sialidase formulation in the composition for food or beverage or the food or beverage of the present disclosure is not limited particularly, and can be optionally set by referring to the dosage in the case of the above-mentioned pharmaceuticals. In some aspects, the amount of sialidase formulations per unit body weight of 1 kg is from about 1 mg to about 40 mg, or from about 2 mg to about 20 mg, or from about 3 mg to about 13 mg for eating or drinking once.

In some aspects, the disclosed foods and beverages can be so-called health foods, health beverages, foods for specified health use, functional foods, nutritive supplements, and/or food supplements.

In one aspect, the disclosed pharmaceuticals, dietary supplements, and foods or beverages are present in a form that allows the active ingredients (e.g. sialidase formulations) to be absorbed via the digestive tract, preferably via an oral cavity or an intestine (for example, in a form of a sublingual tablet or an enteric coating drug).

In some aspects, the disclosed foods or beverages food can contain from about 0.1-10% by weight, or from about 1-5% by weight, of the disclosed sialidase formulations. In another aspect, a dietary supplement and/or a dietary food for medical purposes (for supplementing medical treatments) for humans comprising the disclosed sialidase formulation is also provided herein. In a further aspect, the dietary supplement and/or dietary food can contain from about 10-98% or from about 50-90% by weight the disclosed sialidase formulation. In one aspect, the sialidase formulation can be added to the food directly or premixed with other vitamins, minerals, and/or known dietary or feed supplements.

In one aspect, various other additives which are conventionally added to enzyme food supplement compositions, such as diluents, binders, lubricants, disintegrators, coloring or flavoring agents, preservatives and the like, can also be included in the pharmaceutical compositions, dietary supplements, foods, and beverages disclosed herein.

In one aspect, the disclosed sialidase compositions can also be used in connection with other enzyme supplement compositions, or can be combined with other enzymes in a single enzyme composition, to be used for example as a digestive aid. In one aspect, additional enzymes that may be incorporated include, but are not limited to, β-fructofuranosidase, cellulase, hemicellulase, acid protease, and combinations thereof.

In one aspect, the disclosed polypeptides and compositions can advantageously be incorporated into a food directly ingestible by a user, e.g., as nutrient supplements, health drink, probiotic food, or the like. In one aspect, the food product can be a cooked product. In a further aspect, the food can incorporate meat or animal-derived material (such as beef, chicken, turkey, lamb, fish, blood plasma, marrowbone, or any combination thereof). In another aspect, the product can be meat-free (preferably including a meat substitute such as soy, wheat gluten, or a textured vegetable protein product) in order to provide a protein source. In still another aspect, the product can contain additional protein sources such as soy protein concentrate, milk protein, gluten, or the like. In a still further aspect, the product can also contain a starch source such as, for example, one or more grains (e.g., wheat, corn, rice, oats, barley, etc.) or can be starch-free. In some aspects, the product can incorporate or be a gelatinized starch matrix. In still another aspect, the product can incorporate one or more types of fiber such as sugar beet pulp, chicory pulp, chicory, coconut endosperm fiber, wheat fiber, or the like. In some aspects, dairy products may be suitable.

In one aspect, dry or liquid flavoring agents may be added to the formulation. In another aspect, these include, but are not limited to, cocoa, vanilla, chocolate, coconut, peppermint, pineapple, cherry, nuts, spices, salts, flavor enhancers, and combinations thereof. In one aspect, acidulants commonly added to foods include lactic acid, citric acid, tartaric acid, malic acid, acetic acid, phosphoric acid, hydrochloric acid, and combinations thereof. In still another aspect, other added agents can include anti-oxidants, pH buffers, flavor masking agents, odor masking agents, preservatives, timed-release mechanisms, vitamins, minerals, electrolytes, hormones, herbal material, botanicals, amino acids, carbohydrates, fats, or any combination thereof.

Method for Removing Neu5Gc from Consumable Products

In one aspect, disclosed herein is a method for removing Neu5Gc from a consumable product, the method including the steps of contacting the consumable product with the enzyme composition or sialidase formulation disclosed herein and optionally removing the enzyme composition. In another aspect, the consumable product can be red meat, dairy, or a combination thereof.

### Pre- and Probiotic Supplements

In one aspect, the disclosed composition is in the form of a pre- or probiotic formulation, wherein the pre- or probiotic formulation includes one or more bacteria expressing a disclosed sialidase.

In accordance with yet another aspect of the disclosure, provided herein is a prebiotic or probiotic formulation that includes viable, metabolically active probiotic bacteria in a non-dairy liquid substrate. In yet another aspect, disclosed herein is a prebiotic or probiotic for use in the treatment of inflammatory conditions, cardiovascular diseases, and cancer.

In one aspect, disclosed herein is a method of reducing or eliminating Neu5Gc in a human patient, the method including administering to a patient in need thereof an effective amount of a probiotic preparation comprising viable, metabolically active probiotic bacteria in a non-dairy liquid substrate. In one aspect, the probiotic formulation is capable of delivering viable, metabolically active probiotic bacteria to the intestinal tract of a human subject without triggering digestion.

In accordance with another aspect, provide herein is a pre- and/or probiotic preparation that includes viable, metabolically active probiotic bacteria in a non-dairy liquid substrate including a mixture of bacteria expressing a disclosed sialidase with a preference for Neu5Gc.

In another aspect, disclosed herein is a method of reducing or eliminating Neu5Gc in a human patient including at least the steps of administering to a patient in need thereof, or preparing red meat prior to digestion by the human patient, with an effective amount of a pre- and/or probiotic preparation including viable, metabolically active probiotic bacteria in a non-dairy liquid substrate including a mixture of one or more sialidase expressing bacteria as disclosed herein.

In still another aspect, disclosed herein is a method of reducing or eliminating Neu5Gc in a human patient including at least the step of administering to a patient in need thereof an effective amount of a pre- and/or probiotic preparation that includes viable, metabolically active sialidase producing bacteria in a non-dairy liquid substrate, wherein the sialidase has a preference for Neu5Gc.

In some aspects, the prebiotic or probiotic formulation can include one or more bacteria of the orders: Bacteroidales, Planctomycetales, Micrococcales, Vibrionales, or Enterobacterales. In yet another exemplary embodiment, the probiotic bacteria can be selected from the families: Bacteroidaceae, Rikenellaceae, Verrucomicrobiaceae, Planctomycetaceae, Micrococcaceae, Vibrionaceae, and Enterobacterdaceae.

In other exemplary aspects, disclosed herein is a probiotic formulation including a combination of two or three species selected from Bacteroidales, Planctomycetales, Micrococcales, Vibrionales, or Enterobacterales. In some aspects, the bacteria can be selected from Bacteroides caccae, Bacteroides fragilis, Bacteroides thetaiotamicron, Bacteroides plebeius, Prevotella bivia, Galbibacter marinus, or Planctomycetacea bacterium.

In one aspect, a key factor in achieving therapeutic efficacy in reducing or eliminating Neu5Gc in human diet, as measured as a reduction of directly or indirectly reducing inflammatory symptoms due to a red meat diet, is the ability to deliver a high count of viable, metabolically active probiotic bacteria to the gastrointestinal tract of a subject in need thereof. In some aspects, the total population of metabolically active bacteria in the pre- and/or probiotic preparation can be from about 1.0×10⁸ to about 1.0×10⁹ viable cells per mL, or from about 1.0×10⁷ to about 1.0×10⁹ viable cells per mL. In another aspect, each individual strain of metabolically active bacteria present in the disclosed formulations can be present in the range of from about 1.0×10⁵ to about 1.0×10⁹ viable cells per mL, or from about 1.0×10⁷ to about 1.0×10⁹ viable cells per mL.

In one aspect, the preparation can include a combination of Bacteroides caccae, Bacteroides fragilis, Bacteroides thetaiotamicron, Bacteroides plebeius, Prevotella bivia, or any combination thereof, wherein the bacterial count for each included bacterial strain is in the range of from about 1.0×10⁵ to about 1.0×10⁹ viable cells per mL, or from about 1.0×10⁷ to about 1.0×10⁹ viable cells per mL, and can optionally include Neu5Gc preferring bacteria. In some aspects, the population of such bacteria, if included, can be lower than 1.0×10⁵ viable cells per mL.

In one aspect, although the total bacterial count in the presently described preparation can appear to be lower than that of typical freeze-dried probiotic preparations, it is important to note that the bacteria are viable. As noted elsewhere herein, in one aspect, the viable nature of the bacteria in the probiotic preparation described herein allows them to establish more rapidly in a patient's gastrointestinal tract. In one aspect, the bacteria can be established within 15 to 20 minutes of ingestion. In a further aspect, such rapid establishment contributes to the beneficial effects of the preparation in the treatment of Neu5Gc reaction, including inflammation, CVD, or cancer.

In one aspect, the disclosed probiotic strains are significantly more stable at pH 3 (i.e., the acidic pH experienced in the human stomach) than proprietary milk-based or freeze-dried pre- and/or probiotic products, and therefore more likely to be capable of tolerating the harsh conditions encountered in the human GI tract. In particular, in one aspect, the probiotic bacteria in the preparation described herein are stable from about pH 3 to about pH 6. In one aspect, the probiotic bacteria in the disclosed formulation are stable when maintained in culture at pH 3 for a period of at least 6 hours. In this context “stable” can be taken to mean that when the bacteria are cultured at pH 3, 37° C. in a standard culture medium (e.g. MRS broth), over a period of at least 6 hours the bacterial count (cfu/mL) does not fall by more than 0.5 log₁₀ units below the bacterial count (cfu/mL) at time zero. In specific aspects, the bacterial count should remain above 10⁶ cfu/mL for at least 6 hours when cultured at pH 3, 37° C. in standard MRS broth. In fact, in one aspect, the bacterial count may be observed to increase when cultured under these conditions.

In one aspect, disclosed herein is a probiotic composition that includes a bacterium and at least one excipient, wherein the bacterium expresses one or more sialidases having a preference for Neu5Gc over Neu5Ac. In another aspect, the bacterium can be a Bacteroidales species, a Planctomycetales species, a Micrococcales species, a Vibrionales species, an Enterobacterales species, or a combination thereof. In yet another aspect, the bacterium can be Bacteroides caccae, Bacteroides fragilis, Bacteroides thetaiotamicron, Bacteroides xylanosolvens, Bacteroides plebius, Bifidobacterium longum, Prevotella bivia, Galbibacter marinus, Planctomycetacea bacterium, or a combination thereof.

### Methods for Treating or Preventing an Inflammatory Condition

In another aspect, disclosed herein is a method of treating and/or preventing an inflammatory condition or cancer comprising administering a sialidase formulation, the disclosed pharmaceutical compositions, the disclosed consumable products, and/or the disclosed probiotic compositions to a subject in need thereof.

In another aspect, the inflammatory condition can be cancer, cardiovascular disease, an inflammatory bowel disease, an autoimmune disease, serum sickness, or a combination thereof.

In one aspect, the cancer can be selected from non-Hodgkins lymphoma, neuroblastoma, sarcoma, metastatic brain cancers, ovarian cancer, prostate cancer, breast cancers including triple-negative breast cancer, lymphoma, non-small cell lung carcinoma, gastric cancer, gastroesophageal junction adenocarcinoma, hematological cancers, melanoma, squamous cell carcinoma, Hodgkin's lymphoma, anaplastic large-cell lymphoma, pancreatic cancer, acute lymphoblastic leukemia, acute myeloid leukemia, hepatocellular carcinoma, colorectal cancer, angiosarcoma, head and neck cancer, ovarian cancer, solid tumors, multiple myeloma, glioblastoma, testicular cancer, B-cell malignancies, urothelial cancer, chronic lymphocytic leukemia, adrenocortical carcinoma, acute myelogenous leukemia, clear cell renal cell carcinoma, chronic myelomonocytic leukemia, juvenile myelomonocytic leukemia, small cell lung carcinoma, hairy cell leukemia, renal cell carcinoma, nasopharyngeal cancer, glioma, chronic lymphatic leukemia, diffuse large B-cell lymphoma, gall bladder cancer, thyroid tumor, bone cancer, cervical cancer, uterine cancer, endometrial cancer, vulvar cancer, bladder cancer, colon cancer, colorectal cancer, pancreatic cancer, neuronal cancers, mesothelioma, cholangiocarcinoma, small bowel adenocarcinoma, pediatric malignancies, epidermoid carcinoma, cancer of the pleural or peritoneal membranes, or another cancer.

In another aspect, the cardiovascular disease can be selected from an abnormal heart rhythm or arrhythmia, aorta disease, coronary artery disease, deep vein thrombosis, pulmonary embolism, or another clotting disorder, heart attack, heart failure, cardiomyopathy, heart valve disease, pericardial disease, peripheral vascular disease, rheumatic disease, stroke, vascular disease, atherosclerosis, inflammatory heart disease, endocarditis, valvular heart disease, peripheral artery disease, cardiomyopathy, or another cardiovascular disease.

In still another aspect, the inflammatory bowel disease can be selected from Crohn's disease, ulcerative colitis, collagenous colitis, lymphocytic colitis, ischemic colitis, diversion colitis, Behçet's disease, indeterminate colitis, or another inflammatory bowel disease.

In yet another aspect, the autoimmune disease can be selected from Addison's disease, celiac disease, dermatomyositis, Graves disease, Hashimoto thyroiditis, multiple sclerosis, myasthenia gravis, pernicious anemia, reactive arthritis, rheumatoid arthritis, Sjogren syndrome, systemic lupus erythematosus, type I diabetes, or another autoimmune disease. In addition to autoimmune types of arthritis already listed, the arthritis can include childhood arthritis or osteoarthritis.

In another aspect, disclosed herein is a method for treating or preventing an inflammatory condition in a subject, the method including administering to the subject an effective amount of a sialidase having a preference for cleaving Neu5Gc from glycoconjugates over cleaving Neu5Ac. In some aspects, the sialidase is or includes a sequence selected from SEQ ID NOs. 1-86 or any combination thereof. In a further aspect, the subject has a diet that includes Neu5Gc.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Microbiome Screening and Identification of Sialidase Genes

Materials and Methods

Neu5Gc and Neu5Ac were purchased from Inalco Spa (Milano, Italy) and Pfanstiehl Laboratories, Inc (Waukegan, Ill.), respectively. 1,2-diamino-4,5-methylene dioxybenzene (DMB), chlorpromazine, genistein, nystatin, amiloride, and saponin were from Sigma-Aldrich (St Louis, Mo.). Premium Human Serum type AB was from Irvine Scientific (Santa Ana, Calif.). Neu5Ac aldolase came from ICN (Costa Mesa, Calif.). All reagents used were HPLC grade.

### Mouse Fecal Content Analysis

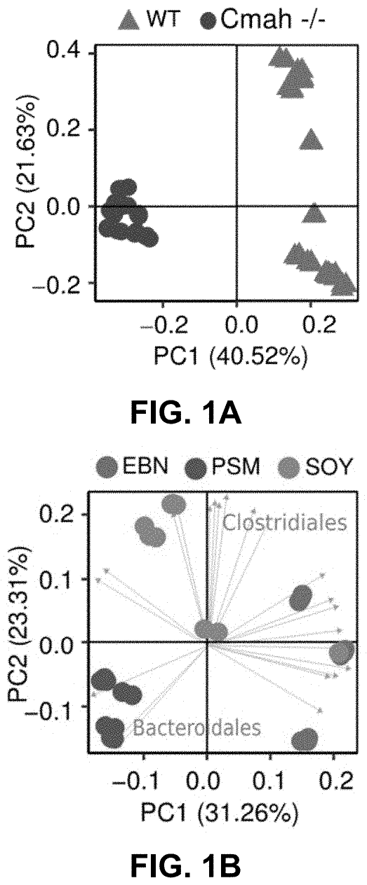

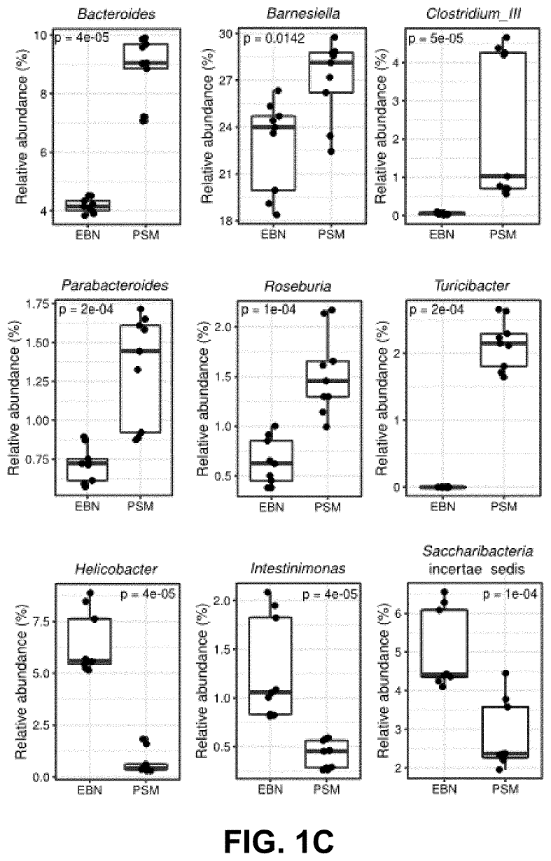

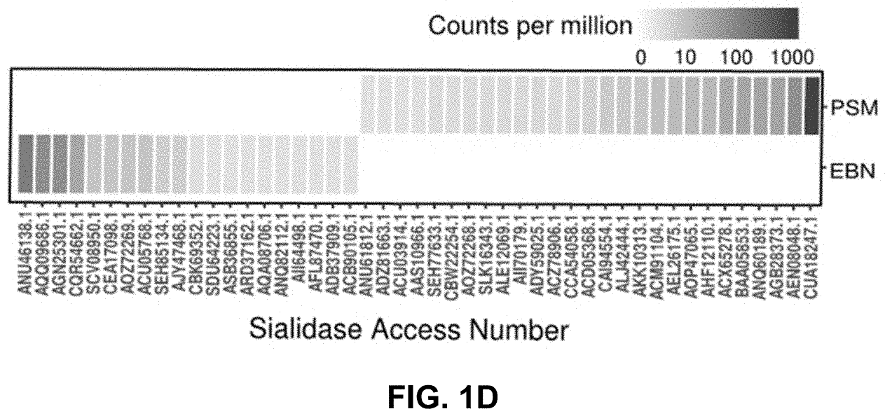

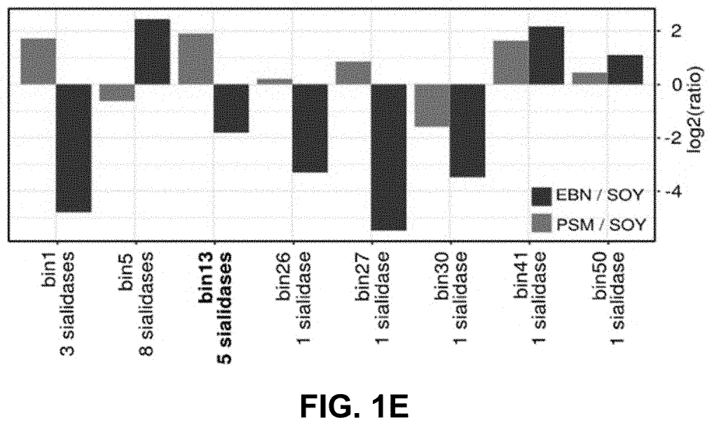

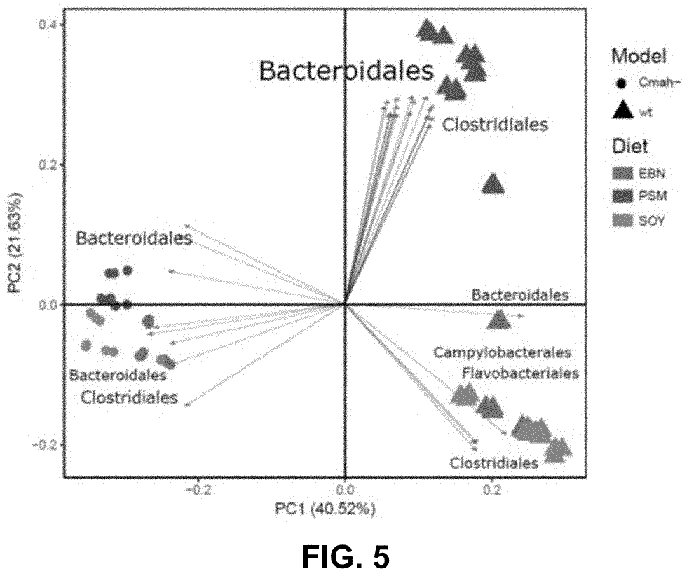

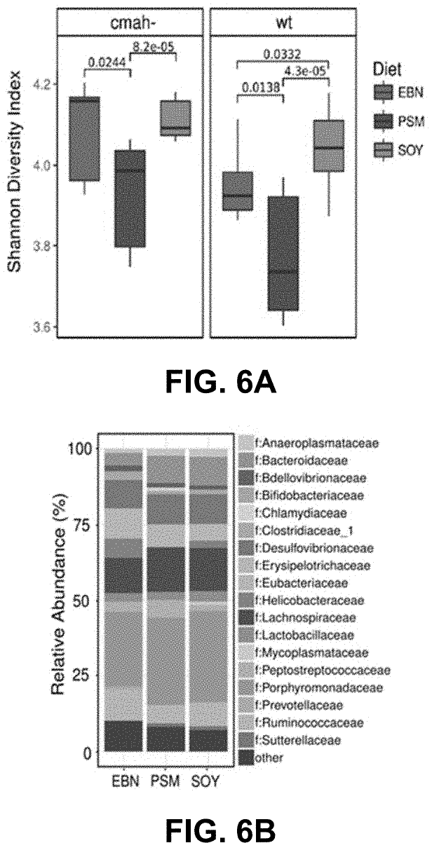

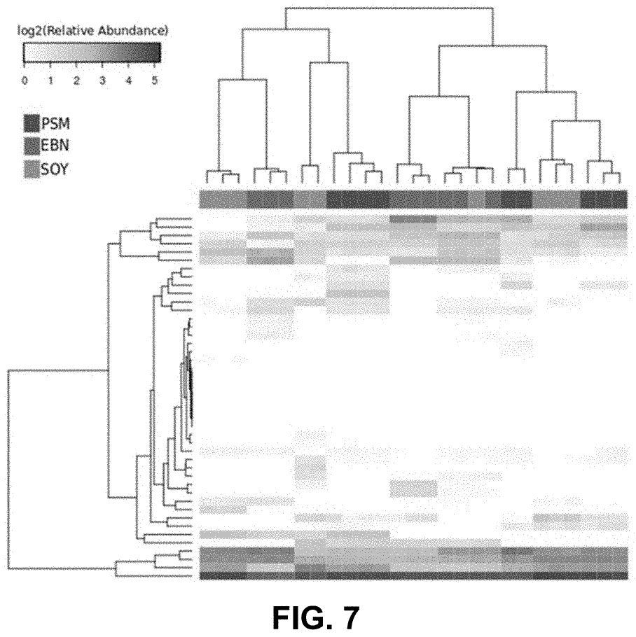

Fecal contents were scraped from the mouse colons and the microbiome of Cmah^{-/-} mice and WT mice fed soy, PSM, or EBN diets was determined through 16S rRNA gene amplicon sequencing. Bray-Curtis dissimilarity showed a significant difference in the bacterial genotypes present in Cmah^{-/-} and WT mice, indicating that a mouse's inability to synthesize endogenous Neu5Gc significantly impacted microbiome composition (FIG. 1A) and substantiated the need for a human-like Cmah^{-/-} mouse model in our experimentation. Pairwise Bray-Curtis dissimilarities were plotted against the first and second principal coordinates (ANOSIM R=0.979, p-value=0.001). WT samples enclose n=5 biologically independent animals and n=15 independent experiments per diet. Cmah^{-/-} samples enclose n=3 biologically independent animals and n=9 independent experiments. We found that changes in the microbial composition in Cmah^{-/-} mice were diet-dependent, with Clostridiales and Bacteroidales contributing significantly to the variations observed amongst the diets (FIGS. 1B, 5). Pairwise Bray-Curtis dissimilarities were plotted against the first and second principal coordinates (ANOSIM R=0.831, p-value=0.001). Gray arrows represent the significant vector fitting with each of the PCoA ordinations. The most representative taxa are indicated on the plot. Independent of genotype, the microbiome of PSM-fed mice was significantly less diverse compared to the microbiome of those fed soy and EBN diets (p-value<0.05) (FIG. 6A). Human-like Cmah^{-/-} mice revealed similar taxonomic profiles at the family level amongst the three diet groups (FIG. 6B). However, at the genus level, Helicobacter, Intestinimonas, and Candidatus Saccharibacteria genera incertae sedis were significantly enriched in the EBN group compared to PSM (FIG. 1C). Moreover, Bacteroides, Barnesiella, Clostridium group II, Parabacteroides, Roseburia, and Turicibacter were significantly enriched in the PSM group compared to EBN (p-value<0.05) (FIGS. 1C, 7). FIG. 1C shows differentially abundant bacterial genera on PSM and EBN diet. Boxes and whiskers indicate quartiles and middle maker indicate the median (n=9). p-values were determined using the two-sided Wilcoxon rank sum test with Holm correction for multiple hypotheses. Bacteroides were previously associated to efficiently metabolize carbohydrates from plant—as well as animal-based food due to their diverse enzymatic repertoire.

### Computational Simulation Results

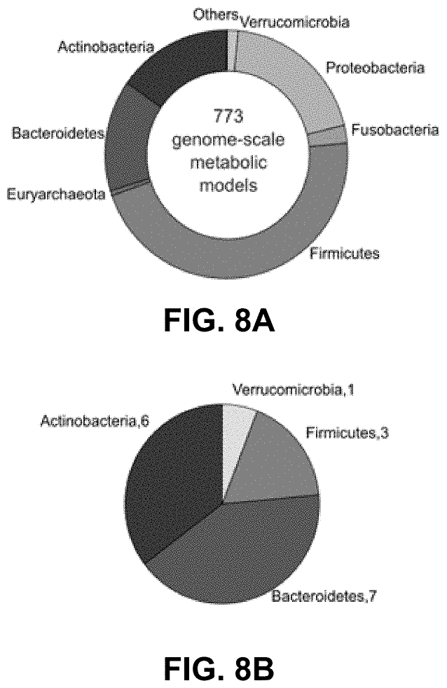

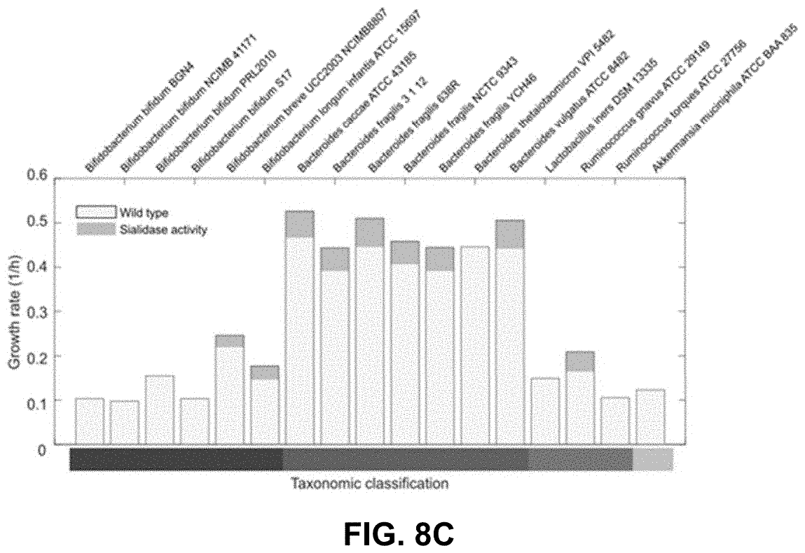

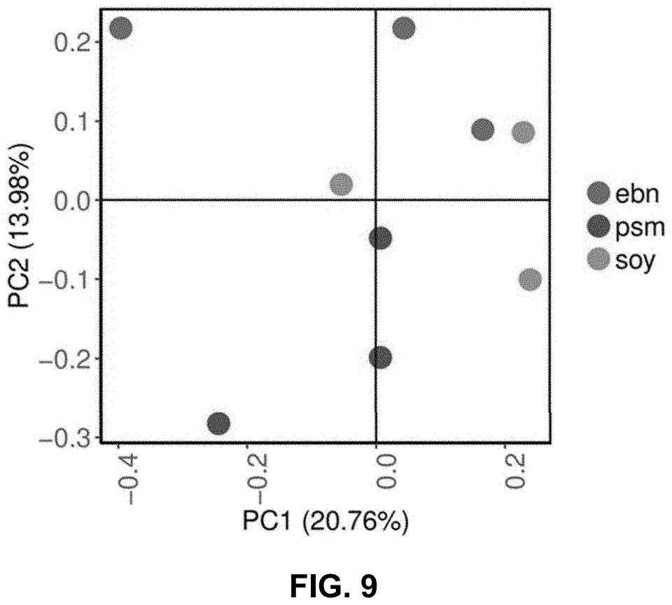

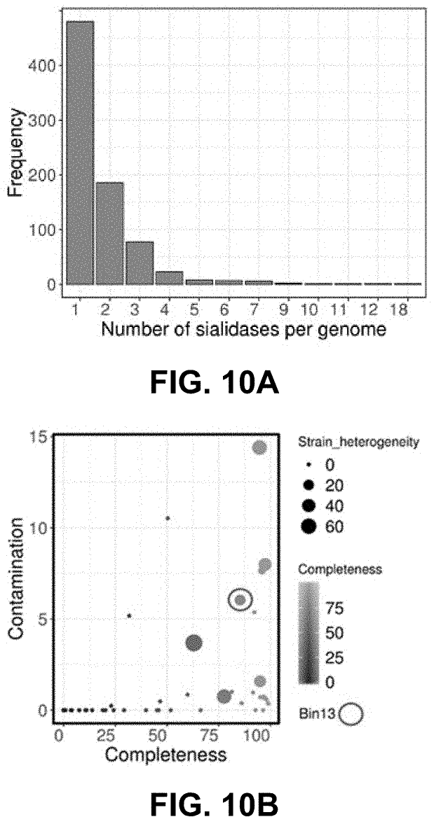

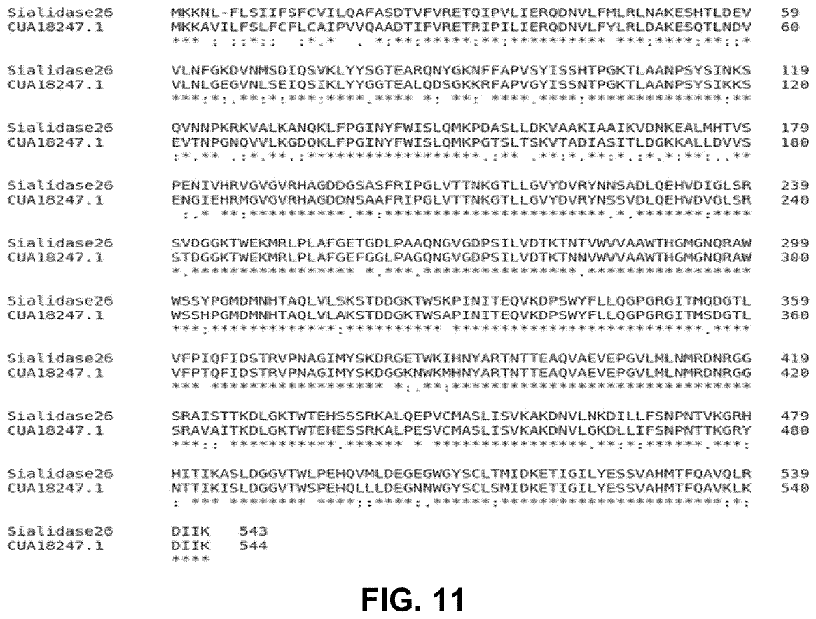

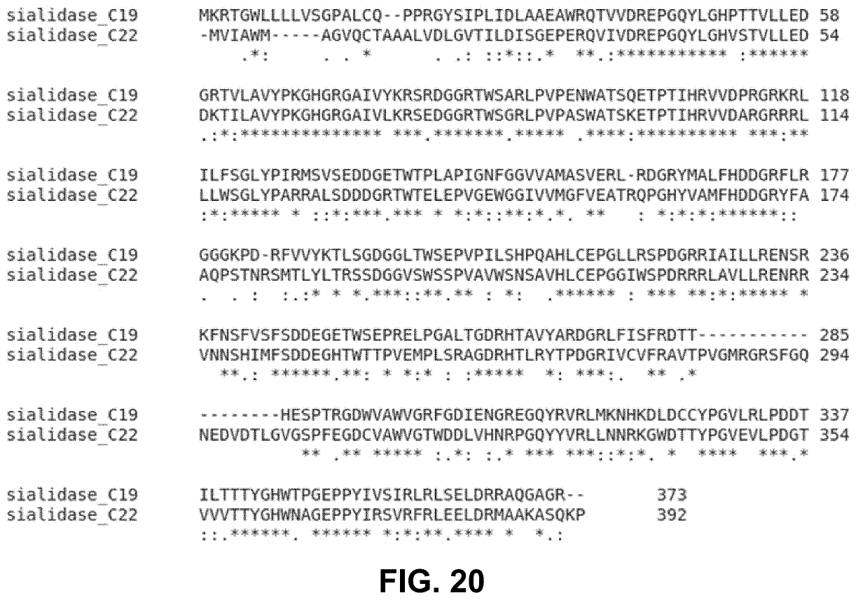

Additionally, by computationally simulating 773 metabolic models from human gut microbiome members, we found that member of the Bacteroidetes, including B. fragilis, B. cacae, B. thetaiotaomicron, were one of the most efficient microorganisms utilizing sialic-acids as a carbon source (FIGS. 8A-8C, Table 1). Shotgun metagenomic DNA sequencing was performed to evaluate the enzymatic repertoire for utilizing carbohydrates in the microbial community from Cmah^{-/-} mice fed soy, PSM, or EBN diets, and raw reads were aligned to a carbohydrate active enzymes (CAZymes) dbCAN-seq database. A Principal Coordinate Analysis revealed a near significant clustering in gene function between diet types (ANOSIM R=0.246, p-value=0.06), with a higher similarity between the CAZymes on soy and EBN diets (FIG. 9). Additionally, the examination of individual sialidase genes revealed several diet-dependent sialidases (FIG. 1D). To evaluate the sialidases genes present in the microbiome, the combined metagenomes were co-assembled and 51 genome bins containing 21 sialidase genes were identified (FIGS. 10A-10B, Table 2). Amongst the bins with annotated sialidase genes, bin13, whose closest relative was Bacteroides thetaiotaomicron (Table 2), was the most abundant in PSM compared to EBN diets (FIG. 1E). Bin13 contains five sialidases (sialidase23, sialidase24, sialidase26, sialidase60, and sialidase65). Sialidase26 exhibited high amino acid sequence conservation (81% identity) to sialidase CUA18247.1 from B. fragilis, the most abundant sialidase protein in the PSM diet (FIGS. 1D, 11). Sequence variation in additional identified sialidase proteins is shown in FIG. 20.

TABLE 1
Metabolic Model Simulation Results

| | | | Adjusted | |
|---|---|---|---|---|
| | | Growth | Growth | Sialidase |
| Microorganism | Taxonomy | Rate | Rate | Activity |
| Bifidobacterium_bifidum_BGN4 | Actinobacteria | 0.1021 | 0.1021 | 0 |
| Bifidobacterium_bifidum_NCIMB_41171 | Actinobacteria | 0.0964 | 0.0964 | 0 |
| Bifidobacterium_bifidum_PRL2010 | Actinobacteria | 0.1541 | 0.1541 | 0 |
| Bifidobacterium_bifidum_S17 | Actinobacteria | 0.1024 | 0.1024 | 0 |
| Bifidobacterium_breve_UCC2003_NCIMB8807 | Actinobacteria | 0.247 | 0.2208 | 0.0262 |
| Bifidobacterium_longum_infantis_ATCC_15697 | Actinobacteria | 0.178 | 0.1468 | 0.0312 |
| Bacteroides_caccae_ATCC_43185 | Bacteroidetes | 0.5272 | 0.4662 | 0.061 |
| Bacteroides_fragilis_3_1_12 | Bacteroidetes | 0.4443 | 0.3908 | 0.0535 |
| Bacteroides_fragilis_638R | Bacteroidetes | 0.5099 | 0.447 | 0.063 |
| Bacteroides_fragilis_NCTC_9343 | Bacteroidetes | 0.4591 | 0.4056 | 0.0534 |
| Bacteroides_fragilis_YCH46 | Bacteroidetes | 0.4441 | 0.3916 | 0.0526 |
| Bacteroides_thetaiotaomicron_VPI_5482 | Bacteroidetes | 0.4454 | 0.4454 | 0 |
| Bacteroides_vulgatus_ATCC_8482 | Bacteroidetes | 0.5071 | 0.4424 | 0.0647 |
| Lactobacillus_iners_DSM_13335 | Firmicutes | 0.1479 | 0.1479 | 0 |
| Ruminococcus_gnavus_ATCC_29149 | Firmicutes | 0.2075 | 0.1643 | 0.0432 |
| Ruminococcus_torques_ATCC_27756 | Firmicutes | 0.1049 | 0.1049 | 0 |
| Akkermansia_muciniphila_ATCC_BAA_835 | Verrucomicrobia | 0.1222 | 0.1222 | 0 |

TABLE 2
Genome Bins for Mouse Metagenome

| Bin_ID | Size | GC % | N50 | L50 | Completeness | Contamination | Strain Heterogeneity | Closest Neighbor |
|---|---|---|---|---|---|---|---|---|
| bin. 21 | 1850494 | 33.2 | 24064 | 23 | 99.03 | 0.35 | 0 | Helicobacter winghamensis ATCC BAA-430 |
| bin. 27 | 2269403 | 54.9 | 80475 | 9 | 98.87 | 0.38 | 0 | Porphyromonas gingivalis ATCC 33277 |
| bin. 47 | 2568469 | 50.8 | 93332 | 10 | 98.68 | 0.38 | 0 | Bacteroides vulgatus ATCC 8482 |
| bin. 25 | 3208820 | 47.9 | 41655 | 25 | 98.18 | 0.57 | 0 | Porphyromonas gingivalis ATCC 33277 |
| bin. 50 | 2102753 | 38.6 | 16945 | 37 | 97.54 | 0.67 | 0 | Anaerotruncus colihominis DSM 17241 |
| bin. 13 | 6594969 | 42.9 | 14791 | 130 | 97.4 | 7.99 | 36.36 | Bacteroides thetaiotaomicron VPI-5482 |
| bin. 48 | 2449761 | 51.6 | 57972 | 15 | 96.67 | 0.75 | 0 | Porphyromonas gingivalis ATCC 33277 |
| bin. 19 | 4078864 | 45.1 | 47956 | 27 | 96.5 | 0.68 | 0 | Coprococcus comes ATCC 27758 |
| bin. 40 | 2326744 | 52.8 | 77390 | 9 | 96.42 | 0 | 0 | Bacteroides fragilis ATCC 25285 |
| bin. 23 | 1180283 | 40.3 | 9017 | 40 | 95.85 | 7.65 | 5.88 | Chlamydia muridarum Nigg |
| bin. 41 | 2668268 | 48.8 | 66931 | 12 | 95 | 0.71 | 0 | Alistipes putredinis DSM 17216 |
| bin. 26 | 2635792 | 52.3 | 18023 | 40 | 94.95 | 1.58 | 28.57 | Bacteroides thetaiotaomicron VPI-5482 |
| bin. 33 | 4542794 | 59.3 | 17331 | 81 | 94.7 | 14.4 | 52.17 | Clostridium cellulolyticum H10 |
| bin. 44 | 1546950 | 34 | 20898 | 25 | 92.74 | 0 | 0 | Clostridium cellulolyticum H10 |
| bin. 18 | 3999932 | 59.1 | 12835 | 90 | 92.23 | 5.37 | 0 | Eubacterium siraeum VIOSc8a |
| bin. 5 | 2531697 | 46 | 35719 | 25 | 91.55 | 0.97 | 0 | Dorea formicigenerans ATCC 27755 |
| bin. 36 | 2123123 | 55.1 | 47701 | 13 | 86.04 | 0.38 | 0 | Bacteroides fragilis ATCC 25285 |
| bin. 16 | 2765019 | 60.2 | 9836 | 83 | 85.3 | 6.04 | 21.43 | Clostridium cellulo&ticum H10 |
| bin. 1 | 1743578 | 53.6 | 16637 | 33 | 81.33 | 1.01 | 0 | Bacteroides vulgatus ATCC 8482 |
| bin. 24 | 1958324 | 53.8 | 79876 | 9 | 77.55 | 0.75 | 50 | Porphyromonas gingivalis ATCC 33277 |
| bin. 3 | 1438390 | 50.8 | 84511 | 7 | 66.23 | 0 | 0 | Bacteroides fragilis ATCC 25285 |
| bin. 38 | 2554082 | 58.8 | 9223 | 81 | 62.94 | 3.69 | 77.78 | Anaerotruncus colihominis DSM 17241 |
| bin. 31 | 839425 | 46.7 | 141422 | 3 | 59.89 | 0.85 | 0 | Geobacillus kaustophilus HTA426 |
| bin. 4 | 2121811 | 47.5 | 17632 | 24 | 51.75 | 0 | 0 | Bryantella formatexigens DSM 14469 |
| bin. 45 | 5361881 | 42.9 | 4933 | 270 | 50.29 | 10.52 | 0 | Coprococcus comes ATCC 27758 |
| bin. 49 | 737196 | 33.7 | 17545 | 14 | 46.67 | 0.48 | 0 | Acholeplasma laidlawii PG-8A |
| bin. 29 | 821437 | 34 | 6183 | 41 | 45.87 | 0 | 0 | Thermincola sp. JR |
| bin. 15 | 875044 | 47.6 | 94357 | 3 | 45.09 | 0 | 0 | Anaerotruncus colihominis DSM 17241 |
| bin. 14 | 822161 | 42.9 | 58321 | 5 | 39.81 | 0 | 0 | Clostridium perfringens ATCC 13124 |
| bin. 28 | 1108277 | 51.4 | 3959 | 89 | 31.72 | 5.17 | 0 | Bacteroides vulgatus ATCC 8482 |
| bin. 12 | 1293986 | 47.1 | 48036 | 9 | 29.18 | 0 | 0 | Bacteroides eggerthii DSM |
| bin. 30 | 627666 | 44.3 | 64511 | 3 | 24.14 | 0 | 0 | Porphyromonas gingivalis ATCC 33277 |
| bin. 37 | 444059 | 39 | 2631 | 53 | 22.99 | 0.24 | 0 | Erysipelotrichaceae bacterium 5_2_54FAA |
| bin. 11 | 443662 | 46.2 | 3658 | 38 | 20.18 | 0 | 0 | Erysipelotrichaceae bacterium 5_2_54FAA |
| bin. 20 | 1150156 | 47.7 | 12470 | 20 | 18.97 | 0 | 0 | Bacteroides vulgatus ATCC 8482 |
| bin. 43 | 216870 | 53.5 | 39370 | 3 | 13.79 | 0 | 0 | Bacteroides vulgatus ATCC 8482 |
| bin. 10 | 547001 | 55.5 | 5527 | 33 | 11.03 | 0 | 0 | Ethanoligenens harbinense YUAN-3 |
| bin. 8 | 270586 | 57.5 | 12196 | 8 | 10.58 | 0 | 0 | Alistipes putredinis DSM 17216 |
| bin. 42 | 243180 | 61.1 | 3209 | 27 | 7.52 | 0 | 0 | Ethanoligenens harbinense YUAN-3 |
| bin. 39 | 513508 | 48.1 | 27413 | 7 | 3.93 | 0 | 0 | Symbiobacterium thermophilum IAM 14863 |
| bin. 9 | 514825 | 44.5 | 15600 | 9 | 3.45 | 0 | 0 | Bacteroides intestinalis DSM 17393 |
| bin. 34 | 502466 | 45.9 | 32140 | 6 | 0.86 | 0 | 0 | Bacteroides thetaiotaomicron VPI-5482 |
| bin. 7 | 584478 | 47.5 | 4526 | 35 | 0 | 0 | 0 | Dorea formicigenerans ATCC 27755 |
| bin. 6 | 298825 | 47.2 | 5605 | 12 | 0 | 0 | 0 | Clostridium thermocellum ATCC 27405 |
| bin. 51 | 340372 | 48.7 | 5053 | 18 | 0 | 0 | 0 | Clostridium saccharolyticum WMI |
| bin. 46 | 299296 | 50.2 | 20062 | 5 | 0 | 0 | 0 | Desulfitobacterium sp. Y51 |
| bin. 35 | 466484 | 49.3 | 7616 | 17 | 0 | 0 | 0 | Eubacterium rectale ATCC 33656 |
| bin. 32 | 322877 | 43.7 | 14422 | 5 | 0 | 0 | 0 | Bacteroides intestinalis DSM 17393 |
| bin. 22 | 301236 | 49.3 | 26150 | 4 | 0 | 0 | 0 | Erysipelotrichaceae bacterium 5_2_54FAA |
| bin. 2 | 390337 | 49 | 7096 | 17 | 0 | 0 | 0 | Bacteroides pectinophilus ATCC 43243 |
| bin. 17 | 251249 | 43.4 | 13443 | 5 | 0 | 0 | 0 | Bacteroides intestinalis DSM 17393 |

### Example 2: Neu5Gc Release Screening

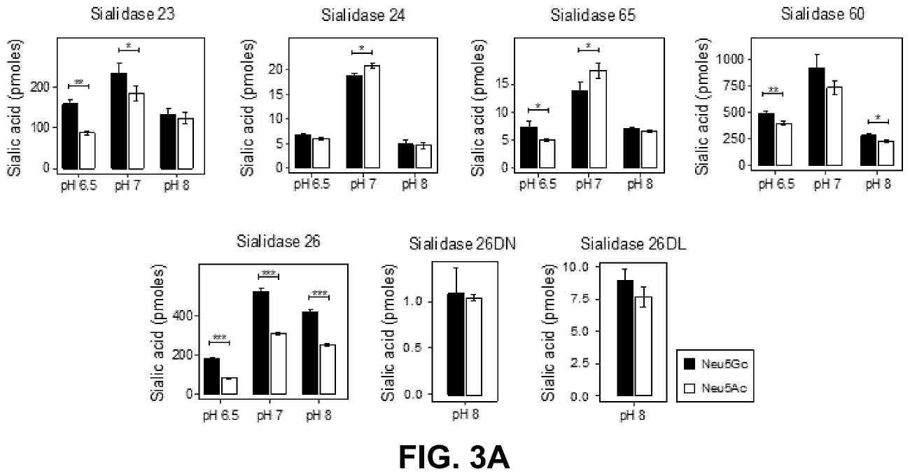

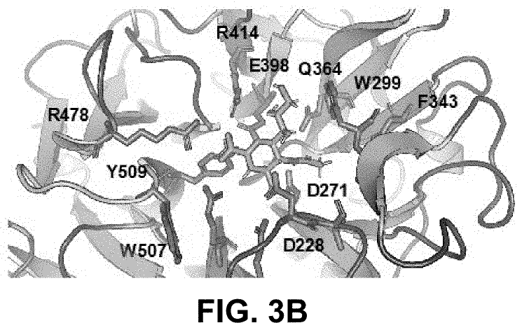

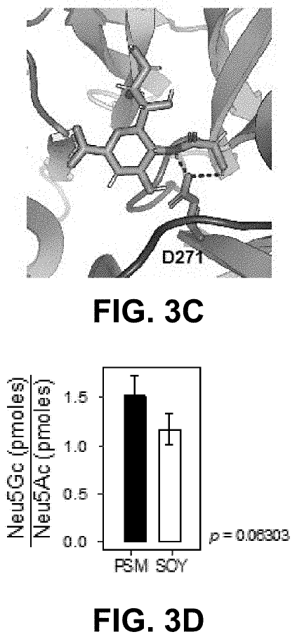

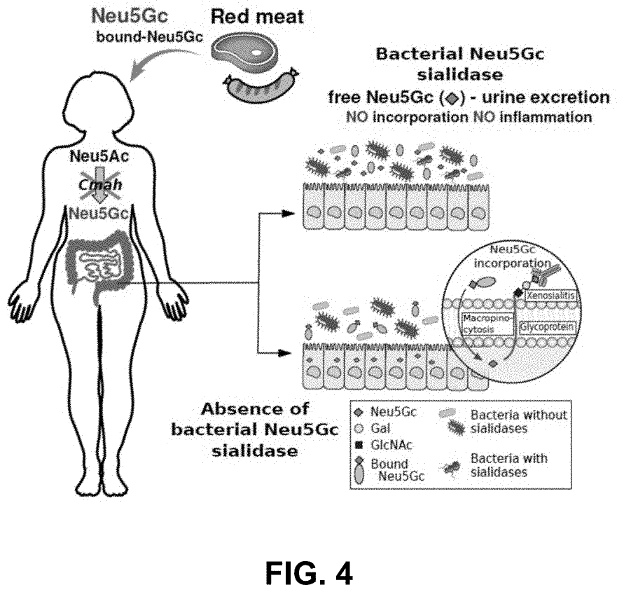

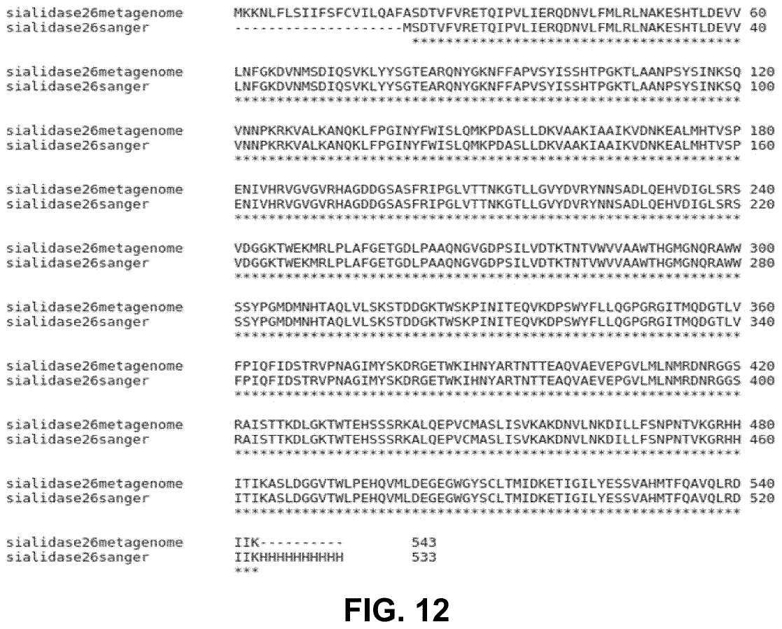

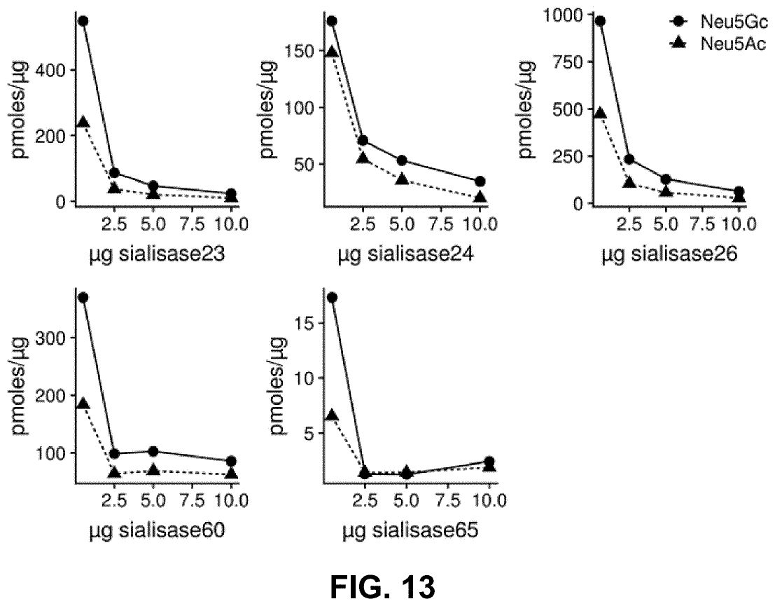

Bin13 sialidases were tested for Neu5Gc release. Each of the five sialidases genes in bin13 were PCR amplified (FIG. 12) and heterologously expressed in vitro, purified, and assayed for its substrate preference. Sialidase activity measurements were performed using different enzyme concentrations (0.5-10 μg) at three different pH levels (6.5, 7.0, and 8) (FIGS. 3A, 13). Four out of five sialidases showed preferential Neu5Gc activity in at least one of the pH levels tested (FIG. 3A). To the best of our knowledge, no previously characterized exo-sialidases have been shown to prefer Neu5Gc over Neu5Ac. Additionally, we tested in vivo sialidase activity in fresh fecal samples. Clarified fecal pellets from mice fed with PSM tend to preferentially release Neu5Gc compared to clarified fecal pellets from mice fed with Soy (FIG. 3D).

### Example 3: X-Ray Crystallography

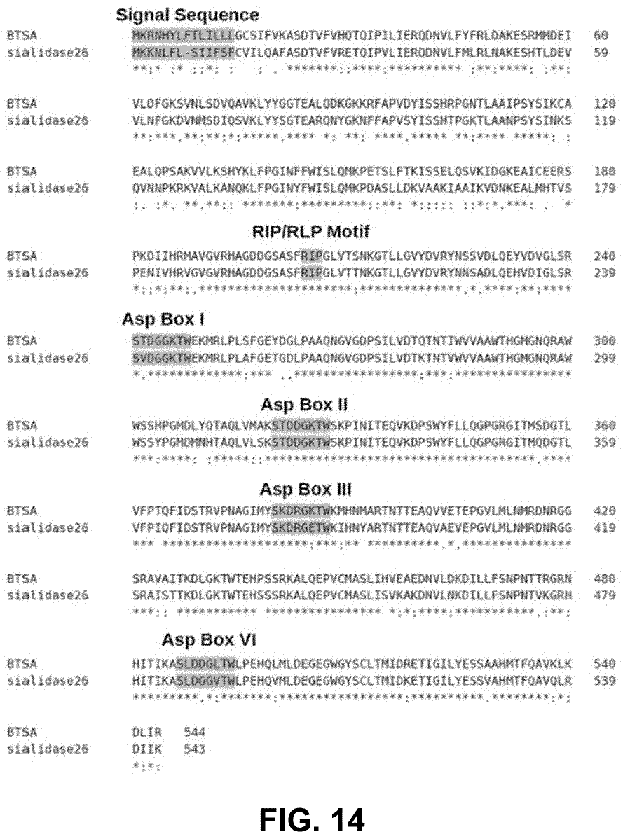

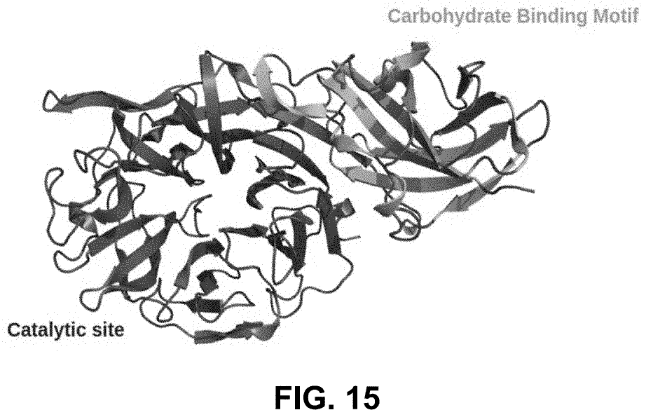

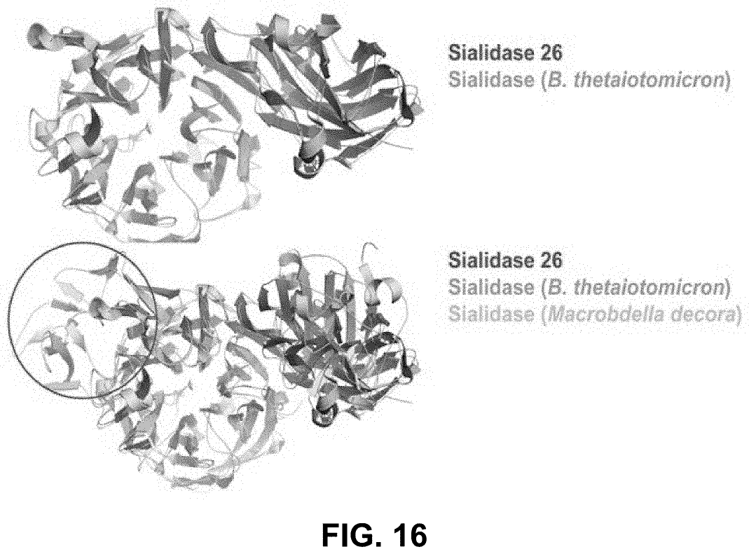

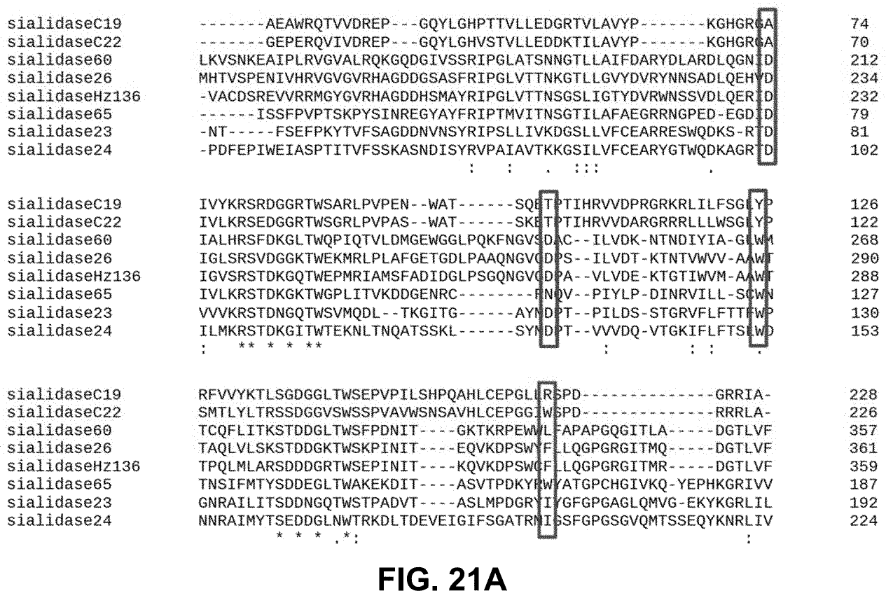

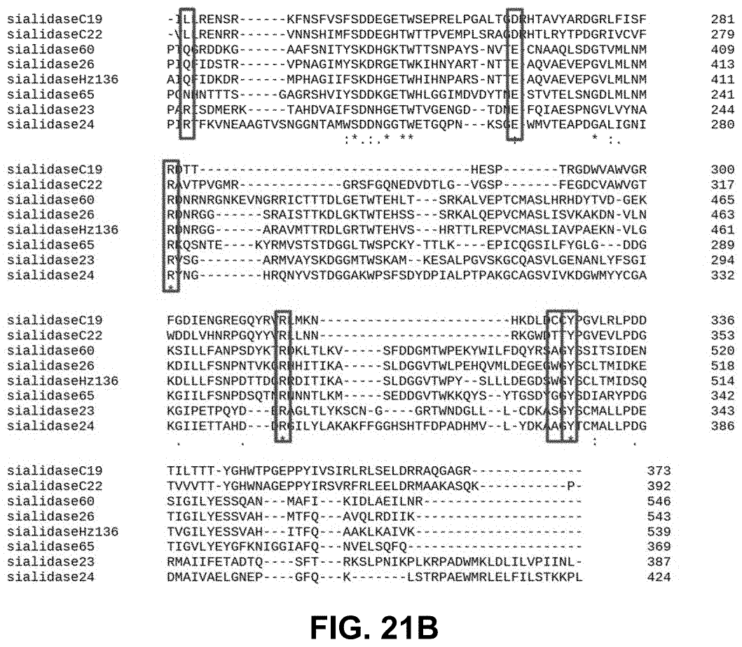

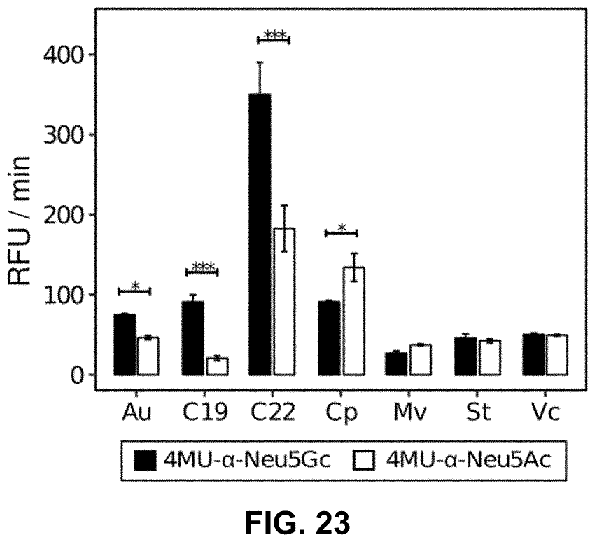

Sialidase26 possessed protein sequence motifs characteristic of the GH33 family of sialidases (FIGS. 14-15). Despite this substrate preference, sequence residues that are predicted to interact with terminal sialic-acids in the catalytic site are highly conserved with structurally studied sialidases exhibiting unknown Neu5Gc preference over Neu5Ac (FIGS. 16, 23). To elucidate the structural underpinnings of Neu5Gc preference, we used X-ray crystallography to determine the structure of sialidase26 both alone (PDB 6MRX, 2.0 Å resolution) and in complex with the inhibitors DANA-Ac and DANA-Gc (PDB 6MRV, 1.8 Å resolution, and PDB 6MYV, 2.2 Å resolution, respectively) (Table 3). Sialidase26 structure is common to GH33 sialidases (FIG. 3B), including Y509 nucleophilic engagement of C5 following E398 charge activation, D228 acid-base catalysis of the glycosidic bond at C5, and C2 stabilization by an Arg triad (R203, R414, R478). However, typical sialic-acid stabilizing interactions are lost, including Glu engagement of the glycerol moiety C7-C9 (T397 in sialidase26), C10 stability by an Arg residue (now a Leu), and inward movement of W507 into the binding pocket (though this is restored in the DANA-Gc co-crystal structure). Co-crystallization of sialidase26 with DANA-Gc indicated an overall fold and ligand placement similar to that of DANA-Ac (FIG. 3C). The hydroxyl-group at the end of the C5 acetamido-group of DANA-Gc is pointed towards the binding pocket residues, forming H contacts with D271 and likely increasing its stability. Amino acid substitutions at this position to Leu (D271L) or Asn (D271N) significantly lowered sialidase protein activity and eliminated the Neu5Gc preferential cleavage (FIG. 3A). This, in combination with the changes described above, likely explains sialidase26's preference for Neu5Gc. A multiple sequence alignment showing conserved features in several identified sialidase proteins is presented in FIGS. 21A-21B.

TABLE 3
X-Ray Crystallography Data Collection and Refinement

| | | | |
|---|---|---|---|
| Sia26 (6MRX) | SIA26:DANA (6MRV) | Sia26:DANA-Gc (6MYV) | SiaHz136 (6MNJ) |

Data collection

| | | | | |
|---|---|---|---|---|
| Space group | /121 | /121 | P1 | /222 |

Cell dimensions

| | | | | |
|---|---|---|---|---|
| a, b, c (Å) | 79.81, 115.48, 260.81 | 92.04, 116.70, 113.64 | 85.64, 89.75, 95.81 | 119.67, 145.39, 150.29 |
| α, β, γ (°) | 90.00, 100.92, 90.00 | 90.00, 106.34, 90.00 | 64.70, 75.65, 88.58 | 90.00, 90.00, 90.00 |
| Resolution (Å) | 48.42(2.00) * | 80.82(1.80) * | 48.50(2.20) * | 75.14(2.20) * |
| R_{sym} or R_{merge} | 11.0(92.6) | 10.3(81.9) | 13.5(87.2) | 21.1(87.3) |
| I/o/ | 6.9(1.2) | 6.6(1.1) | 6.0(1.3) | 10.5(3.7) |
| Completeness (%) | 100.0(100.0) | 99.7(99.9) | 98.3(97.2) | 100.0(99.8) |
| Redundancy | 3.5(3.2) | 3.1(3.1) | 3.9(3.9) | 12.7(11.3) |

Refinement

| | | | | |
|---|---|---|---|---|
| Resolution (Å) | 48.42-2.00 | 60.78-1.80 | 48.50-2.20 | 75.14-2.20 |
| No. reflections | 157,315 | 106,029 | 124,154 | 66,624 |
| R_{work}/R_{free} | 0.22/0.24 | 0.20/0.23 | 0.23/0.26 | 0.21/0.24 |
| No. atoms | | | | |
| Protein | 16,280 | 8.309 | 8,309 | 8,225 |
| Ligand/ion | — | 72 | 148 | — |
| Water | 282 | 421 | 789 | 142 |
| B-factors | | | | |
| Protein | 28.23 | 21.64 | 34.33 | 27.60 |
| Ligand/ion | — | 18.88 | 38.32 | — |
| Water | 24.71 | 20.94 | 33.75 | 22.38 |

R.m.s. deviations

| | | | | |
|---|---|---|---|---|
| Bond lengths (Å) | 0.002 | 0.004 | 0.006 | 0.002 |
| Bond angles (°) | 0.591 | 0.780 | 0.823 | 0.593 |

### Example 4: Fecal Shotgun Metagenomes for a Hunter-Gatherer Population

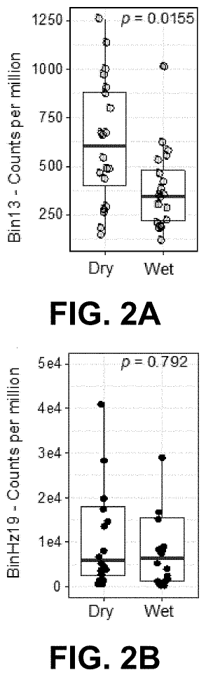

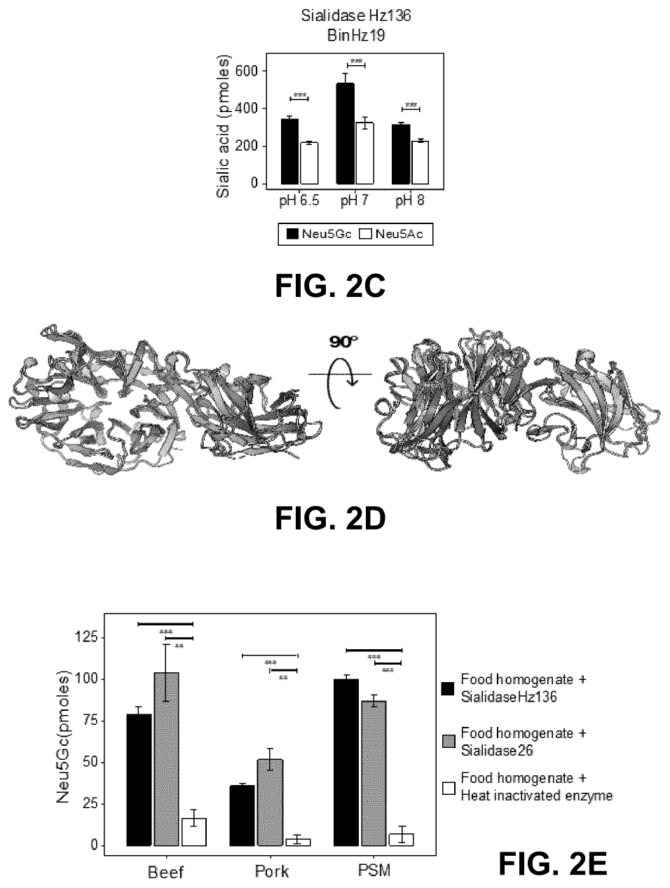

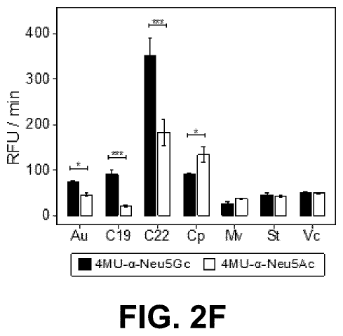

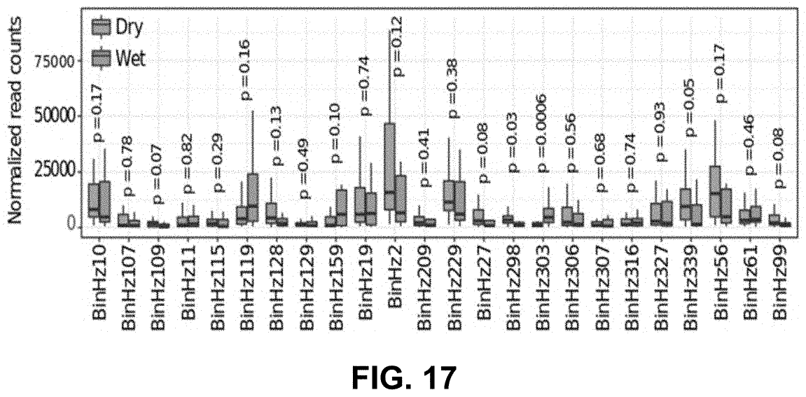

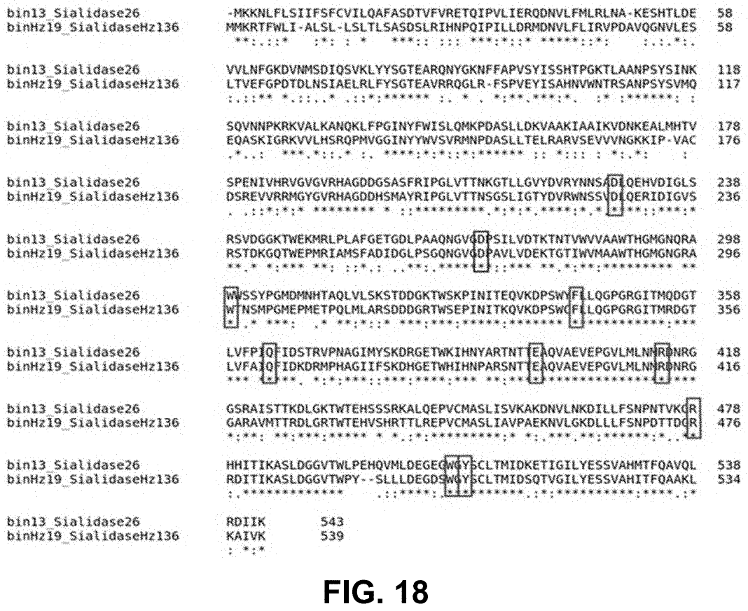

Fecal shotgun metagenomes from the Hadza people (Table 4), a genetically distinct indigenous ethnic group that resides in remote Tanzania was reanalyzed. The Hadza are hunter-gatherers and change their diet periodically throughout the year according to food availability. In dry seasons, their diet is enriched in meat and tubers, whereas in wet seasons it consists largely of honey and berries. Raw reads from Hadza metagenomes were mapped to bin13. We observed that bin13 was significantly more abundant in microbiome samples taken during the dry season compared to the wet season (FIG. 2A). To evaluate the sialidases genes present in the Hadza microbiome, the combined metagenomes were co-assembled and 24 genome bins containing 51 sialidase genes were identified (FIG. 17, Table 5). The binHz19, whose closest relative was Alistipes sp., was equally abundant in microbiomes from both seasons (FIG. 2B, Table 5). BinHz19 also contained a sialidase (sialidaseHz136) with the greatest sequence similarity to sialidase26 identified in our mouse study, suggesting widespread distribution of this sialidase homolog amongst mammals (FIG. 18). SialidaseHz136 showed preferential activity for Neu5Gc over Neu5Ac in all tested conditions (FIG. 2C). X-ray crystallography of SialidaseHz136 (PDB 6MNJ, 2.2 Å resolution) exhibited similar predicted engagement of conserved residues with Sia substrate when compared to sialidase26 (FIG. 2D), despite a shift in the acetamido-interacting Asp residue. This difference supports the notion that sialidaseHz136 is capable of metabolizing structurally diverse glycans, a concept that would be of benefit to an individual with seasonal variations in diet. To additionally determine if sialidase26 and sialidaseHz136 can release Neu5Gc from food sources directly, we test their activity on beef, pork, and PSM chow as substrate. Both enzymes showed pronounced sialidase activity by releasing Neu5Gc from all food tested (FIG. 2E).

TABLE 4
Relative Abundance of Hadza Data Aligned to bin.13

| | | | | | Reads | |
|---|---|---|---|---|---|---|
| | | | | Total # Trimmed | mapped | |
| Accession | SRA | Season | Experiment | Nonhuman Reads | bin.13 | Normalized |
| SRX2963153 | SRR5763445 | dry | TZ_47979_R1 | 6273252 | 7900 | 1259.31 |
| SRX2963152 | SRR5763446 | wet | TZ_93321_R1 | 11025059 | 3919 | 355.46 |
| SRX2963151 | SRR5763447 | wet | TZ_67706_R1 | 6178345 | 3427 | 554.68 |
| SRX2963150 | SRR5763448 | dry | TZ_21460_R1 | 3330172 | 977 | 293.38 |
| SRX2963149 | SRR5763449 | wet | TZ_41014_R1 | 9107663 | 3216 | 353.11 |
| SRX2963148 | SRR5763450 | wet | TZ_10742_R1 | 7466471 | 2867 | 383.98 |
| SRX2963147 | SRR5763451 | wet | TZ_73414_R1 | 12370910 | 7183 | 580.64 |
| SRX2963146 | SRR5763452 | wet | TZ_83893_R1 | 7635604 | 2318 | 303.58 |
| SRX2963145 | SRR5763453 | wet | TZ_65172_R1 | 13353637 | 3822 | 286.21 |
| SRX2963144 | SRR5763454 | wet | TZ_65642_R1 | 18721999 | 18973 | 1013.41 |
| SRX2963143 | SRR5763455 | dry | TZ_90808_R1 | 10356039 | 6961 | 672.17 |
| SRX2963142 | SRR5763456 | dry | TZ_16581_R1 | 2529052 | 701 | 277.18 |
| SRX2963141 | SRR5763457 | wet | TZ_24827_R1 | 4383172 | 791 | 180.46 |
| SRX2963140 | SRR5763458 | wet | TZ_70090_R1 | 2510510 | 761 | 303.13 |
| SRX2963139 | SRR5763459 | dry | TZ_86768_R1 | 4155868 | 1938 | 466.33 |
| SRX2963138 | SRR5763460 | dry | TZ_37609_R1 | 3947819 | 1929 | 488.62 |
| SRX2963137 | SRR5763461 | dry | TZ_39227_R1 | 3207470 | 1398 | 435.86 |
| SRX2963136 | SRR5763462 | dry | TZ_81781_R1 | 3040744 | 1484 | 488.04 |
| SRX2963135 | SRR5763463 | dry | TZ_42864_R1 | 2708678 | 2452 | 905.24 |
| SRX2963134 | SRR5763464 | dry | TZ_29321_R1 | 2563465 | 2915 | 1137.13 |
| SRX2963133 | SRR5763465 | dry | TZ_87532_R1 | 3758101 | 555 | 147.68 |
| SRX2963132 | SRR5763466 | dry | TZ_76219_R1 | 3081596 | 2994 | 971.57 |
| SRX2963131 | SRR5763467 | dry | TZ_83985_R1 | 2619653 | 1731 | 660.77 |
| SRX2963130 | SRR5763468 | dry | TZ_25806_R1 | 2097908 | 1833 | 873.73 |
| SRX2963129 | SRR5763469 | dry | TZ_83788_R1 | 4712957 | 4714 | 1000.22 |
| SRX2963128 | SRR5763470 | dry | TZ_38244_R1 | 2205033 | 1480 | 671.19 |
| SRX2963127 | SRR5763471 | dry | TZ_43638_R1 | 3806024 | 3041 | 799.00 |
| SRX2963126 | SRR5763472 | dry | TZ_99300_R1 | 3325735 | 607 | 182.52 |
| SRX2963125 | SRR5763473 | dry | TZ_42041_R1 | 4596873 | 1210 | 263.22 |
| SRX2963124 | SRR5763474 | wet | TZ_86771_R1 | 3953617 | 886 | 224.10 |
| SRX2963123 | SRR5763475 | wet | TZ_60579_R1 | 4494910 | 1534 | 341.27 |
| SRX2963122 | SRR5763476 | wet | TZ_83627_R1 | 4292298 | 2283 | 531.88 |
| SRX2963121 | SRR5763477 | wet | TZ_91827_R1 | 2797766 | 1294 | 462.51 |
| SRX2963120 | SRR5763478 | wet | TZ_40311_R1 | 2204802 | 263 | 119.29 |
| SRX2963119 | SRR5763479 | wet | TZ_39371_R1 | 3293821 | 1388 | 421.40 |
| SRX2963118 | SRR5763480 | wet | TZ_21618_R1 | 3602167 | 677 | 187.94 |
| SRX2963117 | SRR5763481 | wet | TZ_14139_R1 | 3024516 | 633 | 209.29 |
| SRX2963116 | SRR5763482 | wet | TZ_12739_R1 | 3207143 | 2003 | 624.54 |
| SRX2963115 | SRR5763483 | wet | TZ_61717_R1 | 3764610 | 741 | 196.83 |
| SRX2963114 | SRR5763484 | dry | TZ_27689_R1 | 5648186 | 3072 | 543.89 |

TABLE 5
Genome Bins for Hadza Metagenome with Annotated Sialidase Gene

| | | | | | | | Strain | |
|---|---|---|---|---|---|---|---|---|
| Bin_ID | Size | GC % | N50 | L50 | Completeness | Contamination | Heterogeneity | Closest Neighbor |
| binHz107 | 2820482 | 47.19 | 8077 | 115 | 70.86 | 5.43 | 76.92 | Bacteroides fragilis ATCC 25285 |
| binHz109 | 264436 | 47.46 | 7540 | 13 | 0 | 0 | 0 | Bacteroides thetaiotaomicron VPI- |
| | | | | | | | | 5482 |
| binHz10 | 2925305 | 47.19 | 9453 | 98 | 58.62 | 9.48 | 83.33 | Prevotella ruminicola 23 |
| binHz115 | 2033063 | 55.06 | 16916 | 36 | 70.78 | 0.77 | 75 | Prevotella ruminicola 23 |
| binHz119 | 2936672 | 47.61 | 9285 | 99 | 90.53 | 5.6 | 72.22 | Bacteroides thetaiotaomicron VPI- |
| | | | | | | | | 5482 |
| binHz11 | 2663757 | 49.62 | 10264 | 81 | 88.7 | 6.64 | 63.33 | Alistipes putredinis DSM 17216 |
| binHz128 | 2793467 | 46.76 | 15277 | 57 | 86.29 | 4.7 | 66.67 | Bacteroides intestinalis DSM 17393 |
| binHz129 | 2691906 | 43.31 | 9809 | 88 | 91.36 | 3.39 | 41.67 | Bacteroides caccae ATCC 43185 |
| binHz159 | 2314390 | 27.67 | 8565 | 88 | 92.16 | 2.11 | 0 | Clostridium perfringens ATCC 13124 |
| binHz19 | 2253882 | 51.55 | 11736 | 57 | 62.71 | 3.97 | 70 | Alistipes sp. |
| binHz209 | 2568830 | 47.68 | 13754 | 59 | 83.81 | 2.6 | 45.45 | Prevotella ruminicola 23 |
| binHz229 | 3246489 | 44.17 | 5551 | 166 | 27.03 | 5.87 | 0 | Bacteroides thetaiotaomicron VPI- |
| | | | | | | | | 5482 |
| binHz27 | 2394901 | 52.12 | 16214 | 49 | 70.69 | 8.62 | 60 | Prevotella ruminicola 23 |
| binHz298 | 2959682 | 48.18 | 16224 | 57 | 94.1 | 2.76 | 71.43 | Prevotella ruminicola 23 |
| binHz2 | 3207967 | 46.21 | 7641 | 131 | 52.35 | 6.03 | 100 | Prevotella ruminicola 23 |
| binHz303 | 2261674 | 64.46 | 21132 | 33 | 97.58 | 3.35 | 18.18 | Atopobium parvulum DSM 20469 |
| binHz306 | 1649945 | 53.8 | 12205 | 44 | 68.29 | 1.43 | 60 | Bacteroides vulgatus ATCC 8482 |
| binHz307 | 2438559 | 50.63 | 9789 | 82 | 82.81 | 2.58 | 60 | Prevotella ruminicola 23 |
| binHz316 | 1888527 | 28.91 | 5338 | 128 | 79.6 | 4 | 16.67 | Brachyspira murdochii DSM 12563 |
| binHz327 | 1038399 | 51.49 | 13669 | 28 | 29.76 | 0.95 | 100 | Bacteroides vulgatus ATCC 8482 |
| binHz339 | 3266281 | 45.62 | 17634 | 58 | 94.94 | 3.33 | 56.25 | Prevotella ruminicola 23 |
| binHz56 | 3289260 | 43.4 | 11369 | 92 | 91.94 | 8.59 | 45.24 | Prevotella ruminicola 23 |
| binHz61 | 4226746 | 40.58 | 5623 | 221 | 17.54 | 1.12 | 14.29 | Roseburia intestinalis XB6B4 |
| binHz99 | 2863804 | 45.31 | 20114 | 46 | 91.36 | 5.19 | 80 | Prevotella ruminicola 23 |

### Example 5: Functional Metagenomic Screening for Environmental Exo-Sialidases

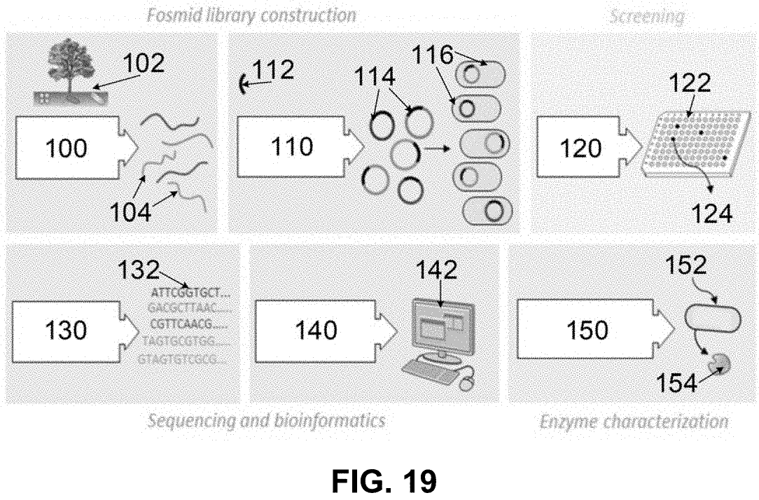

To determine if Neu5Gc-specific sialidases are restricted to enteric bacteria, we utilized functional metagenomic screening to identify exo-sialidases with a Neu5Gc specificity in a terrestrial environment. A fosmid library containing ˜40 kb inserts of total environmental DNA isolated from soil from an organic composting facility was constructed in E. coli Lysates from 5,376 clones were screened for hydrolysis of 4MU-α-Neu5Gc. An overview of the process is presented in FIG. 19. During environmental DNA extraction 100, environmental samples 102 are collected and environmental DNA 104 is extracted. DNA extraction is followed by metagenomics library production 110 where fosmid vectors 112 are ligated with environmental DNA to produce plasmids 114 that are transfected into E. coli cells 116. During functional screening 120, one clone 122 is present per well in a well plate and hits 124 are selected by a means known in the art. Following functional screening 120, hits 132 are sequenced during a sequencing step 130 using any method for sequencing known in the art. Gene prediction and database comparison 140 is used to identify enzyme candidates 142; the enzyme candidates are expressed and characterized 150 in plasmids 152, which are transfected into E. coli and expressed as enzymes 154 for further characterization and/or use.

Following the enzyme selection process, clones showing activity were tested subsequently and re-tested for their ability to cleave 4MU-α-Neu5Ac. Two clones (C19 and C22) showed significant hydrolysis of 4MU-α-Neu5Gc but only minor activity on 4MU-α-Neu5Ac (FIG. 2F). The DNA sequence of cloned inserts from C19 and C22 each encoded a single bacterial sialidase gene. Both enzymes showed exo-sialidase activity and had a marked preference for Neu5Gc hydrolysis in vitro compared to several other known bacterial exo-sialidases (FIG. 2F). Analysis of the deduced amino acid sequences of C19 and C22 showed they are highly similar to each other (56% identity), both belong to the GH33 family of bacterial exo-sialidases (FIG. 19), and are most similar to proteins from terrestrial bacteria of the genera Rhodopirellula and Verrucomicrobia.

### Example 6: Analysis of Reconstructed Metabolic Models

Genome-scale network reconstructions combine detailed biochemical and physiological information, providing insights into the metabolism for subsequent manipulation strategies or to control metabolism. The scope of these models encompasses the characterization of the metabolic behaviour of target microorganisms. We evaluated growth phenotypes of microorganisms of the gut microbiota associated with sialidase metabolism (EC: 3.2.1.18). Seventeen microorganisms of the gut microbiota containing sialidases were identified by scanning the repository of the gut microbiome metabolic models. Growth rates were simulated using flux balance analysis (FBA). All metabolic models were constrained using a western diet (45% fat. 35% carbohydrate, 20% protein), containing experimental constraints for 20 sugars, 24 fiber-related metabolites, 12 fatty acids, 20 amino acids, and 88 minerals, vitamins and other metabolite. Experimental constraints are reported in great detail in the gut microbiota repository. All metabolic models were simulated using the Gurobi Optimizer Version 5.6.3 (Gurobi Optimization Inc., Houston, Tex.) solver in MATLAB (The MathWorks Inc., Natick, Mass.) with the COBRA Toolbox. Additionally, the contribution to growth of the metabolites associated with sialidase activity (e.g. N-acetylneuraminate) was determined using shadow prices simulations.

### Example 7: Statistics and Reproducibility

Statistical analysis was performed using the R programming language. The statistical significance of differential relative abundance (16S rRNA amplicon and shotgun metagenomics) was computed using no-parametric two-sided Wilcoxon rank sum test with Holm correction for multiple hypotheses. The statistical significance of sialidase activity was computed using student's 2-tailed t test. The significance levels are indicated as follow: p-value≤0.05 (*). p-value≤0.01 (**), and p-value≤0001 (***). All analysis was performed in biologically independent animals or independent experiments as indicated in the text.

### Example 8: In Vivo Sampling, Feeding, and Animal Diet

Wild type (WT) lineage C57BL/6 was purchased from Harlan Laboratories and human-like Cmah^{-/-} mice generated as previously described. In brief, the generation of the human-like Cmah^{-/-} transgenic mice was performed by target deletion of exon 6 of the cmah gene (similar deletion that evolutionary occurred in humans) using loxP sites and Cre recombinase expression in embryonic stem cells. Transgenic mice were generated by the University of California, San Diego (UCSD), Transgenic Mouse Core. All animal experiments were approved by the UC San Diego Institutional Animal Care and Use Committee (IACUC) under the protocol number S01227. All animals were maintained in the University of California, San Diego vivarium according to Institutional Animal Care and Use Committee (IACUC) guidelines, with 12 hours diurnal lighting and access to food and water ad libitum. Sample processing and analysis were not blinded at any step. Samples size was chosen based on the cost, mice, diet and vivarium availability, and the need for an N to offer sufficient statistical power. WT and Cmah^{-/-} mice were raised in the same vivarium room and fed with the same water source. The cages were kept side by side in the same cage rack to minimize external influence on the gut microbiome. Age and sex matched female mice used in the study were maintained in sialic acid free soy-based diet (Dyets, Inc.: 110951) from weaning until 8 to 10 weeks of age to prevent previous exposure to sialic acid. To evaluate the effect of the dietary Neu5Gc in the gut microbiome, sex matched Cmah^{-/-} mice with 10 weeks of age were caged in three groups of 5 mice each and fed during 4 weeks with the same soy based diet either enriched in Neu5Gc (porcine submaxillary mucin—PSM), Neu5Ac (edible bird nest—EBN), or kept in sialic acid free-soy only as control. The animals were euthanized in a CO₂ chamber and the colon tissues were cut open with blunted scissors for fresh collection of fecal samples by scraping it straight from the tissue. Colonic fecal scrapings from five mice of each diet type were used for 16S rRNA gene amplicon and 3 mice of each diet type were used for metagenomic shotgun sequencing analysis. The feeding protocol was chosen based on previous evidence from our group showing that feeding Cmah^{-/-} mice with PSM over a period of weeks can cause mouse tissue incorporation of Neu5Gc at levels histologically similar to the levels seen in adult humans who have eaten red meat for many years. The PSM diet was prepared to contain 250 μg of Neu5Gc per gram of chow to mimic the amount of Neu5Gc present in beef, the most consumed form of red meat in the western diet.

### Example 9: Monosaccharide and Amino Acid Composition of the Diets

Monosaccharide composition of PSM and EBN diets were analyzed by High-Performance Anion-Exchange Chromatography Coupled with Pulsed Electrochemical Detection (HPAEC-PAD) using Dionex ICS-3000 system (ThermoFisher Scientific) equipped with CarboPac PA1 column 4 mm×250 mm, 4 μm, with a 4 mm×50 mm Guard. Briefly, 500 μg of each diet were dissolved in 200 μL of Milli-Q water. The samples were hydrolyzed by adding an equal volume of 4N Trifluoroacetic acid (final concentration of 2N TFA) at 100° C. for 4 hours. The hydrolyzed samples were centrifuged at 2000 rpm for 2 min and evaporated under a flow of dry nitrogen. Once dried, samples were resuspended in 200 μL of Milli-Q water and 50% of each sample was injected. The separation of monosaccharide peaks was achieved by using mobile water as mobile phase A, 100 mM NaOH with 5 mM NaOAc as mobile phase B, and 100 mM NaOH with 250 mM NaOAc as mobile phase C and gradient conditions as described in Table 6 below:

TABLE 6
HPLC Gradient for Monosaccharide and Amino Acid Separation

| Time | % A | % B | % C |
|---|---|---|---|
| 0 | 84 | 16 | 0 |
| 20 | 84 | 16 | 0 |
| 21 | 0 | 100 | 0 |
| 31 | 0 | 100 | 0 |
| 32 | 84 | 16 | 0 |
| 50 | 84 | 16 | 0 |

The amino acid composition of both diets was performed by GC-MS tBDMS derivatives quantitation as previously described in the literature. Both HPAEC-PAD and GC-MS analysis were performed by the Glycotechnology Core at the Glycobiology Research and Training Center—UCSD. A comparison of amino acids, neutral monosaccharides, and sialic acid levels in the diets is presented in Table 7.

TABLE 7
Comparison of Amino Acids, Neutral Monosaccharides,
and Sialic Acid between EBN and PSM
% of Amino Acids Per Microgram

| Amino Acid | EBN | PSM |
|---|---|---|
| Ala | 5.35 | 11.57 |
| Gly | 7.82 | 5.51 |
| Val | 13.15 | 9.63 |
| Leu | 11.44 | 10.3 |
| Ile | 4.42 | 4.72 |
| Pro | 11.64 | 6.96 |
| Ser | 13.99 | 14.35 |
| Thr | 5.85 | 8.46 |
| Phe | 6.5 | 3.82 |
| Asp | 9.99 | 8.82 |
| Glu | 5.28 | 12.74 |
| Lys | 0.95 | 1.83 |
| Tyr | 3.63 | 1.29 |

Monosaccharide per μg of Diet (in ng)

| Monosaccharide | EBN | PSM |
|---|---|---|
| Fuc | 3,166 | 16,958 |
| GalNAc | 44,498 | 61,266 |
| GlcNAc | 57,294 | 4,102 |
| Gal | 57,838 | 38.14 |
| Glc | 306.4 | 315 |
| Man | 5,956 | 0 |

Sialic Acid per Gram of Diet (in mg)

| Sialic Acid | EBN | PSM |
|---|---|---|
| Neu5Ac | 0.25 | 0 |
| Neu5Gc | 0 | 0.25 |

### Example 10: 16S rRNA Sequencing and Analysis

Total genomic DNA were extracted using MoBio PowerFecal DNA isolation kit (MoBio, Carlsbad, Calif., USA) following the manufacturer's instructions. Purified DNA was amplified and processed according to a protocol provided by the manufacturer. 16S rRNA libraries were generated from 3 or 5 biologic replicates and 3 independent experiments per diet group. Libraries were quality assessed using quantitative PCR (qPCR) and Bioanalyzer (Agilent Technologies, Palo Alto, Calif., USA), and subsequently sequenced using two MiSeq 600 cycle kits (Illumina). Adapters were trimmed from the Illumina data using Trimmomatic version 0.36. 16S analysis was performed with Usearch denoising and the RDP 16S rRNA database version 16. Sequences were analyzed using the Usearch v10 following the MiSeq 2×250 16S V4 pipeline. In brief, paired-end reads were merged using fastq_mergepairs (-fastq_maxdiffs 10; -fastq_pctid 10). Sequences with a distance-based similarity of 97% or greater were grouped into OTUs using cluster_otus (-minsize 2). OTU table were rarefied to 10,000 observations per sample. OTU-based microbial diversity and dissimilarity metrics were estimated using R package vegan v2.5-2. Bray-Curtis distance were used to beta-diversity analysis. Vector fitting with each of the PCoA ordinations was performed using the function envfit from R package vegan v2.5-2. Significant vectors were selected following the criteria (R>=0.7 and p-values=<0.01). Bacteria genus with relative abundance below than 1% were not considered for differential abundance analysis. Statistical differences in abundance between diets were calculated using the nonparametric Wilcoxon rank sum test with Holm correction for multiple hypotheses when appropriated.

### Example 11: Shotgun Metagenome Sequencing and Analysis

As described above, total genomic DNA were extracted using MoBio PowerFecal DNA isolation kit (MoBio, Carlsbad, Calif., USA) following the manufacturer's instructions. Purified DNA from three biological replicates per diet group was prepared for shotgun metagenomic sequencing using the Nextera XT library preparation method with the average fragment size of 450 bp (Illumina, San Diego, Calif. USA). Libraries were quality assessed using quantitative PCR (qPCR) and a Bioanalyzer (Agilent Technologies, Palo Alto, Calif., USA) and subsequently sequenced using MiSeq 2×250 bp cycle kits (Illumina). In average, 2.5 million non-mouse reads were generated per library. Adapters were trimmed from the Illumina data using Trimmomatic v0.36. Samples were filtered of possible human and mouse contamination by aligning the trimmed reads against reference databases using Bowtie2 v2-2.2.3 with the following parameters (-D 20 -R 3 -N 1 -L 20 -very-sensitive-local). Overlapped reads were merged using Flash version 1.2.11. Merged and unmerged reads were assembled using Spades v3.12.0 with the following parameters (-k 21,33,55,77,99,127 --meta --merge). Differential binning was performed using MetaBat2 v2.12.1, with minimum contig length of 1500 bp. Bin quality (completeness and contamination) was evaluated using CheckM v1.0.7. Taxonomic classification (closest phylogenetic neighbor) was assessed using RAST online tool. In brief, RAST uses a set of unique proteins to assign the closest related neighbor. Genome annotations were performed using Prokka v1.11 with default parameters. Amino acid sequences of all genes identified using Prokka were aligned to sialidase sequences obtained from Uniprot until up to October 2018. The alignment was performed using Diamond v0.8.24 with the following parameters (blastx -k 5 -f 6 --evalue 0.001). Relative bin abundance was obtained by dividing the total number of reads aligned per bin by the total number of reads aligned in all bins. For this analysis, non-mouse trimmed reads were aligned to the binned genomes using Bowtie2 v2-2.2.3 with the parameters set by the flag --very-sensitive. Bins with relevant genes were compared for relative abundance between sample groups using the Wilcoxon rank sum test. Carbohydrate active enzymes (CAZymes) database were used to identify diet-dependent sialidases. Non-mouse trimmed reads were aligned against sialidase amino acid sequences using Diamond v0.8.24 using the follow parameters: -k 5 -f 6 --evalue 0.001. When indicated, counts per million were used to normalize the number reads aligned by protein sequence. The total number of reads by protein sequence were rarefied to 5,000 observations per sample using the R function rrarefy from R package vegan v2.5-2. Bray-Curtis distance were used to CAZyme diversity analysis. Amino acid sequence similarities were evaluated using BLAST online tool and pairwise sequence alignments were generated using Clustal Omega online tool. Sialidases per bacterial genome were obtained from PATRIC database using command-line interface P3-scripts.

### Example 12: Analysis of Previously Published Shotgun Metagenomic Data

Previously published stool shotgun metagenomic data from Hadza hunter-gatherer individuals were obtained from Sequence Read Archive (SRA) repository under the project IDs PRJNA392012 and PRJNA392180. 40 shotgun metagenomic data from individuals were analyzed, in which 20 samples were collected during the wet season and 20 during the dry season (Table 4). All published shotgun metagenomic data were processed as described previously. Sialidase sequences derived from these stool shotgun metagenomic analyses are included herein as SEQ ID NOs. 64-86.

### Example 13: Protein Expression and Assay

Target sialidase sequences from shotgun metagenomic data were PCR amplified from genomic DNA isolated as described above or ordered from Integrated DNA Technologies (IDT), subcloned into a pET19b expression vector with a C-terminal 10× His tag and N-terminal truncation to remove any signal peptide sequence (predicted by SignalP 4.1, CBS), and transformed into BL21(DE3) E. coli(MilliporeSigma) using established heat-shock methods. Cells were grown to OD 0.6-0.8 in multiple 1 L cultures at 37° C. and induced overnight at 25° C. with 1 mM isopropyl-β-D-1-thiogalactopyranoside (IPTG). Harvested cells were resuspended in lysis buffer (50 mM HEPES pH 8.0, 50 mM NaCl, and 1 mM TCEP) with DNaseI and hen egg white lysozyme, lysed with a TS-Series cell disruptor (Constant Systems, Inc.) at 15 KPSI, and spun for 45 minutes at 186,000×g with a Ti45 ultracentrifugation rotor (Beckman Coulter, Inc.) to remove cell debris. Purification was performed as below and based on purification of a putative Bacteroides neuraminidase as provided by the Protein Structure Initiative (BACCAC_01090, Joint Center for Structural Genomics, to be published), with modifications to imidazole stringency based on the sialidase purified. Supernatant was loaded on a 5-mL HisTrap Ni affinity column on an Akta Explorer purification system (GE Healthcare Life Sciences) with 20-40 mM imidazole added, washed with Running Buffer (50 mM HEPES pH 8.0, 300 mM NaCl. 40-60 mM imidazole, 10% glycerol, and 1 mM TCEP), and eluted with Elution Buffer (20 mM HEPES pH 8.0, 300 mM imidazole, 10% glycerol, and 1 mM TCEP). Samples were concentrated using 10-30 kDa Amicon centrifugal filters (MilliporeSigma) at 1500×g to 1 mL and desalted over a 5-mL. Desalting column using the Akta system into Desalting Buffer (20 mM HEPES pH 8.0, 200 mM NaCl). Resulting protein sample was diluted as needed for functional studies.

### Example 14: Assay for Sialidase Activity

### In Vitro Activity

Sialidases purified as described above were quantified using SDS-PAGE image analysis with BSA references (Bio-Rad) and absorbance using the Nanophotometer P330 (Implen), with extinction coefficients calculated using ExPASy Translate. Sialidase activity assays were performed in a dilution series; 0.5 μg, 2.5 μg, 5 μg, and 10 μg of each enzyme were incubated with equal amount of human-like Cmah^{-/-} mouse serum (Neu5Ac: 1428.64 pmoles/μL. Neu5Gc: 0 pmoles/μL) and WT mouse serum (Neu5Ac: 196.29 pmoles/μL, Neu5Gc: 1337.45 pmoles/μL) for 1 hour at 37° C. An additional 10 μg of each enzyme was inactivated by heat for 5 minutes at 95° C. The samples were kept at −20° C. until derivatization and analyzed by HPLC as described below. The DMB reagent was made with the following recipe: 14 mM DMB (Sigma D4787), 18 mM sodium hydrosulfite (Sigma 157953), 1.0 M 2-mercaptoethanol (Sigma M3148), and 40 mM trifluoroacetic acid (Sigma T6508), and it was incubated at 50° C. for 2.5 h. The DMB-derivatized samples were analyzed on a Dionex Ultra3000 HPLC System using a Phenomenex Gemini 5μ C18 250×4.6-mm HPLC column at room temperature, eluted in isocratic mode with 85% water, 7% methanol, 8% acetonitrile. The same protocol was utilized to evaluate sialidase activity in three different pH ranges.

### In Vivo Activity Using Clarified Fecal Samples

In vivo activity using clarified fecal samples: Fecal pellets were collected from Cmah^{-/-} mice with 10 weeks of age caged in two groups of 4 mice and 3 mice each, fed during 2 weeks with PSM or sialic acid free soy, respectively. All the animals' information and maintenance are the same as described in the section “n vivo sampling”. Fresh fecal pellets were homogenized (10% w/v) in Tris-HCL pH 7 buffer with protease inhibitor (Roche 11836170001) using Pellet Pestle Cordless Motor (Kimble 749540). Samples were spun down, 4.000 g for 3 minutes and the supernatant was transferred to SpinX filters (Costar 8160). Samples were centrifuged 14,000×g for 10 minutes and the flow through was transferred to a clean tube. Total protein concentration was determined using Pierce BCA Protein Assay (Thermo Scientific 23225). To measure sialidase activity in clarified fecal samples, 2 μg of total protein per sample were incubated with equal amounts of human-like Cmah^{-/-} mouse serum and WT mouse serum for two hours at 37° C. An additional 2 μg of total protein per sample was inactivated by heat for 5 minutes at 95° C. Samples were cleaned up using 10 kDa Amicon centrifugal filters (MilliporeSigma), 14,000×g for 25 min at 4° C. Samples were lyophilized and resuspended in 50 μL of sterile water. Derivatization and HPLC analysis was performed as described in the section “In vitro activity”.

### Food Sources

Beef (New York Steak) and pork (breakfast sausage) were purchased at Whole Foods Market and cut in small pieces using sterile blades. PSM chow was powdered using mortar and pestle. Beef, pork, and PSM were homogenized (10% w/v) in Tris-HCL pH 7 buffer using Pellet Pestle Cordless Motor (Kimble 749540). 50 μL of each suspension was incubated with 2 μg of either sialidase26 or sialidaseHz136 for 2 h at 37° C. An additional 2 μg of each sialidase were inactivated by heat for 5 minutes at 95° C. Reactions were performed in triplicates. Samples were cleaned using 10 kDa Amicon centrifugal filters (MilliporeSigma), 14,000×g for 25 min at 4° C. Derivatization and HPLC analysis was performed as described above.

### Example 15: Neu5Gc2en Synthesis

Neu5Gc2en (DANA-Gc) was synthesized as previously published with minor modifications. Briefly, Neu5Gc (Sigma-Aldrich) was treated with Dowex 50W-X8 (H⁺) resin in MeOH for 20 hours at 20° C. to form the methyl ester. This ester was then treated with acetic anhydride and pyridine for 42 h at 20° C. to generate the peracetylated methyl ester, which was purified by column chromatography (50:1 CHCl₃-MeOH). This sample was treated with TMSOTf under dry nitrogen at 0° C. in MeCN for 6 h to induce elimination. The unsaturated compound was purified by chromatography (toluene/acetone, 3:1-2:1). The acetyl groups were cleaved by treatment with NaOH in MeOH over 12 h followed by neutralization with H⁺ resin.

### Example 16: Crystallization and Structure Determination of Sialidase26 and SialidaseHz136

Purified sialidase26 was concentrated to 8 g/L and set on sitting drop trays in 1:1 volume ratios with mother liquor (20% PEG 6000, 0.1 M Tris-HCl pH 8.0) at 16° C. For ligand co-crystals, concentrated sialidase26 was incubated for one hour at room temperature with 5 mM N-Acetyl-2,3-dehydro-2-deoxyneuraminic acid (Neu5Ac2en or DANA, Sigma-Aldrich) or DANA-Gc (synthesized). Crystals appeared after 3-4 days and grew to full size in 8-10 days. Crystals were soaked briefly in mother liquor supplemented with 10% glycerol and flash-frozen with liquid nitrogen. Purified sialidaseHz136 was crystallized similarly (20% PEG 3350, 0.1 M Bis-Tris-Propane pH 7.5, 0.2 M sodium citrate). X-ray diffraction data were collected at 100 K at the Lawrence Berkeley National Laboratory Advanced Light Source (8.2.1 and 8.2.2) at a single wavelength. Preliminary diffraction data were collected at the Stanford Synchrotron Radiation Lightsource and Advanced Photon Source. All diffraction data were indexed and integrated with XDS (2018 Jun. 8) or MOSFLM, processed with AIMLESS v0.7.2, and truncated with CTRUNCATE within the CCP4 v7.0.073 suite of programs. Phases were estimated via molecular replacement in PHENIX.PHASER v1.13, using a previously published model of an uncharacterized Bacteroides-derived sialidase with high sequence homology to sialidase26 (PDB 4q6k) as a search model. Models underwent rigid-body and restrained positional refinement using PHENIX.REFINE in the PHENIX software suite v1.13 against a maximum likelihood target function, alternated with manual inspection against electron density maps in Coot v0.8.9.1. Geometry restraints for DANA were generated using PHENIX.eLBOW, with manual inspections in Coot and refined in the final rounds of refinement, which also included the application of hydrogens to their riding positions and simulated annealing. For the DANA-Gc-containing co-crystal structure, diffraction data from two crystals with similar unit cell parameters were processed together in AIMLESS and multi-crystal averaging performed with PHENIX.MULTI_CRYSTAL_AVERAGE for five cycles following Phaser of each before refinement proceeded with PHENIX.REFINE. Figures displaying crystal packing were prepared using PyMOL v1.8.3.2 and atomic coordinates and structure factors were deposited with the Protein Data Bank (accession codes, 6MNJ, 6MRV, 6MRX, and 6MYV). Ramachandran statistics for Sialidase26 alone (6MRX), with DANA (6MRV), or with DANA-Gc (6MYV), and SiaHz136 (6MNJ), are, respectively, outliers (0.39, 0.19, 0.24, and 0.39%) and favored (95.67, 96.06, 95.87, 95.27%). ALS beamline v.8.2.2 were used for all datasets save 6MYV, which was collected on 8.2.1. Wavelengths used for the same datasets are, respectively. 0.99999, 0.99995, 1.00003, and 0.99994 Å. All were collected at 100.0 K. No Co deviations were observed for any dataset.

### Example 17: Enzyme Discovery—Metagenomic DNA Library Construction

Environmental DNA was isolated from soil obtained from a commercial organic composting facility in Hamilton, Mass. (Brick End Farms) by phenol:chloroform extraction and isopropanol precipitation. A fosmid library was produced using the CopyControl™ Fosmid Library Production Kits (Lucigen Corporation, Middleton, Wis.) as recommended. Briefly, DNA was end-repaired and size-selected using a 1% Low Melting Point agarose gel run overnight at 35 V. DNA fragments from 30-70 kb were isolated from the gel using 1 U of β-agarase I (New England Biolabs, Ipswich, Mass.) for each 100 μL of melted agarose. The end-repaired and size-selected DNA was ligated to the pCC1 FOS cloning vector. Resulting clones were packaged in phage particles. Escherichia coli EP1300 T1R cells were transfected with the packaging reaction and plated on LB agar medium (10 g tryptone, 5 g yeast extract, 10 g NaCl. 1 g dextrose, 1 g MgCl2.6H2O, chloramphenicol 12.5 μg/mL, 2 mL 2M NaOH and 20 g of agar per liter) and incubated overnight at 37° C. A total of 5376 colonies were archived in fourteen 384-well plates in sterile 20% (v/v) glycerol.

### Example 18: Enzyme Discovery—Screening for Sialidase Activity

The compost metagenomic library (theoretically encoding ˜215,000 environmental genes) was differentially screened with fluorogenic 2′-(4-methylumbelliferyl)-α-D-N-glycolylneuraminic acid (4MU-α-Neu5Gc) (Sussex Research, Ottawa, CA) and 2′-(4-methylumbelliferyl)-α-D-N-acetyineuraminic acid 4MU-α-Neu5Ac (Toronto Research Chemicals, Toronto, CA) substrates. In a primary screen, library clones were grown in 384-well plates containing 50 μL LB liquid cultures (10 g tryptone. 5 g yeast extract, 10 g NaCl, 1 g dextrose, 1 g MgCl2-6H2O, 2 mL of 2M NaOH per liter, containing chloramphenicol 12.5 μg/mL and 1× inducing solution (Lucigen Corporation)) overnight at 37° C. Fifty microliters of Y-per lysis buffer (Thermo Fischer Scientific, Waltham, Mass.) containing 40 μg/mL of 4MU-α-Neu5Gc was added to each well. The mixtures were incubated overnight at 37° C. in a static incubator. Fluorescence at λₑₓ=365 nm and λₑₘ=445 nm was read with a SpectraMax Plus 384 Microplate Reader (Molecular Devices, Sunnyvale, Calif.) at 6 h, 24 h and 48 h. Positive clones were defined as those showing fluorescence greater than 3 standard deviations above the mean background, and each was re-archived in a fresh 384-well plate in sterile 20% (v/v) glycerol. Each of the positive clones was grown and comparatively re-screened in separate assays containing 4MU-α-Neu5Ac and 4MU-α-Neu5Gc substrates (reactions run in duplicate). Two clones designated C19 and C22 showed significant activity on 4MU-α-Neu5Gc but only minor activity on 4MU-α-Neu5Ac and were subjected to further study.

Sequencing data supporting the findings of this study are available under accession number PRJNA505660. X-ray crystallographic data that support the findings of this study have been deposited in the RCSB Protein Data Bank (accession codes: 6MRX, 6MRV, 6MYV, and 6MNJ).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Adams, P. D. et al. PHENIX: A comprehensive Python-based system for macromolecular structure solution. Acta Crystallogr. Sect. D Biol. Crystallogr. 66, 213-221 (2010).
2. Alisson-Silva, F., Kawanishi, K. & Varki, A. Human risk of diseases associated with red meat intake: Analysis of current theories and proposed role for metabolic incorporation of a non-human sialic acid. Mol. Aspects Med. 51, 16-30 (2016).
3. Almagro-Moreno, S. & Boyd, E. F. Bacterial catabolism of nonulosonic (sialic) acid and fitness in the gut. Gut Microbes 1, 45-50 (2010).
4. Almagro-Moreno, S. & Boyd, E. F. Insights into the evolution of sialic acid catabolism among bacteria. BMC Evol. Biol. 9, 118 (2009).
5. Almagro-Moreno, S. & Boyd, E. F. Sialic acid catabolism confers a competitive advantage to pathogenic Vibrio cholerae in the mouse intestine. Infect. Immun. 77, 3807-3816 (2009).
6. Aziz, R. K. et al. The RAST Server: Rapid annotations using subsystems technology. BMC Genomics 9, 75 (2008).
7. Banda, K., Gregg, C. J., Chow, R., Varki, N. M. & Varki, A. Metabolism of vertebrate amino sugars with N-glycolyl groups: Mechanisms underlying gastrointestinal incorporation of the non-human sialic acid xeno-autoantigen N-glycolylneuraminic acid. J. Biol. Chem. 287, 28852-28864 (2012).
8. Battye, T. G. G., Kontogiannis, L., Johnson, O., Powell, H. R. & Leslie, A. G. W. iMOSFLM: A new graphical interface for diffraction-image processing with MOSFLM. Acta Crystallogr. Sect. D Biol. Crystallogr. 67, 271-281 (2011).
9. Bill Cai, T., Lu, D., Landerholm, M. & Wang, P. G. Sialated diazeniumdiolate: A new sialidase-activated nitric oxide donor. Org. Lett. 6, 4203-4205 (2004).
10. Bolger, A. M., Lohse, M. & Usadel, B. Trimmomatic: A flexible trimmer for Illumina sequence data. Bioinformatics 30, 2114-2120 (2014).
11. Canto J G, Kiefe C I, Rogers W J, Peterson E D, Frederick P D, French W J, Gibson C M, Pollack C V, Jr., Ornato J P, Zalenski R J, Penney J, Tiefenbrunn A J, Greenland P, Investigators N. 2011. Number of coronary heart disease risk factors and mortality in patients with first myocardial infarction. JAMA 306:2120-2127.
12. Chokhawala, H. A., Yu, H. & Chen, X. High-throughput substrate specificity studies of sialidases by using chemoenzymatically synthesized sialoside libraries. ChemBioChem 8, 194-201 (2007).
13. Chou H H, Hayakawa T, Diaz S, Krings M, Indriati E, Leakey M, Paabo S, Satta Y, Takahata N, Varki A. 2002. Inactivation of CMP-N-acetylneuraminic acid hydroxylase occurred prior to brain expansion during human evolution. Proc Natl Acad Sci USA 99:11736-11741.
14. Chou H H, Takematsu H, Diaz S, Iber J, Nickerson E, Wright K L, Muchmore E A, Nelson D L, Warren S T, Varki A. 1998. A mutation in human CMP-sialic acid hydroxylase occurred after the Homo-Pan divergence. Proc Natl Acad Sci USA 95:11751-11756.
15. David, L. A. et al. Diet rapidly and reproducibly alters the human gut microbiome. Nature 505, 559-563 (2014).
16. Davies, L. R. L. et al. Metabolism of vertebrate amino sugars with N-glycolyl groups: Resistance of α2-8-linked N-glycolylneuraminic acid to enzymatic cleavage. J. Biol. Chem. 287, 28917-28931 (2012).
17. Dhar, C., Sasmal, A. & Varki, A. From “serum sickness” to “xenosialitis”: Past, present, and future significance of the non-human sialic acid Neu5Gc. Front. Immunol. 10, 807 (2019).
18. Edgar, R. C. Search and clustering orders of magnitude faster than BLAST. Bioinformatics 26, 2460-2461 (2010).
19. Emsley, P., Lohkamp, B., Scott, W. G. & Cowtan, K. Features and development of Coot. Acta Crystallogr. Sect. D Biol. Crystallogr. 66, 486-501 (2010).
20. Ercégovic, T. & Magnusson, G. Highly stereoselective α-sialylation. Synthesis of GM3-saccharide and a bis-sialic acid unit. J. Org. Chem. 60, 3378-3384 (1995).
21. Etemadi, A. et al. Mortality from different causes associated with meat, heme iron, nitrates, and nitrites in the NIH-AARP Diet and Health Study: Population based cohort study. BMJ 357, 1-11 (2017).
22. Evans, P. Scaling and assessment of data quality. Acta Crystallogr. Sect. D Biol. Crystallogr. 62, 72-82 (2006).
23. Flint, H. J., Scott, K. P., Duncan, S. H., Louis, P. & Forano, E. Microbial degradation of complex carbohydrates in the gut. Gut Microbes 3, 289-306 (2012).
24. Hall, A. B., Tolonen, A. C. & Xavier, R. J. Human genetic variation and the gut microbiome in disease. Nat. Rev. Genet. 18, 690-699 (2017).
25. Hayakawa T, Aki I, Varki A, Satta Y, Takahata N. 2006. Fixation of the human-specific CMP-N-acetylneuraminic acid hydroxylase pseudogene and implications of haplotype diversity for human evolution. Genetics 172:1139-1146.
26. Hedlund M, Padler-Karavani V, Varki N M, Varki A. 2008. Evidence for a human-specific mechanism for diet and antibody-mediated inflammation in carcinoma progression. Proc Natl Acad Sci USA 105:18936-18941.
27. Hedlund, M. et al. N-glycolylneuraminic acid deficiency in mice: implications for human biology and evolution. Mol. Cell. Biol. 27, 4340-4346 (2007).
28. Huang, L. et al. DbCAN-seq: A database of carbohydrate-active enzyme (CAZyme) sequence and annotation. Nucleic Acids Res. 46, D516-D521 (2018).
29. Huang, Y. L., Chassard, C., Hausmann, M., Von Itzstein, M. & Hennet, T. Sialic acid catabolism drives intestinal inflammation and microbial dysbiosis in mice. Nat. Commun. 6, 8141 (2015).
30. Inoue, S. et al. A unique sialidase that cleaves the Neu5Gcα2→5-OglycolylNeu5Gc linkage: Comparison of its specificity with that of three microbial sialidases toward four sialic acid dimers. Biochem. Biophys. Res. Commun. 280, 104-109 (2001).
31. Juge, N., Tailford, L. & Owen, C. D. Sialidases from gut bacteria: a mini-review. Biochem. Soc. Trans. 44, 166-175 (2016).
32. Kabsch, W. XDS. Acta Crystallogr. Sect. D Biol. Crystallogr. 66, 125-132 (2010).
33. Kang, D. D., Froula, J., Egan, R. & Wang, Z. MetaBAT, an efficient tool for accurately reconstructing single genomes from complex microbial communities. PeerJ 3, e1165 (2015).
34. Kawanishi K, Dhar C, Do R, Varki N, Gordts P, Varki A. 2019. Human species-specific loss of CMP-N-acetylneuraminic acid hydroxylase enhances atherosclerosis via intrinsic and extrinsic mechanisms. Proc Natl Acad Sci USA 116:16036-16045.
35. Kim, S., Oh, D. B., Kang, H. A. & Kwon, O. Features and applications of bacterial sialidases. Appl. Microbiol. Biotechnol. 91, 1-15 (2011).
36. Langmead, B. & Salzberg, S. L. Fast gapped-read alignment with Bowtie 2. Nat. Methods 9, 357-359 (2012).
37. Laurence H, Kumar S, Owston M A, Lanford R E, Hubbard G B, Dick E J, Jr. 2017. Natural mortality and cause of death analysis of the captive chimpanzee (Pan troglodytes): A 35-year review. J Med Primatol 46:106-115.
38. Lewis, A. L. & Lewis, W. G. Host sialoglycans and bacterial sialidases: A mucosal perspective. Cell. Microbiol. 14, 1174-1182 (2012).
39. Li, J. & McClane, B. A. NanI sialidase can support the growth and survival of Clostridium perfringens strain F4969 in the presence of sialyated host macromolecules (mucin) or Caco-2 cells. Infect. Immun. 86, e00547-17 (2018).
40. Li, Y. et al. Identifying selective inhibitors against the human cytosolic sialidase NEU2 by substrate specificity studies. Mol. Biosyst. 7, 1060-1072 (2011).
41. Magnúsdóttir, S. et al. Generation of genome-scale metabolic reconstructions for 773 members of the human gut microbiota. Nat. Biotechnol. 35, 81-89 (2017).
42. Magoč, T. & Salzberg, S. L. FLASH: Fast length adjustment of short reads to improve genome assemblies. Bioinformatics 27, 2957-2963 (2011).
43. Mann, N. Dietary lean red meat and human evolution. Eur. J. Nutr. 39, 71-79 (2000).
44. McDonald, N. D., Lubin, J.-B., Chowdhury, N. & Boyd, E. F. Host-derived sialic acids are an important nutrient source required for optimal bacterial fitness in vivo. MBio 7, e02237-15 (2016).
45. Moriarty, N. W., Grosse-Kunstleve, R. W. & Adams, P. D. electronic Ligand Builder and Optimization Workbench (eLBOW): A tool for ligand coordinate and restraint generation. Acta Crystallogr. Sect. D Biol. Crystallogr. 65, 1074-1080 (2009).
46. Newstead, S. L. et al. The structure of Clostridium perfringens NanI sialidase and its catalytic intermediates. J. Biol. Chem. 283, 9080-9088 (2008).
47. Numata, M., Sugimoto, M., Shibayama, S. & Ogawa, T. A total synthesis of hematoside, α-NeuGc-(2→3)-β-Gal-(1→4)-β-Glc-(1→1)-Cer. Carbohydr. Res. 174, 73-85 (1988).
48. Nurk, S., Meleshko, D., Korobeynikov, A. & Pevzner, P. A. MetaSPAdes: A new versatile metagenomic assembler. Genome Res. 27, 824-834 (2017).
49. Owen, C. D. et al. Unravelling the specificity and mechanism of sialic acid recognition by the gut symbiont Ruminococcus gnavus. Nat. Commun. 8, 2196 (2017).
50. Parks, D. H., Imelfort, M., Skennerton, C. T., Hugenholtz, P. & Tyson, G. W. CheckM: Assessing the quality of microbial genomes recovered from isolates, single cells, and metagenomes. Genome Res. 25, 1043-1055 (2015).
51. Pearce O M, Laubli H, Verhagen A, Secrest P, Zhang J, Varki N M, Crocker P R, Bui J D, Varki A. 2014. Inverse hormesis of cancer growth mediated by narrow ranges of tumor-directed antibodies. Proc Natl Acad Sci USA 111:5998-6003.
52. Samraj A N, Pearce O M, Laubli H, Crittenden A N, Bergfeld A K, Banda K, Gregg C J, Bingman A E, Secrest P, Diaz S L, Varki N M, Varki A. 2015. A red meat-derived glycan promotes inflammation and cancer progression. Proc Natl Acad Sci USA 112:542-547.
53. Samraj, A. N. et al. A red meat-derived glycan promotes inflammation and cancer progression. Proc. Natl. Acad. Sci. 112, 542-547 (2015).
54. Samraj, A. N., Laubli, H., Varki, N. & Varki, A. Involvement of a non-human sialic acid in human cancer. Front. Oncol. 4, 1-13 (2014).
55. Schellenberger, J. et al. Quantitative prediction of cellular metabolism with constraint-based models: The COBRA Toolbox v2.0. Nat. Protoc. 6, 1290-1307 (2011).
56. Schnorr, S. L. et al. Gut microbiome of the Hadza hunter-gatherers. Nat. Commun. 5, 3654 (2014).
57. Smits, S. A. et al. Seasonal cycling in the gut microbiome of the Hadza hunter-gatherers of Tanzania. Science (80-.). 357, 802-805 (2017).
58. Tailford, L. E. et al. Discovery of intramolecular trans-sialidases in human gut microbiota suggests novel mechanisms of mucosal adaptation. Nat. Commun. 6, 7624 (2015).
59. Tan, J., Zuniga, C. & Zengler, K. Unraveling interactions in microbial communities—from co-cultures to microbiomes. J. Microbiol. 53, 295-305 (2015).
60. Tangvoranuntakul, P. et al. Human uptake and incorporation of an immunogenic nonhuman dietary sialic acid. Proc. Natl. Acad. Sci. 100, 12045-12050 (2003).
61. Varki N, Anderson D, Herndon J G, Pham T, Gregg C J, Cheriyan M, Murphy J, Strobert E, Fritz J, Else J G, Varki A. 2009. Heart disease is common in humans and chimpanzees, but is caused by different pathological processes. Evolutionary Applications 2:101-112.
62. Varki N M, Strobert E, Dick E J, Benirschke K, Varki A. 2011. Biomedical Differences Between Human and Nonhuman Hominids: Potential Roles for Uniquely Human Aspects of Sialic Acid Biology. Annual Review of Pathology: Mechanisms of Disease, Vol 6 6:365-393.
63. Varki, A. Uniquely human evolution of sialic acid genetics and biology. Proc. Natl. Acad. Sci. 107, 8939-8946 (2010).
64. Wattam, A. R. et al. Improvements to PATRIC, the all-bacterial bioinformatics database and analysis resource center. Nucleic Acids Res. 45, D535-D542 (2017).
65. Winn, M. D. et al. Overview of the CCP4 suite and current developments. Acta Crystallogr. Sect. D Biol. Crystallogr. 67, 235-242 (2011).
66. Wood, P. L., Khan, M. A. & Moskal, J. R. Neurochemical analysis of amino acids, polyamines and carboxylic acids: GC-MS quantitation of tBDMS derivatives using ammonia positive chemical ionization. J. Chromatogr. B Anal. Technol. Biomed. Life Sci. 831, 313-319 (2006).
67. Xu, G. et al. Crystal structure of the NanB Sialidase from Streptococcus pneumoniae. J. Mol. Biol. 384, 436-449 (2008).
68. Xu, G., Li, X., Andrew, P. W. & Taylor, G. L. Structure of the catalytic domain of Streptococcus pneumoniae sialidase NanA. Acta Crystallogr. Sect. F Struct. Biol. Cryst. Commun. 64, 772-775 (2008).
69. Yang, L., Connaris, H., Potter, J. A. & Taylor, G. L. Structural characterization of the carbohydrate-binding module of NanA sialidase, a pneumococcal virulence factor. BMC Struct. Biol. 15, 15 (2015).
70. Zengler, K. & Zaramela, L. S. The social network of microorganisms—How auxotrophies shape complex communities. Nat. Rev. Microbiol. 16, 383-390 (2018).
71. Zuñiga, C., Zaramela, L. & Zengler, K. Elucidation of complexity and prediction of interactions in microbial communities. Microb. Biotechnol. 10, 1500-1522 (2017).

What is claimed is:

1. A sialidase exhibiting a preference for cleaving N-glycolylneuraminic acid (Neu5Gc) from glycoconjugates over cleaving N-acetylneuraminic acid (Neu5Ac), wherein said sialidase further comprises: an Asp-box comprising RIP/RLP motif and four Asp containing consensus sequences (Ser/Thr-Xaa-Asp-[Xaa]-Gly-XaaThr-Trp/Phe), which electrostatically interact with the carboxylate groups of sialic acids.

2. The sialidase of claim 1, wherein said consensus sequence comprises a conserved nucleophilic tyrosine and acid/based glutamic acid residues at C-terminal portion of said sialidase, a N-terminal signal peptide for secretion across the membrane, and at least one catalytic pocket, with Neu5Gc preferential activity.

3. The sialidase of claim 1, wherein said sialidase comprises at least one polypeptide sequence selected from SEQ ID NOs. 1-86.

4. The sialidase of claim 3, wherein said sialidase is selected from SEQ ID NOs. 1-8.

5. The sialidase of claim 1, wherein the novel sialidase comprises a binding pocket comprising:
a first binding residue comprising aspartic acid or alanine,
a second binding residue comprising aspartic acid, asparagine, or threonine,
a third binding residue comprising tyrosine or tryptophan,
a fourth binding residue comprises arginine, tryptophan, leucine, phenylalanine, or isoleucine,
a fifth binding residue comprises leucine, glutamine, asparagine, or arginine,
a sixth binding residue comprises aspartic acid or glutamic acid,
a seventh binding residue comprises arginine,
a eighth binding residue comprises arginine, and
a ninth binding residue comprises tyrosine.

6. The sialidase of claim 5, wherein the first binding residue is separated by from 25 to 40 amino acids from the second binding residue.

7. The sialidase of claim 5, wherein the second binding residue is separated by from 15 to 25 amino acids from the third binding residue.

8. The sialidase of claim 5, wherein the third binding residue is separated by from 30 to 40 amino acids from the fourth binding residue.

9. The sialidase of claim 5, wherein the fourth binding residue is separated by from 5 to 30 amino acids from the fifth binding residue.

10. The sialidase of claim 5, wherein the fifth binding residue is separated by from 30 to 50 amino acids from the sixth binding residue.

11. The sialidase of claim 5, wherein the sixth binding residue is separated by from 10 to 20 amino acids from the seventh binding residue.

12. The sialidase of claim 5, wherein the seventh binding residue is separated by from 25 to 80 amino acids from the eighth binding residue,

13. The sialidase of claim 5, wherein the eighth binding residue is separated by from 5 to 40 amino acids from the ninth binding residue.

14. A polynucleotide sequence encoding the sialidase of claim 1.

15. A vector comprising the polynucleotide sequence of claim 14.

16. A recombinant host cell comprising the vector of claim 15.

17. The recombinant host cell of claim 16, wherein the recombinant host cell comprises E. coli.

18. An enzyme composition comprising the sialidase of claim 1.

19. A consumable product comprising the enzyme composition of claim 18.

20. The consumable product of claim 19, wherein the consumable product comprises a food, a beverage, a dietary supplement, a pharmaceutical composition, or a combination thereof.

21. A method for treating or preventing an inflammatory condition in a subject, the method comprising administering to the subject the consumable product of claim 19.

22. The method of claim 21, wherein the inflammatory condition comprises brain trauma, depression, attention deficit hyperactivity disorder, attention deficit disorder, Parkinson's disease, Alzheimer's disease, neuropathy, age-related macular degeneration, dry-eye syndrome, optic nerve damage, diabetic retinopathy, colorectal cancer, another cancer, cardiovascular disease, colitis, leaky gut syndrome, Crohn's disease, irritable bowel disease, or another inflammatory bowel disease, arthritis, type 2 diabetes, metabolic syndrome, obesity, asthma, allergies, type 1 disease, lupus, another autoimmune disease, serum sickness, atherosclerosis, inflammatory heart disease, endocarditis, valvular heart disease, peripheral arterial disease, cardiomyopathy, heart failure, or a combination thereof.

23. A probiotic composition comprising a bacterium and at least one excipient, wherein the bacterium expresses one or more sialidases having at least 60% polypeptide sequence identity with the sialidases of claim 1.

24. The probiotic composition of claim 23, wherein the bacterium comprises a Bacteroidales species, a Planctomycetales species, a Micrococcales species, a Vibrionales species, an Enterobacterales species, or a combination thereof.

25. The probiotic composition of claim 23, wherein the bacterium comprises Bacteroides caccae, Bacteroides fragilis, Bacteroides thetaiotamicron, Bacteroides xylanosolvens, Bacteroides plebius, Bifidobacterium longum, Prevotella bivia, Galbibacter marinus, Planctomycetacea bacterium, or a combination thereof.

26. A method for treating or preventing an inflammatory condition in a subject, the method comprising administering to the subject the probiotic composition of claim 23.

27. The method of claim 26, wherein the inflammatory condition comprises brain trauma, depression, attention deficit hyperactivity disorder, attention deficit disorder, Parkinson's disease, Alzheimer's disease, neuropathy, age-related macular degeneration, dry-eye syndrome, optic nerve damage, diabetic retinopathy, colorectal cancer, another cancer, cardiovascular disease, colitis, leaky gut syndrome, Crohn's disease, irritable bowel disease, or another inflammatory bowel disease, arthritis, type 2 diabetes, metabolic syndrome, obesity, asthma, allergies, type 1 disease, lupus, another autoimmune disease, serum sickness, atherosclerosis, inflammatory heart disease, endocarditis, valvular heart disease, peripheral arterial disease, cardiomyopathy, heart failure, or a combination thereof.

28. A method for removing Neu5Gc from a food product, the method comprising contacting the consumable product with the enzyme composition of claim 18 and optionally removing the enzyme composition.

29. The method of claim 28, wherein the food product comprises red meat, dairy, or a combination thereof.

30. A method of treating or preventing an inflammatory condition in a subject, the method comprising administering to the subject an effective amount of the sialidase of claim 1.

31. The method of claim 30, wherein the subject has a diet comprising N-glycolylneuraminic acid.

32. The method of claim 30, wherein the inflammatory condition comprises brain trauma, depression, attention deficit hyperactivity disorder, attention deficit disorder, Parkinson's disease, Alzheimer's disease, neuropathy, age-related macular degeneration, dry-eye syndrome, optic nerve damage, diabetic retinopathy, colorectal cancer, another cancer, cardiovascular disease, colitis, leaky gut syndrome, Crohn's disease, irritable bowel disease, or another inflammatory bowel disease, arthritis, type 2 diabetes, metabolic syndrome, obesity, asthma, allergies, type 1 disease, lupus, another autoimmune disease, serum sickness, atherosclerosis, inflammatory heart disease, endocarditis, valvular heart disease, peripheral arterial disease, cardiomyopathy, heart failure, or a combination thereof.
